SEQUENCE LISTING (9 sequences)

SEQ ID NO: 1 - DNA, 1731 bp, Homo sapiens
  CDS (124)..(1527)
  ref <308>: DATABASE ACCESSION NUMBER: Genbank/S74349
  ref <309>: DATABASE ENTRY DATE: 1995-04-12
  ref <313>: RELEVANT RESIDUES: (1)..(1681)
gttctggaggctgggaagttcaagatcaaagtgccagcagattcagtgtcatgtgaggacgtgcttcctgcttcatagataagagcttggagctcggcgcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctg

SEQ ID NO: 2 - protein, 468 aa, Homo sapiens
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 3 - DNA, 867 bp, Homo sapiens
  CDS (1)..(867): Residues 192-468, preceded by His tag
  ref <308>: DATABASE ACCESSION NUMBER: Genbank/S74349
  ref <309>: DATABASE ENTRY DATE: 1995-04-12
  ref <313>: RELEVANT RESIDUES: (192)..(468)
atgaaaaaaggtcatcatcatcatcatcatggtgaacacgacctgaaagattcggaaactgcagacctcaaatctctgggcaagagaatccacgaagcctacctgaagaacttcaacatgaacaaggtcaaggcccgggtcatactcgcgggaaagaccagcaacaacccgccttttgtcatacatgacatggagaccttgtgtatggccgagaagacgcttgtggccaagatggtggccaacggcgtcgaagacaaagaggcagaggtccgattcttccactgctgccagtgcatgtccgtggagaccgtcacggagctcacagaatttgccaaggctatcccaggctttgcaaacttggacttgaacgaccaagtcaccttgctaaagtacggtgtgtatgaagccatcttcacgatgctgtcctccttgatgaacaaagacgggatgctgatcgcgtacggcaatggctttatcacacgcgagttccttaagaacctgaggaagccgttctgtgacatcatggaacccaagtttgacttcgctatgaagttcaatgccttagaactggatgacagtgacatttccctgtttgtggctgctataatttgctgtggagatcggcctggccttctaaacataggctacattgagaagttgcaggaggggattgtgcacgtgcttaagctccacctgcagagcaaccatccagatgacaccttcctcttcccaaagctccttcaaaaaatggtggaccttcggcagctggtcacggagcatgcgcagctcgtacaggtcatcaagaagaccgagtccgacgcagcgctgcacccactgttgcaagagatctacagagacatgtactaa

SEQ ID NO: 4 - protein, 288 aa, Homo sapiens
MKKGHHHHHHGEHDLKDSETADLKSLGKRIHEAYLKNFNMNKVKARVILAGKTSNNPPFVIHDMETLCMAEKTLVAKMVANGVEDKEAEVRFFHCCQCMSVETVTELTEFAKAIPGFANLDLNDQVTLLKYGVYEAIFTMLSSLMNKDGMLIAYGNGFITREFLKNLRKPFCDIMEPKFDFAMKFNALELDDSDISLFVAAIICCGDRPGLLNIGYIEKLQEGIVHVLKLHLQSNHPDDTFLFPKLLQKMVDLRQLVTEHAQLVQVIKKTESDAALHPLLQEIYRDMY

SEQ ID NO: 5 - DNA, 2961 bp, Homo sapiens
  CDS (338)..(1660)
  ref <308>: DATABASE ACCESSION NUMBER: Genbank/L07592
  ref <309>: DATABASE ENTRY DATE: 1993-04-27
  ref <313>: RELEVANT RESIDUES: (338)..(1660)
gaattctgcggagcctgcgggacggcggcgggttggcccgtaggcagccgggacagtgttgtacagtgttttgggcatgcacgtgatactcacacagtggcttctgctcaccaacagatgaagacagatgcaccaacgagggtctggaatggtctggagtggtctggaaagcagggtcagatacccctggaaaactgaagcccgtggagcaatgatctctacaggactgcttcaaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagctctcttcctgtctttgttgtctccctctttctcagttcctctttcttttctaattcctgttgctctgtttcttcctttctgtaggtttctctcttcccttctcccttctcccttgccctccctttctctctcctatccccacgtctgtcctcctttcttattctgtgagatgttttgtattatttcaccagcagcatagaacaggacctctgcttttgcacaccttttccccaggagcagaagagagtgggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtggcagaccccggcagtagagccgagatgcctccccaagactgtcattgcccctccgatcgtgaggccacccactgacccaatgatcctctccagcagcacacctcagccccactgacacccagtgtccttccatcttcacactggtttgccaggccaatgttgctgatggcccctccagcacacacacataagcactgaaatcactttacctgcaggcaccatgcacctcccttccctccctgaggcaggtgagaacccagagagaggggcctgcaggtgagcaggcagggctgggccaggtctccggggaggcaggggtcctgcaggtcctggtgggtcagcccagcacctcgcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctatgtgactctgggtggaagtgcccagcccctgcctg

SEQ ID NO: 6 - protein, 441 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 7 - DNA, 825 bp, Homo sapiens
  CDS (1)..(825)
cagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtac

SEQ ID NO: 8 - protein, 275 aa, Homo sapiens
QYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 9 - protein, 11 aa, Artificial Sequence
  Synthetic Construct
MKKGHHHHHHG

(19) United States
(12) Patent Application Publication
(10) Pub. No.: US 2004/0137518 A1
(43) Pub. Date: Jul. 15, 2004

(54) CRYSTALLIZED PPARa LIGAND BINDING DOMAIN POLYPEPTIDE AND SCREENING METHODS EMPLOYING SAME

(76) Inventors: Millard Hurst Lambert III, Durham, NC (US); Valerie Gail Montana, Durham, NC (US); Huaqiang Eric Xu, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/467,048

(22) PCT Filed: Jan. 31, 2002
(86) PCT No.: PCT/US02/03278

(51) Int. Cl. G01N 33/53; G06F 19/00; G01N 33/48; G01N 33/50

(52) U.S. Cl. 435/7.1; 530/388.22; 702/19

(57) ABSTRACT

A solved three-dimensional crystal structure of a PPARα ligand binding domain polypeptide is disclosed, along with a crystal form of the PPARα ligand binding domain polypeptide. Methods of designing modulators of the biological activity of PPARα and other PPAR ligand binding domain polypeptides are also disclosed.

### TECHNICAL FIELD

The present invention relates generally to the structure of the ligand binding domain of PPARα, and more particularly to the structure of the ligand binding domain of PPARα in complex with a ligand. The invention further relates to methods by which modulators and ligands of PPARα and other PPARs can be identified.

Abbreviations

| | |
|---|---|
| ATP | adenosine triphosphate |
| ADP | adenosine diphosphate |
| BSA | bovine serum albumin |
| cDNA | complementary DNA |
| DBD | DNA binding domain |
| DMSO | dimethyl sulfoxide |
| DNA | deoxyribonucleic acid |
| DTT | dithiothreitol |
| EDTA | ethylenediaminetetraacetic acid |
| HEPES | N-2-Hydroxyethylpiperazine-N'-2-ethanesulfonic |
| | acid |
| kDa | kilodalton(s) |
| LBD | ligand binding domain |
| mPPAR | mouse peroxisome proliferator activated receptor |
| NDP | nucleotide diphosphate |
| NTP | nucleotide triphosphate |
| PAGE | polyacrylamide gel electrophoresis |
| PCR | polymerase chain reaction |
| pl | isoelectric point |
| PPAR | peroxisome proliferator-activated receptor |
| PPARα | peroxide proliferator-activated receptor alpha |
| PPRE | PPAR response element |
| rPPAR | rat peroxisome proliferator activated receptor |
| RXR | retinoid X receptor |
| SDS | sodium dodecyl sulfate |
| SDS-PAGE | sodium dodecyl sulfate polyacrylamide gel |
| | electrophoresis |

Amino Acid Abbreviations

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| | |
|---|---|
| Amino Acid | Codons |

| | | | |
|---|---|---|---|
| Alanine | Ala | A | GCA GCC GCG GCU |
| Cysteine | Cys | C | UGC UGU |
| Aspartic Acid | Asp | 0 | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | lie | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | GIn | Q | CAA GAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC |
| | | | CUG CUU |
| Arginine | Arg | R | AGA AGG OGA CGC |
| | | | CGG CGU |
| Serine | Ser | S | ACG AGU UCA UCC |
| | | | UCG UCU |

### BACKGROUND ART

Nuclear receptors reside in either the cytoplasm or nucleus of eukaryotic cells and represent a superfamily of proteins that specifically bind a physiologically relevant small molecule, such as a hormone or vitamin. As a result of a molecule binding to a nuclear receptor, the nuclear receptor changes the ability of a cell to transcribe DNA, i.e. nuclear receptors modulate the transcription of DNA. However, they can also have transcription independent actions.

Unlike integral membrane receptors and membrane-associated receptors, nuclear receptors reside in either the cytoplasm or nucleus of eukaryotic cells. Thus, nuclear receptors comprise a class of intracellular, soluble, ligand-regulated transcription factors. Nuclear receptors include but are not limited to receptors for glucocorticoids, androgens, mineralcorticoids, progestins, estrogens, thyroid hormones, vitamin D, retinoids, icosanoids and pertinently, peroxisome proliferators. Many nuclear receptors, identified by either sequence homology to known receptors (See, e.g., Drewes et al., (1996) Mol. Cell. Biol. 16:925-31) or based on their affinity for specific DNA binding sites in gene promoters (See, e.g., Sladek et al., Genes Dev. 4:2353-65), have unascertained ligands and are therefore termed “orphan receptors”.

Peroxisomes are organelles that are involved in the β-oxidation of long-chain fatty acids and the catabolism of cholesterol to bile acids (See, e.g., Vamecq & Draye, (1989) Essays Biochem. 24: 115-225). Peroxisome proliferators are a structurally diverse group of compounds which, when administered to rodents, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow & Fujiki, (1985) Ann. Rev. Cell Biol. 1: 489-530; Vamecq & Draye, (1989) Essays Biochem. 24: 115-225; and Nelali et al., (1988) Cancer Res. 48: 5316-5324). Chemicals of this group include the fibrate class of hypolipidermic drugs, herbicides, phthalate plasticizers, unsaturated fatty acids, and leukotriene antagonists (reviewed in Green, (1992) Biochem. Pharmacol. 43: 393-401). Peroxisome proliferation can also be elicited by dietary or physiological factors such as a high-fat diet and cold acclimatization.

Peroxisome proliferator activated receptors (PPARs) are activated by one of a group of compounds known as peroxisome proliferators. Insight into the mechanism by which peroxisome proliferators exert their pleiotropic effects was provided by the identification of a member of the nuclear hormone receptor superfamily activated by the chemicals described above (Isseman & Green, (1990) Nature 347: 645-50). This receptor, termed peroxisome proliferator activated receptor alpha (PPARα), was subsequently shown to be activated by a variety of medium and long-chain fatty acids. PPARα was also shown to modulate expression of a variety of genes containing one or more PPAR responsive elements found in their promoter regions.

It appears that PPARα has a role in the regulation of virtually the entire oxidative pathway of fatty acids and their derivatives (See, Lemberger et al., (1996) Ann. Rev. Cell. Dev. Biol. 12: 335-63). It has also been observed that PPARα expression is closely tied to conditions that induce elevated glucocorticoid levels such as fasting, diurnal rhythm (Lemberger et al., (1996) J. Biol. Chem. 271: 1764-69) and stress.

Structurally, PPARα comprises three functional domains, the N terminus region, the DNA binding domain and the ligand binding domain. These domains retain their functional autonomy when they are expressed as a chimeric or fusion protein (Göttlicher et al., (1992) Proc. Natl. Acad. Sci. U.S.A. 89: 4653-57).

PPARα activates transcription by binding to DNA sequence elements, termed PPAR response elements (PPRE), as heterodimers (dimerization is essential for the activity of PPARs) with the retinoid X receptors (RXR) (See, Keller & Whali, (1993) Trends Endocrin. Met. 4: 291-96), which are themselves activated by 9-cis retinoic acid (See, Kliewer et al., (1992) Nature 358: 771-74; Gearing et al., (1993) Proc. Natl. Acad. Sci. U.S.A. 90: 1440-44; Keller et al., (1993) Proc. Natl. Acad. Sci. U.S.A. 90: 2160-2164; Heyman et al., (1992) Cell 68: 397-406 and Levin et al., (1992) Nature 355: 359-61). Since the PPARα-RXR complex can be activated by peroxisome proliferators and/or 9-cis retinoic acid, the retinoid and fatty acid signaling pathways are seen to converge in modulating lipid metabolism.

PPREs have been identified in the enhancers of a number of genes that encode proteins that regulate lipid metabolism, including: (1) the three enzymes required for peroxisomal β-oxidation of fatty acids; (2) medium-chain acyl-CoA dehydrogenase, a key enzyme in mitochondrial β-oxidation; and (3) aP2, a lipid binding protein expressed exclusively in adipocytes. Thus, the nature of the PPAR target genes coupled with the activation of PPARs by fatty acids and hypolipidemic drugs suggests a physiological role for the PPARs in a variety of physiological phenomena, including lipid homeostasis (See, e.g., Keller & Whali, (1993) Trends Endocrin. Met. 4: 291-96). PPARs have also been implicated in glucose homeostasis disorders and in atherosclerosis. These conditions may exist alone or together in a complex phenotype of metabolic disorders known as syndrome X.

Since the discovery of PPARα, additional subtypes of PPAR have been identified, e.g. PPARγ and PPARδ, which are spatially differentially expressed. Because there are several subtypes of PPAR, it is desirable to identify compounds that are capable of selectively interacting with only one of the PPAR subtypes, notably PPARα. Compounds capable of interacting with PPARα exclusively would find a wide variety of applications, for example, in the prevention of obesity, for the treatment of diabetes, and other deleterious conditions, as noted above. Development of such compounds, however, has been hindered by a lack of detailed structural information on the ligand binding domain of PPARα and particularly by a lack of structural information on the conformation of the ligand binding domain of PPARα as it binds a modulating compound.

It is believed that PPARα regulates some of the same genes as PPARγ and PPARδ. However, some genes might be upregulated by one PPAR and downregulated by another PPAR. Up- or down-regulation of certain genes by a PPARγ agonist might cause detrimental side effects. It might be possible to use a PPARα or PPARδ agonist, partial agonist or antagonist to down- or up-regulate (respectively) these same genes, and thereby reduce the detrimental side-effects. More generally, it might be possible to individually up- and down-regulate specific genes to achieve a specific therapeutic goal by administering a PPAR activator (or partial activator) that activates (or deactivates) each PPAR to the appropriate extent. Design or discovery of such a compound would be greatly facilitated by three-dimensional structures for each of the three target receptors, PPARα, PPARγ and PPARδ.

Polypeptides, including the ligand binding domain of PPARα, have a three-dimensional structure determined by the primary amino acid sequence and the environment surrounding the polypeptide. This three-dimensional structure establishes the polypeptide's activity, stability, binding affinity, binding specificity, and other biochemical attributes. Thus, knowledge of a protein's three-dimensional structure can provide much guidance in designing agents that mimic, inhibit, or improve its biological activity.

The three-dimensional structure of a polypeptide can be determined in a number of ways. Many of the most precise methods employ X-ray crystallography (See, e.g., Van Holde, (1971) Physical Biochemistry, Prentice-Hall, N.J., 221-39). This technique relies on the ability of crystalline lattices to diffract X-rays or other forms of radiation. Diffraction experiments suitable for determining the three-dimensional structure of macromolecules typically require high-quality crystals. Unfortunately, such crystals have been unavailable for the ligand binding domain of PPARα, as well as many other proteins of interest. Thus, high-quality diffracting crystals of the ligand binding domain of PPARα in complex with a ligand would greatly assist in the elucidation of its three-dimensional structure.

Clearly, the solved crystal structure of the PPARα ligand binding domain polypeptide would be useful in the design of modulators of activity mediated by all of the PPARs. Evaluation of the available sequence data has made it clear that PPARα shares significant sequence homology with the other PPARs. Further, PPARα shows structural homology with the three-dimensional fold of the other PPARs. Thus, in theory, it might be considered feasible to design modulators of PPARα based exclusively on the sequence and three-dimensional fold of a different PPAR, for example, PPARγ. This method, however, would likely be unproductive and certainly hindered by a lack of subtle structural details of the various binding sites and pertinent residues of PPARα involved in the binding event. A solved crystal structure would provide these structural details.

The solved PPARα-ligand crystal structure would provide structural details and insights necessary to design a modulator of PPARα that maximizes preferred requirements for any modulator, i.e. potency and specificity. By exploiting the structural details obtained from a PPAR-ligand crystal structure, it would be possible to design a PPAR modulator that, despite PPARα's similarity with other PPARs, exploits the unique structural features of PPARα. A PPAR modulator developed using structure-assisted design would take advantage of heretofore unknown PPAR structural considerations and thus be more effective than a modulator developed using homology-based design. Potential or existent homology models cannot provide the necessary degree of specificity. A PPAR modulator designed using the structural coordinates of a crystalline form of PPARα would also provide a starting point for the development of modulators of other PPARs.

What is needed, therefore, is a crystallized form of a PPARα ligand binding domain, preferably in complex with a ligand. Acquisition of crystals of the PPARα ligand binding domain (LBD) polypeptide will permit the three dimensional structure of PPARα LBD polypeptide to be determined. Knowledge of the three dimensional structure will facilitate the design of modulators of PPARα activity. Such modulators can lead to therapeutic compounds to treat a wide range of conditions, including lipid homeostasis disorders, glucose homeostasis disorders, inflammation, atherosclerosis and syndrome X.

### SUMMARY OF THE INVENTION

A substantially pure PPARα ligand binding domain polypeptide in crystalline form is disclosed. Preferably, the crystalline form has lattice constants of a=61.3 Å, b=103.5 Å, c=49.9 Å, α=90°, β=90°, γ=90° or lattice constants of a=95.58 Å, b=122.06 Å, c=122.10 Å, α=90°, β=90°, γ=90°. Preferably, the crystalline form is an orthorhombic crystalline form. More preferably, the crystalline form has a space group of P2₁2₁2 or space group P2₁2₁2₁. Even more preferably, the PPARα ligand binding domain polypeptide has the amino acid sequence shown in SEQ ID NO: 4. Even more preferably, the PPARα ligand binding domain has a crystalline structure further characterized by the coordinates corresponding to Table 2.

Preferably, the PPARα ligand binding domain polypeptide is in complex with a ligand. Optionally, the crystalline form contains one or four PPARα ligand binding domain polypeptides in the asymmetric unit. Preferably, the crystalline form is such that the three-dimensional structure of the crystallized PPARα ligand binding domain polypeptide can be determined to a resolution of about 1.8 Å or better. Even more preferably, the crystalline form contains one or more atoms having a molecular weight of 40 grams/mol or greater.

A method for determining the three-dimensional structure of a crystallized PPARα ligand binding domain polypeptide to a resolution of about 1.8 Å or better is disclosed. The method comprises (a) crystallizing a PPARα ligand binding domain polypeptide; and (b) analyzing the PPARα ligand binding domain polypeptide to determine the three-dimensional structure of the crystallized PPARα ligand binding domain polypeptide, whereby the three-dimensional structure of a crystallized PPARα ligand binding domain polypeptide is determined to a resolution of about 1.8 Å or better. Preferably, the analyzing is by X-ray diffraction. More preferably, the crystallization is accomplished by the hanging drop vapor diffusion method, and wherein the PPARα ligand binding domain is mixed with an equal volume of reservoir. Even more preferably, the reservoir comprises 4-8% PEG 3350, 100-200 mM NaF, and 12-16% 2,5 hexanediol or the reservoir comprises 50 mM bis-tris-propane, 4-6% PEG 3350, 150 mM NaNO₃, 16% 2,5 hexanediol, and 1-3 mM YCI₃.

A method of designing a modulator of a PPAR polypeptide is disclosed. The method comprises (a) designing a potential modulator of a PPAR polypeptide that will form bonds with amino acids in a substrate binding site based upon a crystalline structure of a PPARα ligand binding domain polypeptide; (b) synthesizing the modulator; and (c) determining whether the potential modulator modulates the activity of the PPAR polypeptide, whereby a modulator of a PPAR polypeptide is designed.

A method of designing a modulator that selectively modulates the activity of a PPAR polypeptide is disclosed. The method comprises (a) obtaining a crystalline form of a PPARα ligand binding domain polypeptide; (b) evaluating the three-dimensional structure of the crystallized PPARα ligand binding domain polypeptide; and (c) synthesizing a potential modulator based on the three-dimensional crystal structure of the crystallized PPARα ligand binding domain polypeptide, whereby a modulator that selectively modulates the activity of a PPARα polypeptide is designed. Preferably, the method further comprises contacting a PPARα ligand binding domain polypeptide with the potential modulator; and assaying the PPARα ligand binding domain polypeptide for binding of the potential modulator, for a change in activity of the PPARα ligand binding domain polypeptide, or both. More preferably, the crystalline form is in orthorhombic form. Even more preferably, the crystals are such that the three-dimensional structure of the crystallized PPARα ligand binding domain polypeptide can be determined to a resolution of about 1.8 Å or better.

A method of screening a plurality of compounds for a modulator of a PPAR ligand binding domain polypeptide is disclosed. The method comprises (a) providing a library of test samples; (b) contacting a crystalline PPARα ligand binding domain polypeptide with each test sample; (c) detecting an interaction between a test sample and the crystalline PPARα ligand binding domain polypeptide; (d) identifying a test sample that interacts with the crystalline PPARα ligand binding domain polypeptide; and (e) isolating a test sample that interacts with the crystalline PPARα ligand binding domain polypeptide, whereby a plurality of compounds is screened for a modulator of a PPAR ligand binding domain polypeptide. Preferably, the test samples are bound to a substrate, and more preferably, the test samples are synthesized directly on a substrate.

A method for identifying a PPAR modulator is disclosed. The method comprises (a) providing atomic coordinates of a PPARα ligand binding domain to a computerized modeling system; and (b) modeling ligands that fit spatially into the binding pocket of the PPARα ligand binding domain to thereby identify a PPAR modulator. Preferably, the method further comprises identifying in an assay for PPAR-mediated activity a modeled ligand that increases or decreases the activity of the PPAR.

A method of identifying a PPARα modulator that selectively modulates the activity of a PPARα polypeptide compared to other polypeptides is disclosed. The method comprises (a) providing atomic coordinates of a PPARα ligand binding domain to a computerized modeling system; and (b) modeling a ligand that fits into the binding pocket of a PPARα ligand binding domain and that interacts with conformationally constrained residues of a PPARα conserved among PPAR subtypes to thereby identify a PPARα modulator. Preferably, the method further comprises identifying in a biological assay for PPARα activity a modeled ligand that selectively binds to PPARα and increases or decreases the activity of said PPARα.

A method of designing a modulator of a PPAR polypeptide is disclosed. The method comprises (a) selecting a candidate PPAR ligand; (b) determining which amino acid or amino acids of a PPAR polypeptide interact with the ligand using a three-dimensional model of a crystallized protein comprising a PPARα LBD; (c) identifying in a biological assay for PPAR activity a degree to which the ligand modulates the activity of the PPAR polypeptide; (d) selecting a chemical modification of the ligand wherein the interaction between the amino acids of the PPAR polypeptide and the ligand is predicted to be modulated by the chemical modification; (e) performing the chemical modification on the ligand to form a modified ligand; (f) contacting the modified ligand with the PPAR polypeptide; (g) identifying in a biological assay for PPAR activity a degree to which the modified ligand modulates the biological activity of the PPAR polypeptide; and (h) comparing the biological activity of the PPAR polypeptide in the presence of modified ligand with the biological activity of the PPAR polypeptide in the presence of the unmodified ligand, whereby a modulator of a PPAR polypeptide is designed. Preferably, the PPAR polypeptide is a PPARα polypeptide. More preferably, the three-dimensional model of a crystallized protein is a PPARα LBD polypeptide with a bound ligand. Optionally, the method further comprises repeating steps (a) through (f), if the biological activity of the PPAR polypeptide in the presence of the modified ligand varies from the biological activity of the PPAR polypeptide in the presence of the unmodified ligand.

An assay method for identifying a compound that inhibits binding of a ligand to a PPAR polypeptide is disclosed. The assay method comprises (a) incubating a PPAR polypeptide with a ligand in the presence of a test inhibitor compound; (b) determining an amount of ligand that is bound to the PPAR polypeptide, wherein decreased binding of ligand to the PPAR protein in the presence of the test inhibitor compound relative to binding of ligand in the absence of the test inhibitor compound is indicative of inhibition; and (c) identifying the test compound as an inhibitor of ligand binding if decreased ligand binding is observed.

A method of identifying a PPAR modulator that selectively modulates the biological activity of one PPAR subtype compared to PPARα is disclosed. The method comprises: (a) providing an atomic structure coordinate set describing a PPARα ligand binding domain structure and at least one other atomic structure coordinate set describing a PPAR ligand binding domain, each ligand binding domain comprising a ligand binding site; (b) comparing the PPAR atomic structure coordinate sets to identify at least one difference between the sets; (c) designing a candidate ligand predicted to interact with the difference of step (b); (d) synthesizing the candidate ligand; and (e) testing the synthesized candidate ligand for an ability to selectively modulate a PPAR subtype as compared to PPARα, whereby a PPAR modulator that selectively modulates the biological activity of one PPAR subtype compared to PPARα is identified.

Accordingly, it is an object of the present invention to provide a three dimensional structure of the ligand binding domain of PPARα. The object is achieved in whole or in part by the present invention.

An object of the invention having been stated hereinabove, other objects will be evident as the description proceeds, when taken in connection with the accompanying Drawings and Laboratory Examples as best described hereinbelow.

### BRIEF DESCRIPTION OF THE DRAWINGS

FIG. 1 is a ribbon diagram depicting the PPARα LBD in complex with Compound 1. The PPARα LBD is presented as a ribbon diagram and Compound 1 is presented as a spacefilling model.

FIG. 2 is a schematic drawing depicting interactions between PPARα and Compound 1. Residues that lie within 5.5 Å of heavy atoms in the ligand are shown.

FIG. 3 depicts overlayed ball and stick diagrams of PPARα, PPARγ and PPARδ. PPARα is shown in dark gray, PPARγ in medium gray and PPARδ in light gray. PPARδ was used as a template in the molecular replacement solution of PPARα.

FIG. 4 is a ribbon diagram of the PPARα backbone conformation, where the ligand binding pocket of PPARα is identified by a smooth solid surface.

FIG. 5 is a ribbon diagram of the PPARγ backbone conformation, where the ligand binding pocket of PPARγ is identified by a smooth solid surface.

FIG. 6 is a ribbon diagram of the PPARδ backbone conformation, where the ligand binding pocket of PPARδ is identified by a smooth solid surface.

FIG. 7 is a ball-and-stick model depicting hydrogen bonding interactions between Compound 1 and PPARα. Water molecules are shown as octahedral (six-pointed) crosses.

### DETAILED DESCRIPTION OF THE INVENTION

Until disclosure of the present invention presented herein, the ability to obtain crystalline forms of a PPARα LBD has not been realized. And until disclosure of the present invention presented herein, a detailed three-dimensional crystal structure of a PPARα polypeptide has not been solved.

In addition to providing structural information, crystalline polypeptides provide other advantages. For example, the crystallization process itself further purifies the polypeptide, and satisfies one of the classical criteria for homogeneity. In fact, crystallization frequently provides unparalleled purification quality, removing impurities that are not removed by other purification methods such as HPLC, dialysis, conventional column chromatography, etc. Moreover, crystalline polypeptides are often stable at ambient temperatures and free of protease contamination and other degradation associated with solution storage. Crystalline polypeptides can also be useful as pharmaceutical preparations. Finally, crystallization techniques in general are largely free of problems such as denaturation associated with other stabilization methods (e.g., lyophilization). Once crystallization has been accomplished, crystallographic data provides useful structural information that can assist the design of compounds that can serve as agonists or antagonists, as described herein below. In addition, the crystal structure provides information useful to map a receptor binding domain, which could then be mimicked by a small non-peptide molecule that would serve as an antagonist or agonist.

### I. Definitions

Following long-standing patent law convention, the terms “a” and “an” mean “one or more” when used in this application, including the claims.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “labeled” means the attachment of a moiety, capable of detection by spectroscopic, radiologic or other methods, to a probe molecule.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “expression” generally refers to the cellular processes by which a biologically active polypeptide is produced.

As used herein, the term “transcription factor” means a cytoplasmic or nuclear protein which binds to such gene, or binds to an RNA transcript of such gene, or binds to another protein which binds to such gene or such RNA transcript or another protein which in turn binds to such gene or such RNA transcript, so as to thereby modulate expression of the gene. Such modulation can additionally be achieved by other mechanisms; the essence of “transcription factor for a gene” is that the level of transcription of the gene is altered in some way.

As used herein, the term “hybridization” means the binding of a probe molecule, a molecule to which a detectable moiety has been bound, to a target sample.

As used herein, the term “detecting” means confirming the presence of a target entity by observing the occurrence of a detectable signal, such as a radiologic or spectroscopic signal that will appear exclusively in the presence of the target entity.

As used herein, the term “sequencing” means the determining the ordered linear sequence of nucleic acids or amino acids of a DNA or protein target sample, using conventional manual or automated laboratory techniques.

As used herein, the term “isolated” means oligonucleotides substantially free of other nucleic acids, proteins, lipids, carbohydrates or other materials with which they can be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “substantially pure” means that the polynucleotide or polypeptide is substantially free of the sequences and molecules with which it is associated in its natural state, and those molecules used in the isolation procedure. The term “substantially free” means that the sample is at least 50%, preferably at least 70%, more preferably 80% and most preferably 90% free of the materials and compounds with which is it associated in nature.

As used herein, the term “primer” means a sequence comprising two or more deoxyribonucleotides or ribonucleotides, preferably more than three, and more preferably more than eight and most preferably at least about 20 nucleotides of an exonic or intronic region. Such oligonucleotides are preferably between ten and thirty bases in length.

As used herein, the term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

As used herein, the term “DNA segment” means a DNA molecule that has been isolated free of total genomic DNA of a particular species. In a preferred embodiment, a DNA segment encoding a PPARα polypeptide refers to a DNA segment that comprises SEQ ID NOs: 1 and 3, but can optionally comprise fewer or additional nucleic acids, yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

As used herein, the phrase “enhancer-promoter” means a composite unit that contains both enhancer and promoter elements. An enhancer-promoter is operatively linked to a coding sequence that encodes at least one gene product.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

As used herein, the terms “candidate substance” and “candidate compound” are used interchangeably and refer to a substance that is believed to interact with another moiety, for example a given ligand that is believed to interact with a complete, or a fragment of, a PPAR polypeptide, and which can be subsequently evaluated for such an interaction. Representative candidate substances or compounds include xenobiotics such as drugs and other therapeutic agents, carcinogens and environmental pollutants, natural products and extracts, as well as endobiotics such as steroids, fatty acids and prostaglandins. Other examples of candidate compounds that can be investigated using the methods of the present invention include, but are not restricted to, agonists and antagonists of a PPAR polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the term “biological activity” means any observable effect flowing from interaction between a PPAR polypeptide and a ligand. Representative, but non-limiting, examples of biological activity in the context of the present invention include dimerization of PPARα with RXR, phosphorylation, and association of PPARα with DNA.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the terms “structure coordinates” and “structural coordinates” mean mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of structure coordinates determined by X-ray crystallography is not without standard error. For the purpose of this invention, any set of structure coordinates for PPARα or a PPARα mutant that have a root mean square (RMS) deviation of no more than 1.0 Å when superimposed, using the polypeptide backbone atoms, on the structure coordinates listed in Table 2 shall be considered identical.

As used herein, the term “space group” means the arrangement of symmetry elements of a crystal.

As used herein, the term “molecular replacement” means a method that involves generating a preliminary model of the wild-type PPARα ligand binding domain, or a PPARα mutant crystal whose structure coordinates are unknown, by orienting and positioning a molecule whose structure coordinates are known (e.g., PPARδ) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. (Lattman, (1985) Method Enzymol., 115: 55-77; Rossmann, ed, (1972) The Molecular Replacement Method, Gordon & Breach, New York.) Using the structure coordinates of the ligand binding domain of PPARα provided by this invention, molecular replacement can be used to determine the structure coordinates of a crystalline mutant or homologue of the PPARα ligand binding domain, or of a different crystal form of the PPARα ligand binding domain.

As used herein, the terms “β-sheet” and “beta-sheet” mean the conformation of a polypeptide chain stretched into an extended zig-zig conformation. Portions of polypeptide chains that run “parallel” all run in the same direction. Polypeptide chains that are “antiparallel” run in the opposite direction from the parallel chains.

As used herein, the terms “α-helix” and “alpha-helix” mean the conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turnoff the helix, which extends about 0.56 nm along the long axis.

As used herein, the term “unit cell” means a basic parallelipiped shaped block. The entire volume of a crystal can be constructed by regular assembly of such blocks. Each unit cell comprises a complete representation of the unit of pattern, the repetition of which builds up the crystal. Thus, the term “unit cell” means the fundamental portion of a crystal structure that is repeated infinitely by translation in three dimensions. A unit cell is characterized by three vectors a, b, and c, not located in one plane, which form the edges of a parallelepiped. Angles α, β and γ define the angles between the vectors: angle α is the angle between vectors b and c; angle β is the angle between vectors a and c; and angle γ is the angle between vectors a and b. The entire volume of a crystal can be constructed by regular assembly of unit cells; each unit cell comprises a complete representation of the unit of pattern, the repetition of which builds up the crystal.

As used herein, “orthorhombic unit cell” means a unit cell wherein a≠b≠c; and α=β=γ=90°. The vectors a, b and c describe the unit cell edges and the angles α, β, and γ describe the unit cell angles.

As used herein, the term “crystal lattice” means the array of points defined by the vertices of packed unit cells.

As used herein, the term “PPAR” means any polypeptide sequence that can be aligned with at least one of human PPARα, PPARγ or PPARδ such that at least 50% of the amino acids are identical to the corresponding amino acid in the human PPARα, PPARγ or PPARδ. The term “PPAR” also encompasses nucleic acid sequences where the corresponding translated protein sequence can be considered to be a PPAR. The term “PPAR” encompasses at least the PPARα, PPARγ and PPARδ subtypes. The term “PPAR” includes invertebrate homologs; preferably, PPAR nucleic acids and polypeptides are isolated from eukaryotic sources. “PPAR” further includes vertebrate homologs of PPAR family members, including, but not limited to, mammalian and avian homolog. Representative mammalian homologs of PPAR family members include, but are not limited to, murine and human homologs.

As used herein, the terms “PPARα gene product”, “PPARα protein”, “PPARα polypeptide”, and “PPARα peptide” are used interchangeably and mean peptides having amino acid sequences which are substantially identical to native amino acid sequences from the organism of interest and which are biologically active in that they comprise all or a part of the amino acid sequence of a PPARα polypeptide, or cross-react with antibodies raised against a PPARα polypeptide, or retain all or some of the biological activity (e.g., DNA or ligand binding ability and/or dimerization ability) of the native amino acid sequence or protein. Such biological activity can include immunogenicity.

In the present invention, the terms “PPARα gene product”, “PPARα protein”, “PPARα polypeptide”, and “PPARα peptide” are used interchangeably and mean to the preferred subtype of the PPAR family, namely PPARα, which comprises the amino acid sequence of SEQ ID NO: 2.

As used herein, the terms “PPARα gene product”, “PPARα protein”, “PPARα polypeptide”, and “PPARα peptide” also include analogs of a PPARα polypeptide. By “analog@ is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct PPARα analogs. There is no need for a “PPARα gene product”, “PPARα protein”, “PPARα polypeptide”, or “PPARα peptide” to comprise all or substantially all of the amino acid sequence of a PPARα polypeptide gene product. Shorter or longer sequences are anticipated to be of use in the invention; shorter sequences are herein referred to as “segments”. Thus, the terms “PPARα gene product”, “PPARα protein”, “PPARα polypeptide”, and “PPARα peptide” also include fusion or recombinant PPARα polypeptides and proteins comprising sequences of the present invention. Methods of preparing such proteins are disclosed herein and are known in the art.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the term “modulate” means an increase, decrease, or other alteration of any or all chemical and biological activities or properties of a wild-type or mutant PPAR polypeptide, preferably a wild-type or mutant PPARα polypeptide. The term “modulation” as used herein refers to both upregulation (i.e., activation or stimulation) and downregulation (i.e. inhibition or suppression) of a response.

As used herein, the terms “binding pocket of the PPARα ligand binding domain”, “PPARα ligand binding pocket” and “PPARα binding pocket” are used interchangeably, and refer to the large cavity within the PPARα ligand binding domain where Compound 1 binds. This cavity may be empty, or may contain water molecules or other molecules from the solvent, or may contain ligand atoms. The “main” binding pocket includes the region of space not occupied by atoms of PPARα that comprises residues Ile-241, Leu-247, Ala-250, Glu-251, Leu-254, Val-255, Ile-272, Phe-273, Cys-275, Cys-276, Gln-277, Thr-279, Ser-280, Tyr-314, Ile-317, Phe-318, Leu-321, Met-330, Val-332, Ala-333, Ile-339, Leu-344, Ile-354, Met-355, His-440, Val-444, Leu-456, Leu-460 and Tyr-464. The binding pocket also includes regions of space near the “main” binding pocket that not occupied by atoms of PPARα but that are near the “main” binding pocket, and that are contiguous with the “main” binding pocket.

As used herein, the terms “PPAR gene” and “recombinant PPAR gene” mean a nucleic acid molecule comprising an open reading frame encoding a PPAR polypeptide of the present invention, including both exon and (optionally) intron sequences.

As used herein, the term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

As used herein, the term “DNA sequence encoding a PPAR polypeptide” can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the term “intron” means a DNA sequence present in a given gene that is not translated into protein.

As used herein, the term “interact” means detectable interactions between molecules, such as can be detected using, for example, a yeast two hybrid assay. The term “interact” is also meant to include “binding” interactions between molecules. Interactions can, for example, be protein-protein or protein-nucleic acid in nature.

As used herein, the terms “cells,” “host cells” or “recombinant host cells” are used interchangeably and mean not only to the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the term “agonist” means an agent that supplements or potentiates the bioactivity of a functional PPAR gene or protein or of a polypeptide encoded by a gene that is up- or down-regulated by a PPAR polypeptide and/or a polypeptide encoded by a gene that contains a PPAR binding site or response element in its promoter region.

As used herein, the term “antagonist” means an agent that decreases or inhibits the bioactivity of a functional PPAR gene or protein, or that supplements or potentiates the bioactivity of a naturally occurring or engineered non-functional PPAR gene or protein. Alternatively, an antagonist can decrease or inhibit the bioactivity of a functional gene or polypeptide encoded by a gene that is up- or down-regulated by a PPAR polypeptide and/or contains a PPAR binding site or response element in its promoter region. An antagonist can also supplement or potentiate the bioactivity of a naturally occurring or engineered non-functional gene or polypeptide encoded by a gene that is up- or down-regulated by a PPAR polypeptide, and/or contains a PPAR binding site or response element in its promoter region.

As used herein, the terms “chimeric protein” or “fusion protein” are used interchangeably and mean a fusion of a first amino acid sequence encoding a PPAR polypeptide with a second amino acid sequence defining a polypeptide domain foreign to, and not homologous with, any domain of a PPAR polypeptide. A chimeric protein can include a foreign domain that is found in an organism that also expresses the first protein, or it can be an “interspecies” or “intergenic” fusion of protein structures expressed by different kinds of organisms. In general, a fusion protein can be represented by the general formula X-PPAR-Y, wherein PPAR represents a portion of the protein which is derived from a PPAR polypeptide, and X and Y are independently absent or represent amino acid sequences which are not related to a PPAR sequence in an organism, which includes naturally occurring mutants.

### II. Description of Tables

Table 1 is a table summarizing the crystal and data statistics obtained from the crystallized ligand binding domain of PPARα. Data on the unit cell are presented, including data on the crystal space group, unit cell dimensions, molecules per asymmetric cell and crystal resolution.

Table 2 is a table of the atomic structure coordinate data obtained from X-ray diffraction from the ligand binding domain of PPARα in complex with a ligand.

Table 3 is a table of the atomic structure coordinate data obtained from X-ray diffraction from the ligand binding domain (residues 207-441) of a PPARδ crystal (Xu et al., (1999) Mol. Cell 3: 397-403, PDB ID: 1GWX; Genbank Accession No. L07592; available online at http://www.rcsb.org/pdb/). The coordinate data from the PPARδ ligand binding domain were used in the molecular replacement solution of the PPARα ligand binding domain crystal form.

Table 4 is a sequence alignment which shows sequence similarities between the PPARα, PPARγ and PPARδ sequences. The binding site residues are denoted by small boxes and represent those residues lying within 5.0 angstroms of the ligand.

### III. General Considerations

The present invention will usually be applicable mutatis mutandis to all PPARs, as discussed herein based, in part, on the patterns of PPAR structure and modulation that have emerged as a consequence of determining the three dimensional structure of PPARα with bound ligand. Analysis and alignment of amino acid sequences, and X-ray and NMR structure determinations, have shown that nuclear receptors have a modular architecture with three main domains:

1) a variable amino-terminal domain;

2) a highly conserved DNA-binding domain (DBD); and

3) a less conserved carboxy-terminal ligand binding domain (LBD).

In addition, nuclear receptors may have linker segments of variable length between these major domains. Sequence analysis and X-ray crystallography, including the work of the present invention, have confirmed that PPARs also have the same general modular architecture, with the same three domains. The function of the PPARs in human cells presumably requires all three domains in a single amino acid sequence. However, the modularity of the PPARs permits different domains of each protein to separately accomplish certain functions. Some of the functions of a domain within the full-length receptor are preserved when that particular domain is isolated from the remainder of the protein. Using conventional protein chemistry techniques, a modular domain can sometimes be separated from the parent protein. Using conventional molecular biology techniques, each domain can usually be separately expressed with its original function intact or, as discussed herein below, chimeras comprising two different proteins can be constructed, wherein the chimeras retain the properties of the individual functional domains of the respective nuclear receptors from which the chimeras were generated.

The amino terminal domain of the PPAR subtypes is the least conserved of the three domains. This domain is involved in transcriptional activation and, in some cases, its uniqueness may dictate selective receptor-DNA binding and activation of target genes by PPAR subtypes. This domain can display synergistic and antagonistic interactions with the domains of the LBD.

The DNA binding domain has the most highly conserved amino acid sequence amongst the PPARs. It typically contains about 70 amino acids that fold into two zinc finger motifs, wherein a zinc atom coordinates four cysteines. The DBD contains two perpendicularly oriented α-helixes that extend from the base of the first and second zinc fingers. The two zinc fingers function in concert along with non-zinc finger residues to direct the PPAR to specific target sites on DNA and to align receptor heterodimer interfaces. Various amino acids in the DBD influence spacing between two half-sites (which usually comprises six nucleotides) for receptor heterodimerization. The optimal spacings facilitate cooperative interactions between DBDs, and D box residues are part of the dimerization interface. Other regions of the DBD facilitate DNA-protein and protein-protein interactions required for RXR-PPAR heterodimerization.

The LBD is the second most highly conserved domain in these receptors. As its name suggest, the LBD binds ligands. With many nuclear receptors, including the PPARs, binding of the ligand can induce a conformational change in the LBD that can, in turn, activate transcription of certain target genes. The LBD also participates in other functions, including dimerization and nuclear translocation.

X-ray structures have shown that most nuclear receptor LBDs adopt the same general folding pattern. This fold consists of 10-12 alpha-helices arranged in a bundle, together with several beta-strands, additional alpha-helices and linking segments. The major alpha helices and beta-strands have been numbered differently in different publications. This patent will follow the numbering scheme of Nolte et al., (Nolte et al., (1998) Nature 395:137-43), where the major alpha-helices and beta-strands are designated sequentially through the amino acid sequence as H₁, H2, S1, H2′, H3, H3′, H4, H5, S2, S3, S4, H6, H7, H8, H9, H10 and HAF. The alpha-helix at the C-terminal end, HAF, is also called “helix-AF”, “helix-AF2” or the “AF2 helix”. Structural studies have shown that most of the alpha-helices and beta-strands have the same general position and orientation in all nuclear receptor structures, whether ligand is bound or not. However, the AF2 helix has been found in different positions and orientations relative to the main bundle, depending on the presence or absence of the ligand, and also on the chemical nature of the ligand. These structural studies have suggested that many nuclear receptors share a common mechanism of activation, where binding of activating ligands helps to stabilize the AF2 helix in a position and orientation adjacent to helices-3, -4, and -10, covering an opening to the ligand binding site. This position and orientation of the AF2 helix, which will be called the “active conformation”, creates a binding site for coactivators. See, e.g., Nolte et al., (1998) Nature 395:137-43; Shiau et al., (1 998) Cell 95: 927-37. This coactivator binding site has a central lipophilic pocket that can accommodate feucine side-chains from coactivators, as well as a “charge-clamp” structure consisting primarily of a lysine residue from helix-3 and a glutamic acid residue from the AF2 helix. Structural studies have shown that coactivator peptides containing the sequence LXXLL (where L is leucine and X can be a different amino acid in different cases) can bind to this coactivator binding site by making interactions with the charge clamp lysine and glutamic acid residues, as well as the central lipophilic region. This coactivator binding site is disrupted when the AF2 helix is shifted into other positions and orientations. In PPAR-γ, activating ligands such as rosiglitazone (BRL49653) make a hydrogen bonding interaction with tyrosine-473 in the AF2 helix. Nolte et al., (1998) Nature 395:137-43; Gampe et al., (2000) Mol. Cell 5: 545-55. This interaction is believed to stabilize the AF2 helix in the active conformation, thereby allowing coactivators to bind and thus activating transcription from target genes. With certain antagonist ligands, or in the absence of any ligand, the AF2 helix may be held less tightly in the active conformation, or may be free to adopt other conformations. This would either destabilize or disrupt the coactivator binding site, thereby reducing or eliminating coactivator binding and transcription from certain target genes. Some of the functions of the PPAR protein depend on having the full-length amino acid sequence and certain partner molecules, such as coactivators and DNA. However, other functions, including ligand binding and ligand-dependent conformational changes, may be observed experimentally using isolated domains, chimeras and mutant molecules.

As described herein, the LBD of a PPAR can be expressed, crystallized, its three dimensional structure determined with a ligand bound as disclosed in the present invention, and computational methods can be used to design ligands to its LBD.

### IV. Synthesis of Compound 1 and Intermediates

Compound 1, which was co-crystallized with the PPARα LBD in the present invention, can be conveniently prepared by a general process wherein a moiety like (A) is coupled to an acid (B) using a peptide coupling reaction or by alkylation of (A) using a suitable non nucleophilic amine with an acid chloride (C). Preferably, R is 1-6 alkyl, which can be hydrolyzed off or, is readily hydrolyzable.

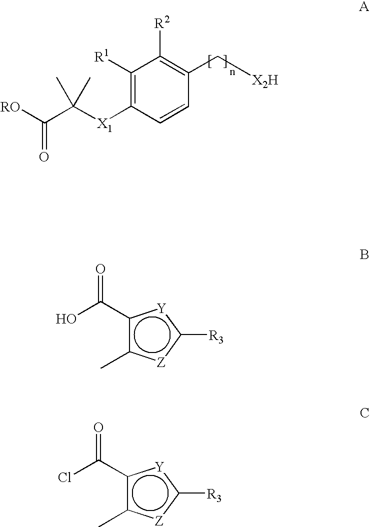

A preferred synthesis of (A) when X₁ is O and X₂ is NH (and R¹ and R² are H) is:

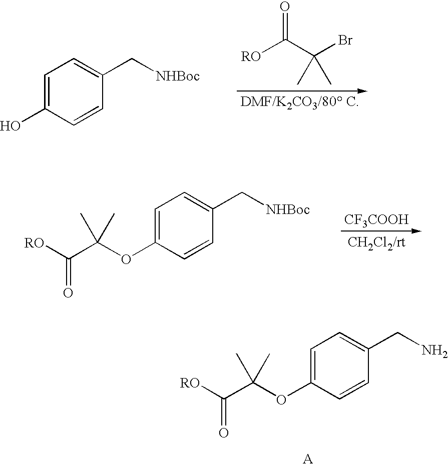

Note that this synthesis is preferably carried out with the amine where the alcohol function is already alkylated with the acid side chain protected by R. For example, when n is 1, X₁ is O, X₂ is NH, Y is S, Z is N, R¹ and R² are H, and R³ is 4-F₃C-phenyl:

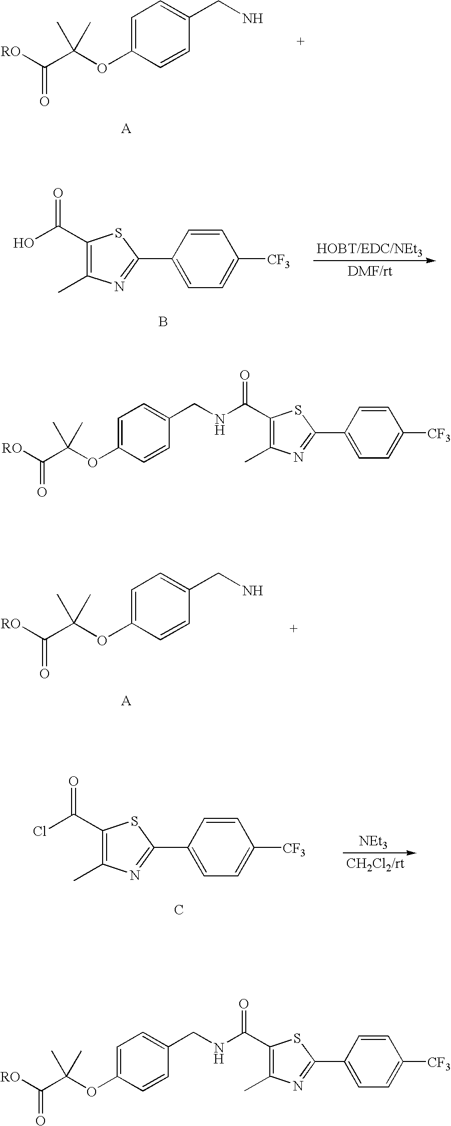

Some of the intermediates of type A are commercially available while others can be synthesized by techniques apparent to a person skilled in the art. The synthesis of intermediates of type B is illustrated below.

Compound 1 can be made by an alternative method in which compounds of formula (D) are reacted with ethyl 2-bromo-2 methyl propionate to produce an ethyl ester, which may be hydrolyzed to produce the free acid.

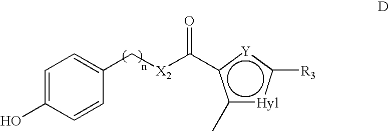

Compounds of formula (D) may be prepared from the reaction between compounds of formula (B) and compounds of formula (E) with HOBT/EDC/NEt₃ when X₂ is NH or NCH₃ or DIC/DMAP/NEt₃ when X₂ is O.

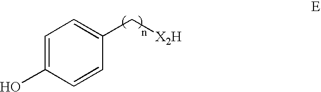

The invention is further illustrated by the following examples which should not be construed as constituting a limitation thereto.

IV.A. Synthesis of Intermediate 1

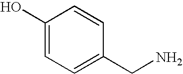

Formation of Intermediate 1 follows the procedure described by Stout (Stout, (1983) J. Med. Chem. 26(6): 808-13). To 4-methoxybenzyl amine (25 g, 0.18 mol; Aldrich) is added 46% HBr in H₂O (106 ml, 0.9 mol; Aldrich). The reaction is refluxed overnight, then the reaction is cooled to 0° C. and neutralized to pH7 slowly with KOH₍ₛ₎. The reaction is allowed to stir for ˜30 min, then the solid filtered and dried. The solid is redisolved in hot MeOH, filtered and the solution cooled to afford 19 g (85%) intermediate 1.

IV.B. Synthesis of Intermediate 2

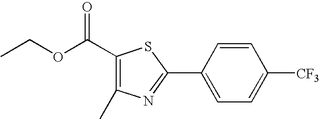

A solution of ethyl 2-chloroacetoacetate (35.3 g, 29.7 mL, 0.21 mol) and 4-(trifluoromethyl)thiobenzamide (44 g, 0.21 mol) in EtOH (300 mL) is refluxed overnight. After cooling to room temperature the solvent is removed in vacuo. The final product (Intermediate 2) is recrystallized from a minimum of MeOH to afford 40 g (59%) of final product as a white solid.

IV.C. Synthesis of Intermediate 3

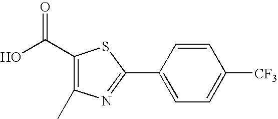

To Intermediate 2 (1.84 g, 5.8 mmol) in THF is added 1N LiOH (6 mL, 6 mmol) and the reaction stirred at room temperature. After ˜3 h, the reaction is neutralized with 1N HCl, extracted 3×100 mL EtOAc, dried over Na₂SO₄, filtered and the solvent removed under vaccum to afford 1.5 g (89%) of Intermediate 3 as a white solid.

IV.D. Synthesis of Intermediate 4

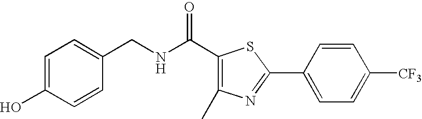

To intermediate 3 (1 g, 7 mmol) in CH₂Cl₂/DMF (1:1) is added HOBT (565 mg, 4.2 mmol; Aldrich), EDC (800 mg, 4.2 mmol; Aldrich) and Intermediate 1 (860 mg, 7 mmol). The reaction is stirred at room temperature for 18 h. The solvent is then removed in vacuo, treated with H₂O and extracted 3×100 mL CH₂Cl₂. The organic phases are then combined and washed with 1N HCl, dried over Na₂SO₄, filtered and evaporated to afford a mixture (N-substituted and N,O-substituted). The mixture is disolved in MeOH and treated with 1N NaOH. The reaction is stirred 18 h at 50° C. The solvent removed in vacuo, dissolved in CH₂Cl₂, washed with H₂O, and dried over Na₂SO₄. The solvent evaporated the residue chromatographed (CH₂Cl₂/MeOH: 99/1) to afford 610 mg (47%) of Intermediate 4 as a white solid.

IV.E. Synthesis of Intermediate 5

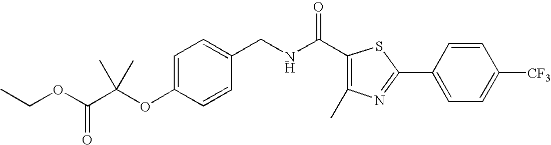

2-methyl-2-[4-{[(4-methyl-2-[4-trifluoromethylphenyl]thiazol-5 ylcarbonyl)amino]methyl}phenoxy]propionic acid ethyl ester

To Intermediate 4 (710 mg, 1.81 mmol) in DMF (50 mL) is added the K₂CO₃ (275 mg, 1.99 mmol) followed by the ethyl 2-bromo-2-methylpropanate (280 μL, 1.91 mmol; Aldrich) and the reaction is heated to 80° C. After 18 h, the reaction is cooled to room temperature and the solvent removed in vacuo. The residue is treated with water (200 mL), extracted 3×50 mL CH₂Cl₂, dried over Na₂SO₄, filtered and the solvent removed under vaccum to afford 680 mg (77%) of Intermediate 5 as a clear oil.

IV.F. Synthesis of Compound 1

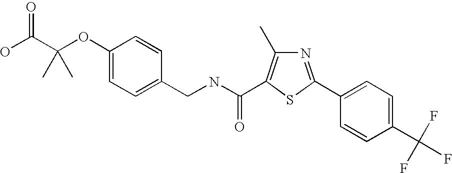

2-methyl-2-[4-{[(4-methyl-2-[4-trifluoromethylphenyl]-thiazol-5-ylcarbonyl)amino]methyl}phenoxy]propionic acid

To Intermediate 5 (680 mg, 1.39 mmol) in MeOH is added 1N NaOH (1.6 mL, 1.6 mmol) and the reaction is stirred at 60° C. After 18 h, the reaction is cooled to room temperature and the solvent evaporated. The residue treated with 1N HCl, extracted 3×20 mL THF and the solvent is removed under vacuum to afford 500 mg (75%) of Compound 1.

V. Production of PPAR Polypeptides

The native and mutated PPAR polypeptides, and fragments thereof, of the present invention can be chemically synthesized in whole or part using techniques that are well-known in the art (See, e.g., Creighton, (1983) Proteins: Structures and Molecular Principles, W.H. Freeman & Co., New York, incorporated herein in its entirety). Alternatively, methods which are well known to those skilled in the art can be used to construct expression vectors containing a partial or the entire native or mutated PPAR polypeptide coding sequence and appropriate transcriptional/translational control signals. These methods include in vitro recombinant DNA techniques, synthetic techniques and in vivo recombination/genetic recombination. See, for example, the techniques described throughout Sambrook et al., (1989) Molecular Cloning: A Laboratory Manual, Cold Spring Harbor Laboratory, New York, and Ausubel et al., (1989) Current Protocols in Molecular Biology, Greene Publishing Associates and Wiley Interscience, New York, both incorporated herein in their entirety.

A variety of host-expression vector systems can be utilized to express a PPAR coding sequence. These include but are not limited to microorganisms such as bacteria transformed with recombinant bacteriophage DNA, plasmid DNA or cosmid DNA expression vectors containing a PPAR coding sequence; yeast transformed with recombinant yeast expression vectors containing a PPAR coding sequence; insect cell systems infected with recombinant virus expression vectors (e.g., baculovirus) containing a PPAR coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a PPAR coding sequence; or animal cell systems. The expression elements of these systems vary in their strength and specificities.

Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used. When cloning in insect cell systems, promoters such as the baculovirus polyhedrin promoter can be used. When cloning in plant cell systems, promoters derived from the genome of plant cells, such as heat shock promoters; the promoter for the small subunit of RUBISCO; the promoter for the chlorophyll a/b binding protein) or from plant viruses (e.g., the 35S RNA promoter of CaMV; the coat protein promoter of TMV) can be used. When cloning in mammalian cell systems, promoters derived from the genome of mammalian cells (e.g., metallothionein promoter) or from mammalian viruses (e.g., the adenovirus late promoter; the vaccinia virus 7.5K promoter) can be used. When generating cell lines that contain multiple copies of the tyrosine kinase domain DNA, SV40-, BPV- and EBV-based vectors can be used with an appropriate selectable marker.

VI. Formation of PPARα Ligand Binding Domain Crystals

In one embodiment, the present invention provides crystals of PPARα LBD. The crystals were obtained using the methodology disclosed in the Examples. The PPARα LBD crystals, which can be native crystals, derivative crystals or co-crystals, have orthorhombic unit cells (an orthorhombic unit cell is a unit cell wherein a≠b≠c, and wherein α=β=γ=90°) and space group symmetry P2₁2₁2. There is one PPARα LBD molecule in the asymmetric unit. In this PPARα crystalline form, the unit cell has dimensions of a=61.3 Å, b=103.5 Å, c=49.9 Å, and α=β=γ=90°. This crystal form can be formed in a crystallization reservoir comprising 4-8% PEG 3350, 100-200 mM NaF, and 12-16% 2,5 hexanediol.

In another PPARα crystal form, the unit cell has dimensions of a=95.58 Å, b=122.06 Å, c=122.10 Å, α=β=γ=90° and belongs to the space group P2₁2₁2₁. There are four PPARα LBD molecules in the asymmetric unit of this crystal form. This crystal form can be formed in a crystallization reservoir comprising 50 mM bis-tris-propane, 4-6% PEG 3350, 150 mM NaNO₃, 16% 2,5 hexanediol, and 1-3 mM YCl₃.

VI.A. Preparation of PPAR Crystals

The native and derivative co-crystals, and fragments thereof, disclosed in the present invention can be obtained by a variety of techniques, including batch, liquid bridge, dialysis, vapor diffusion and hanging drop methods (See, eg., McPherson, (1982) Preparation and Analysis of Protein Crystals, John Wiley, New York.; McPherson, (1990) Eur. J. Biochem. 189:1-23.; Weber, (1991) Adv. Protein Chem. 41:1-36). In a preferred embodiment, the vapor diffusion and hanging drop methods are used for the crystallization of PPAR polypeptides and fragments thereof.

In general, native crystals of the present invention are grown by dissolving substantially pure PPAR polypeptide or a fragment thereof in an aqueous buffer containing a precipitant at a concentration just below that necessary to precipitate the protein. Water is removed by controlled evaporation to produce precipitating conditions, which are maintained until crystal growth ceases.

In a preferred embodiment of the invention, native crystals are grown by vapor diffusion (See, e.g., McPherson, (1982) Preparation and Analysis of Protein Crystals, John Wiley, New York.; McPherson, (1990) Eur. J. Biochem. 189:1-23). In this method, the polypeptide/precipitant solution is allowed to equilibrate in a closed container with a larger aqueous reservoir having a precipitant concentration optimal for producing crystals. Generally, less than about 25 μL of PPAR polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand, until crystals grow. Crystals generally form within two to six weeks, and are suitable for data collection within approximately seven to ten weeks. Of course, those of skill in the art will recognize that the above-described crystallization procedures and conditions can be varied.

VI.B. Preparation of Derivative Crystals

Derivative crystals of the present invention, e.g. heavy atom derivative crystals, can be obtained by soaking native crystals in mother liquor containing salts of heavy metal atoms. Such derivative crystals are useful for phase analysis in the solution of crystals of the present invention. In a preferred embodiment of the present invention, for example, soaking a native crystal in a solution containing methyl-mercury chloride provides derivative crystals suitable for use as isomorphous replacements in determining the X-ray crystal structure of a PPAR polypeptide. Additional reagents useful for the preparation of the derivative crystals of the present invention will be apparent to those of skill in the art after review of the disclosure of the present invention presented herein.

VI.C. Preparation of Co-crystals

Co-crystals of the present invention can be obtained by soaking a native crystal in mother liquor containing compounds known or predicted to bind the LBD of a PPAR, or a fragment thereof. Alternatively, co-crystals can be obtained by co-crystallizing a PPAR LBD polypeptide or a fragment thereof in the presence of one or more compounds known or predicted to bind the polypeptide. In a preferred embodiment, such a compound is Compound 1.

VI.D. Solving a Crystal Structure of the Present Invention

Crystal structures of the present invention can be solved using a variety of techniques including, but not limited to, isomorphous replacement, anomalous scattering or molecular replacement methods. Computer software packages will also be helpful in solving a crystal structure of the present invention. Applicable software packages include but are not limited to X-PLOR™ program (Brünger, (1992) X-PLOR, Version 3.1. A System for X-ray Crystallography and NMR, Yale University Press, New Haven, Conn.; X-PLOR is available from Molecular Simulations, Inc., San Diego, Calif.), Xtal View (McRee, (1992) J. Mol. Graphics 10: 44-47; X-tal View is available from the San Diego Supercomputer Center). SHELXS 97 (Sheldrick (1990) Acta Cryst. A46: 467; SHELX 97 is available from the Institute of Inorganic Chemistry, Georg-August-Universität, Göttingen, Germany), HEAVY (Terwilliger, Los Alamos National Laboratory) and SHAKE-AND-BAKE (Hauptman, (1997) Curr. Opin. Struct. Biol. 7: 672-80; Weeks et al., (1993) Acta Cryst. D49: 179; available from the Hauptman-Woodward Medical Research Institute, Buffalo, N.Y.) can be used. See also, Ducruix & Geige, (1992) Crystallization of Nucleic Acids and Proteins: A Practical Approach, IRL Press, Oxford, England, and references cited therein.

VII. Characterization and Solution of a PPARα Ligand Binding Domain Crystal

VII.A Unique Structural Differences Between PPARα and Other PPARs

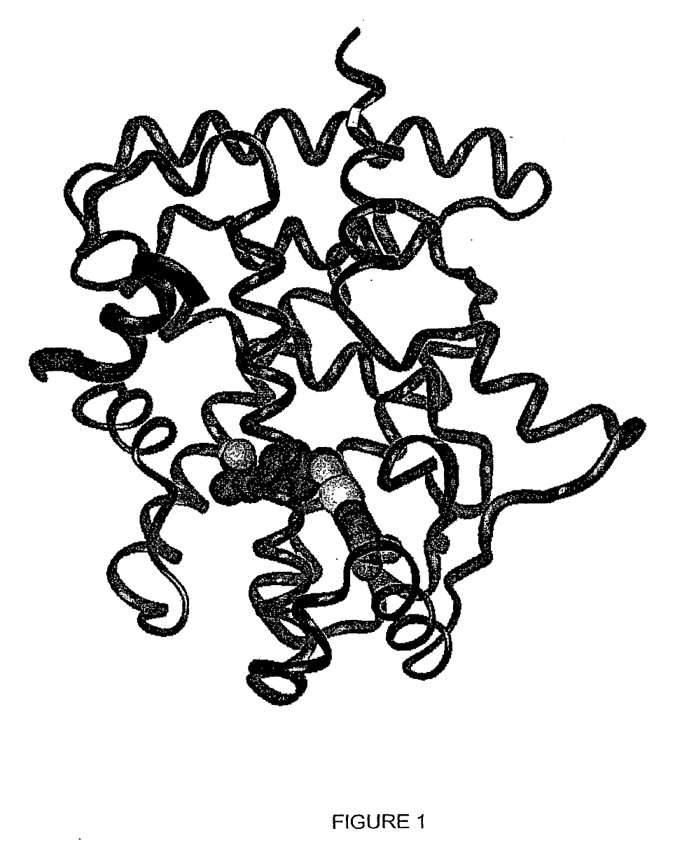

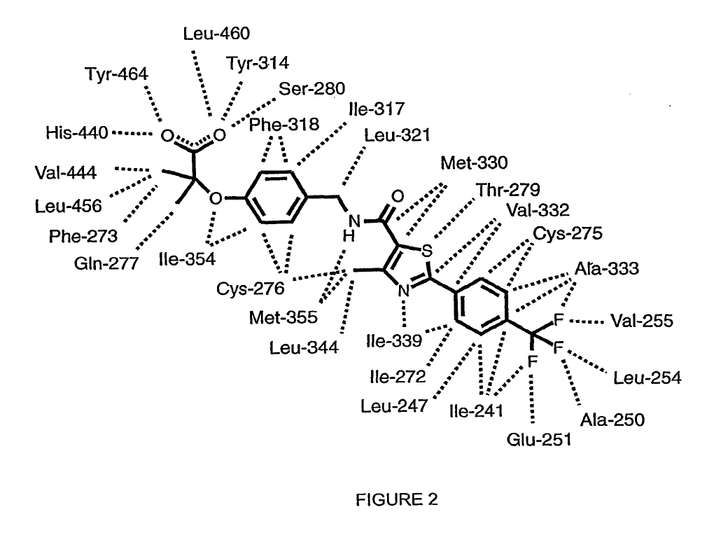

The PPARα LBD-ligand structure was solved here using molecular replacement techniques. The overall folding of the protein backbone, and the binding mode of Compound 1, are shown in the ribbon diagram of FIG. 1. Specific interactions between Compound 1 and the protein are shown schematically in FIG. 2. The structure of the PPARγ LBD was solved previously in the apo form, i.e., with no ligand, and also with a thiazolidinedione ligand (rosiglitazone) and a coactivator peptide (Nolte et al., (1998) Nature 395:137-43). The apo structure has also been determined independently (Uppenberg et al., (1998) J. Biol. Chem. 273: 31108-12). In addition, the structure of the PPARγ LBD has been determined with a partial agonist, GW0072 (Oberfield et al., (1999) Proc. Nat. Acad. Sci. 96: 6102-106). The structure of the PPARγ LBD has also been determined in the heterodimeric complex with RXRα, together with coactivator peptides, with rosiglitazone, and also with a carboxylic acid ligand, GI262570 (Gampe et al., (2000) Mol. Cell 5: 545-55). The structure of the PPARδ LBD has been determined in the apo form, and with eicosapentaenoic acid, and with the synthetic compound GW2433 (Xu et al., (1999) Mol. Cell 3: 397-403).

To facilitate comparison, the structures were first translated and rotated into a common position and orientation as shown in FIG. 1. This superimposition operation was done with the MVP program (Lambert, (1997) in Practical Application of Computer-Aided Drug Design, (Charifson, ed.), pp. 243-303, Marcel-Dekker, New York) by first aligning the amino acid sequences to identify corresponding residues in the three different PPARs, and then rotating and translating so as to superimpose corresponding C₆₀ or backbone atoms from the aligned residues. With this translation and rotation, most of the major alpha-helices and beta-strands of the three PPARs are closely superimposed. The AF2 helix is well superimposed in most of the structures, but is shifted into a different position in certain subunits of structures of PPARγ either in the absence of ligand (apo), or in the presence of the partial agonist GW0072. This shift provides evidence that the PPAR AF2 helix can shift out of the active conformation, and that it is more likely to do so in the absence of a strongly activating ligand. In the above listed PPAR X-ray structures, the ligands that act as strong agonists (rosiglitazone, GI262570, eicosapentaenoic acid, GW2433, Compound 1) are all oriented with the acid group near the AF2 helix, and they all make a hydrogen bond with a tyrosine residue in the AF2 helix (Tyr464 in PPARα, Tyr473 in PPARγ, Tyr437 in PPARδ). The partial agonist, GW0072, also has a carboxyl group, but it is oriented differently, and fails to make any hydrogen bond with the AF2 tyrosine. This suggests that the hydrogen bond between the ligand and the AF2 tyrosine helps to hold the AF2 helix in the active conformation and thereby facilitate coactivator binding. See, Xu et al., (1999) Mol. Cell 3: 397-403 and Oberfield et al., (1999) Proc. Nat Acad. Sci. 96: 6102-106.

To facilitate discussion, it is useful to establish a nomenclature for regions of the ligand and the ligand binding pocket. The strongly activating ligands in these X-ray structures all have an acid group that binds near the AF2 tyrosine, and a lipophilic “tail” that extends into a lipophilic pocket. The acid group can be called the acid “headgroup”, and the site near the AF2 helix into which it binds can be called the “headgroup binding site”. In rosiglitazone, GI262570 and Compound 1, the lipophilic tail is directed into a lipophilic pocket delineated by helix-2′, helix-3 and the beta sheet. This will be called the “lower tail pocket” or “lower pocket”. In the structure of PPARδ bound to eicosapentaenoic acid, the eicosapentaenoic acid was found in two different binding modes with roughly equal occupancy (Xu et al., (1999) Mol. Cell 3: 397-403). In both binding modes, the acid headgroup bound in the headgroup binding site near Tyr 437. In one binding mode, the lipophilic tail was directed into the lower tail pocket. However, in the other binding mode, the lipophilic tail was directed into a different pocket, delineated by helix-3, helix-5, the beta sheet and the loop between helix-1 and helix-2. This will be called the “upper tail pocket.” GW2433 has a branched tail, and was found to bind with one branch in the lower pocket, and with the other branch in the upper pocket (Xu et al., (1999) Mol. Cell 3: 397-403). GI262570 has an additional lipophilic benzophenone group near the acid headgroup. This benzophenone group was found to be directed into a lipophilic pocket delineated by helix-3, helix-7, helix-10 and the loop between helix-10 and the AF2 helix. This will be subsequently referred to as the “benzophenone pocket.”

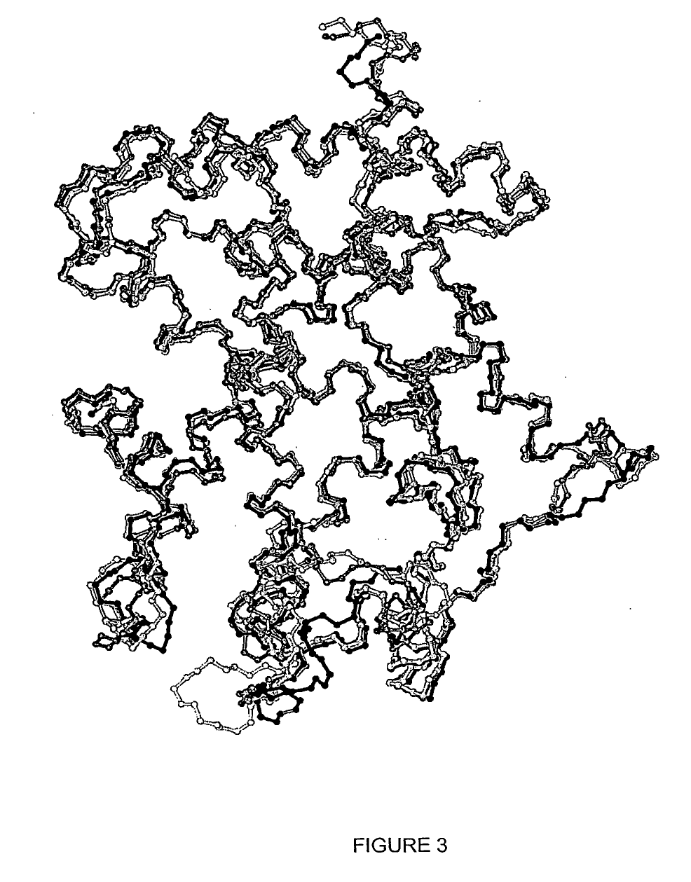

Comparing the backbone structures, there are substantial differences between PPARα, PPARγ and PPARδ in the “loop” region between helix-2 and strand-1, the loop region between helix-2′ and helix-3, and the loop region between helix-10 and the AF2 helix. There are also smaller differences in the loop region between helix-1 and helix-2, and the loop region between helix-6 and helix-7. Helices 2, 2′ and 3′ are closely superimposed, but have slightly different lengths, as indicated in Table 4. Helix-10 has the same length in all structures, but bends differently over the ligand binding pocket in PPARα, PPARγ and PPARδ. Some of these structural differences are visible in FIG. 3. Many of these structural differences lie close to the ligand binding pocket, and could therefore be important in the receptor selectivity of different ligands.

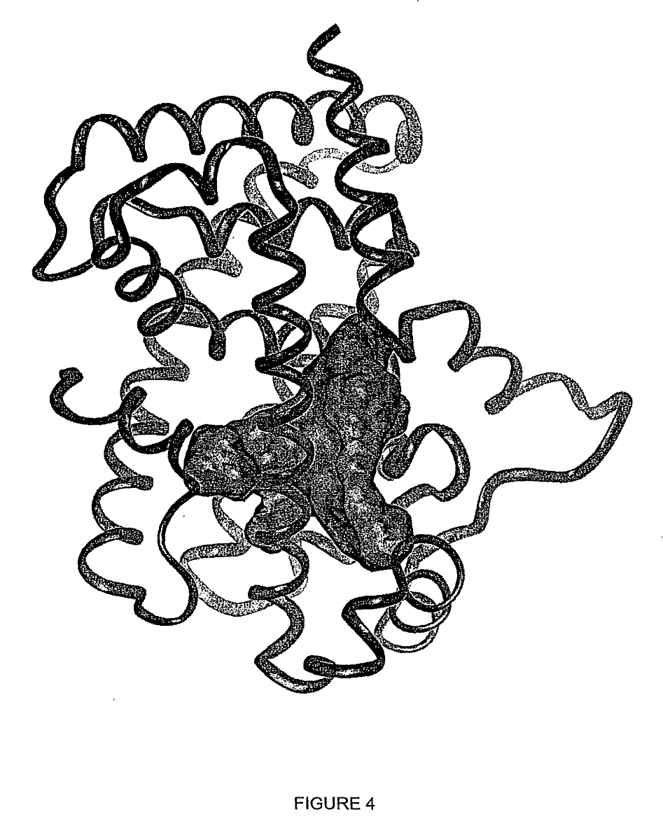

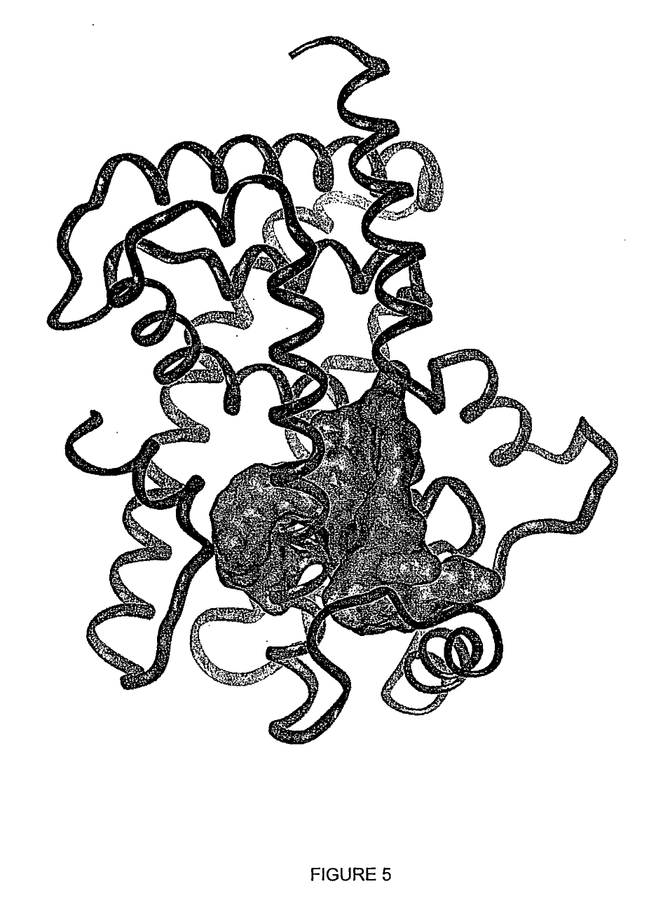

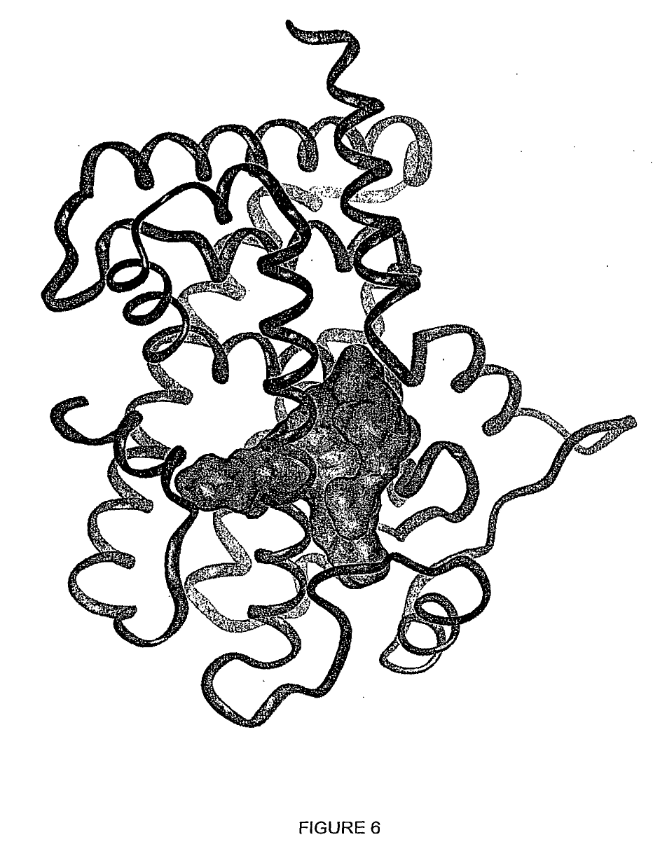

Helix-2′ and the loop between helix-2′ and helix-3 (the 2′-3 loop) together serve as one wall for the “lower tail pocket” in all three PPARs. Helix-2′, and the 2′-3 loop, adopt different conformations in some of the different PPARγ structures. In particular, helix-2′ is shorter in the homodimer structures of PPARγ bound to rosiglitazone and GW0072, and in the absence of ligand, than it is in the RXRα/PPARγ heterodimer structures where PPARγ is bound to rosiglitazone or GI262570. Some of these conformational differences might result from differences in crystal packing. However, the RXRα/PPARγ heterodimer represents the closer approximation to the biologically active complex, and will be used here to represent the most relevant conformation of PPARγ. By contrast, helix-2′ and the 2′-3 loop adopt more nearly equivalent backbone conformations in the available X-ray structures of PPARα and PPARδ. Considering the conformation in the RXRα/PPARγ/GI262570 heterodimer structure, helix-2′ is longer in PPARα and PPARδ, and the C-terminal end of the helix adopts different conformations in the three PPARs. In PPARα, the last residue of the helix, Leu254, makes the expected alpha-helical hydrogen bonds, but bulges away from the axis of the helix towards the ligand. This bulged conformation allows Leu254 and Val255 to cover the “bottom” of the lower tail pocket, effectively narrowing the mouth of the pocket. In PPARδ, the C-terminal end of helix-2′ is wound more loosely, such that the corresponding residue, Leu226, cannot cover the bottom of the ligand binding pocket. Instead, the 2′-3 loop adopts a different conformation that places the side-chain of Trp228 in the bottom of the lower tail pocket. This shortens the lower tail pocket slightly, and also constricts the opening to solvent. In PPARγ, helix-2′ is bent such that its corresponding residue, Ile262, is shifted farther from the pocket. Also, the next two residues, Lys263 and Phe264, adopt the helical conformation, effectively extending helix-2 by two residues, such that Lys263 is far from the ligand and cannot cover the solvent channel. This leaves a very wide opening to solvent at this position in PPARγ, as depicted by the large, wide cavity region above the 2′-3 loop in FIG. 5. The opening is much narrower in PPARα and PPARδ, as depicted in FIGS. 4 and 6. These variations in the backbone conformation would be difficult or impossible to predict from the previously available X-ray structures with any homology modeling procedure. Nonetheless, very accurate models for helix-2′ and the 2′-3 loop in all three PPARs would be essential for understanding the binding mode and receptor selectivity of ligands that occupy the lower tail pocket.

The loop between helix-1 and helix-2 (the “1-2 loop”) serves as a wall at the far end of the upper tail pocket. Residues in this 1-2 loop come close to the tail of eicosapentaenoic acid (in its upper tail pocket binding mode), and close to the fluoro-,chloro-phenyl ring of GW2433 in PPARδ. Several backbone amide CO and NH groups are directed towards the ligand binding site, and could serve as hydrogen bonding partners with an appropriately designed ligand. However, the backbone conformation in the 1-2 loop is slightly different in PPARα, PPARγ and PPARδ. In particular, the backbone amide CO and NH groups have slightly different orientations in PPARα, PPARγ and PPARδ. Some of these differences may result from the presence of a proline at position 227 in PPARγ, whereas PPARα and PPARδ have asparagines at this position. These subtle differences would be difficult or impossible to predict to high accuracy using the previously available X-ray structures with standard homology modeling procedures. Nonetheless, a very accurate model of this loop conformation would be essential for understanding the receptor selectivity of ligands that interact with this loop.

The C-terminal end of helix-10 and the loop between helix-10 and the AF2 helix (the “10-AF2 loop”) together serve as one wall for the headgroup binding site and the benzophenone pocket. An additional wall is provided by a glutamine residue in helix-3 (Gln277 in PPARα, Gln286 in PPARγ, Gln250 in PPARδ) that reaches across the benzophenone binding site to make hydrogen bonds with backbone CO and NH groups from the 10-AF2 loop. This glutamine side-chain, and the 10-AF2 loop itself, adopt different conformations in the three PPARs. In PPARα and PPARδ, the glutamine side-chain adopts conformations that narrow the benzophenone pocket substantially. In addition, in PPARα and PPARδ, the 10-AF2 loop adopts a conformation that crowds a phenylalanine from helix-3 (Phe273 in PPARα, Phe282 in PPARγ, Phe246 in PPARδ) more in PPARα and PPARδ than in PPARγ. These two structural differences cause a slight narrowing of the benzophenone pocket in PPARα, and a more substantial narrowing in PPARδ. This can be seen by comparison of PPARα and PPARδ in FIGS. 4 and 6 with PPARγ in FIG. 5. The narrowing is sufficient to significantly reduce the binding affinity of ligands that have a benzophenone group at this position. The narrowing in PPARα and PPARδ suggests that smaller groups should be used at this position to obtain good binding to PPARα, and substantially smaller groups should be used to obtain good binding to PPARδ. Aside from these changes that narrow the pocket in PPARα and PPARδ, there are also structural changes that modulate the shape of the pocket and the position and orientation of potential hydrogen bonding groups. For example, the C-terminal end of helix-10 bends inwards toward the ligand binding site in all three PPARs. However, the bend is slightly different in the different PPARs, placing corresponding side-chains in slightly different positions. These variations in the backbone conformation, and corresponding changes in side-chain conformation, would be difficult or impossible to predict from the previously available X-ray structures with any available homology modeling procedure. Nonetheless, very accurate models for helix-10, the 10-AF2 loop and the side-chains in this region would be essential for understanding the binding mode and receptor selectivity of ligands that occupy the benzophenone pocket.

Aside from these unexpected differences in the backbone structure of PPARα, the present X-ray structure also revealed differences involving side-chains. In some cases, these differences involve residue positions where the amino acid is different in PPARα from that in either PPARγ or PPARδ or both. In other cases, PPARα may have the same amino acid at a particular residue position as PPARγ or PPARδ or both, but the amino acid side-chain adopts a different conformation in PPARα.

One of the most important side-chain differences is Tyr314 in PPARα, a position that corresponds to His323 in PPARγ and His287 in PPARδ. In PPARγ and PPARδ, this histidine residue makes a hydrogen bond with the acidic headgroup of the ligand for all strongly activating ligands for which structures are available. The present PPARα structures shows that Tyr314 also makes a hydrogen bond with the acidic headgroup of Compound 1. However, the tyrosine OH lies farther from the protein backbone than the corresponding hydrogen bonding atoms in histidine. Consequently, changing histidine to tyrosine could potentially require a change in the position and/or orientation and/or conformation of the ligand, or changes in the conformation of the PPARα protein. The present PPARα crystal structure showed that the protein backbone is essentially unchanged in this region of PPARα, compared with PPARγ and PPARδ. Instead, the acid headgroup of the ligand is shifted and rotated in such a way that it can still make hydrogen bonds with Ser280, Tyr314, His440 and Tyr464. This involves small changes in the conformations of Tyr314, His440 and Tyr464, relative to PPARγ and PPARδ. Also, this involves a larger change in the conformation of Ser280, which unexpectedly adopts a conformation different from that of Ser289 in PPARγ bound to GI262570. The PPARα conformation places the Ser280 side-chain oxygen near the position of the side-chain oxygen of Thr253 in PPARδ bound to GW2433. The exact shift and rotation, and the exact conformations of the side-chains of Ser280, Tyr314, His440 and Tyr464, would be difficult or impossible to predict without this X-ray crystal structure of PPARα. However, this shift and rotation do significantly affect the position of the whole ligand within the ligand binding site. The position and orientation of the ligand carboxylate group revealed in this PPARα X-ray structure, and the interactions it makes with Ser280, Tyr314, His440 and Tyr464, can serve as a template for docking other compounds into PPARα using molecular modeling procedures.

This PPARα X-ray structure also revealed numerous other side-chains where the conformation in PPARα was different from that in PPARγ and/or PPARδ, and where the diffidence could affect the shape of the ligand binding pocket, and the position, orientation and/or conformation of the ligand. These additional residues with differences include, but are not limited to, Gln277, Phe273, His274, Ile354, Leu321, Met320, Met330 and Glu251. The side-chain chain conformational differences involving Gln277, Phe273 and Ile354 affect the volume and detailed shape of the headgroup binding site and/or the benzophenone pocket. His274 may affect these pockets indirectly, through its effect on Gln277 and Phe273. Leu321, Met320, Met330 and Glu251 affect the shape and volume of the upper and lower tail pockets. Numerous other side-chains also affect the size, shape and electrostatic character of the ligand binding site, and the position, orientation and conformation of the ligand within the ligand binding site. An understanding of the PPARα, PPARγ and PPARδ selectivity of various ligands would depend on having an accurate structure of each of the three PPARs, as well as an accurate position of the whole ligand within the pocket. This PPARα X-ray structure provides an accurate protein structure, as well as a template for modeling alternative ligands.

VII.B Characterization of the PPARα Binding Pocket

The ligand binding domain of PPARα was co-crystallized with Compound 1, which has the IUPAC name 2-methyl-2-[4-{[(4-methyl-2-[4-trifluoromethylphenyl] thiazol-5-yl-carbonyl) amino] methyl} phenoxy] propionic acid.

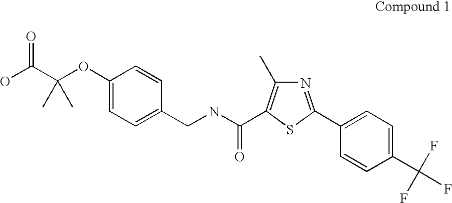

Compound 1 is an agonist of hPPARα and is useful for treatment of hPPARα mediated diseases or conditions including dyslipidemia, syndrome X, heart failure, hypercholesteremia, cardiovascular disease, type II diabetes mellitus, type I diabetes, insulin resistance, hyperlipidemia, obesity, inflammation, anorexia bulimia and anorexia nervosa.

FIGS. 1, 2 and 7 depict the conformation and orientation of the ligand Compound 1 in the binding site. FIG. 1 depicts the overall orientation of the α helices and β strands of the ligand binding domain of PPARα as it binds Compound 1. Compound 1 is presented as a spacefilling model.

A more specific graphical description of the residues of the PPARα LBD involved in ligand binding is presented in FIG. 2. FIG. 2 is a schematic diagram depicting those residues of the PPARα LBD that interact with Compound 1 as it is bound in the binding pocket of the PPARα LBD. Note that FIG. 2 is a schematic diagram, and highlights residues that interact with the ligand and does not indicate intermolecular distances.

VII.C. Hydrogen Bonding in the Binding Pocket of the PPARα LBD

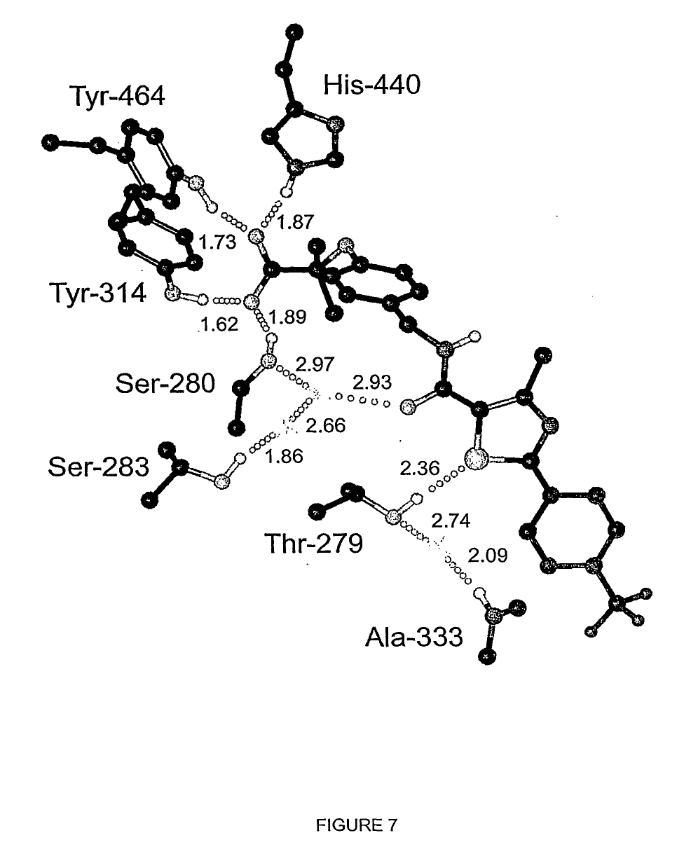

The hydrogen bonding scheme of the solvated binding pocket is presented in FIG. 7. In FIG. 7, atoms are shaded according to element, with carbon, fluorine, nitrogen, oxygen, sulfur and hydrogen in progressively lighter shades of gray. Sulfur is depicted with a slightly bigger ball, while hydrogen, fluorine and oxygen are depicted with slightly smaller balls. The hydrogen atoms shown here were not visible in the electron density, and were instead modeled into reasonable conformations (using standard bond lenghts and angles) to obtain possible hydrogen bond interactions, shown here with strings of small white balls. Each interaction is annotated with its distance in angstroms. Residues of the PPARα LBD binding pocket involved in hydrogen bonding are labeled. It is observed that hydrogen bonding relationships exist between the side chains of PPARα LBD residues and the ligand, between the side chains of PPARα LBD residues and solvent molecules, and between the ligand and solvent molecules. The hydrogen bonding pairs are identified by dotted lines.

FIG. 7 highlights hydrogen bonding that occurs between solvent molecules (indicated as crosses in FIG. 7) and the side chains of PPARα LBD residues in the binding pocket. Residues that hydrogen bond to solvent and are visible in FIG. 7 include Ser-280, Ser-283, Thr-279 and Ala-333.

Additionally, FIG. 7 highlights those residues of the PPARα LBD that hydrogen bond directly to the ligand. Residues of the PPARα LBD in which side chains hydrogen bond directly to the ligand are visible in FIG. 7, and include His-440, Tyr-464, Tyr-314, Ser 280 and Thr-279.

VII.D. Generation of Easily-Solved PPAR Crystals

The present invention discloses a substantially pure PPAR LBD polypeptide in crystalline form. In a preferred embodiment, exemplified in the Figures and Laboratory Examples, PPARα is crystallized with bound ligand. Crystals are formed from PPAR LBD polypeptides that are usually expressed by a cell culture, such as E. coli. Bromo-, iodo- and substitutions can be included during the preparation of crystal forms and can act as heavy atom substitutions in PPAR ligands and crystals of PPARs. This method can be advantageous for the phasing of the crystal, which is a crucial, and sometimes limiting, step in solving the three-dimensional structure of a crystallized entity. Thus, the need for generating the heavy metal derivatives traditionally employed in crystallography can be eliminated. After the three-dimensional structure of a PPAR or PPAR LBD with or without a ligand bound is determined, the resultant three-dimensional structure can be used in computational methods to design synthetic ligands for PPARα and other PPAR polypeptides. Further activity structure relationships can be determined through routine testing, using assays disclosed herein and known in the art.

VIII. Uses of PPARα Crystals and the Three-Dimensional Structure of the Ligand Binding Domain of PPARα

VIII.A. Design and Development of PPAR Modulators

The knowledge of the structure of the PPARα ligand binding domain (LBD), an aspect of the present invention, provides a tool for investigating the mechanism of action of PPARα and other PPAR polypeptides in a subject. For example, various computer modelleing programs, as described herein, can predict the binding of various ligand molecules to the LBD of PPARα, PPARγ or PPARδ. Upon discovering that such binding in fact takes place, knowledge of the protein structure then allows design and synthesis of small molecules that mimic the functional binding of the ligand to the LBD of PPARα, and to the LBDs of other PPAR polypeptides. This is the method of “rational” drug design, further described herein.

Use of the isolated and purified PPARα crystalline structure of the present invention in rational drug design is thus provided in accordance with the present invention. Additional rational drug design techniques are described in U.S. Pat. Nos. 5,834,228 and 5,872,011, incorporated herein in their entirety.

Thus, in addition to the compounds described herein, other sterically similar compounds can be formulated to interact with the key structural regions of a PPAR in general, or of PPARα in particular. The generation of a structural functional equivalent can be achieved by the techniques of modeling and chemical design known to those of skill in the art and described herein. It will be understood that all such sterically similar constructs fall within the scope of the present invention.

VIII.A.1. Rational Drug Design

The three-dimensional structure of ligand-binding PPARα is unprecedented and will greatly aid in the development of new synthetic ligands for a PPAR polypeptide, such as PPAR agonists and antagonists, including those that bind exclusively to any one of the PPAR subtypes. In addition, the PPARs are well suited to modem methods, including three-dimensional structure elucidation and combinatorial chemistry, such as those disclosed in U.S. Pat. No. 5,463,564, incorporated herein by reference. Structure determination using X-ray crystallography is possible because of the solubility properties of the PPARs. Computer programs that use crystallography data when practicing the present invention will enable the rational design of ligands to these receptors. Programs such as RASMOL (Biomolecular Structures Group, Glaxo Wellcome Research & Development Stevenage, Hertfordshire, UK Version 2.6, August 1995, Version 2.6.4, December 1998, Copyright© Roger Sayle 1992-1999) can be used with the atomic structural coordinates from crystals generated by practicing the invention or used to practice the invention by generating three-dimensional models and/or determining the structures involved in ligand binding. Computer programs such as those sold under the registered trademark INSIGHT II® and such as GRASP (Nicholls et al., (1991) Proteins 11: 282) allow for further manipulations and the ability to introduce new structures. In addition, high throughput binding and bioactivity assays can be devised using purified recombinant protein and modern reporter gene transcription assays known to those of skill in the art in order to refine the activity of a designed ligand.

A method of identifying modulators of the activity of a PPAR polypeptide using rational drug design is thus provided in accordance with the present invention. The method comprises designing a potential modulator for a PPAR polypeptide of the present invention that will form non-covalent interactions with amino acids in the ligand binding pocket based upon the crystalline structure of the PPARα LBD polypeptide; synthesizing the modulator; and determining whether the potential modulator modulates the activity of the PPAR polypeptide. In a preferred embodiment, the modulator is designed for a PPARα polypeptide. Preferably, the PPARα polypeptide comprises the nucleic acid sequence of SEQ ID NO:1, and the PPARα LBD comprises the nucleic acid sequence SEQ ID NO:3. The determination of whether the modulator modulates the biological activity of a PPAR polypeptide is made in accordance with the screening methods disclosed herein, or by other screening methods known to those of skill in the art. Modulators can be synthesized using techniques known to those of ordinary skill in the art.

In an alternative embodiment, a method of designing a modulator of a PPAR polypeptide in accordance with the present invention is disclosed comprising: (a) selecting a candidate PPAR ligand; (b) determining which amino acid or amino acids of an PPAR polypeptide interact with the ligand using a three-dimensional model of a crystallized PPARα LBD; (c) identifying in a biological assay for PPAR activity a degree to which the ligand modulates the activity of the PPAR polypeptide; (d) selecting a chemical modification of the ligand wherein the interaction between the amino acids of the PPAR polypeptide and the ligand is predicted to be modulated by the chemical modification; (e) performing the chemical modification on the ligand to form a modified ligand; (f) contacting the modified ligand with the PPAR polypeptide; (g) identifying in a biological assay for PPAR activity a degree to which the modified ligand modulates the biological activity of the PPAR polypeptide; and (h) comparing the biological activity of the PPAR polypeptide in the presence of modified ligand with the biological activity of the PPAR polypeptide in the presence of the unmodified ligand, whereby a modulator of an PPAR polypeptide is designed.

VIII.A.2. Methods for Using the PPARα LBD Structural Coordinates For Molecular Design

For the first time, the present invention permits the use of molecular design techniques to design, select and synthesize chemical entities and compounds, including modulatory compounds, capable of binding to the ligand binding pocket or an accessory binding site of PPARα and the PPARα LBD, in whole or in part. Correspondingly, the present invention also provides for the application of similar techniques in the design of modulators of any PPAR polypeptide.

In accordance with a preferred embodiment of the present invention, the structure coordinates of a crystalline PPARα LBD can be used to design compounds that bind to a PPAR LBD (more preferably a PPARα LBD) and alter the properties of a PPAR LBD (for example, the dimerization or ligand binding ability) in different ways. One aspect of the present invention provides for the design of compounds that can compete with natural or engineered ligands of a PPAR polypeptide by binding to all, or a portion of, the binding sites on a PPAR LBD. The present invention also provides for the design of compounds that can bind to all, or a portion of, an accessory binding site on a PPAR that is already binding a ligand. Similarly, non-competitive agonists/ligands that bind to and modulate PPAR LBD activity, whether or not it is bound to another chemical entity, can be designed using the PPAR LBD structure coordinates of this invention.

A second design approach is to probe a PPAR or PPAR LBD (preferably a PPARα or PPARα LBD) crystal with molecules comprising a variety of different chemical entities to determine optimal sites for interaction between candidate PPAR or PPAR LBD modulators and the polypeptide. For example, high resolution X-ray diffraction data collected from crystals saturated with solvent allows the determination of the site where each type of solvent molecule adheres. Small molecules that bind tightly to those sites can then be designed and synthesized and tested for their PPARα modulator activity. Representative designs are also disclosed in published PCT application WO 99/26966.

Once a computationally-designed ligand is synthesized using the methods of the present invention or other methods known to those of skill in the art, assays can be used to establish its efficacy of the ligand as a modulator of PPAR (preferably PPARα) activity. After such assays, the ligands can be further refined by generating intact PPAR, or PPAR LBD, crystals with a ligand bound to the LBD. The structure of the ligand can then be further refined using the chemical modification methods described herein and known to those of skill in the art, in order to improve the modulation activity or the binding affinity of the ligand. This process can lead to second generation ligands with improved properties.

Ligands also can be selected that modulate PPAR responsive gene transcription by the method of altering the interaction of co-activators and co-repressors with their cognate PPAR. For example, agonistic ligands can be selected that block or dissociate a co-repressor from interacting with the PPAR, and/or that promote binding or association of a co-activator. Antagonistic ligands can be selected that block co-activator interaction and/or promote co-repressor interaction with a target receptor. Selection can be done via binding assays that screen for designed ligands having the desired modulatory properties. Preferably, interactions of a PPARα polypeptide are targeted. Suitable assays for screening that can be employed, mutatis mutandis in the present invention, are described in as described in Nichols et al., (1998) Anal. Biochem. 257: 112-19 and Xu et al., (1999) Mol. Cell 3: 397-403, which are incorporated herein in their entirety by reference.

VIII.A.3. Methods of Designing PPARα LBD Modulator Compounds

The design of candidate substances, also referred to as “compounds” or “candidate compounds”, that bind to or inhibit PPAR LBD-mediated activity according to the present invention generally involves consideration of two factors. First, the compound must be capable of physically and structurally associating with a PPAR LBD. Non-covalent molecular interactions important in the association of a PPAR LBD with its substrate include hydrogen bonding, van der Waals interactions and hydrophobic interactions.

Second, the compound must be able to assume a conformation that allows it to associate with a PPAR LBD. Although certain portions of the compound will not directly participate in this association with a PPAR LBD, those portions can still influence the overall conformation of the molecule. This, in turn, can have a significant impact on potency. Such conformational requirements include the overall three-dimensional structure and orientation of the chemical entity or compound in relation to all or a portion of the binding site, e.g., the ligand binding pocket or an accessory binding site of a PPAR LBD, or the spacing between functional groups of a compound comprising several chemical entities that directly interact with a PPAR LBD.

The potential modulatory or binding effect of a chemical compound on a PPAR LBD can be analyzed prior to its actual synthesis and testing by the use of computer modeling techniques that employ the coordinates of a crystalline PPARα LBD polypeptide of the present invention. If the theoretical structure of the given compound suggests insufficient interaction and association between it and a PPAR LBD, synthesis and testing of the compound is obviated. However, if computer modeling indicates a strong interaction, the molecule can then be synthesized and tested for its ability to bind and modulate the activity of a PPAR LBD. In this manner, synthesis of unproductive or inoperative compounds can be avoided.

A modulatory or other binding compound of a PPAR LBD polypeptide (preferably a PPARα LBD) can be computationally evaluated and designed via a series of steps in which chemical entities or fragments are screened and selected for their ability to associate with the individual binding sites or other areas of a crystalline PPARαLBD polypeptide of the present invention.

One of several methods can be used to screen chemical entities or fragments for their ability to associate with a PPAR LBD and, more particularly, with the individual binding sites of a PPAR LBD, such as ligand binding pocket or an accessory binding site. This process can begin by visual inspection of, for example, the ligand binding pocket on a computer screen based on the PPARα LBD atomic coordinates in Table 2. Selected fragments or chemical entities can then be positioned in a variety of orientations, or docked, within an individual binding site of a PPARα LBD as defined herein above. Docking can be accomplished using software programs such as those available under the tradenames QUANTA™ (Molecular Simulations Inc., San Diego, Calif.) and SYBYL™ (Tripos, Inc., St. Louis, Miss.), followed by energy minimization and molecular dynamics with standard molecular mechanics forcefields, such as CHARM (Brooks et al., (1983) J. Comp. Chem., 8: 132) and AMBER 5 (Case et al., (1997), AMBER 5, University of California, San Francisco; Pearlman et al., (1995) Comput. Phys. Commun. 91: 1-41).

Specialized computer programs can also assist in the process of selecting fragments or chemical entities. These include:

1. GRID™ program, version 17 (Goodford, (1985) J. Med. Chem. 28: 849-57), which is available from Molecular Discovery Ltd., Oxford, UK;

2. MCSS™ program (Miranker & Karplus, (1991) Proteins 11: 29-34), which is available from Molecular Simulations, Inc., San Diego, Calif.;

3. AUTODOCK™ 3.0 program (Goodsell & Olsen, (1990) Proteins 8: 195-202), which is available from the Scripps Research Institute, La Jolla, Calif.;

4. DOCK™ 4.0 program (Kuntz et al., (1992) J. Mol. Biol. 161: 269-88), which is available from the University of California, San Francisco, Calif.;

5. FLEX-X™ program (See, Rarey et al., (1996) J. Comput. Aid. Mol. Des. 10:41-54), which is available from Tripos, Inc., St. Louis, Miss.;

6. MVP program (Lambert, (1997) in Practical Application of Computer-Aided Drug Design, (Charifson, ed.) Marcel-Dekker, New York, pp. 243-303); and

7. LUDI™ program (Bohm, (1992) J. Comput. Aid. Mol. Des., 6: 61-78), which is available from Molecular Simulations, Inc., San Diego, Calif.

Once suitable chemical entities or fragments have been selected, they can be assembled into a single compound or modulator. Assembly can proceed by visual inspection of the relationship of the fragments to each other on the three-dimensional image displayed on a computer screen in relation to the structure coordinates of a PPARα LBD. Manual model building using software such as QUANTA™ or SYBYL™ typically follows.

Useful programs to aid one of ordinary skill in the art in connecting the individual chemical entities or fragments include:

1. CAVEAT™ program (Bartlett et al., (1989) Special Pub:, Royal Chem. Soc. 78: 182-96), which is available from the University of California, Berkeley, Calif.;

2. 3D Database systems, such as MACCS-3D™ system program, which is available from MDL Information Systems, San Leandro, Calif. This area is reviewed in Martin, (1992) J. Med. Chem. 35: 2145-54; and

3. HOOK™ program (Eisen et al., (1994). Proteins 19: 199-221), which is available from Molecular Simulations, Inc., San Diego, Calif.

Instead of proceeding to build a PPAR LBD modulator (preferably a PPARα LBD modulator) in a step-wise fashion one fragment or chemical entity at a time as described above, modulatory or other binding compounds can be designed as a whole or de novo using the structural coordinates of a crystalline PPARα LBD polypeptide of the present invention and either an empty binding site or optionally including some portion(s) of a known modulator(s). Applicable methods can employ the following software programs:

1. LUDI™ program (Bohm, (1992) J. Comput. Aid. Mol. Des., 6: 61-78), which is available from Molecular Simulations, Inc., San Diego, Calif.;

2. LEGEND™ program (Nishibata & Itai, (1991) Tetrahedron 47: 8985); and

3. LEAPFROG™, which is available from Tripos Associates, St. Louis, Miss.

Other molecular modeling techniques can also be employed in accordance with this invention. See, e.g., Cohen et al., (1990) J. Med. Chem. 33: 883-94. See also, Navia & Murcko, (1992) Curr. Opin. Struc. Biol. 2: 202-10; U.S. Pat. No. 6,008,033, herein incorporated by reference.

Once a compound has been designed or selected by the above methods, the efficiency with which that compound can bind to a PPARα LBD can be tested and optimized by computational evaluation. By way of particular example, a compound that has been designed or selected to function as a PPARα LBD modulator should also preferably traverse a volume not overlapping that occupied by the binding site when it is bound to its native ligand. Additionally, an effective PPAR LBD modulator should preferably demonstrate a relatively small difference in energy between its bound and free states (i.e., a small deformation energy of binding). Thus, the most efficient PPAR LBD modulators should preferably be designed with a deformation energy of binding of not greater than about 10 kcal/mole, and preferably, not greater than 7 kcal/mole. It is possible for PPAR LBD modulators to interact with the polypeptide in more than one conformation that is similar in overall binding energy. In those cases, the deformation energy of binding is taken to be the difference between the energy of the free compound and the average energy of the conformations observed when the modulator binds to the polypeptide.

A compound designed or selected as binding to a PPAR polypeptide (preferably a PPARα LBD polypeptide) can be further computationally optimized so that in its bound state it would preferably lack repulsive electrostatic interaction with the target polypeptide. Such non-complementary (e.g., electrostatic) interactions include repulsive charge-charge, dipole-dipole and charge-dipole interactions. Specifically, the sum of all electrostatic interactions between the modulator and the polypeptide when the modulator is bound to a PPAR LBD preferably make a neutral or favorable contribution to the enthalpy of binding.

Specific computer software is available in the art to evaluate compound deformation energy and electrostatic interaction. Examples of programs designed for such uses include:

1. Gaussian 98™, which is available from Gaussian, Inc., Pittsburgh, Pa.;

2. AMBER™ program, version 6.0, which is available from the University of California at San Francisco;

3. QUANTA™ program, which is available from Molecular Simulations, Inc., San Diego, Calif.;

4. CHARM® program, which is available from Molecular Simulations, Inc., San Diego, Calif.; and

4. Insight II® program, which is available from Molecular Simulations, Inc., San Diego, Calif.

These programs can be implemented using a suitable computer system. Other hardware systems and software packages will be apparent to those skilled in the art after review of the disclosure of the present invention presented herein.

Once a PPAR LBD modulating compound has been optimally selected or designed, as described above, substitutions can then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds can then be analyzed for efficiency of fit to a PPAR LBD binding site using the same computer-based approaches described in detail above.

VIII.B. Distinguishing Between PPAR Subtypes

The present invention discloses the ability to generate new synthetic ligands to distinguish between PPAR subtypes. As described herein, computer-designed ligands can be generated that distinguish between PPAR subtypes, thereby allowing the generation of either tissue specific or function specific ligands. The atomic structural coordinates disclosed in the present invention reveal structural details unique to PPARα. These structural details can be exploited when a novel ligand is designed using the methods of the present invention or other ligand design methods known in the art. The structural features that differentiate, for example, a PPARα from a PPARγ can be targeted in ligand design. Thus, for example, a ligand can be designed that will recognize PPARα, while not interacting with other PPARs or even with moieties having similar structural features. Prior to the disclosure of the present invention, the ability to target a PPAR subtype was unattainable.

The present invention also pertains to a method for designing an agonist or modulator with desired levels of activity on the three subtypes, PPARα, PPARγ and PPARδ. In a preferred embodiment, the method comprises obtaining atomic coordinates for structures of the PPARα, PPARγ and/or PPARδ ligand binding domains. The structures can comprise PPARα, PPARγ and PPARδ each bound to various different ligands, and also can comprise structures where no ligand is present. The structures can also comprise models where a compound has been docked into a particular PPAR using a molecular docking procedure, such as the MVP program disclosed herein. Optionally, the structures comprise rotated and translated so as to superimpose corresponding Cα or backbone atoms; this facilitates the comparison of structures.

The PPARα, PPARγ and PPARδ structures can also be compared using a computer graphics system to identify regions of the ligand binding site that have similar shape and electrostatic character, and to identify regions of the ligand binding site that are narrowed or constricted in one or two of the PPARs compared with the other(s). Since these three PPARs are subject to conformational changes, attention is paid to the range of motion observed for each protein atom over the whole collection of structures. The ligand structures, including both those determined by X-ray crystallography and those modeled using molecular docking procedures, can be examined using a computer graphics system to identify ligands where a chemical modification could increase or decrease binding to a particular PPAR, or decrease activity against a particular PPAR. Additionally or alternatively, the chemical modification can introduce a group into a volume that is normally occupied by an atom of that PPAR.

Optionally, to selectively decrease activity against a particular PPAR, the chemical modification can be made so as to occupy volume that is normally occupied by atoms of that particular PPAR, but not by atoms of the other PPARs. To increase activity against a particular PPAR, a chemical modification can be made that improves interactions with that particular PPAR. To selectively increase activity against a particular PPAR, a chemical modification can be made that improves the interactions with that particular PPAR, but does not improve the interactions with the other PPARs. Other design principles can also be used to increase or decrease activity on a particular PPAR.

Thus, various possible compounds and chemical modifications can be considered and compared graphically, and with molecular modeling tools, for synthetic feasibility and likelihood of achieving the desired profile of activation of PPARα, PPARγ and PPARδ. Compounds that appear synthetically feasible and that have a good likelihood of achieving the desired profile are synthesized. The compounds can then be tested for binding and/or activation of PPARα, PPARγ and PPARδ, and tested for their overall biological effect.

VIII.C. Method of Screening for Chemical and Biological Modulators of the Biological Activity of PPARα

A candidate substance identified according to a screening assay of the present invention has an ability to modulate the biological activity of a PPAR or a PPAR LBD polypeptide. In a preferred embodiment, such a candidate compound can have utility in the treatment of disorders and conditions associated with the biological activity of a PPARα or a PPARα LBD polypeptide, including lipid homeostasis.

In a cell-free system, the method comprises the steps of establishing a control system comprising a crystalline PPARα polypeptide and a ligand which is capable of binding to the polypeptide; establishing a test system comprising a crystalline PPARα polypeptide, the ligand, and a candidate compound; and determining whether the candidate compound modulates the activity of the polypeptide by comparison of the test and control systems. A representative ligand comprises Compound 1 or other small molecule, and in this embodiment, the biological activity or property screened includes binding affinity.

In another embodiment of the invention, a crystalline form of a PPARα polypeptide or a catalytic or immunogenic fragment or oligopeptide thereof, can be used for screening libraries of compounds in any of a variety of drug screening techniques. The fragment employed in such a screening can be affixed to a solid support. The formation of binding complexes, between a crystalline PPARα polypeptide and the agent being tested, will be detected. In a preferred embodiment, the crystalline PPARα polypeptide has an amino acid sequence of SEQ ID NO:2. When a PPARα LBD polypeptide is employed, a preferred embodiment will include a crystalline PPARα polypeptide having the amino acid sequence of SEQ ID NO:4.

Another technique for drug screening which can be used provides for high throughput screening of compounds having suitable binding affinity to the protein of interest as described in published PCT application WO 84/03564, herein incorporated by reference. In this method, as applied to a crystalline polypeptide of the present invention, large numbers of different small test compounds are synthesized on a solid substrate, such as plastic pins or some other surface. The test compounds are reacted with the crystalline polypeptide, or fragments thereof. Bound polypeptide is then detected by methods well known to those of skill in the art. The crystalline polypeptide can also be placed directly onto plates for use in the aforementioned drug screening techniques.

In yet another embodiment, a method of screening for a modulator of a PPARα or a PPARα LBD polypeptide comprises: providing a library of test samples; contacting a crystalline form of PPARα or a crystalline form of an PPARα LBD polypeptide with each test sample; detecting an interaction between a test sample and a crystalline form of a PPARα or a crystalline form of a PPARα LBD polypeptide; identifying a test sample that interacts with a crystalline form of a PPARα or a crystalline form of a PPARα LBD polypeptide; and isolating a test sample that interacts with a crystalline form of a PPARα or a crystalline form of a PPARα LBD polypeptide.

In each of the foregoing embodiments, an interaction can be detected spectrophotometrically, radiologically or immunologically. An interaction between a crystalline form of a PPARα or a crystalline form of a PPARα LBD polypeptide and a test sample can also be quantified using methodology known to those of skill in the art.

In accordance with the present invention there is also provided a rapid and high throughput screening method that relies on the methods described above. This screening method comprises separately contacting each of a plurality of substantially identical samples with crystalline form of a PPARα or a crystalline form of a PPARα LBD and detecting a resulting binding complex. In such a screening method the plurality of samples preferably comprises more than about 10⁴ samples, or more preferably comprises more than about 5×10⁴ samples.

VIII.D. Method of Identifying Compounds which Inhibit Ligand Binding

In one aspect of the present invention, an assay method for identifying a compound that inhibits binding of a ligand to a PPAR polypeptide is disclosed. A ligand of PPARα, such as Compound 1, can be used in the assay method as the ligand against which the inhibition by a test compound is gauged. The method comprises (a) incubating a PPAR polypeptide with a ligand in the presence of a test inhibitor compound; (b) determining an amount of ligand that is bound to the PPAR polypeptide, wherein decreased binding of ligand to the PPAR polypeptide in the presence of the test inhibitor compound relative to binding in the absence of the test inhibitor compound is indicative of inhibition; and (c) identifying the test compound as an inhibitor of ligand binding if decreased ligand binding is observed. Preferably, the ligand is Compound 1.

In another aspect of the present invention, the disclosed assay method can be used in the structural refinement of candidate PPAR inhibitors. For example, multiple rounds of optimization can be followed by gradual structural changes in a strategy of inhibitor design. A strategy such as this is made possible by the disclosure of the coordinates of the PPARα LBD.

IX. Design, Preparation and Structural Analysis of Additional PPARα and PPARα LBD Mutants and Structural Equivalents

The present invention provides for the generation of PPAR and PPAR mutants (preferably PPARα and PPARα LBD mutants), and the ability to solve the crystal structures of those that crystallize. More particularly, through the provision of the three-dimensional structure of a PPARα LBD, desirable sites for mutation can be identified.

The structure coordinates of a PPARα LBD provided in accordance with the present invention also facilitate the identification of related proteins or enzymes analogous to PPARα in function, structure or both, (for example, a PPARγ) which can lead to novel therapeutic modes for treating or preventing a range of disease states.

IX.A. Sterically Similar Compounds

A further aspect of the present invention is that sterically similar compounds can be formulated to mimic the key portions of a PPAR LBD structure. Such compounds are functional equivalents. The generation of a structural functional equivalent can be achieved by the techniques of modeling and chemical design known to those of skill in the art and described herein. Modeling and chemical design of PPAR and PPAR LBD structural equivalents can be based on the structure coordinates of a crystalline PPARα LBD polypeptide of the present invention. It will be understood that all such sterically similar constructs fall within the scope of the present invention.

IX.B. PPARα Polypeptides

The generation of chimeric PPAR polypeptides is also an aspect of the present invention. Such a chimeric polypeptide can comprise a PPAR LBD polypeptide or a portion of a PPAR LBD, (e.g. a PPARα LBD) that is fused to a candidate polypeptide or a suitable region of the candidate polypeptide, for example PPARγ. Throughout the present disclosure it is intended that the term “mutant” encompass not only mutants of a PPAR LBD polypeptide but chimeric proteins generated using a PPAR LBD as well. It is thus intended that the following discussion of mutant PPAR LBDs apply mutatis mutandis to chimeric PPAR and PPAR LBD polypeptides and to structural equivalents thereof.

In accordance with the present invention, a mutation can be directed to a particular site or combination of sites of a wild-type PPAR LBD. For example, an accessory binding site or the binding pocket can be chosen for mutagenesis. Similarly, a residue having a location on, at or near the surface of the polypeptide can be replaced, resulting in an altered surface charge of one or more charge units, as compared to the wild-type PPAR and PPAR LBD. Alternatively, an amino acid residue in a PPAR or a PPAR LBD can be chosen for replacement based on its hydrophilic or hydrophobic characteristics.

Such mutants can be characterized by any one of several different properties as compared with the wild-type PPAR LBD. For example, such mutants can have an altered surface charge of one or more charge units, or can have an increase in overall stability. Other mutants can have altered substrate specificity in comparison with, or a higher specific activity than, a wild-type PPAR or PPAR LBD.

PPAR and PPAR LBD mutants of the present invention can be generated in a number of ways. For example, the wild-type sequence of a PPAR or a PPAR LBD can be mutated at those sites identified using this invention as desirable for mutation, by means of oligonucleotide-directed mutagenesis or other conventional methods, such as deletion. Alternatively, mutants of a PPAR or a PPAR LBD can be generated by the site-specific replacement of a particular amino acid with an unnaturally occurring amino acid. In addition, PPAR or PPAR LBD mutants can be generated through replacement of an amino acid residue, for example, a particular cysteine or methionine residue, with selenocysteine or selenomethionine. This can be achieved by growing a host organism capable of expressing either the wild-type or mutant polypeptide on a growth medium depleted of either natural cysteine or methionine (or both) but enriched in selenocysteine or selenomethionine (or both).

Mutations can be introduced into a DNA sequence coding for a PPAR or a PPAR LBD using synthetic oligonucleotides. These oligonucleotides contain nucleotide sequences flanking the desired mutation sites. Mutations can be generated in the full-length DNA sequence of a PPAR or a PPAR LBD or in any sequence coding for polypeptide fragments of a PPAR or a PPAR LBD.

According to the present invention, a mutated PPAR or PPAR LBD DNA sequence produced by the methods described above, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired PPAR or PPAR LBD mutant coding sequence, an expression vector also will include control sequences encoding a promoter, operator, ribosome binding site, translation initiation signal, and, optionally, a repressor gene or various activator genes and a signal for termination. In some embodiments, where secretion of the produced mutant is desired, nucleotides encoding a “signal sequence” can be inserted prior to a PPAR or a PPAR LBD mutant coding sequence. For expression under the direction of the control sequences, a desired DNA sequence must be operatively linked to the control sequences; that is, the sequence must have an appropriate start signal in front of the DNA sequence encoding the PPAR or PPAR LBD mutant, and the correct reading frame to permit expression of that sequence under the control of the control sequences and production of the desired product encoded by that PPAR or PPAR LBD sequence must be maintained.

Any of a wide variety of well-known available expression vectors can be useful to express a mutated PPAR or PPAR LBD coding sequences of this invention. These include for example, vectors consisting of segments of chromosomal, non-chromosomal and synthetic DNA sequences, such as various known derivatives of SV40, known bacterial plasmids, e.g., plasmids from E. coli including col E1, pCR1, pBR322, pMB9 and their derivatives, wider host range plasmids, e.g., RP4, phage DNAs, e.g., the numerous derivatives of phage λ, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences. In the preferred embodiments of this invention, vectors amenable to expression in a pRSETA-based expression system are employed. The pRSETA expression system is available from Invitrogen, Inc., Carlsbad, Calif.

In addition, any of a wide variety of expression control sequences—sequences that control the expression of a DNA sequence when operatively linked to it—can be used in these vectors to express the mutated DNA sequences according to this invention. Such useful expression control sequences, include, for example, the early and late promoters of SV40 for animal cells, the lac system, the trp system the TAC or TRC system, the major operator and promoter regions of phage λ, the control regions of fd coat protein, all for E. coli, the promoter for 3-phosphoglycerate kinase or other glycolytic enzymes, the promoters of acid phosphatase, e.g., Pho5, the promoters of the yeast α-mating factors for yeast, and other sequences known to control the expression of genes of prokaryotic or eukaryotic cells or their viruses, and various combinations thereof.

A wide variety of hosts are also useful for producing mutated PPARα and PPARα LBD polypeptides according to this invention. These hosts include, for example, bacteria, such as E. coli, Bacillus and Streptomyces, fungi, such as yeasts, and animal cells, such as CHO and COS-1 cells, plant cells, insect cells, such as Sf9 cells, and transgenic host cells.

It should be understood that not all expression vectors and expression systems function in the same way to express mutated DNA sequences of this invention, and to produce modified PPAR and PPAR LBD polypeptides or PPAR or PPAR LBD mutants. Neither do all hosts function equally well with the same expression system. One of skill in the art can, however, make a selection among these vectors, expression control sequences and hosts without undue experimentation and without departing from the scope of this invention. For example, an important consideration in selecting a vector will be the ability of the vector to replicate in a given host. The copy number of the vector, the ability to control that copy number, and the expression of any other proteins encoded by the vector, such as antibiotic markers, should also be considered.

In selecting an expression control sequence, a variety of factors should also be considered. These include, for example, the relative strength of the system, its controllability and its compatibility with the DNA sequence encoding a modified PPAR or PPAR LBD polypeptide of this invention, with particular regard to the formation of potential secondary and tertiary structures.

Hosts should be selected by consideration of their compatibility with the chosen vector, the toxicity of a modified PPAR or PPAR LBD to them, their ability to express mature products, their ability to fold proteins correctly, their fermentation requirements, the ease of purification of a modified PPAR or PPAR LBD and safety. Within these parameters, one of skill in the art can select various vector/expression control system/host combinations that will produce useful amounts of a mutant PPAR or PPAR LBD. A mutant PPAR or PPAR LBD produced in these systems can be purified by a variety of conventional steps and strategies, including those used to purify the wild-type PPAR or PPAR LBD.

Once a PPAR LBD mutation(s) has been generated in the desired location, such as an active site or dimerization site, the mutants can be tested for any one of several properties of interest. For example, mutants can be screened for an altered charge at physiological pH. This is determined by measuring the mutant PPAR or PPAR LBD isoelectric point (pl) and comparing the observed value with that of the wild-type parent. Isoelectric point can be measured by gel-electrophoresis according to the method of Wellner (Wellner, (1971) Anal. Chem. 43: 597). A mutant PPAR or PPAR LBD polypeptide containing a replacement amino acid located at the surface of the enzyme, as provided by the structural information of this invention, can lead to an altered surface charge and an altered pl.

IX.C. Generation of an Engineered PPARα LBD or PPARα LBD Mutant

In another aspect of the present invention, a unique PPAR or PPAR LBD polypeptide can be generated. Such a mutant can facilitate purification and the study of the ligand-binding abilities of a PPAR polypeptide.

As used in the following discussion, the terms “engineered PPAR”, “engineered PPAR LDB”, “PPAR mutant”, and “PPAR LBD mutant” refers to polypeptides having amino acid sequences which contain at least one mutation in the wild-type sequence. The terms also refer to PPAR and PPAR LBD polypeptides which are capable of exerting a biological effect in that they comprise all or a part of the amino acid sequence of an engineered PPAR or PPAR LBD mutant polypeptide of the present invention, or cross-react with antibodies raised against an engineered PPAR or PPAR LBD mutant polypeptide, or retain all or some or an enhanced degree of the biological activity of the engineered PPAR or PPAR LBD mutant amino acid sequence or protein. Such biological activity can include the binding of small molecules in general, and the binding of Compound 1 in particular.

The terms “engineered PPAR LBD” and “PPAR LBD mutant” also includes analogs of an engineered PPAR LBD or PPAR LBD mutant polypeptide. By uanalog is intended that a DNA or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct PPAR LBD or PPAR LBD mutant analogs. There is no need for an engineered PPAR LBD or PPAR LBD mutant polypeptide to comprise all or substantially all of the amino acid sequence of SEQ ID NOs:2 or 4. Shorter or longer sequences are anticipated to be of use in the invention; shorter sequences are herein referred to as “segments”. Thus, the terms “engineered PPAR LBD” and “PPAR LBD mutant” also includes fusion, chimeric or recombinant engineered PPAR LBD or PPAR LBD mutant polypeptides and proteins comprising sequences of the present invention. Methods of preparing such proteins are disclosed herein above and are known in the art.

IX.D. Sequence Similarity and Identity

As used herein, the term “substantially similar” means that a particular sequence varies from nucleic acid sequence of SEQ ID NOs:1 or 3, or the amino acid sequence of SEQ ID NOs:2 or 4 by one or more deletions, substitutions, or additions, the net effect of which is to retain at least some of biological activity of the natural gene, gene product, or sequence. Such sequences include “mutant” or “polymorphic” sequences, or sequences in which the biological activity and/or the physical properties are altered to some degree but retains at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

IX.D.1. Sequences that are Substantially Identical to an Engineered PPAR or PPAR LBD Mutant Sequence of the Present Invention

Nucleic acids that are substantially identical to a nucleic acid sequence of an engineered PPAR or PPAR LBD mutant of the present invention, e.g. allelic variants, genetically altered versions of the gene, etc., bind to an engineered PPAR or PPAR LBD mutant sequence under stringent hybridization conditions. By using probes, particularly labeled probes of DNA sequences, one can isolate homologous or related genes. The source of homologous genes can be any species, e.g. primate species; rodents, such as rats and mice, canines, felines, bovines, equines, yeast, nematodes, etc.

Between mammalian species, e.g. human and mouse, homologs have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. A reference sequence will usually be at least about 18 nt long, more usually at least about 30 nt long, and can extend to the complete sequence that is being compared. Algorithms for sequence analysis are known in the art, such as BLAST, described in Altschul et al., (1990) J. Mol. Biol. 215: 403-10.

Percent identity or percent similarity of a DNA or peptide sequence can be determined, for example, by comparing sequence information using the GAP computer program, available from the University of Wisconsin Geneticist Computer Group. The GAP program utilizes the alignment method of Needleman et al., (1970) J. Mol. Biol. 48: 443, as revised by Smith et al., (1981) Adv. Appl. Math. 2:482. Briefly, the GAP program defines similarity as the number of aligned symbols (i.e., nucleotides or amino acids) which are similar, divided by the total number of symbols in the shorter of the two sequences. The preferred parameters for the GAP program are the default parameters, which do not impose a penalty for end gaps. See, e.g., Schwartz et al., eds., (1979), Atlas of Protein Sequence and Structure, National Biomedical Research Foundation, pp. 357-358, and Gribskov et al., (1986) Nucl. Acids. Res. 14: 6745.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NOs:1 or 3; or (b) the DNA analog sequence is capable of hybridization with DNA sequences of (a) under stringent conditions and which encode a biologically active PPARα or PPARα LBD gene product; or (c) the DNA sequences are degenerate as a result of alternative genetic code to the DNA analog sequences defined in (a) and/or (b). Substantially identical analog proteins and nucleic acids will have between about 70% and 80%, preferably between about 81% to about 90% or even more preferably between about 91% and 99% sequence identity with the corresponding sequence of the native protein or nucleic acid. Sequences having lesser degrees of identity but comparable biological activity are considered to be equivalents.

As used herein, “stringent conditions” means conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. For the purposes of specifying additional conditions of high stringency, preferred conditions are salt concentration of about 200 mM and temperature of about 45° C. One example of such stringent conditions is hybridization at 4×SSC, at 65° C., followed by a washing in 0.1×SSC at 65° C. for one hour. Another exemplary stringent hybridization scheme uses 50% formamide, 4×SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

IX.D.2. Complementarity and Hybridization to an Engineered PPARα or PPARα LBD Mutant Sequence

As used herein, the term “complementary sequences” means nucleic acid sequences which are base-paired according to the standard Watson-Crick complementarity rules. The present invention also encompasses the use of nucleotide segments that are complementary to the sequences of the present invention.

Hybridization can also be used for assessing complementary sequences and/or isolating complementary nucleotide sequences. As discussed above, nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and preferably in excess of about 45° C. Stringent salt conditions will ordinarily be less than about 1,000 mM, typically less than about 500 mM, and preferably less than about 200 mM. However, the combination of parameters is much more important than the measure of any single parameter. See, e.g., Wetmur & Davidson, (1968) J. Mol. Biol. 31: 349-70. Determining appropriate hybridization conditions to identify and/or isolate sequences containing high levels of homology is well known in the art. See, e.g., Sambrook et al., (1989) Molecular Cloning: A Laboratory Manual, Cold Spring Harbor, New York.

IX.D.3. Functional Equivalents of an Engineered PPARα or PPARα LBD Mutant Nucleic Acid Sequence of the Present Invention

As used herein, the term “functionally equivalent codon” is used to refer to codons that encode the same amino acid, such as the ACG and AGU codons for serine. PPARα or PPARα LBD-encoding nucleic acid sequences comprising SEQ ID NOs:1 and 3 which have functionally equivalent codons are covered by the present invention. Thus, when referring to the sequence example presented in SEQ ID NOs:1 and 3, applicants contemplate substitution of functionally equivalent codons into the sequence example of SEQ ID NOs:1 and 3. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N-or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

IX.D.4. Biological Equivalents

The present invention envisions and includes biological equivalents of a engineered PPAR or PPAR LBD mutant polypeptide of the present invention. The term “biological equivalent” refers to proteins having amino acid sequences which are substantially identical to the amino acid sequence of an engineered PPAR LBD mutant of the present invention and which are capable of exerting a biological effect in that they are capable of binding small molecules or cross-reacting with anti- PPAR or PPAR LBD mutant antibodies raised against an engineered mutant PPAR or PPAR LBD polypeptide of the present invention.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of an engineered PPAR or PPAR LBD mutant polypeptide of the present invention or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in SEQ ID NOs:1 and 3, applicants envision substitution of codons that encode biologically equivalent amino acids, as described herein, into the sequence example of SEQ ID NOs:2 and 4, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g. substitution of lie for Leu. Changes designed by man can be introduced through the application of site-directed mutagenesis techniques, e.g., to introduce improvements to the antigenicity of the protein or to test an engineered PPAR or PPAR LBD mutant polypeptide of the present invention in order to modulate lipid-binding or other activity, at the molecular level.

Amino acid substitutions, such as those which might be employed in modifying an engineered PPAR or PPAR LBD mutant polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of skill in the art. It is implicit in the above discussion, however, that one of skill in the art can appreciate that a radical, rather than a conservative substitution is warranted in a given situation. Non-conservative substitutions in engineered mutant PPAR or PPAR LBD polypeptides of the present invention are also an aspect of the present invention.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, (1982), J. Mol. Biol. 157: 105-132, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs:1-4. Recombinant vectors and isolated DNA segments can therefore variously include an engineered PPARα or PPARα LBD mutant polypeptide-encoding region itself, include coding regions bearing selected alterations or modifications in the basic coding region, or include larger polypeptides which nevertheless comprise an PPARα or PPARα LBD mutant polypeptide-encoding regions or can encode biologically functional equivalent proteins or polypeptides which have variant amino acid sequences. Biological activity of an engineered PPARα or PPARα LBD mutant polypeptide can be determined, for example, by lipid-binding assays known to those of skill in the art.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in SEQ ID NOs:1 and 3, such as about 10 nucleotides, and which are up to 10,000 or 5,000 base pairs in length. DNA segments with total lengths of about 4,000, 3,000, 2,000, 1,000, 500, 200, 100, and about 50 base pairs in length are also useful.

The DNA segments of the present invention encompass biologically functional equivalents of engineered PPAR or PPAR LBD mutant polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test variants of an engineered PPAR or PPAR LBD mutant of the present invention in order to examine the degree of lipid-binding activity, or other activity at the molecular level. Various site-directed mutagenesis techniques are known to those of skill in the art and can be employed in the present invention.

The invention further encompasses fusion proteins and peptides wherein an engineered PPAR or PPAR LBD mutant coding region of the present invention is aligned within the same expression unit with other proteins or peptides having desired functions, such as for purification or immunodetection purposes.

Recombinant vectors form important further aspects of the present invention. Particularly useful vectors are those in which the coding portion of the DNA segment is positioned under the control of a promoter. The promoter can be that naturally associated with a PPAR gene, as can be obtained by isolating the 5′ non-coding sequences located upstream of the coding segment or exon, for example, using recombinant cloning and/or PCR technology and/or other methods known in the art, in conjunction with the compositions disclosed herein.

In other embodiments, certain advantages will be gained by positioning the coding DNA segment under the control of a recombinant, or heterologous, promoter. As used herein, a recombinant or heterologous promoter is a promoter that is not normally associated with a PPAR gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (See, e.g., Sambrook et al., (1989) Molecular Cloning: A Laboratory Manual, Cold Spring Harbor Laboratory, New York, specifically incorporated herein by reference). The promoters employed can be constitutive or inducible and can be used under the appropriate conditions to direct high level expression of the introduced DNA segment, such as is advantageous in the large-scale production of recombinant proteins or peptides. One preferred promoter system contemplated for use in high-level expression is a T7 promoter-based system.

X. The Role of the Three-Dimensional Structure of the PPARα LDB in Solving Additional PPAR Crystals

Because polypeptides can crystallize in more than one crystal form, the structural coordinates of a PPARα LBD, or portions thereof, as provided by the present invention, are particularly useful in solving the structure of other crystal forms of PPARα and the crystalline forms of other PPARs. The coordinates provided in the present invention can also be used to solve the structure of PPAR or PPAR LBD mutants (such as those described in Section IX above), PPAR LDB co-complexes, or of the crystalline form of any other protein with significant amino acid sequence homology to any functional domain of PPAR.

X.A. Determining the Three-Dimensional Structure of a Polypeptide Using the Three-Dimensional Structure of the PPARα LBD as a Template in Molecular Replacement

One method that can be employed for the purpose of solving additional PPAR crystal structures is molecular replacement. See generally, Rossmann, ed, (1972) The Molecular Replacement Method, Gordon & Breach, New York. In the molecular replacement method, the unknown crystal structure, whether it is another crystal form of a PPARα or a PPARα LBD, (i.e. a PPARα or a PPARα LBD mutant), or a PPARα or a PPARα LBD polypeptide complexed with another compound (a “co-complex”), or the crystal of some other protein with significant amino acid sequence homology to any functional region of the PPARα LBD, can be determined using the PPARα LBD structure coordinates provided in Table 2. This method provides an accurate structural form for the unknown crystal more quickly and efficiently than attempting to determine such information ab initio.

In addition, in accordance with this invention, PPARα or PPARα LBD mutants can be crystallized in complex with known modulators. The crystal structures of a series of such complexes can then be solved by molecular replacement and compared with that of wild-type PPARα or the wild-type PPARα LBD. Potential sites for modification within the various binding sites of the enzyme can thus be identified. This information provides an additional tool for determining the most efficient binding interactions, for example, increased hydrophobic interactions, between the PPARα LBD and a chemical entity or compound.

All of the complexes referred to in the present disclosure can be studied using X-ray diffraction techniques (See, e.g., Blundell & Johnson (1985) Method.Enzymol., 114A & 115B, (Wyckoff et al., eds.), Academic Press) and can be refined using computer software, such as the X-PLOR™ program (Brünger, (1992) X-PLOR, Version 3.1. A System for X-ray Crystallography and NMR, Yale University Press, New Haven, Conn.; X-PLOR is available from Molecular Simulations, Inc., San Diego, Calif.). This information can thus be used to optimize known classes of PPAR and PPAR LBD modulators, and more importantly, to design and synthesize novel classes of PPAR and PPAR LBD modulators.

### LABORATORY EXAMPLES

The following Laboratory Examples have been included to illustrate preferred modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### Laboratory Example 1

### Protein Preparation

The nucleic acid sequence encoding the PPARα ligand binding domain (amino acids 192-468) tagged with MKKGHHHHHHG (SEQ ID NO: 9) was operatively linked to and expressed using the T7 promoter of plasmid vector pRSETA. BL21 (DE3) E. coli cells transformed with this expression vector were grown at 24° C. in shaker flasks for 66 hours on 2xYT medium (16 g/L Bacto-Tryptone, 10 g/L yeast extract, 5 g/L NaCl, QC with distilled water) with 50 mg/L carbenicillin to an OD₆₀₀ of approximately 9.0. The cells were harvested, resuspended with 20 ml extract Buffer (20 mM HEPES, pH 7.5, 50 mM imidozle, 250 mM NaCl and a pinch of lysozyme) per liter of cells and were lysed by sonication for 20 minutes on ice. The lysed cells were centrifuged at 40,000 g for 40 minutes and the supematant was loaded on a 100 ml Ni-agarose column.

The column was washed with 150 ml Buffer A (10% glycerol, 20 mM HEPES pH 7.5, 25 mM imidizole) and the protein was eluted with a 450 ml gradient of Buffer B (10% glycerol, 20 mM HEPES pH 7.5, 500 mM imidozle). The protein, which eluted at 20% Buffer B, was diluted with one volume of Buffer C (20 mM HEPES, pH 7.5, 1 mM EDTA), and loaded on an 100 ml S-Sepharose™ (Pharmacia, Peapack, N.J.) column. The column was washed with a 100 ml Buffer C and the PPARα LBD protein was eluted with a 200 ml gradient of Buffer D (20 mM HEPES, pH 7.5, 10 mM DTT, 1 M ammonium acetate). The PPAR alpha LBD eluted from the column at 43% Buffer D. The protein yield was 9 mg/L of cells grown and was >95% pure, as determined by SDS-PAGE analysis.

The protein was then diluted to 1 mg/ml with Buffer C such that the final buffer composition was 220 mM ammonium acetate, 20 mM HEPES pH 7.5, 1 mM EDTA and 1 mM DTT. The diluted protein was aliquoted into 9 ml aliquots, flash frozen with liquid nitrogen and stored at −80° C. To prepare complexes an individual aliquot was thawed for each compound. The peptide SRC1 (See, Xu et al., (1999) Mol. Cell 3: 397-403) was added in a mol ratio of 1.5 as a 2 mg/100 μl DMSO stock. The peptide was then added in a mol ratio of 5:1 as a 2 mg/100 μl DMSO stock and spun at 4K for 20 min to clarify the solution before concentrating in Centriprep™ 30 filtration units (Millipore, Bedford, Mass.). The solution containing the PPARα LBD-SRC1 complexes was concentrated to approximately 10 mg/ml with 80% yield. The complexes were then aliquoted in single use aliquots of 30 μl, flash frozen in liquid nitrogen and stored at −80° C.

### Laboratory Example 2

### Crystallization and Data Collection

The crystals disclosed in this invention were grown at room temperature using the hanging drop vapor diffusion method. The hanging drops comprised 1 μl of the above protein-ligand solutions, and were mixed with an equal volume (1 μl) of well buffer comprising 7% PEG 3350, 200 mM NaF, and 12% 2,5 hexanediol.

Before data collection, crystals were transiently mixed with well buffer that contained an additional 10% hexanediol as a cryoprotectant, and then flash frozen in liquid nitrogen.

The PPARα crystals formed in the P2₁2₁2 space group, with a=61.3 Å, b=103.5 Å, c=49.9 Å. Each asymmetry unit contained a single PPARα LBD with 45% solvent content. Crystals contained Compound 1, the PPARα LBD and SRC1 peptide, in a ratio of 1:1:1. Data were collected with a Rigaku R-Axis II (Rigaku, Tokyo, Japan) detector in house, or with a MAR CCD detector in the IMCA 17ID beam line at the Argonne National Laboratory (Argonne, Ill.), and the observed reflections were reduced, merged and scaled with DENZO™ and SCALEPACK™ in the HKL2000 package (Otwinowski, (1993) in Proceedings of the CCP4 Study Weekend: Data Collection and Processing. (Sawyer et al., eds.), pp.56-62, SERC Daresbury Laboratory, England).

### Laboratory Example 3

### Structure Determination and Refinement

The structure was determined by molecular replacement methods with the CCP4 AmoRe program (Collaborative Computational Project Number 4, 1994; Navaza, (1994) Acta. Cryst. A50: 157-163) using the structure coordinates for the PPARδ LBD (Xu et al., (1999) Mol. Cell 3: 397-403), residues 167-441, as the initial model (Table 3). The best fitting solution gave a correlation coefficiency of 70% and an R-factor of 33%. Model building was performed with the software program QUANTA™, and structure refinement was achieved using the CNS software program (Brünger et al., (1998) Acta. Crystallogr. D54: 905-921). Structure refinement involved multiple cycles of manual rebuilding. The final structure (Table 2 and FIGS. 1, 2, 4 and 7) includes one PPARα LBD, one SRC1 peptide and Compound 1. The statistics of the structures are summarized in Table 1.

### Laboratory Example 4

### Computational Analysis

Surface areas were calculated using both the Connolly MS program (Connolly, (1983) Science 221: 709-713) and the MVP program (Lambert, (1997) in Practical Application of Computer-Aided Drug Design, (Charifson, ed.), pp. 243-303, Marcel-Dekker, New York). The C2-symmetry axis, sequence alignments and binding site accessible waters were calculated using the software program MVP.

### REFERENCES

The references listed below as well as all references cited in the specification are incorporated herein by reference to the extent that they supplement, explain, provide a background for or teach methodology, techniques and/or compositions employed herein.

Altschul et al., (1990) J. Mol. Biol. 215: 403-10

Bartleft et al., (1989) Special Pub., Royal Chem. Soc. 78: 182-196

Blundell & Johnson (1985) Method.Enzymol. 114A & 115B, (Wyckoff et al., eds.), Academic Press

Bohm, (1992) J. Comput. Aid. Mol. Des. 6: 61-78

Braissant et al., (1996) Endocrinology 137: 354-366

Brooks et al., (1983) J. Comp. Chem 8: 132

Brünger et al., (1998) Acta. Crystallogr. D54: 905-921

Brünger, (1992) X-PLOR, Version 3.1. A System for X-ray Crystallography and NMR, Yale University Press, New Haven, Conn.

Chang et al., (1997) Proc. Natl. Acad. Sci. U.S.A. 94(17): 9040-45

Chien et al., (1991), Proc. Natl. Acad. Sci. U.S.A., 88: 9578-82

Cohen et al., (1990) J. Med. Chem. 33: 883-894

Connolly, (1983) Science 221: 709-713

Drewes et al., (1996) Mol. Cell. Biol. 16:925-31

Dreyer et al., (1992) Cell 68: 879-887

Eisen et al., (1994) Proteins 19: 199-221

Gampe et al., (2000) Mol. Cell 5: 545-55

Gearing et al., (1993) Proc. Natl. Acad. Sci. U.S.A. 90: 1440-44

Goodford, (1985) J. Med. Chem. 28: 849-857

Goodsell & Olsen, (1990) Proteins 8: 195-202

Gösttlicher et al., (1992) Proc. Nat Acad. Sci. U.S.A. 89: 4653-57

Green, (1992) Biochem. Pharmacol. 43: 393-401

Gribskov etal., (1986) Nucl. Acids. Res. 14: 6745

Gulick et al., (1994) Proc. Natl. Acad. Sci. U.S.A. 91: 11012-16

Hauptman, (1997) Curr. Opin. Struct. Biol. 7: 672-80

Havel & Kane, (1973) Annu. Rev. Pharmacol. 13: 287-308

Hertz et al., (1995) J. Biol. Chem. 270:13470-75

Heyman et al., (1992) Cell 68: 397-406

Isseman & Green, (1990) Nature 347: 645-650

Kakizawa et al., (1997) J. Biol. Chem. 272 (38): 23799-23804

Keller & Whali, (1993) Trends Endocrin. Met. 4: 291-296

Keller et al., (1993) Proc. Natl. Acad. Sci. U.S.A. 90: 2160-64

Kuntz et al., (1992) J. Mol. Biol. 161: 269-288

Kyte & Doolittle, (1982), J. Mol. Biol. 157: 105-132

Lambert, (1997) in Practical Application of Computer-Aided Drug Design, (Charifson, ed.), pp. 243-303, Marcel-Dekker, New York

Lattman, (1985) in Method. Enzymol., 115: 55-77

Lazarow & Fujiki, (1985) Ann. Rev. Cell Biol. 1: 489-530

Lemberger et al., (1996) J. Biol. Chem. 271: 1764-1769

Lemberger et al., (1996) Ann. Rev. Cell. Dev. Biol. 12: 335-63

Levin et al., (1992) Nature 355: 359-361

Lin et al., (1997) Mol. Cell Biol. 17 (10): 6131-38

Martin, (1992) J. Med. Chem. 35: 2145-2154

McPherson et al., (1989) Preparation and Analysis of Protein Crystals, Robert E. Krieger Publishing Company, Malabar, Fla.

Miranker & Karplus, (1991) Proteins 11: 29-34

Muerhoff et al., (1992) J. Biol. Chem. 267: 19051-53

Navaza, (1994) Acta. Cryst. A50: 157-163

Navia & Murcko, (1992) Curr. Opin. Struc. Biol. 2: 202-210

Needleman et al., (1970) J. Mol. Biol. 48: 443

Nelali et al., (1988) Cancer Res. 48: 5316-5324

Nicholls et al., (1991) Proteins 11: 282

Nichols et al., (1998) Anal. Biochem. 257: 112-19

Nishibata & Itai, (1991) Tetrahedron 47: 8985

Nolte et al., (1998) Nature 395:137-43

Oberfield et al., (1999) Proc. Nat. Acad. Sci. 96: 6102-106

Otwinowski, (1993) in Proceedings of the CCP4 Study Weekend: Data Collection and Processing. (Sawyer et al., eds.), pp.56-62, SERC Daresbury Laboratory, England

Pearlman et al., (1995) Comput Phys. Commun. 91: 141

Rodriquez et al., (1994) J. Biol. Chem. 269: 18767-72

Rossmann, ed, (1972) The Molecular Replacement Method, Gordon & Breach, New York

Sambrook et al., (1992) Molecular Cloning: A Laboratory Manual, Cold Spring Harbor, New York

Schoonjans et al., (1995) J. Biol. Chem. 270: 19269-19276

Schwartz et al., eds., (1979), Atlas of Protein Sequence and Structure, National Biomedical Research Foundation, pp. 357-358

Sher et al., (1993) Biochem. 32: 5598-5604

Shiau et al., (1998) Cell 95: 927-37

Shibata et al., (1997) Recent Prog. Horm. Res. 52: 141-64

Sladek et al., Genes Dev. 4:2353-65

Smith et al., (1981) Adv. AppL. Math. 2:482

Stout, (1983) J. Med. Chem. 26(6): 808-13

Tagami et al., (1997) Mol. Cell Biol. 17(5): 2643-48

Tugwood et al., (1992) EMBO J. 11: 433-439

Uppenberg et al., (1998) J.Biol. Chem. 273: 31108-12

Vamecq & Draye, (1989) Essays Biochem 24: 115-225

Van Holde, (1971) Physical Biochemistry, Prentice-Hall, N. J., pp. 221-39

Vu-Dac et al., (1994) J. Biol. Chem. 269: 31012-18

Vu-Dac et al., (1995) J. Clin. Invest. 96: 741-750

Weber, (1991)Adv. Protein Chem. 41:1-36

Weeks et al., (1993) Acta Cryst. D49: 179

Wellner, (1971) Anal. Chem. 43: 597

Wetmur & Davidson, (1968) J. Mol. Biol. 31: 349-70

Xu et al., (1999) Mol. Cell 3: 397-403

Zhu et al., (1997) J. Biol. Chem. 272 (14): 9048-54

U.S. Pat. No. 4,554,101

U.S. Pat. No. 4,672,108

U.S. Pat. No. 4,833,233

U.S. Pat. No. 5,463,564

U.S. Pat. No. 5,834,228

U.S. Pat. No. 5,872,011

U.S. Pat. No. 6,008,033

WO 84/03564

WO 99/26966

TABLE 1
PARAMETERS FOR THE CRYSTALLINE FORM
OF THE LIGAND BINDING DOMAIN (RESIDUES
207-441) OF PPARα IN COMPLEX WITH
COMPOUND 1

| X-ray Source | Glaxo Facility | IMCA |
|---|---|---|
| Space Group | P2₁2₁2 | P2₁2₁2 |
| Resolution (Å) | 20.0-2.4 | 20.0-1.8 |
| Unique Reflections (N) | 64,772 | 30,147 |
| Completeness (%) | 95.2 | 99.4 |
| I/σ(last shell) | 25.4 (3.3) | 39.5 (5.2) |
| R_{sym}^{a} (%) | 9.4 | 4.5 |
| Refinement Statistics | | |
| R factor^{b} (%)(2σ) | | 22.9 |
| R free^{c} (%)(2σ) | | 26.9 |
| R.M.S.D. Bond | | |
| Lengths (Å) | | 0.010 |
| R.M.S.D. Bond | | |
| Angles (degrees) | | 1.357 |
| Number of H₂O Molecules | | 396 |
| Total Non-hydrogen Atoms | | 2659 |

r.m.s.d. is the root mean square deviation from ideal geometry.
^{a}R_{sym} = Σ | I_{avg} − Iᵢ |/Σ Iᵢ
^{b}R_{factor} = Σ | F_{P} − F_{Pcalc} |/Σ Fₚ, where Fₚ and F_{pcalc} are observed and calculated structure factors, respectively
^{c}R_{free} is calculated from a randomly chosen 10% of reflections that have never been used in refinement, and R_{factor} is calculated for the remaining 90% of reflections.

TABLE 2
ATOMIC STRUCTURE COORDINATE DATA OBTAINED FROM X-RAY
DIFRACTION FROM THE LIGAND BINDING DOMAIN OF PPARα in
COMPLEX WITH COUPOUND 1

| | | | | | | | | | |
|---|---|---|---|---|---|---|---|---|---|
| | ATOM | | | | | | | | |
| ATOM | TYPE | RESIDUE | PROTEIN # | # | X | Y | Z | OCC | B |

| | | | | | | | | | |
|---|---|---|---|---|---|---|---|---|---|
| 1 | CB | ASP | A | 202 | 14.533 | 5.575 | 19.857 | 1.00 | 78.46 |
| 2 | CG | ASP | A | 202 | 14.270 | 4.099 | 20.079 | 1.00 | 79.53 |
| 3 | OD1 | ASP | A | 202 | 13.370 | 3.547 | 19.410 | 1.00 | 80.23 |
| 4 | OD2 | ASP | A | 202 | 14.963 | 3.492 | 20.921 | 1.00 | 80.14 |
| 5 | C | ASP | A | 202 | 15.243 | 7.375 | 18.274 | 1.00 | 76.33 |
| 6 | O | ASP | A | 202 | 14.377 | 8.174 | 17.918 | 1.00 | 76.50 |
| 7 | N | ASP | A | 202 | 16.101 | 5.066 | 18.013 | 1.00 | 77.36 |
| 8 | CA | ASP | A | 202 | 14.925 | 5.890 | 18.412 | 1.00 | 77.08 |
| 9 | N | LEU | A | 203 | 16.488 | 7.739 | 18.565 | 1.00 | 74.80 |
| 10 | CA | LEU | A | 203 | 16.927 | 9.125 | 18.460 | 1.00 | 72.96 |
| 11 | CB | LEU | A | 203 | 18.150 | 9.378 | 19.344 | 1.00 | 73.58 |
| 12 | CG | LEU | A | 203 | 18.821 | 10.740 | 19.132 | 1.00 | 73.64 |
| 13 | CD1 | LEU | A | 203 | 18.061 | 11.815 | 19.895 | 1.00 | 73.44 |
| 14 | CD2 | LEU | A | 203 | 20.265 | 10.680 | 19.597 | 1.00 | 73.65 |
| 15 | C | LEU | A | 203 | 17.304 | 9.418 | 17.019 | 1.00 | 71.55 |
| 16 | O | LEU | A | 203 | 17.251 | 10.561 | 16.574 | 1.00 | 71.54 |
| 17 | N | LYS | A | 204 | 17.694 | 8.379 | 16.292 | 1.00 | 69.51 |
| 18 | CA | LYS | A | 204 | 18.088 | 8.548 | 14.905 | 1.00 | 67.63 |
| 19 | CB | LYS | A | 204 | 18.876 | 7.324 | 14.439 | 1.00 | 68.39 |
| 20 | CG | LYS | A | 204 | 19.491 | 7.454 | 13.057 | 1.00 | 68.94 |
| 21 | CD | LYS | A | 204 | 20.927 | 6.946 | 13.047 | 1.00 | 69.41 |
| 22 | CE | LYS | A | 204 | 21.072 | 5.632 | 13.805 | 1.00 | 69.58 |
| 23 | NZ | LYS | A | 204 | 22.447 | 5.088 | 13.716 | 1.00 | 70.30 |
| 24 | C | LYS | A | 204 | 16.866 | 8.787 | 14.025 | 1.00 | 65.91 |
| 25 | O | LYS | A | 204 | 16.995 | 9.160 | 12.858 | 1.00 | 66.04 |
| 26 | N | SER | A | 205 | 15.680 | 8.574 | 14.590 | 1.00 | 62.82 |
| 27 | CA | SER | A | 205 | 14.445 | 8.814 | 13.854 | 1.00 | 59.48 |
| 28 | CB | SER | A | 205 | 13.240 | 8.252 | 14.613 | 1.00 | 59.65 |
| 29 | OG | SER | A | 205 | 12.994 | 8.984 | 15.802 | 1.00 | 59.47 |
| 30 | C | SER | A | 205 | 14.330 | 10.331 | 13.751 | 1.00 | 56.82 |
| 31 | O | SER | A | 205 | 13.495 | 10.861 | 13.016 | 1.00 | 56.27 |
| 32 | N | LEU | A | 206 | 15.186 | 11.015 | 14.510 | 1.00 | 53.62 |
| 33 | CA | LEU | A | 206 | 15.240 | 12.473 | 14.540 | 1.00 | 50.75 |
| 34 | CB | LEU | A | 206 | 16.362 | 12.935 | 15.477 | 1.00 | 50.59 |
| 35 | CG | LEU | A | 206 | 16.728 | 14.421 | 15.542 | 1.00 | 49.94 |
| 36 | CD1 | LEU | A | 206 | 15.575 | 15.224 | 16.114 | 1.00 | 49.18 |
| 37 | CD2 | LEU | A | 206 | 17.972 | 14.595 | 16.403 | 1.00 | 49.63 |
| 38 | C | LEU | A | 206 | 15.490 | 13.018 | 13.142 | 1.00 | 48.97 |
| 39 | O | LEU | A | 206 | 15.015 | 14.098 | 12.795 | 1.00 | 49.11 |
| 40 | N | ALA | A | 207 | 16.239 | 12.262 | 12.344 | 1.00 | 46.47 |
| 41 | CA | ALA | A | 207 | 16.555 | 12.671 | 10.983 | 1.00 | 45.03 |
| 42 | CB | ALA | A | 207 | 17.428 | 11.622 | 10.313 | 1.00 | 44.87 |
| 43 | C | ALA | A | 207 | 15.291 | 12.907 | 10.157 | 1.00 | 44.53 |
| 44 | O | ALA | A | 207 | 15.077 | 14.006 | 9.646 | 1.00 | 43.47 |
| 45 | N | LYS | A | 208 | 14.454 | 11.881 | 10.029 | 1.00 | 43.76 |
| 46 | CA | LYS | A | 208 | 13.226 | 12.011 | 9.251 | 1.00 | 42.20 |
| 47 | CB | LYS | A | 208 | 12.471 | 10.680 | 9.194 | 1.00 | 44.47 |
| 48 | CG | LYS | A | 208 | 11.537 | 10.576 | 7.990 | 1.00 | 45.43 |
| 49 | CD | LYS | A | 208 | 10.715 | 9.300 | 7.999 | 1.00 | 46.92 |
| 50 | CE | LYS | A | 208 | 9.660 | 9.328 | 9.093 | 1.00 | 48.09 |
| 51 | NZ | LYS | A | 208 | 8.799 | 8.114 | 9.059 | 1.00 | 49.24 |
| 52 | C | LYS | A | 208 | 12.330 | 13.086 | 9.852 | 1.00 | 41.19 |
| 53 | O | LYS | A | 208 | 11.658 | 13.818 | 9.128 | 1.00 | 40.18 |
| 54 | N | ARG | A | 209 | 12.328 | 13.174 | 11.179 | 1.00 | 38.94 |
| 55 | CA | ARG | A | 209 | 11.535 | 14.172 | 11.887 | 1.00 | 37.26 |
| 56 | CB | ARG | A | 209 | 11.745 | 14.022 | 13.398 | 1.00 | 39.88 |
| 57 | CG | ARG | A | 209 | 11.394 | 15.257 | 14.226 | 1.00 | 43.03 |
| 58 | CD | ARG | A | 209 | 9.901 | 15.543 | 14.240 | 1.00 | 45.71 |
| 59 | NE | ARG | A | 209 | 9.622 | 16.898 | 14.711 | 1.00 | 46.98 |
| 60 | CZ | ARG | A | 209 | 8.403 | 17.416 | 14.828 | 1.00 | 47.56 |
| 61 | NH1 | ARG | A | 209 | 7.337 | 16.691 | 14.511 | 1.00 | 48.63 |
| 62 | NH2 | ARG | A | 209 | 8.251 | 18.662 | 15.255 | 1.00 | 47.03 |
| 63 | C | ARG | A | 209 | 11.945 | 15.575 | 11.437 | 1.00 | 34.87 |
| 64 | O | ARG | A | 209 | 11.104 | 16.388 | 11.050 | 1.00 | 33.63 |
| 65 | N | ILE | A | 210 | 13.243 | 15.855 | 11.496 | 1.00 | 31.60 |
| 66 | CA | ILE | A | 210 | 13.759 | 17.156 | 11.086 | 1.00 | 28.45 |
| 67 | CB | ILE | A | 210 | 15.283 | 17.260 | 11.355 | 1.00 | 27.35 |
| 68 | CG2 | ILE | A | 210 | 15.873 | 18.457 | 10.625 | 1.00 | 26.86 |
| 69 | CG1 | ILE | A | 210 | 15.532 | 17.376 | 12.863 | 1.00 | 27.32 |
| 70 | CD1 | ILE | A | 210 | 17.002 | 17.319 | 13.251 | 1.00 | 26.22 |
| 71 | C | ILE | A | 210 | 13.485 | 17.354 | 9.599 | 1.00 | 27.44 |
| 72 | O | ILE | A | 210 | 13.158 | 18.453 | 9.157 | 1.00 | 26.71 |
| 73 | N | TYR | A | 211 | 13.614 | 16.277 | 8.836 | 1.00 | 25.68 |
| 74 | CA | TYR | A | 211 | 13.370 | 16.335 | 7.403 | 1.00 | 26.73 |
| 75 | CB | TYR | A | 211 | 13.782 | 15.011 | 6.748 | 1.00 | 25.17 |
| 76 | OG | TYR | A | 211 | 13.657 | 14.971 | 5.236 | 1.00 | 27.92 |
| 77 | CD1 | TYR | A | 211 | 13.862 | 16.116 | 4.464 | 1.00 | 27.10 |
| 78 | CE1 | TYR | A | 211 | 13.787 | 16.071 | 3.073 | 1.00 | 27.40 |
| 79 | CD2 | TYR | A | 211 | 13.372 | 13.775 | 4.573 | 1.00 | 28.20 |
| 80 | CE2 | TYR | A | 211 | 13.296 | 13.721 | 3.180 | 1.00 | 29.60 |
| 81 | CZ | TYR | A | 211 | 13.506 | 14.875 | 2.437 | 1.00 | 28.67 |
| 82 | OH | TYR | A | 211 | 13.446 | 14.829 | 1.061 | 1.00 | 30.22 |
| 83 | C | TYR | A | 211 | 11.892 | 16.637 | 7.152 | 1.00 | 26.35 |
| 84 | O | TYR | A | 211 | 11.552 | 17.402 | 6.253 | 1.00 | 22.94 |
| 85 | N | GLU | A | 212 | 11.010 | 16.046 | 7.951 | 1.00 | 27.41 |
| 86 | CA | GLU | A | 212 | 9.586 | 16.299 | 7.778 | 1.00 | 27.15 |
| 87 | CB | GLU | A | 212 | 8.760 | 15.392 | 8.700 | 1.00 | 29.37 |
| 88 | CG | GLU | A | 212 | 8.870 | 13.917 | 8.324 | 1.00 | 32.72 |
| 89 | CD | GLU | A | 212 | 7.948 | 13.014 | 9.129 | 1.00 | 34.56 |
| 90 | OE1 | GLU | A | 212 | 8.053 | 12.998 | 10.372 | 1.00 | 36.22 |
| 91 | OE2 | GLU | A | 212 | 7.121 | 12.314 | 8.510 | 1.00 | 36.44 |
| 92 | C | GLU | A | 212 | 9.297 | 17.771 | 8.057 | 1.00 | 25.89 |
| 93 | O | GLU | A | 212 | 8.535 | 18.407 | 7.331 | 1.00 | 25.26 |
| 94 | N | ALA | A | 213 | 9.926 | 18.312 | 9.099 | 1.00 | 23.75 |
| 95 | CA | ALA | A | 213 | 9.746 | 19.712 | 9.461 | 1.00 | 22.66 |
| 96 | CB | ALA | A | 213 | 10.524 | 20.026 | 10.731 | 1.00 | 24.94 |
| 97 | C | ALA | A | 213 | 10.221 | 20.609 | 8.321 | 1.00 | 21.68 |
| 98 | O | ALA | A | 213 | 9.618 | 21.642 | 8.033 | 1.00 | 20.08 |
| 99 | N | TYR | A | 214 | 11.308 | 20.203 | 7.676 | 1.00 | 20.59 |
| 100 | CA | TYR | A | 214 | 11.873 | 20.956 | 6.563 | 1.00 | 20.21 |
| 101 | CB | TYR | A | 214 | 13.226 | 20.343 | 6.204 | 1.00 | 21.59 |
| 102 | CG | TYR | A | 214 | 13.895 | 20.850 | 4.950 | 1.00 | 20.23 |
| 103 | CD1 | TYR | A | 214 | 13.565 | 20.322 | 3.701 | 1.00 | 21.49 |
| 104 | CE1 | TYR | A | 214 | 14.259 | 20.692 | 2.557 | 1.00 | 20.65 |
| 105 | CD2 | TYR | A | 214 | 14.933 | 21.779 | 5.019 | 1.00 | 19.83 |
| 106 | CE2 | TYR | A | 214 | 15.635 | 22.156 | 3.877 | 1.00 | 20.40 |
| 107 | CZ | TYR | A | 214 | 15.295 | 21.603 | 2.651 | 1.00 | 20.09 |
| 108 | OH | TYR | A | 214 | 16.013 | 21.932 | 1.522 | 1.00 | 22.28 |
| 109 | C | TYR | A | 214 | 10.920 | 20.963 | 5.364 | 1.00 | 20.36 |
| 110 | O | TYR | A | 214 | 10.663 | 22.009 | 4.768 | 1.00 | 18.08 |
| 111 | N | LEU | A | 215 | 10.370 | 19.801 | 5.027 | 1.00 | 19.65 |
| 112 | CA | LEU | A | 215 | 9.453 | 19.720 | 3.896 | 1.00 | 21.64 |
| 113 | CB | LEU | A | 215 | 9.161 | 18.255 | 3.563 | 1.00 | 22.15 |
| 114 | CG | LEU | A | 215 | 10.347 | 17.450 | 3.031 | 1.00 | 23.81 |
| 115 | CD1 | LEU | A | 215 | 9.934 | 15.993 | 2.840 | 1.00 | 24.83 |
| 116 | CD2 | LEU | A | 215 | 10.824 | 18.055 | 1.711 | 1.00 | 24.52 |
| 117 | C | LEU | A | 215 | 8.144 | 20.462 | 4.161 | 1.00 | 21.59 |
| 118 | O | LEU | A | 215 | 7.521 | 20.994 | 3.246 | 1.00 | 22.21 |
| 119 | N | LYS | A | 216 | 7.730 | 20.509 | 5.419 | 1.00 | 21.23 |
| 120 | CA | LYS | A | 216 | 6.484 | 21.177 | 5.773 | 1.00 | 24.07 |
| 121 | CB | LYS | A | 216 | 5.974 | 20.611 | 7.107 | 1.00 | 27.04 |
| 122 | CG | LYS | A | 216 | 4.766 | 21.320 | 7.718 | 1.00 | 32.48 |
| 123 | CD | LYS | A | 216 | 5.177 | 22.541 | 8.536 | 1.00 | 34.35 |
| 124 | CE | LYS | A | 216 | 3.978 | 23.177 | 9.227 | 1.00 | 36.46 |
| 125 | NZ | LYS | A | 216 | 4.372 | 24.346 | 10.068 | 1.00 | 35.63 |
| 126 | C | LYS | A | 216 | 6.574 | 22.701 | 5.863 | 1.00 | 23.63 |
| 127 | O | LYS | A | 216 | 5.584 | 23.401 | 5.619 | 1.00 | 22.64 |
| 128 | N | ASN | A | 217 | 7.758 | 23.216 | 6.178 | 1.00 | 21.43 |
| 129 | CA | ASN | A | 217 | 7.922 | 24.655 | 6.371 | 1.00 | 21.60 |
| 130 | CB | ASN | A | 217 | 8.627 | 24.894 | 7.708 | 1.00 | 21.30 |
| 131 | CG | ASN | A | 217 | 7.734 | 24.576 | 8.887 | 1.00 | 21.85 |
| 132 | OD1 | ASN | A | 217 | 6.763 | 25.286 | 9.144 | 1.00 | 21.90 |
| 133 | ND2 | ASN | A | 217 | 8.041 | 23.493 | 9.596 | 1.00 | 21.00 |
| 134 | C | ASN | A | 217 | 8.576 | 25.515 | 5.304 | 1.00 | 20.24 |
| 135 | O | ASN | A | 217 | 8.485 | 26.741 | 5.369 | 1.00 | 21.01 |
| 136 | N | PHE | A | 218 | 9.226 | 24.902 | 4.324 | 1.00 | 19.71 |
| 137 | CA | PHE | A | 218 | 9.880 | 25.682 | 3.275 | 1.00 | 20.43 |
| 138 | CB | PHE | A | 218 | 11.352 | 25.283 | 3.158 | 1.00 | 19.13 |
| 139 | CG | PHE | A | 218 | 12.161 | 25.595 | 4.388 | 1.00 | 18.17 |
| 140 | CD1 | PHE | A | 218 | 12.350 | 26.917 | 4.793 | 1.00 | 16.25 |
| 141 | CD2 | PHE | A | 218 | 12.734 | 24.574 | 5.136 | 1.00 | 16.60 |
| 142 | CE1 | PHE | A | 218 | 13.098 | 27.211 | 5.925 | 1.00 | 16.71 |
| 143 | CE2 | PHE | A | 218 | 13.486 | 24.856 | 6.273 | 1.00 | 17.69 |
| 144 | CZ | PHE | A | 218 | 13.671 | 26.177 | 6.671 | 1.00 | 18.05 |
| 145 | C | PHE | A | 218 | 9.185 | 25.502 | 1.935 | 1.00 | 21.87 |
| 146 | O | PHE | A | 218 | 9.159 | 24.408 | 1.388 | 1.00 | 23.21 |
| 147 | N | ASN | A | 219 | 8.632 | 26.587 | 1.407 | 1.00 | 22.66 |
| 148 | CA | ASN | A | 219 | 7.927 | 26.530 | 0.136 | 1.00 | 23.56 |
| 149 | CB | ASN | A | 219 | 7.238 | 27.869 | −0.135 | 1.00 | 26.95 |
| 150 | CG | ASN | A | 219 | 6.044 | 28.101 | 0.785 | 1.00 | 29.92 |
| 151 | OD1 | ASN | A | 219 | 5.119 | 27.283 | 0.836 | 1.00 | 33.43 |
| 152 | ND2 | ASN | A | 219 | 6.058 | 29.211 | 1.513 | 1.00 | 32.80 |
| 153 | C | ASN | A | 219 | 8.836 | 26.142 | −1.026 | 1.00 | 23.59 |
| 154 | O | ASN | A | 219 | 8.374 | 25.604 | −2.029 | 1.00 | 22.24 |
| 155 | N | MET | A | 220 | 10.129 | 26.413 | −0.888 | 1.00 | 22.41 |
| 156 | CA | MET | A | 220 | 11.083 | 26.059 | −1.927 | 1.00 | 23.18 |
| 157 | CB | MET | A | 220 | 11.713 | 27.316 | −2.535 | 1.00 | 23.66 |
| 158 | CG | MET | A | 220 | 12.713 | 27.051 | −3.664 | 1.00 | 25.31 |
| 159 | SD | MET | A | 220 | 11.971 | 26.305 | −5.126 | 1.00 | 26.48 |
| 160 | CE | MET | A | 220 | 10.935 | 27.658 | −5.694 | 1.00 | 24.83 |
| 161 | C | MET | A | 220 | 12.173 | 25.185 | −1.329 | 1.00 | 23.23 |
| 162 | O | MET | A | 220 | 12.639 | 25.434 | −0.214 | 1.00 | 23.23 |
| 163 | N | ASN | A | 221 | 12.549 | 24.147 | −2.064 | 1.00 | 21.71 |
| 164 | CA | ASN | A | 221 | 13.610 | 23.245 | −1.638 | 1.00 | 22.22 |
| 165 | CB | ASN | A | 221 | 13.049 | 22.029 | −0.891 | 1.00 | 23.15 |
| 166 | CG | ASN | A | 221 | 12.074 | 21.224 | −1.719 | 1.00 | 24.30 |
| 167 | OD1 | ASN | A | 221 | 12.329 | 20.923 | −2.880 | 1.00 | 25.96 |
| 168 | ND2 | ASN | A | 221 | 10.954 | 20.851 | −1.113 | 1.00 | 26.83 |
| 169 | C | ASN | A | 221 | 14.366 | 22.825 | −2.889 | 1.00 | 22.09 |
| 170 | O | ASN | A | 221 | 13.994 | 23.218 | −3.995 | 1.00 | 20.57 |
| 171 | N | LYS | A | 222 | 15.415 | 22.028 | −2.724 | 1.00 | 20.59 |
| 172 | CA | LYS | A | 222 | 16.232 | 21.614 | −3.858 | 1.00 | 20.89 |
| 173 | CB | LYS | A | 222 | 17.466 | 20.859 | −3.361 | 1.00 | 21.48 |
| 174 | CG | LYS | A | 222 | 18.653 | 20.954 | −4.297 | 1.00 | 20.77 |
| 175 | CD | LYS | A | 222 | 19.922 | 20.502 | −3.602 | 1.00 | 21.19 |
| 176 | CE | LYS | A | 222 | 21.125 | 20.628 | −4.507 | 1.00 | 21.09 |
| 177 | NZ | LYS | A | 222 | 22.361 | 20.180 | −3.823 | 1.00 | 19.55 |
| 178 | C | LYS | A | 222 | 15.502 | 20.789 | −4.914 | 1.00 | 20.69 |
| 179 | O | LYS | A | 222 | 15.678 | 21.022 | −6.109 | 1.00 | 19.10 |
| 180 | N | VAL | A | 223 | 14.689 | 19.827 | −4.493 | 1.00 | 20.29 |
| 181 | CA | VAL | A | 223 | 13.954 | 19.023 | −5.463 | 1.00 | 23.64 |
| 182 | CB | VAL | A | 223 | 13.004 | 18.016 | −4.776 | 1.00 | 25.30 |
| 183 | CG1 | VAL | A | 223 | 12.236 | 17.229 | −5.831 | 1.00 | 28.94 |
| 184 | CG2 | VAL | A | 223 | 13.795 | 17.073 | −3.900 | 1.00 | 28.35 |
| 185 | C | VAL | A | 223 | 13.125 | 19.930 | −6.375 | 1.00 | 22.27 |
| 186 | O | VAL | A | 223 | 13.228 | 19.844 | −7.600 | 1.00 | 22.87 |
| 187 | N | LYS | A | 224 | 12.314 | 20.797 | −5.769 | 1.00 | 22.90 |
| 188 | CA | LYS | A | 224 | 11.460 | 21.724 | −6.518 | 1.00 | 22.56 |
| 189 | CB | LYS | A | 224 | 10.640 | 22.604 | −5.563 | 1.00 | 25.17 |
| 190 | CG | LYS | A | 224 | 9.512 | 21.908 | −4.818 | 1.00 | 25.33 |
| 191 | CD | LYS | A | 224 | 8.688 | 22.947 | −4.060 | 1.00 | 27.14 |
| 192 | CE | LYS | A | 224 | 7.467 | 22.352 | −3.391 | 1.00 | 27.11 |
| 193 | NZ | LYS | A | 224 | 6.651 | 23.419 | −2.738 | 1.00 | 25.12 |
| 194 | C | LYS | A | 224 | 12.254 | 22.635 | −7.453 | 1.00 | 23.61 |
| 195 | O | LYS | A | 224 | 11.896 | 22.815 | −8.622 | 1.00 | 22.28 |
| 196 | N | ALA | A | 225 | 13.329 | 23.215 | −6.929 | 1.00 | 22.21 |
| 197 | CA | ALA | A | 225 | 14.174 | 24.112 | −7.709 | 1.00 | 21.88 |
| 198 | CB | ALA | A | 225 | 15.237 | 24.738 | −6.808 | 1.00 | 19.12 |
| 199 | C | ALA | A | 225 | 14.843 | 23.420 | −8.896 | 1.00 | 22.78 |
| 200 | O | ALA | A | 225 | 14.863 | 23.960 | −10.000 | 1.00 | 23.11 |
| 201 | N | ARG | A | 226 | 15.397 | 22.231 | −8.675 | 1.00 | 23.76 |
| 202 | CA | ARG | A | 226 | 16.067 | 21.514 | −9.753 | 1.00 | 26.20 |
| 203 | CB | ARG | A | 226 | 16.732 | 20.242 | −9.215 | 1.00 | 27.83 |
| 204 | CG | ARG | A | 226 | 18.056 | 20.526 | −8.517 | 1.00 | 28.58 |
| 205 | CD | ARG | A | 226 | 19.012 | 21.239 | −9.467 | 1.00 | 32.23 |
| 206 | NE | ARG | A | 226 | 19.998 | 22.056 | −8.766 | 1.00 | 34.36 |
| 207 | CZ | ARG | A | 226 | 19.683 | 23.053 | −7.942 | 1.00 | 37.04 |
| 208 | NH1 | ARG | A | 226 | 18.409 | 23.345 | −7.713 | 1.00 | 39.09 |
| 209 | NH2 | ARG | A | 226 | 20.636 | 23.772 | −7.365 | 1.00 | 35.24 |
| 210 | C | ARG | A | 226 | 15.150 | 21.185 | −10.926 | 1.00 | 27.67 |
| 211 | O | ARG | A | 226 | 15.590 | 21.176 | −12.079 | 1.00 | 29.64 |
| 212 | N | VAL | A | 227 | 13.879 | 20.922 | −10.639 | 1.00 | 27.53 |
| 213 | CA | VAL | A | 227 | 12.918 | 20.627 | −11.697 | 1.00 | 28.83 |
| 214 | CB | VAL | A | 227 | 11.548 | 20.219 | −11.120 | 1.00 | 30.09 |
| 215 | CG1 | VAL | A | 227 | 10.500 | 20.186 | −12.229 | 1.00 | 31.75 |
| 216 | CG2 | VAL | A | 227 | 11.653 | 18.851 | −10.464 | 1.00 | 31.01 |
| 217 | C | VAL | A | 227 | 12.728 | 21.878 | −12.543 | 1.00 | 28.74 |
| 218 | O | VAL | A | 227 | 12.736 | 21.825 | −13.775 | 1.00 | 27.81 |
| 219 | N | ILE | A | 228 | 12.561 | 23.010 | −11.868 | 1.00 | 26.31 |
| 220 | CA | ILE | A | 228 | 12.365 | 24.283 | −12.548 | 1.00 | 26.14 |
| 221 | CB | ILE | A | 228 | 12.050 | 25.398 | −11.520 | 1.00 | 24.59 |
| 222 | CG2 | ILE | A | 228 | 11.998 | 26.764 | −12.200 | 1.00 | 23.06 |
| 223 | CG1 | ILE | A | 228 | 10.720 | 25.094 | −10.831 | 1.00 | 23.57 |
| 224 | CD1 | ILE | A | 228 | 10.401 | 26.010 | −9.661 | 1.00 | 25.78 |
| 225 | C | ILE | A | 228 | 13.592 | 24.671 | −13.373 | 1.00 | 28.37 |
| 226 | O | ILE | A | 228 | 13.465 | 25.234 | −14.462 | 1.00 | 27.38 |
| 227 | N | LEU | A | 229 | 14.776 | 24.355 | −12.856 | 1.00 | 28.44 |
| 228 | CA | LEU | A | 229 | 16.027 | 24.684 | −13.533 | 1.00 | 31.74 |
| 229 | CB | LEU | A | 229 | 17.156 | 24.801 | −12.509 | 1.00 | 30.50 |
| 230 | CG | LEU | A | 229 | 17.045 | 25.984 | −11.544 | 1.00 | 30.22 |
| 231 | CD1 | LEU | A | 229 | 18.042 | 25.823 | −10.405 | 1.00 | 29.46 |
| 232 | CD2 | LEU | A | 229 | 17.290 | 27.279 | −12.304 | 1.00 | 29.52 |
| 233 | C | LEU | A | 229 | 16.424 | 23.685 | −14.616 | 1.00 | 34.68 |
| 234 | O | LEU | A | 229 | 17.281 | 23.974 | −15.450 | 1.00 | 35.11 |
| 235 | N | SER | A | 230 | 15.808 | 22.509 | −14.591 | 1.00 | 37.91 |
| 236 | CA | SER | A | 230 | 16.096 | 21.472 | −15.574 | 1.00 | 42.67 |
| 237 | CB | SER | A | 230 | 17.416 | 20.768 | −15.261 | 1.00 | 42.74 |
| 238 | OG | SER | A | 230 | 18.460 | 21.252 | −16.085 | 1.00 | 44.54 |
| 239 | C | SER | A | 230 | 14.987 | 20.443 | −15.629 | 1.00 | 45.44 |
| 240 | O | SER | A | 230 | 14.592 | 19.875 | −14.613 | 1.00 | 46.52 |
| 241 | N | GLY | A | 231 | 14.491 | 20.203 | −16.832 | 1.00 | 48.97 |
| 242 | CA | GLY | A | 231 | 13.427 | 19.242 | −17.011 | 1.00 | 52.46 |
| 243 | C | GLY | A | 231 | 12.702 | 19.563 | −18.294 | 1.00 | 55.01 |
| 244 | O | GLY | A | 231 | 12.343 | 18.658 | −19.040 | 1.00 | 55.62 |
| 245 | N | LYS | A | 232 | 12.510 | 20.860 | −18.542 | 1.00 | 57.05 |
| 246 | CA | LYS | A | 232 | 11.828 | 21.367 | −19.732 | 1.00 | 58.42 |
| 247 | CB | LYS | A | 232 | 12.757 | 21.290 | −20.945 | 1.00 | 59.37 |
| 248 | CG | LYS | A | 232 | 13.834 | 22.365 | −20.980 | 1.00 | 58.74 |
| 249 | CD | LYS | A | 232 | 14.914 | 22.004 | −21.989 | 1.00 | 59.57 |
| 250 | CE | LYS | A | 232 | 15.909 | 23.134 | −22.205 | 1.00 | 59.20 |
| 251 | NZ | LYS | A | 232 | 15.454 | 24.073 | −23.261 | 1.00 | 60.11 |
| 252 | C | LYS | A | 232 | 10.518 | 20.651 | −20.036 | 1.00 | 59.28 |
| 253 | O | LYS | A | 232 | 9.537 | 21.285 | −20.416 | 1.00 | 59.75 |
| 254 | N | ALA | A | 233 | 10.525 | 19.331 | −19.869 | 1.00 | 60.20 |
| 255 | CA | ALA | A | 233 | 9.371 | 18.469 | −20.097 | 1.00 | 60.65 |
| 256 | CB | ALA | A | 233 | 9.187 | 17.529 | −18.907 | 1.00 | 60.60 |
| 257 | C | ALA | A | 233 | 8.110 | 19.284 | −20.312 | 1.00 | 61.11 |
| 258 | O | ALA | A | 233 | 7.648 | 19.450 | −21.442 | 1.00 | 61.12 |
| 259 | N | SER | A | 234 | 7.564 | 19.799 | −19.217 | 1.00 | 61.07 |
| 260 | CA | SER | A | 234 | 6.360 | 20.609 | −19.281 | 1.00 | 61.06 |
| 261 | CB | SER | A | 234 | 5.742 | 20.748 | −17.885 | 1.00 | 61.00 |
| 262 | OG | SER | A | 234 | 4.582 | 21.563 | −17.917 | 1.00 | 59.89 |
| 263 | C | SER | A | 234 | 6.687 | 21.988 | −19.847 | 1.00 | 61.10 |
| 264 | O | SER | A | 234 | 7.344 | 22.107 | −20.883 | 1.00 | 61.74 |
| 265 | N | ASN | A | 235 | 6.230 | 23.026 | −19.157 | 1.00 | 60.20 |
| 266 | CA | ASN | A | 235 | 6.452 | 24.396 | −19.590 | 1.00 | 59.10 |
| 267 | CD | ASN | A | 235 | 5.740 | 24.614 | −20.934 | 1.00 | 58.65 |
| 268 | CG | ASN | A | 235 | 5.584 | 26.076 | −21.296 | 1.00 | 58.90 |
| 269 | OD1 | ASN | A | 235 | 4.628 | 26.728 | −20.872 | 1.00 | 59.73 |
| 270 | ND2 | ASN | A | 235 | 6.521 | 26.601 | −22.081 | 1.00 | 58.39 |
| 271 | C | ASN | A | 235 | 5.952 | 25.349 | −18.502 | 1.00 | 58.52 |
| 272 | O | ASN | A | 235 | 5.697 | 24.917 | −17.377 | 1.00 | 59.67 |
| 273 | N | ASN | A | 236 | 5.807 | 26.629 | −18.840 | 1.00 | 56.14 |
| 274 | CA | ASN | A | 236 | 5.393 | 27.666 | −17.896 | 1.00 | 51.26 |
| 275 | CB | ASN | A | 236 | 4.356 | 27.117 | −16.912 | 1.00 | 52.89 |
| 276 | CG | ASN | A | 236 | 4.033 | 28.087 | −15.799 | 1.00 | 52.48 |
| 277 | OD1 | ASN | A | 236 | 3.453 | 29.148 | −16.028 | 1.00 | 52.78 |
| 278 | ND2 | ASN | A | 236 | 4.413 | 27.729 | −14.580 | 1.00 | 52.90 |
| 279 | C | ASN | A | 236 | 6.701 | 28.005 | −17.179 | 1.00 | 47.02 |
| 280 | O | ASN | A | 236 | 6.751 | 28.137 | −15.956 | 1.00 | 47.14 |
| 281 | N | PRO | A | 237 | 7.783 | 28.170 | −17.966 | 1.00 | 42.04 |
| 282 | CD | PRO | A | 237 | 7.651 | 28.434 | −19.410 | 1.00 | 41.47 |
| 283 | CA | PRO | A | 237 | 9.145 | 28.475 | −17.529 | 1.00 | 38.16 |
| 284 | CD | PRO | A | 237 | 9.906 | 28.523 | −18.846 | 1.00 | 38.68 |
| 285 | CG | PRO | A | 237 | 8.920 | 29.198 | −19.729 | 1.00 | 39.97 |
| 286 | C | PRO | A | 237 | 9.338 | 29.755 | −16.740 | 1.00 | 34.87 |
| 287 | O | PRO | A | 237 | 8.615 | 30.734 | −16.921 | 1.00 | 34.21 |
| 288 | N | PRO | A | 238 | 10.338 | 29.760 | −15.848 | 1.00 | 32.12 |
| 289 | CD | PRO | A | 238 | 11.273 | 28.667 | −15.531 | 1.00 | 32.31 |
| 290 | CA | PRO | A | 238 | 10.617 | 30.943 | −15.038 | 1.00 | 29.30 |
| 291 | CB | PRO | A | 238 | 11.741 | 30.470 | −14.114 | 1.00 | 29.79 |
| 292 | CG | PRO | A | 238 | 12.436 | 29.411 | −14.933 | 1.00 | 32.45 |
| 293 | C | PRO | A | 238 | 11.045 | 32.080 | −15.960 | 1.00 | 27.36 |
| 294 | O | PRO | A | 238 | 11.745 | 31.861 | −16.952 | 1.00 | 25.06 |
| 295 | N | PHE | A | 239 | 10.606 | 33.289 | −15.637 | 1.00 | 24.30 |
| 296 | CA | PHE | A | 239 | 10.939 | 34.458 | −16.431 | 1.00 | 23.14 |
| 297 | CB | PHE | A | 239 | 10.004 | 35.612 | −16.083 | 1.00 | 25.05 |
| 298 | CG | PHE | A | 239 | 10.111 | 36.768 | −17.022 | 1.00 | 26.31 |
| 299 | CD1 | PHE | A | 239 | 9.483 | 36.729 | −18.262 | 1.00 | 27.99 |
| 300 | CD2 | PHE | A | 239 | 10.874 | 37.881 | −16.691 | 1.00 | 27.41 |
| 301 | CE1 | PHE | A | 239 | 9.615 | 37.784 | −19.162 | 1.00 | 28.47 |
| 302 | CE2 | PHE | A | 239 | 11.014 | 38.940 | −17.583 | 1.00 | 28.03 |
| 303 | CZ | PHE | A | 239 | 10.382 | 38.891 | −18.823 | 1.00 | 29.16 |
| 304 | C | PHE | A | 239 | 12.370 | 34.872 | −16.133 | 1.00 | 22.55 |
| 305 | O | PHE | A | 239 | 12.723 | 35.087 | −14.976 | 1.00 | 19.83 |
| 306 | N | VAL | A | 240 | 13.189 | 35.002 | −17.171 | 1.00 | 22.23 |
| 307 | CA | VAL | A | 240 | 14.581 | 35.377 | −16.974 | 1.00 | 21.89 |
| 308 | CB | VAL | A | 240 | 15.492 | 34.735 | −18.051 | 1.00 | 23.08 |
| 309 | CG1 | VAL | A | 240 | 16.919 | 35.265 | −17.916 | 1.00 | 22.71 |
| 310 | CG2 | VAL | A | 240 | 15.487 | 33.217 | −17.889 | 1.00 | 22.78 |
| 311 | C | VAL | A | 240 | 14.813 | 36.884 | −16.959 | 1.00 | 22.84 |
| 312 | O | VAL | A | 240 | 14.387 | 37.613 | −17.861 | 1.00 | 22.43 |
| 313 | N | ILE | A | 241 | 15.482 | 37.340 | −15.905 | 1.00 | 20.77 |
| 314 | CA | ILE | A | 241 | 15.817 | 38.745 | −15.740 | 1.00 | 20.91 |
| 315 | CB | ILE | A | 241 | 15.496 | 39.223 | −14.306 | 1.00 | 20.98 |
| 316 | CG2 | ILE | A | 241 | 15.897 | 40.687 | −14.137 | 1.00 | 19.06 |
| 317 | CG1 | ILE | A | 241 | 13.999 | 39.039 | −14.028 | 1.00 | 19.24 |
| 318 | CD1 | ILE | A | 241 | 13.607 | 39.288 | −12.574 | 1.00 | 21.68 |
| 319 | C | ILE | A | 241 | 17.317 | 38.844 | −16.006 | 1.00 | 21.63 |
| 320 | O | ILE | A | 241 | 18.137 | 38.438 | −15.172 | 1.00 | 19.53 |
| 321 | N | HIS | A | 242 | 17.673 | 39.362 | −17.181 | 1.00 | 20.99 |
| 322 | CA | HIS | A | 242 | 19.077 | 39.476 | −17.568 | 1.00 | 22.26 |
| 323 | CB | HIS | A | 242 | 19.355 | 38.567 | −18.769 | 1.00 | 24.28 |
| 324 | CG | HIS | A | 242 | 18.588 | 38.937 | −19.998 | 1.00 | 24.97 |
| 325 | CD2 | HIS | A | 242 | 17.459 | 38.415 | −20.534 | 1.00 | 25.77 |
| 326 | ND1 | HIS | A | 242 | 18.951 | 39.986 | −20.816 | 1.00 | 27.65 |
| 327 | CE1 | HIS | A | 242 | 18.077 | 40.096 | −21.800 | 1.00 | 25.99 |
| 328 | NE2 | HIS | A | 242 | 17.162 | 39.156 | −21.653 | 1.00 | 26.18 |
| 329 | C | HIS | A | 242 | 19.537 | 40.902 | −17.868 | 1.00 | 23.10 |
| 330 | O | HIS | A | 242 | 20.721 | 41.135 | −18.111 | 1.00 | 21.51 |
| 331 | N | ASP | A | 243 | 18.600 | 41.847 | −17.848 | 1.00 | 23.64 |
| 332 | CA | ASP | A | 243 | 18.910 | 43.255 | −18.074 | 1.00 | 26.61 |
| 333 | CB | ASP | A | 243 | 19.170 | 43.546 | −19.561 | 1.00 | 27.87 |
| 334 | CG | ASP | A | 243 | 17.972 | 43.258 | −20.445 | 1.00 | 29.61 |
| 335 | OD1 | ASP | A | 243 | 16.851 | 43.097 | −19.921 | 1.00 | 28.47 |
| 336 | OD2 | ASP | A | 243 | 18.157 | 43.206 | −21.683 | 1.00 | 31.76 |
| 337 | C | ASP | A | 243 | 17.792 | 44.150 | −17.553 | 1.00 | 27.23 |
| 338 | O | ASP | A | 243 | 16.800 | 43.668 | −17.006 | 1.00 | 25.64 |
| 339 | N | MET | A | 244 | 17.953 | 45.457 | −17.727 | 1.00 | 28.20 |
| 340 | CA | MET | A | 244 | 16.964 | 46.414 | −17.243 | 1.00 | 28.88 |
| 341 | CB | MET | A | 244 | 17.466 | 47.838 | −17.483 | 1.00 | 31.16 |
| 342 | CG | MET | A | 244 | 18.749 | 48.140 | −16.733 | 1.00 | 33.46 |
| 343 | SD | MET | A | 244 | 18.511 | 48.081 | −14.932 | 1.00 | 36.77 |
| 344 | CE | MET | A | 244 | 19.258 | 49.667 | −14.476 | 1.00 | 37.18 |
| 345 | C | MET | A | 244 | 15.571 | 46.241 | −17.837 | 1.00 | 28.34 |
| 346 | O | MET | A | 244 | 14.567 | 46.411 | −17.138 | 1.00 | 27.10 |
| 347 | N | GLU | A | 245 | 15.500 | 45.895 | −19.118 | 1.00 | 28.20 |
| 348 | CA | GLU | A | 245 | 14.206 | 45.709 | −19.760 | 1.00 | 27.38 |
| 349 | CB | GLU | A | 245 | 14.375 | 45.546 | −21.274 | 1.00 | 30.74 |
| 350 | CG | GLU | A | 245 | 13.072 | 45.246 | −21.999 | 1.00 | 34.46 |
| 351 | CD | GLU | A | 245 | 13.226 | 45.229 | −23.510 | 1.00 | 37.18 |
| 352 | OE1 | GLU | A | 245 | 14.109 | 44.505 | −24.016 | 1.00 | 38.99 |
| 353 | OE2 | GLU | A | 245 | 12.455 | 45.936 | −24.191 | 1.00 | 39.82 |
| 354 | C | GLU | A | 245 | 13.452 | 44.508 | −19.189 | 1.00 | 26.77 |
| 355 | O | GLU | A | 245 | 12.274 | 44.615 | −18.844 | 1.00 | 24.55 |
| 356 | N | THR | A | 246 | 14.126 | 43.366 | −19.086 | 1.00 | 25.14 |
| 357 | CA | THR | A | 246 | 13.485 | 42.169 | −18.550 | 1.00 | 22.92 |
| 358 | CB | THR | A | 246 | 14.342 | 40.909 | −18.802 | 1.00 | 22.22 |
| 359 | OG1 | THR | A | 246 | 15.686 | 41.135 | −18.362 | 1.00 | 20.92 |
| 360 | CG2 | THR | A | 246 | 14.352 | 40.571 | −20.288 | 1.00 | 22.08 |
| 361 | C | THR | A | 246 | 13.172 | 42.299 | −17.058 | 1.00 | 22.64 |
| 362 | O | THR | A | 246 | 12.284 | 41.615 | −16.545 | 1.00 | 21.75 |
| 363 | N | LEU | A | 247 | 13.896 | 43.171 | −16.360 | 1.00 | 21.77 |
| 364 | CA | LEU | A | 247 | 13.630 | 43.386 | −14.936 | 1.00 | 20.25 |
| 365 | CB | LEU | A | 247 | 14.698 | 44.278 | −14.289 | 1.00 | 20.20 |
| 366 | CG | LEU | A | 247 | 14.339 | 44.724 | −12.861 | 1.00 | 18.26 |
| 367 | CD1 | LEU | A | 247 | 14.285 | 43.498 | −11.941 | 1.00 | 19.55 |
| 368 | CD2 | LEU | A | 247 | 15.367 | 45.723 | −12.342 | 1.00 | 17.68 |
| 369 | C | LEU | A | 247 | 12.280 | 44.081 | −14.833 | 1.00 | 20.44 |
| 370 | O | LEU | A | 247 | 11.417 | 43.678 | −14.064 | 1.00 | 19.24 |
| 371 | N | CYS | A | 248 | 12.094 | 45.126 | −15.630 | 1.00 | 20.05 |
| 372 | CA | CYS | A | 248 | 10.833 | 45.856 | −15.606 | 1.00 | 22.13 |
| 373 | CB | CYS | A | 248 | 10.904 | 47.076 | −16.536 | 1.00 | 22.88 |
| 374 | SG | CYS | A | 248 | 12.090 | 48.355 | −16.013 | 1.00 | 28.89 |
| 375 | C | CYS | A | 248 | 9.681 | 44.939 | −16.016 | 1.00 | 22.46 |
| 376 | O | CYS | A | 248 | 8.594 | 45.020 | −15.452 | 1.00 | 23.21 |
| 377 | N | MET | A | 249 | 9.921 | 44.067 | −16.994 | 1.00 | 24.21 |
| 378 | CA | MET | A | 249 | 8.889 | 43.139 | −17.465 | 1.00 | 25.70 |
| 379 | CB | MET | A | 249 | 9.392 | 42.333 | −18.668 | 1.00 | 28.67 |
| 380 | CG | MET | A | 249 | 9.874 | 43.175 | −19.847 | 1.00 | 34.04 |
| 381 | SD | MET | A | 249 | 10.480 | 42.192 | −21.255 | 1.00 | 38.98 |
| 382 | CE | MET | A | 249 | 10.112 | 43.318 | −22.621 | 1.00 | 38.27 |
| 383 | C | MET | A | 249 | 8.504 | 42.178 | −16.342 | 1.00 | 25.87 |
| 384 | O | MET | A | 249 | 7.323 | 41.924 | −16.098 | 1.00 | 25.43 |
| 385 | N | ALA | A | 250 | 9.511 | 41.646 | −15.657 | 1.00 | 24.88 |
| 386 | CA | ALA | A | 250 | 9.268 | 40.720 | −14.559 | 1.00 | 23.76 |
| 387 | CB | ALA | A | 250 | 10.590 | 40.188 | −14.022 | 1.00 | 22.98 |
| 388 | C | ALA | A | 250 | 8.482 | 41.406 | −13.446 | 1.00 | 22.99 |
| 389 | O | ALA | A | 250 | 7.546 | 40.829 | −12.889 | 1.00 | 23.42 |
| 390 | N | GLU | A | 251 | 8.863 | 42.639 | −13.121 | 1.00 | 22.90 |
| 391 | CA | GLU | A | 251 | 8.176 | 43.387 | −12.075 | 1.00 | 23.30 |
| 392 | CB | GLU | A | 251 | 8.840 | 44.756 | −11.865 | 1.00 | 23.04 |
| 393 | CG | GLU | A | 251 | 10.269 | 44.676 | −11.336 | 1.00 | 23.77 |
| 394 | CD | GLU | A | 251 | 10.969 | 46.025 | −11.283 | 1.00 | 25.32 |
| 395 | OE1 | GLU | A | 251 | 10.914 | 46.766 | −12.288 | 1.00 | 26.09 |
| 396 | OE2 | GLU | A | 251 | 11.590 | 46.341 | −10.247 | 1.00 | 24.65 |
| 397 | C | GLU | A | 251 | 6.713 | 43.572 | −12.465 | 1.00 | 24.60 |
| 398 | O | GLU | A | 251 | 5.815 | 43.401 | −11.646 | 1.00 | 23.99 |
| 399 | N | LYS | A | 252 | 6.482 | 43.911 | −13.728 | 1.00 | 26.88 |
| 400 | CA | LYS | A | 252 | 5.129 | 44.120 | −14.227 | 1.00 | 29.38 |
| 401 | CB | LYS | A | 252 | 5.163 | 44.406 | −15.729 | 1.00 | 31.11 |
| 402 | CG | LYS | A | 252 | 3.792 | 44.688 | −16.321 | 1.00 | 34.55 |
| 403 | CD | LYS | A | 252 | 3.707 | 46.086 | −16.917 | 1.00 | 38.59 |
| 404 | CE | LYS | A | 252 | 4.142 | 47.146 | −15.921 | 1.00 | 40.59 |
| 405 | NZ | LYS | A | 252 | 5.625 | 47.202 | −15.814 | 1.00 | 42.89 |
| 406 | C | LYS | A | 252 | 4.229 | 42.912 | −13.965 | 1.00 | 30.43 |
| 407 | O | LYS | A | 252 | 3.045 | 43.059 | −13.656 | 1.00 | 30.61 |
| 408 | N | THR | A | 253 | 4.797 | 41.717 | −14.083 | 1.00 | 31.64 |
| 409 | CA | THR | A | 253 | 4.035 | 40.492 | −13.879 | 1.00 | 32.66 |
| 410 | CB | THR | A | 253 | 4.508 | 39.385 | −14.840 | 1.00 | 34.32 |
| 411 | OG1 | THR | A | 253 | 4.522 | 39.884 | −16.185 | 1.00 | 37.49 |
| 412 | CG2 | THR | A | 253 | 3.564 | 38.191 | −14.767 | 1.00 | 34.76 |
| 413 | C | THR | A | 253 | 4.076 | 39.923 | −12.459 | 1.00 | 32.57 |
| 414 | O | THR | A | 253 | 3.033 | 39.621 | −11.876 | 1.00 | 33.27 |
| 415 | N | LEU | A | 254 | 5.275 | 39.784 | −11.904 | 1.00 | 31.54 |
| 416 | CA | LEU | A | 254 | 5.441 | 39.208 | −10.572 | 1.00 | 31.84 |
| 417 | CB | LEU | A | 254 | 6.805 | 38.522 | −10.489 | 1.00 | 32.07 |
| 418 | CG | LEU | A | 254 | 6.933 | 37.131 | −11.126 | 1.00 | 33.07 |
| 419 | CD1 | LEU | A | 254 | 5.781 | 36.849 | −12.083 | 1.00 | 32.88 |
| 420 | CD2 | LEU | A | 254 | 8.271 | 37.040 | −11.833 | 1.00 | 32.53 |
| 421 | C | LEU | A | 254 | 5.256 | 40.128 | −9.367 | 1.00 | 32.52 |
| 422 | O | LEU | A | 254 | 4.770 | 39.690 | −8.321 | 1.00 | 31.50 |
| 423 | N | VAL | A | 255 | 5.654 | 41.389 | −9.501 | 1.00 | 32.08 |
| 424 | CA | VAL | A | 255 | 5.514 | 42.353 | −8.413 | 1.00 | 32.49 |
| 425 | CB | VAL | A | 255 | 6.870 | 42.614 | −7.710 | 1.00 | 32.30 |
| 426 | CG1 | VAL | A | 255 | 6.698 | 43.659 | −6.620 | 1.00 | 33.97 |
| 427 | CG2 | VAL | A | 255 | 7.393 | 41.322 | −7.095 | 1.00 | 33.63 |
| 428 | C | VAL | A | 255 | 4.988 | 43.650 | −9.017 | 1.00 | 33.56 |
| 429 | O | VAL | A | 255 | 5.664 | 44.680 | −9.018 | 1.00 | 32.17 |
| 430 | N | ALA | A | 256 | 3.766 | 43.571 | −9.532 | 1.00 | 34.97 |
| 431 | CA | ALA | A | 256 | 3.092 | 44.685 | −10.192 | 1.00 | 35.76 |
| 432 | CB | ALA | A | 256 | 1.636 | 44.310 | −10.438 | 1.00 | 37.47 |
| 433 | C | ALA | A | 256 | 3.162 | 46.065 | −9.529 | 1.00 | 37.14 |
| 434 | O | ALA | A | 256 | 3.127 | 47.083 | −10.224 | 1.00 | 35.98 |
| 435 | N | LYS | A | 257 | 3.267 | 46.114 | −8.203 | 1.00 | 37.87 |
| 436 | CA | LYS | A | 257 | 3.310 | 47.404 | −7.509 | 1.00 | 39.85 |
| 437 | CB | LYS | A | 257 | 3.154 | 47.210 | −5.995 | 1.00 | 39.30 |
| 438 | GG | LYS | A | 257 | 3.135 | 48.531 | −5.218 | 1.00 | 40.41 |
| 439 | CD | LYS | A | 257 | 3.092 | 48.326 | −3.708 | 1.00 | 41.21 |
| 440 | CE | LYS | A | 257 | 1.717 | 47.890 | −3.228 | 1.00 | 42.93 |
| 441 | NZ | LYS | A | 257 | 0.674 | 48.923 | −3.504 | 1.00 | 44.25 |
| 442 | C | LYS | A | 257 | 4.553 | 48.260 | −7.772 | 1.00 | 40.87 |
| 443 | O | LYS | A | 257 | 4.477 | 49.489 | −7.764 | 1.00 | 41.16 |
| 444 | N | LEU | A | 258 | 5.693 | 47.618 | −7.999 | 1.00 | 41.92 |
| 445 | CA | LEU | A | 258 | 6.934 | 48.347 | −8.241 | 1.00 | 43.90 |
| 446 | CB | LEU | A | 258 | 8.133 | 47.431 | −8.002 | 1.00 | 43.36 |
| 447 | CG | LEU | A | 258 | 8.106 | 46.627 | −6.702 | 1.00 | 42.94 |
| 448 | CD1 | LEU | A | 258 | 9.465 | 45.980 | −6.481 | 1.00 | 42.92 |
| 449 | CD2 | LEU | A | 258 | 7.756 | 47.541 | −5.536 | 1.00 | 43.54 |
| 450 | C | LEU | A | 258 | 7.021 | 48.917 | −9.647 | 1.00 | 45.44 |
| 451 | O | LEU | A | 258 | 8.118 | 49.189 | −10.142 | 1.00 | 45.30 |
| 452 | N | VAL | A | 259 | 5.874 | 49.138 | −10.277 | 1.00 | 47.08 |
| 453 | CA | VAL | A | 259 | 5.899 | 49.643 | −11.634 | 1.00 | 49.10 |
| 454 | CB | VAL | A | 259 | 5.984 | 48.463 | −12.612 | 1.00 | 49.12 |
| 455 | CG1 | VAL | A | 259 | 4.606 | 47.839 | −12.800 | 1.00 | 49.41 |
| 456 | CG2 | VAL | A | 259 | 6.587 | 48.926 | −13.921 | 1.00 | 49.20 |
| 457 | C | VAL | A | 259 | 4.716 | 50.538 | −12.012 | 1.00 | 50.30 |
| 458 | O | VAL | A | 259 | 4.459 | 50.771 | −13.194 | 1.00 | 50.13 |
| 459 | N | ALA | A | 260 | 4.010 | 51.065 | −11.016 | 1.00 | 51.85 |
| 460 | CA | ALA | A | 260 | 2.860 | 51.925 | −11.304 | 1.00 | 53.91 |
| 461 | CB | ALA | A | 260 | 1.627 | 51.065 | −11.582 | 1.00 | 53.84 |
| 462 | C | ALA | A | 260 | 2.542 | 52.938 | −10.207 | 1.00 | 55.04 |
| 463 | O | ALA | A | 260 | 1.443 | 53.496 | −10.173 | 1.00 | 55.68 |
| 464 | N | ASN | A | 261 | 3.496 | 53.182 | −9.314 | 1.00 | 55.70 |
| 465 | CA | ASN | A | 261 | 3.277 | 54.134 | −8.225 | 1.00 | 57.57 |
| 466 | CB | ASN | A | 261 | 3.064 | 53.389 | −6.905 | 1.00 | 20.38 |
| 467 | CG | ASN | A | 261 | 1.795 | 52.578 | −6.902 | 1.00 | 20.38 |
| 468 | OD1 | ASN | A | 261 | 1.812 | 51.370 | −7.162 | 1.00 | 20.38 |
| 469 | ND2 | ASN | A | 261 | 0.670 | 53.245 | −6.633 | 1.00 | 20.38 |
| 470 | C | ASN | A | 261 | 4.414 | 55.132 | −8.048 | 1.00 | 58.36 |
| 471 | O | ASN | A | 261 | 4.672 | 55.595 | −6.937 | 1.00 | 58.74 |
| 472 | N | GLY | A | 262 | 5.081 | 55.473 | −9.145 | 1.00 | 20.38 |
| 473 | CA | GLY | A | 262 | 6.204 | 56.394 | −9.063 | 1.00 | 20.38 |
| 474 | C | GLY | A | 262 | 7.442 | 55.570 | −8.751 | 1.00 | 20.38 |
| 475 | O | GLY | A | 262 | 8.576 | 56.039 | −8.883 | 1.00 | 20.38 |
| 476 | N | ILE | A | 263 | 7.213 | 54.319 | −8.352 | 1.00 | 58.34 |
| 477 | CA | ILE | A | 263 | 8.300 | 53.404 | −8.015 | 1.00 | 57.76 |
| 478 | CB | ILE | A | 263 | 7.787 | 52.149 | −7.293 | 1.00 | 58.40 |
| 479 | CG2 | ILE | A | 263 | 8.946 | 51.405 | −6.661 | 1.00 | 58.51 |
| 480 | CG1 | ILE | A | 263 | 6.764 | 52.524 | −6.225 | 1.00 | 58.87 |
| 481 | CD1 | ILE | A | 263 | 6.170 | 51.325 | −5.498 | 1.00 | 59.43 |
| 482 | C | ILE | A | 263 | 9.054 | 52.942 | −9.254 | 1.00 | 57.27 |
| 483 | O | ILE | A | 263 | 10.193 | 52.497 | −9.157 | 1.00 | 57.93 |
| 484 | N | GLN | A | 264 | 8.410 | 53.027 | −10.415 | 1.00 | 55.75 |
| 485 | CA | GLN | A | 264 | 9.038 | 52.596 | −11.658 | 1.00 | 54.43 |
| 486 | CB | GLN | A | 264 | 8.116 | 52.845 | −12.849 | 1.00 | 54.62 |
| 487 | CG | GLN | A | 264 | 7.654 | 54.270 | −13.006 | 1.00 | 54.62 |
| 488 | CD | GLN | A | 264 | 6.221 | 54.465 | −12.572 | 1.00 | 54.37 |
| 489 | OE1 | GLN | A | 264 | 5.896 | 54.391 | −11.390 | 1.00 | 54.65 |
| 490 | NB2 | GLN | A | 264 | 5.347 | 54.710 | −13.539 | 1.00 | 53.99 |
| 491 | C | GLN | A | 264 | 10.378 | 53.265 | −11.896 | 1.00 | 53.23 |
| 492 | O | GLN | A | 264 | 11.164 | 52.791 | −12.714 | 1.00 | 53.33 |
| 493 | N | ASN | A | 265 | 10.648 | 54.369 | −11.203 | 1.00 | 51.79 |
| 494 | CA | ASN | A | 265 | 11.943 | 54.991 | −11.390 | 1.00 | 50.16 |
| 495 | CB | ASN | A | 265 | 11.889 | 56.162 | −12.357 | 1.00 | 52.29 |
| 496 | CG | ASN | A | 265 | 13.036 | 56.120 | −13.366 | 1.00 | 54.63 |
| 497 | OD1 | ASN | A | 265 | 13.488 | 57.150 | −13.866 | 1.00 | 56.51 |
| 498 | ND2 | ASN | A | 265 | 13.504 | 54.914 | −13.673 | 1.00 | 55.84 |
| 499 | C | ASN | A | 265 | 12.728 | 55.397 | −10.164 | 1.00 | 47.37 |
| 500 | O | ASN | A | 265 | 13.251 | 56.509 | −10.078 | 1.00 | 47.22 |
| 501 | N | LYS | A | 266 | 12.765 | 54.494 | −9.195 | 1.00 | 43.69 |
| 502 | CA | LYS | A | 266 | 13.617 | 54.669 | −8.039 | 1.00 | 38.70 |
| 503 | CB | LYS | A | 266 | 13.077 | 53.921 | −6.817 | 1.00 | 39.95 |
| 504 | CG | LYS | A | 266 | 11.973 | 54.657 | −6.077 | 1.00 | 41.04 |
| 505 | CD | LYS | A | 266 | 11.700 | 54.020 | −4.721 | 1.00 | 42.78 |
| 506 | CE | LYS | A | 266 | 10.676 | 54.817 | −3.920 | 1.00 | 43.67 |
| 507 | NZ | LYS | A | 266 | 10.422 | 54.216 | −2.578 | 1.00 | 44.37 |
| 508 | C | LYS | A | 266 | 14.628 | 53.824 | −8.797 | 1.00 | 35.85 |
| 509 | O | LYS | A | 266 | 14.207 | 52.956 | −9.570 | 1.00 | 32.72 |
| 510 | N | GLU | A | 267 | 15.926 | 54.045 | −8.653 | 1.00 | 33.11 |
| 511 | CA | GLU | A | 267 | 16.809 | 53.225 | −9.468 | 1.00 | 30.98 |
| 512 | CB | GLU | A | 267 | 18.281 | 53.630 | −9.312 | 1.00 | 34.18 |
| 513 | CG | GLU | A | 267 | 18.846 | 53.674 | −7.923 | 1.00 | 36.39 |
| 514 | CD | GLU | A | 267 | 20.153 | 54.445 | −7.890 | 1.00 | 36.02 |
| 515 | OE1 | GLU | A | 267 | 21.030 | 54.183 | −8.740 | 1.00 | 36.54 |
| 516 | OE2 | GLU | A | 267 | 20.305 | 55.317 | −7.014 | 1.00 | 38.61 |
| 517 | C | GLU | A | 267 | 16.604 | 51.732 | −9.252 | 1.00 | 29.49 |
| 518 | O | GLU | A | 267 | 16.183 | 51.288 | −8.184 | 1.00 | 26.80 |
| 519 | N | ALA | A | 268 | 16.868 | 50.970 | −10.306 | 1.00 | 26.80 |
| 520 | CA | ALA | A | 268 | 16.702 | 49.526 | −10.293 | 1.00 | 27.28 |
| 521 | CB | ALA | A | 268 | 17.312 | 48.937 | −11.549 | 1.00 | 26.70 |
| 522 | C | ALA | A | 268 | 17.280 | 48.838 | −9.064 | 1.00 | 24.93 |
| 523 | O | ALA | A | 268 | 16.620 | 47.991 | −8.459 | 1.00 | 23.64 |
| 524 | N | GLU | A | 269 | 18.504 | 49.199 | −8.685 | 1.00 | 22.59 |
| 525 | CA | GLU | A | 269 | 19.126 | 48.561 | −7.536 | 1.00 | 22.23 |
| 526 | CB | GLU | A | 269 | 20.539 | 49.116 | −7.274 | 1.00 | 23.67 |
| 527 | CG | GLU | A | 269 | 20.876 | 50.450 | −7.915 | 1.00 | 27.55 |
| 528 | CD | GLU | A | 269 | 21.024 | 50.367 | −9.423 | 1.00 | 24.59 |
| 529 | OE1 | GLU | A | 269 | 20.048 | 50.679 | −10.116 | 1.00 | 26.10 |
| 530 | OE2 | GLU | A | 269 | 22.109 | 49.985 | −9.918 | 1.00 | 26.97 |
| 531 | C | GLU | A | 269 | 18.291 | 48.654 | −6.263 | 1.00 | 22.57 |
| 532 | O | GLU | A | 269 | 18.307 | 47.737 | −5.447 | 1.00 | 21.72 |
| 533 | N | VAL | A | 270 | 17.553 | 49.746 | −6.095 | 1.00 | 21.59 |
| 534 | CA | VAL | A | 270 | 16.728 | 49.911 | −4.899 | 1.00 | 21.26 |
| 535 | CB | VAL | A | 270 | 16.290 | 51.377 | −4.730 | 1.00 | 23.06 |
| 536 | CG1 | VAL | A | 270 | 15.279 | 51.504 | −3.604 | 1.00 | 25.08 |
| 537 | CG2 | VAL | A | 270 | 17.509 | 52.238 | −4.430 | 1.00 | 25.52 |
| 538 | C | VAL | A | 270 | 15.502 | 49.001 | −4.950 | 1.00 | 19.49 |
| 539 | O | VAL | A | 270 | 15.064 | 48.468 | −3.921 | 1.00 | 18.78 |
| 540 | N | ARG | A | 271 | 14.956 | 48.820 | −6.149 | 1.00 | 18.65 |
| 541 | CA | ARG | A | 271 | 13.799 | 47.948 | −6.340 | 1.00 | 17.90 |
| 542 | CB | ARG | A | 271 | 13.238 | 48.104 | −7.760 | 1.00 | 18.81 |
| 543 | CG | ARG | A | 271 | 12.207 | 49.209 | −7.891 | 1.00 | 23.01 |
| 544 | CD | ARG | A | 271 | 12.325 | 49.944 | −9.206 | 1.00 | 23.63 |
| 545 | NE | ARG | A | 271 | 12.388 | 49.055 | −10.366 | 1.00 | 21.82 |
| 546 | CZ | ARG | A | 271 | 12.893 | 49.422 | −11.539 | 1.00 | 23.69 |
| 547 | NH1 | ARG | A | 271 | 13.374 | 50.649 | −11.693 | 1.00 | 25.06 |
| 548 | NH2 | ARG | A | 271 | 12.923 | 48.573 | −12.554 | 1.00 | 24.09 |
| 549 | C | ARG | A | 271 | 14.219 | 46.498 | −6.102 | 1.00 | 17.66 |
| 550 | O | ARG | A | 271 | 13.528 | 45.743 | −5.420 | 1.00 | 16.97 |
| 551 | N | ILE | A | 272 | 15.361 | 46.118 | −6.662 | 1.00 | 17.24 |
| 552 | CA | ILE | A | 272 | 15.879 | 44.762 | −6.499 | 1.00 | 16.58 |
| 553 | CB | ILE | A | 272 | 17.151 | 44.558 | −7.346 | 1.00 | 17.41 |
| 554 | CG2 | ILE | A | 272 | 17.842 | 43.238 | −6.962 | 1.00 | 17.49 |
| 555 | CG1 | ILE | A | 272 | 16.772 | 44.568 | −8.832 | 1.00 | 17.66 |
| 556 | CD1 | ILE | A | 272 | 17.954 | 44.508 | −9.785 | 1.00 | 19.17 |
| 557 | C | ILE | A | 272 | 16.193 | 44.505 | −5.026 | 1.00 | 16.90 |
| 558 | O | ILE | A | 272 | 15.893 | 43.434 | −4.499 | 1.00 | 16.85 |
| 559 | N | PHE | A | 273 | 16.779 | 45.497 | −4.361 | 1.00 | 14.57 |
| 560 | CA | PHE | A | 273 | 17.114 | 45.364 | −2.943 | 1.00 | 13.82 |
| 561 | CB | PHE | A | 273 | 17.884 | 46.591 | −2.456 | 1.00 | 14.33 |
| 562 | CG | PHE | A | 273 | 18.485 | 46.427 | −1.086 | 1.00 | 16.86 |
| 563 | CD1 | PHE | A | 273 | 19.604 | 45.626 | −0.899 | 1.00 | 17.16 |
| 564 | CD2 | PHE | A | 273 | 17.937 | 47.078 | 0.012 | 1.00 | 16.21 |
| 565 | CE1 | PHE | A | 273 | 20.174 | 45.475 | 0.356 | 1.00 | 18.94 |
| 566 | CE2 | PHE | A | 273 | 18.498 | 46.936 | 1.275 | 1.00 | 19.15 |
| 567 | CZ | PHE | A | 273 | 19.621 | 46.133 | 1.448 | 1.00 | 20.18 |
| 568 | C | PHE | A | 273 | 15.850 | 45.203 | −2.102 | 1.00 | 14.30 |
| 569 | O | PHE | A | 273 | 15.860 | 44.505 | −1.096 | 1.00 | 15.47 |
| 570 | N | HIS | A | 274 | 14.768 | 45.874 | −2.496 | 1.00 | 14.52 |
| 571 | CA | HIS | A | 274 | 13.519 | 45.756 | −1.758 | 1.00 | 15.32 |
| 572 | CB | HIS | A | 274 | 12.455 | 46.695 | −2.330 | 1.00 | 16.74 |
| 573 | CG | HIS | A | 274 | 11.139 | 46.603 | −1.624 | 1.00 | 18.93 |
| 574 | CD2 | HIS | A | 274 | 9.960 | 46.057 | −2.003 | 1.00 | 21.47 |
| 575 | ND1 | HIS | A | 274 | 10.954 | 47.061 | −0.337 | 1.00 | 19.40 |
| 576 | CE1 | HIS | A | 274 | 9.718 | 46.799 | 0.047 | 1.00 | 21.96 |
| 577 | NE2 | HIS | A | 274 | 9.093 | 46.189 | −0.945 | 1.00 | 23.33 |
| 578 | C | HIS | A | 274 | 13.033 | 44.312 | −1.878 | 1.00 | 14.53 |
| 579 | O | HIS | A | 274 | 12.605 | 43.706 | −0.903 | 1.00 | 15.83 |
| 580 | N | CYS | A | 275 | 13.096 | 43.769 | −3.088 | 1.00 | 14.60 |
| 581 | CA | CYS | A | 275 | 12.674 | 42.388 | −3.323 | 1.00 | 14.94 |
| 582 | CB | CYS | A | 275 | 12.721 | 42.064 | −4.816 | 1.00 | 14.09 |
| 583 | SG | CYS | A | 275 | 11.470 | 42.950 | −5.767 | 1.00 | 16.01 |
| 584 | C | CYS | A | 275 | 13.558 | 41.414 | −2.544 | 1.00 | 14.97 |
| 585 | O | CYS | A | 275 | 13.090 | 40.366 | −2.099 | 1.00 | 13.78 |
| 586 | N | CYS | A | 276 | 14.836 | 41.748 | −2.383 | 1.00 | 15.23 |
| 587 | CA | CYS | A | 276 | 15.740 | 40.880 | −1.625 | 1.00 | 13.47 |
| 588 | CB | CYS | A | 276 | 17.180 | 41.409 | −1.667 | 1.00 | 15.89 |
| 589 | SG | CYS | A | 276 | 17.996 | 41.235 | −3.256 | 1.00 | 15.55 |
| 590 | C | CYS | A | 276 | 15.265 | 40.844 | −0.176 | 1.00 | 15.09 |
| 591 | O | CYS | A | 276 | 15.264 | 39.789 | 0.467 | 1.00 | 15.43 |
| 592 | N | GLN | A | 277 | 14.861 | 42.005 | 0.332 | 1.00 | 13.05 |
| 593 | CA | GLN | A | 277 | 14.379 | 42.105 | 1.707 | 1.00 | 14.74 |
| 594 | CB | GLN | A | 277 | 14.150 | 43.564 | 2.100 | 1.00 | 16.57 |
| 595 | CG | GLN | A | 277 | 15.418 | 44.386 | 2.205 | 1.00 | 18.11 |
| 596 | CD | GLN | A | 277 | 15.231 | 45.597 | 3.093 | 1.00 | 20.26 |
| 597 | OE1 | GLN | A | 277 | 14.949 | 45.465 | 4.286 | 1.00 | 22.83 |
| 598 | NE2 | GLN | A | 277 | 15.383 | 46.784 | 2.520 | 1.00 | 22.10 |
| 599 | C | GLN | A | 277 | 13.086 | 41.339 | 1.909 | 1.00 | 14.38 |
| 600 | O | GLN | A | 277 | 12.905 | 40.665 | 2.924 | 1.00 | 14.08 |
| 601 | N | CYS | A | 278 | 12.175 | 41.451 | 0.949 | 1.00 | 14.67 |
| 602 | CA | CYS | A | 278 | 10.911 | 40.741 | 1.062 | 1.00 | 15.04 |
| 603 | CB | CYS | A | 278 | 9.994 | 41.091 | −0.110 | 1.00 | 16.30 |
| 604 | SG | CYS | A | 278 | 9.396 | 42.797 | −0.061 | 1.00 | 22.25 |
| 605 | C | CYS | A | 278 | 11.192 | 39.245 | 1.093 | 1.00 | 14.70 |
| 606 | O | CYS | A | 278 | 10.593 | 38.505 | 1.868 | 1.00 | 14.34 |
| 607 | N | THR | A | 279 | 12.119 | 38.813 | 0.244 | 1.00 | 14.73 |
| 608 | CA | THR | A | 279 | 12.517 | 37.412 | 0.164 | 1.00 | 14.32 |
| 609 | CB | THR | A | 279 | 13.514 | 37.211 | −0.997 | 1.00 | 15.37 |
| 610 | OG1 | THR | A | 279 | 12.888 | 37.603 | −2.230 | 1.00 | 13.87 |
| 611 | GG2 | THR | A | 279 | 13.943 | 35.748 | −1.094 | 1.00 | 14.11 |
| 612 | C | THR | A | 279 | 13.135 | 36.946 | 1.488 | 1.00 | 14.08 |
| 613 | O | THR | A | 279 | 12.771 | 35.897 | 2.029 | 1.00 | 13.41 |
| 614 | N | SER | A | 280 | 14.057 | 37.732 | 2.028 | 1.00 | 12.61 |
| 615 | CA | SER | A | 280 | 14.672 | 37.371 | 3.303 | 1.00 | 12.61 |
| 616 | CB | SER | A | 280 | 15.775 | 38.361 | 3.657 | 1.00 | 12.19 |
| 617 | OG | SER | A | 280 | 16.915 | 38.120 | 2.860 | 1.00 | 11.40 |
| 618 | C | SER | A | 280 | 13.660 | 37.309 | 4.450 | 1.00 | 12.67 |
| 619 | O | SER | A | 280 | 13.726 | 36.413 | 5.283 | 1.00 | 12.90 |
| 620 | N | VAL | A | 281 | 12.720 | 38.249 | 4.492 | 1.00 | 13.57 |
| 621 | CA | VAL | A | 281 | 11.723 | 38.249 | 5.563 | 1.00 | 13.84 |
| 622 | CB | VAL | A | 281 | 10.790 | 39.480 | 5.461 | 1.00 | 15.55 |
| 623 | CG1 | VAL | A | 281 | 9.555 | 39.303 | 6.345 | 1.00 | 17.55 |
| 624 | CG2 | VAL | A | 281 | 11.558 | 40.721 | 5.895 | 1.00 | 16.53 |
| 625 | C | VAL | A | 281 | 10.911 | 36.961 | 5.533 | 1.00 | 15.35 |
| 626 | O | VAL | A | 281 | 10.639 | 36.366 | 6.575 | 1.00 | 14.98 |
| 627 | N | GLU | A | 282 | 10.546 | 36.522 | 4.334 | 1.00 | 16.73 |
| 628 | CA | GLU | A | 282 | 9.777 | 35.292 | 4.182 | 1.00 | 16.47 |
| 629 | CB | GLU | A | 282 | 9.338 | 35.113 | 2.726 | 1.00 | 19.03 |
| 630 | GG | GLU | A | 282 | 8.344 | 36.159 | 2.235 | 1.00 | 22.45 |
| 631 | CD | GLU | A | 282 | 6.924 | 35.908 | 2.727 | 1.00 | 27.72 |
| 632 | OE1 | GLU | A | 282 | 6.724 | 34.990 | 3.558 | 1.00 | 28.03 |
| 633 | OE2 | GLU | A | 282 | 6.009 | 36.635 | 2.279 | 1.00 | 27.23 |
| 634 | C | GLU | A | 282 | 10.603 | 34.088 | 4.612 | 1.00 | 15.29 |
| 635 | O | GLU | A | 282 | 10.099 | 33.192 | 5.282 | 1.00 | 14.93 |
| 636 | N | THR | A | 283 | 11.877 | 34.066 | 4.235 | 1.00 | 13.72 |
| 637 | CA | THR | A | 283 | 12.727 | 32.937 | 4.590 | 1.00 | 13.42 |
| 638 | CB | THR | A | 283 | 14.070 | 33.003 | 3.843 | 1.00 | 13.57 |
| 639 | OG1 | THR | A | 283 | 13.822 | 33.115 | 2.433 | 1.00 | 13.65 |
| 640 | CG2 | THR | A | 283 | 14.878 | 31.738 | 4.091 | 1.00 | 13.97 |
| 641 | C | THR | A | 283 | 12.961 | 32.876 | 6.097 | 1.00 | 13.92 |
| 642 | O | THR | A | 283 | 12.956 | 31.796 | 6.687 | 1.00 | 15.10 |
| 643 | N | VAL | A | 284 | 13.159 | 34.034 | 6.723 | 1.00 | 14.12 |
| 644 | CA | VAL | A | 284 | 13.359 | 34.074 | 8.176 | 1.00 | 14.58 |
| 645 | CB | VAL | A | 284 | 13.612 | 35.508 | 8.674 | 1.00 | 14.45 |
| 646 | 001 | VAL | A | 284 | 13.507 | 35.559 | 10.200 | 1.00 | 15.36 |
| 647 | CG2 | VAL | A | 284 | 14.976 | 35.980 | 8.225 | 1.00 | 15.78 |
| 648 | C | VAL | A | 284 | 12.097 | 33.541 | 8.861 | 1.00 | 14.26 |
| 649 | O | VAL | A | 284 | 12.165 | 32.827 | 9.870 | 1.00 | 14.04 |
| 650 | N | THR | A | 285 | 10.944 | 33.900 | 8.309 | 1.00 | 15.03 |
| 651 | CA | THR | A | 285 | 9.670 | 33.458 | 8.866 | 1.00 | 16.55 |
| 652 | CB | THR | A | 285 | 8.493 | 34.139 | 8.133 | 1.00 | 17.70 |
| 653 | OG1 | THR | A | 285 | 8.641 | 35.566 | 8.224 | 1.00 | 18.26 |
| 654 | CG2 | THR | A | 285 | 7.160 | 33.743 | 8.759 | 1.00 | 18.59 |
| 655 | C | THR | A | 285 | 9.551 | 31.931 | 8.775 | 1.00 | 16.67 |
| 656 | O | THR | A | 285 | 9.108 | 31.279 | 9.719 | 1.00 | 15.85 |
| 657 | N | GLU | A | 286 | 9.959 | 31.354 | 7.648 | 1.00 | 16.45 |
| 658 | CA | GLU | A | 286 | 9.897 | 29.897 | 7.489 | 1.00 | 14.59 |
| 659 | CB | GLU | A | 286 | 10.199 | 29.492 | 6.040 | 1.00 | 17.05 |
| 660 | CG | GLU | A | 286 | 9.201 | 29.983 | 5.025 | 1.00 | 19.53 |
| 661 | CD | GLU | A | 286 | 9.539 | 29.509 | 3.621 | 1.00 | 21.27 |
| 662 | OE1 | GLU | A | 286 | 10.740 | 29.424 | 3.291 | 1.00 | 24.29 |
| 663 | OE2 | GLU | A | 286 | 8.606 | 29.235 | 2.846 | 1.00 | 26.60 |
| 664 | C | GLU | A | 286 | 10.894 | 29.184 | 8.410 | 1.00 | 15.53 |
| 665 | O | GLU | A | 286 | 10.598 | 28.121 | 8.964 | 1.00 | 14.95 |
| 666 | N | LEU | A | 287 | 12.080 | 29.767 | 8.559 | 1.00 | 14.15 |
| 667 | CA | LEU | A | 287 | 13.117 | 29.183 | 9.404 | 1.00 | 14.54 |
| 668 | CB | LEU | A | 287 | 14.418 | 29.977 | 9.244 | 1.00 | 13.35 |
| 669 | CG | LEU | A | 287 | 15.286 | 29.532 | 8.062 | 1.00 | 13.79 |
| 670 | CD1 | LEU | A | 287 | 16.277 | 30.630 | 7.670 | 1.00 | 14.77 |
| 671 | CD2 | LEU | A | 287 | 16.029 | 28.253 | 8.452 | 1.00 | 15.41 |
| 672 | C | LEU | A | 287 | 12.684 | 29.185 | 10.863 | 1.00 | 15.65 |
| 673 | O | LEU | A | 287 | 13.017 | 28.277 | 11.627 | 1.00 | 15.76 |
| 674 | N | THR | A | 288 | 11.946 | 30.219 | 11.245 | 1.00 | 16.74 |
| 675 | CA | THR | A | 288 | 11.468 | 30.342 | 12.616 | 1.00 | 17.31 |
| 676 | CB | THR | A | 288 | 10.814 | 31.724 | 12.830 | 1.00 | 17.24 |
| 677 | OG1 | THR | A | 288 | 11.821 | 32.736 | 12.693 | 1.00 | 18.00 |
| 678 | CG2 | THR | A | 288 | 10.180 | 31.832 | 14.221 | 1.00 | 18.26 |
| 679 | C | THR | A | 288 | 10.484 | 29.211 | 12.913 | 1.00 | 18.94 |
| 680 | O | THR | A | 288 | 10.520 | 28.615 | 13.995 | 1.00 | 18.36 |
| 681 | N | GLU | A | 289 | 9.617 | 28.906 | 11.947 | 1.00 | 18.64 |
| 682 | CA | GLU | A | 289 | 8.654 | 27.824 | 12.120 | 1.00 | 19.64 |
| 683 | CB | GLU | A | 289 | 7.591 | 27.868 | 11.019 | 1.00 | 20.78 |
| 684 | CG | GLU | A | 289 | 6.701 | 29.088 | 11.102 | 1.00 | 24.51 |
| 685 | CD | GLU | A | 289 | 5.978 | 29.170 | 12.429 | 1.00 | 27.16 |
| 686 | OE1 | GLU | A | 289 | 5.151 | 28.276 | 12.705 | 1.00 | 28.65 |
| 687 | OE2 | GLU | A | 289 | 6.245 | 30.118 | 13.197 | 1.00 | 29.29 |
| 688 | C | GLU | A | 289 | 9.378 | 26.482 | 12.094 | 1.00 | 19.12 |
| 689 | O | GLU | A | 289 | 8.998 | 25.550 | 12.802 | 1.00 | 19.43 |
| 690 | N | PHE | A | 290 | 10.414 | 26.381 | 11.263 | 1.00 | 18.63 |
| 691 | CA | PHE | A | 290 | 11.215 | 25.158 | 11.177 | 1.00 | 18.56 |
| 692 | CB | PHE | A | 290 | 12.295 | 25.304 | 10.097 | 1.00 | 18.22 |
| 693 | CG | PHE | A | 290 | 13.295 | 24.178 | 10.074 | 1.00 | 16.52 |
| 694 | CD1 | PHE | A | 290 | 12.913 | 22.894 | 9.702 | 1.00 | 15.73 |
| 695 | CD2 | PHE | A | 290 | 14.628 | 24.411 | 10.407 | 1.00 | 16.62 |
| 696 | CE1 | PHE | A | 290 | 13.844 | 21.854 | 9.657 | 1.00 | 16.62 |
| 697 | CE2 | PHE | A | 290 | 15.564 | 23.387 | 10.367 | 1.00 | 16.63 |
| 698 | CZ | PHE | A | 290 | 15.170 | 22.097 | 9.987 | 1.00 | 16.10 |
| 699 | C | PHE | A | 290 | 11.894 | 24.898 | 12.525 | 1.00 | 19.02 |
| 700 | O | PHE | A | 290 | 11.856 | 23.783 | 13.049 | 1.00 | 19.20 |
| 701 | N | ALA | A | 291 | 12.529 | 25.931 | 13.073 | 1.00 | 19.85 |
| 702 | CA | ALA | A | 291 | 13.226 | 25.800 | 14.353 | 1.00 | 19.89 |
| 703 | CB | ALA | A | 291 | 13.856 | 27.130 | 14.746 | 1.00 | 20.05 |
| 704 | C | ALA | A | 291 | 12.270 | 25.337 | 15.449 | 1.00 | 21.19 |
| 705 | O | A1A | A | 291 | 12.623 | 24.499 | 16.283 | 1.00 | 19.62 |
| 706 | N | LYS | A | 292 | 11.064 | 25.897 | 15.447 | 1.00 | 21.75 |
| 707 | CA | LYS | A | 292 | 10.053 | 25.545 | 16.439 | 1.00 | 23.62 |
| 708 | CB | LYS | A | 292 | 8.802 | 26.407 | 16.245 | 1.00 | 22.82 |
| 709 | CG | LYS | A | 292 | 8.959 | 27.846 | 16.715 | 1.00 | 27.26 |
| 710 | CD | LYS | A | 292 | 7.821 | 28.738 | 16.214 | 1.00 | 30.13 |
| 711 | CE | LYS | A | 292 | 6.452 | 28.165 | 16.544 | 1.00 | 32.35 |
| 712 | NZ | LYS | A | 292 | 6.234 | 28.014 | 18.007 | 1.00 | 36.87 |
| 713 | C | LYS | A | 292 | 9.686 | 24.070 | 16.346 | 1.00 | 24.64 |
| 714 | O | LYS | A | 292 | 9.150 | 23.493 | 17.295 | 1.00 | 24.87 |
| 715 | N | ALA | A | 293 | 9.983 | 23.460 | 15.202 | 1.00 | 24.73 |
| 716 | CA | ALA | A | 293 | 9.684 | 22.051 | 14.985 | 1.00 | 24.93 |
| 717 | CB | ALA | A | 293 | 9.190 | 21.840 | 13.562 | 1.00 | 24.81 |
| 718 | C | ALA | A | 293 | 10.871 | 21.129 | 15.274 | 1.00 | 24.71 |
| 719 | O | ALA | A | 293 | 10.750 | 19.909 | 15.185 | 1.00 | 23.86 |
| 720 | N | ILE | A | 294 | 12.024 | 21.704 | 15.606 | 1.00 | 23.98 |
| 721 | CA | ILE | A | 294 | 13.188 | 20.885 | 15.925 | 1.00 | 22.06 |
| 722 | CB | ILE | A | 294 | 14.511 | 21.661 | 15.764 | 1.00 | 21.52 |
| 723 | CG2 | ILE | A | 294 | 15.684 | 20.769 | 16.166 | 1.00 | 20.29 |
| 724 | CG1 | ILE | A | 294 | 14.687 | 22.112 | 14.310 | 1.00 | 18.78 |
| 725 | CDI | ILE | A | 294 | 15.938 | 22.940 | 14.085 | 1.00 | 20.43 |
| 726 | C | ILE | A | 294 | 13.065 | 20.448 | 17.383 | 1.00 | 23.55 |
| 727 | O | ILE | A | 294 | 13.023 | 21.284 | 18.284 | 1.00 | 22.86 |
| 728 | N | PRO | A | 295 | 13.006 | 19.130 | 17.629 | 1.00 | 24.07 |
| 729 | CD | PRO | A | 295 | 13.131 | 18.036 | 16.648 | 1.00 | 23.94 |
| 730 | CA | PRO | A | 295 | 12.885 | 18.592 | 18.989 | 1.00 | 24.71 |
| 731 | CB | PRO | A | 295 | 13.284 | 17.135 | 18.808 | 1.00 | 24.70 |
| 732 | CG | PRO | A | 295 | 12.728 | 16.824 | 17.458 | 1.00 | 25.44 |
| 733 | C | PRO | A | 295 | 13.757 | 19.301 | 20.023 | 1.00 | 24.62 |
| 734 | O | PRO | A | 295 | 14.985 | 19.321 | 19.906 | 1.00 | 25.02 |
| 735 | N | GLY | A | 296 | 13.114 | 19.889 | 21.029 | 1.00 | 23.49 |
| 736 | CA | GLY | A | 296 | 13.854 | 20.563 | 22.081 | 1.00 | 23.80 |
| 737 | C | GLY | A | 296 | 14.022 | 22.064 | 21.948 | 1.00 | 22.79 |
| 738 | O | GLY | A | 296 | 14.240 | 22.752 | 22.948 | 1.00 | 21.27 |
| 739 | N | PHE | A | 297 | 13.928 | 22.583 | 20.728 | 1.00 | 22.01 |
| 740 | CA | PHE | A | 297 | 14.097 | 24.019 | 20.518 | 1.00 | 22.24 |
| 741 | CB | PHE | A | 297 | 14.011 | 24.358 | 19.025 | 1.00 | 21.13 |
| 742 | CG | PHE | A | 297 | 14.296 | 25.805 | 18.715 | 1.00 | 20.73 |
| 743 | CD1 | PHE | A | 297 | 13.287 | 26.760 | 18.780 | 1.00 | 21.28 |
| 744 | CD2 | PHE | A | 297 | 15.584 | 26.215 | 18.389 | 1.00 | 20.25 |
| 745 | CE1 | PHE | A | 297 | 13.560 | 28.105 | 18.523 | 1.00 | 20.90 |
| 746 | CE2 | PHE | A | 297 | 15.867 | 27.556 | 18.131 | 1.00 | 19.14 |
| 747 | CZ | PHE | A | 297 | 14.856 | 28.500 | 18.199 | 1.00 | 19.75 |
| 748 | C | PHE | A | 297 | 13.080 | 24.854 | 21.289 | 1.00 | 22.26 |
| 749 | O | PHE | A | 297 | 13.439 | 25.835 | 21.945 | 1.00 | 22.51 |
| 750 | N | ALA | A | 298 | 11.813 | 24.464 | 21.217 | 1.00 | 23.86 |
| 751 | CA | ALA | A | 298 | 10.754 | 25.206 | 21.896 | 1.00 | 24.65 |
| 752 | CB | ALA | A | 298 | 9.391 | 24.693 | 21.450 | 1.00 | 25.58 |
| 753 | C | ALA | A | 298 | 10.862 | 25.154 | 23.418 | 1.00 | 25.96 |
| 754 | O | ALA | A | 298 | 10.229 | 25.947 | 24.111 | 1.00 | 26.50 |
| 755 | N | ASN | A | 299 | 11.668 | 24.230 | 23.933 | 1.00 | 26.32 |
| 756 | CA | ASN | A | 299 | 11.856 | 24.088 | 25.377 | 1.00 | 26.91 |
| 757 | CB | ASN | A | 299 | 12.296 | 22.663 | 25.714 | 1.00 | 27.32 |
| 758 | CG | ASN | A | 299 | 11.198 | 21.648 | 25.496 | 1.00 | 27.93 |
| 759 | OD1 | ASN | A | 299 | 11.456 | 20.447 | 25.428 | 1.00 | 31.25 |
| 760 | ND2 | ASN | A | 299 | 9.962 | 22.123 | 25.393 | 1.00 | 27.97 |
| 761 | C | ASN | A | 299 | 12.891 | 25.068 | 25.923 | 1.00 | 27.12 |
| 762 | O | ASN | A | 299 | 12.982 | 25.288 | 27.134 | 1.00 | 26.42 |
| 763 | N | LEU | A | 300 | 13.684 | 25.642 | 25.028 | 1.00 | 24.59 |
| 764 | CA | LEU | A | 300 | 14.705 | 26.596 | 25.433 | 1.00 | 23.00 |
| 765 | CB | LEU | A | 300 | 15.620 | 26.921 | 24.252 | 1.00 | 20.53 |
| 766 | CG | LEU | A | 300 | 16.484 | 25.795 | 23.687 | 1.00 | 20.80 |
| 767 | CD1 | LEU | A | 300 | 17.176 | 26.283 | 22.424 | 1.00 | 20.69 |
| 768 | CD2 | LEU | A | 300 | 17.509 | 25.357 | 24.729 | 1.00 | 21.60 |
| 769 | C | LEU | A | 300 | 14.046 | 27.873 | 25.909 | 1.00 | 21.59 |
| 770 | O | LEU | A | 300 | 12.900 | 28.141 | 25.571 | 1.00 | 20.49 |
| 771 | N | ASP | A | 301 | 14.771 | 28.654 | 26.703 | 1.00 | 23.45 |
| 772 | CA | ASP | A | 301 | 14.252 | 29.931 | 27.172 | 1.00 | 23.40 |
| 773 | CB | ASP | A | 301 | 15.276 | 30.636 | 28.058 | 1.00 | 22.34 |
| 774 | CG | ASP | A | 301 | 14.863 | 32.052 | 28.406 | 1.00 | 23.70 |
| 775 | OD1 | ASP | A | 301 | 13.868 | 32.225 | 29.135 | 1.00 | 24.73 |
| 776 | OD2 | ASP | A | 301 | 15.531 | 33.000 | 27.941 | 1.00 | 24.97 |
| 777 | C | ASP | A | 301 | 14.049 | 30.738 | 25.894 | 1.00 | 23.49 |
| 778 | O | ASP | A | 301 | 14.817 | 30.584 | 24.945 | 1.00 | 22.64 |
| 779 | N | LEU | A | 302 | 13.029 | 31.588 | 25.863 | 1.00 | 24.03 |
| 780 | CA | LEU | A | 302 | 12.760 | 32.376 | 24.665 | 1.00 | 24.61 |
| 781 | CB | LEU | A | 302 | 11.486 | 33.209 | 24.845 | 1.00 | 25.91 |
| 782 | CG | LEU | A | 302 | 11.303 | 34.143 | 26.039 | 1.00 | 29.90 |
| 783 | CD1 | LEU | A | 302 | 12.353 | 35.243 | 26.046 | 1.00 | 31.19 |
| 784 | CD2 | LEU | A | 302 | 9.910 | 34.751 | 25.944 | 1.00 | 32.22 |
| 785 | C | LEU | A | 302 | 13.914 | 33.274 | 24.222 | 1.00 | 23.78 |
| 786 | O | LEU | A | 302 | 14.078 | 33.525 | 23.031 | 1.00 | 22.46 |
| 787 | N | ASN | A | 303 | 14.710 | 33.764 | 25.168 | 1.00 | 22.87 |
| 788 | CA | ASN | A | 303 | 15.842 | 34.615 | 24.814 | 1.00 | 23.57 |
| 789 | CB | ASN | A | 303 | 16.499 | 35.183 | 26.072 | 1.00 | 24.41 |
| 790 | OG | ASN | A | 303 | 15.597 | 36.151 | 26.812 | 1.00 | 25.82 |
| 791 | OD1 | ASN | A | 303 | 15.341 | 37.265 | 26.346 | 1.00 | 24.76 |
| 792 | ND2 | ASN | A | 303 | 15.097 | 35.724 | 27.969 | 1.00 | 25.85 |
| 793 | C | ASN | A | 303 | 16.858 | 33.792 | 24.025 | 1.00 | 23.03 |
| 794 | O | ASN | A | 303 | 17.475 | 34.279 | 23.071 | 1.00 | 22.95 |
| 795 | N | ASP | A | 304 | 17.031 | 32.539 | 24.428 | 1.00 | 21.92 |
| 796 | CA | ASP | A | 304 | 17.965 | 31.659 | 23.742 | 1.00 | 21.37 |
| 797 | CB | ASP | A | 304 | 18.232 | 30.404 | 24.570 | 1.00 | 20.90 |
| 798 | CG | ASP | A | 304 | 19.282 | 30.630 | 25.640 | 1.00 | 20.90 |
| 799 | OD1 | ASP | A | 304 | 19.790 | 31.766 | 25.753 | 1.00 | 22.16 |
| 800 | OD2 | ASP | A | 304 | 19.602 | 29.671 | 26.365 | 1.00 | 22.62 |
| 801 | C | ASP | A | 304 | 17.414 | 31.284 | 22.375 | 1.00 | 20.42 |
| 802 | O | ASP | A | 304 | 18.177 | 31.074 | 21.433 | 1.00 | 21.27 |
| 803 | N | GLN | A | 305 | 16.091 | 31.200 | 22.264 | 1.00 | 19.98 |
| 804 | CA | GLN | A | 305 | 15.478 | 30.871 | 20.978 | 1.00 | 20.73 |
| 805 | CB | GLN | A | 305 | 13.969 | 30.644 | 21.124 | 1.00 | 20.41 |
| 806 | CG | GLN | A | 305 | 13.593 | 29.369 | 21.870 | 1.00 | 22.80 |
| 807 | CD | GLN | A | 305 | 12.093 | 29.135 | 21.911 | 1.00 | 24.47 |
| 808 | OE1 | GLN | A | 305 | 11.420 | 29.157 | 20.880 | 1.00 | 26.99 |
| 809 | NE2 | GLN | A | 305 | 11.562 | 28.903 | 23.107 | 1.00 | 23.97 |
| 810 | C | GLN | A | 305 | 15.738 | 32.036 | 20.027 | 1.00 | 19.47 |
| 811 | O | GLN | A | 305 | 16.093 | 31.838 | 18.865 | 1.00 | 19.20 |
| 812 | N | VAL | A | 306 | 15.561 | 33.250 | 20.539 | 1.00 | 19.92 |
| 813 | CA | VAL | A | 306 | 15.787 | 34.463 | 19.760 | 1.00 | 18.92 |
| 814 | CB | VAL | A | 306 | 15.414 | 35.717 | 20.582 | 1.00 | 19.64 |
| 815 | CG1 | VAL | A | 306 | 15.853 | 36.982 | 19.860 | 1.00 | 20.62 |
| 816 | CG2 | VAL | A | 306 | 13.912 | 35.747 | 20.802 | 1.00 | 19.50 |
| 817 | C | VAL | A | 306 | 17.246 | 34.559 | 19.321 | 1.00 | 18.13 |
| 818 | O | VAL | A | 306 | 17.539 | 34.860 | 18.160 | 1.00 | 17.71 |
| 819 | N | THR | A | 307 | 18.159 | 34.293 | 20.250 | 1.00 | 17.09 |
| 820 | CA | THR | A | 307 | 19.586 | 34.361 | 19.957 | 1.00 | 17.05 |
| 821 | CB | THR | A | 307 | 20.424 | 34.138 | 21.242 | 1.00 | 17.63 |
| 822 | OG1 | THR | A | 307 | 20.153 | 35.197 | 22.171 | 1.00 | 16.06 |
| 823 | CG2 | THR | A | 307 | 21.918 | 34.135 | 20.921 | 1.00 | 16.45 |
| 824 | C | THR | A | 307 | 20.006 | 33.346 | 18.892 | 1.00 | 16.61 |
| 825 | O | THR | A | 307 | 20.766 | 33.672 | 17.986 | 1.00 | 17.34 |
| 826 | N | LEU | A | 308 | 19.503 | 32.121 | 18.994 | 1.00 | 15.88 |
| 827 | CA | LEU | A | 308 | 19.865 | 31.093 | 18.025 | 1.00 | 16.20 |
| 828 | CB | LEU | A | 308 | 19.262 | 29.741 | 18.417 | 1.00 | 17.78 |
| 829 | CG | LEU | A | 308 | 19.884 | 29.111 | 19.664 | 1.00 | 16.63 |
| 830 | CD1 | LEU | A | 308 | 19.285 | 27.720 | 19.912 | 1.00 | 17.89 |
| 831 | CD2 | LEU | A | 308 | 21.393 | 29.016 | 19.472 | 1.00 | 17.96 |
| 832 | C | LEU | A | 308 | 19.422 | 31.479 | 16.622 | 1.00 | 16.44 |
| 833 | O | LEU | A | 308 | 20.154 | 31.263 | 15.650 | 1.00 | 17.08 |
| 834 | N | LEU | A | 309 | 18.224 | 32.043 | 16.511 | 1.00 | 16.59 |
| 835 | CA | LEU | A | 309 | 17.724 | 32.463 | 15.204 | 1.00 | 17.03 |
| 836 | CB | LEU | A | 309 | 16.211 | 32.710 | 15.261 | 1.00 | 16.97 |
| 837 | CG | LEU | A | 309 | 15.373 | 31.426 | 15.326 | 1.00 | 18.35 |
| 838 | CD1 | LEU | A | 309 | 13.914 | 31.777 | 15.589 | 1.00 | 23.00 |
| 839 | CD2 | LEU | A | 309 | 15.506 | 30.657 | 14.020 | 1.00 | 19.92 |
| 840 | C | LEU | A | 309 | 18.447 | 33.726 | 14.751 | 1.00 | 17.00 |
| 841 | O | LEU | A | 309 | 18.825 | 33.855 | 13.587 | 1.00 | 17.06 |
| 842 | N | LYS | A | 310 | 18.649 | 34.657 | 15.675 | 1.00 | 16.85 |
| 843 | CA | LYS | A | 310 | 19.332 | 35.903 | 15.336 | 1.00 | 17.88 |
| 844 | CB | LYS | A | 310 | 19.570 | 36.737 | 16.595 | 1.00 | 19.60 |
| 845 | CG | LYS | A | 310 | 20.250 | 38.079 | 16.325 | 1.00 | 21.39 |
| 846 | CD | LYS | A | 310 | 20.640 | 38.760 | 17.633 | 1.00 | 24.10 |
| 847 | CE | LYS | A | 310 | 21.273 | 40.120 | 17.385 | 1.00 | 24.44 |
| 848 | NZ | LYS | A | 310 | 20.305 | 41.066 | 16.750 | 1.00 | 26.22 |
| 849 | C | LYS | A | 310 | 20.670 | 35.659 | 14.632 | 1.00 | 18.39 |
| 850 | O | LYS | A | 310 | 20.956 | 36.276 | 13.605 | 1.00 | 19.24 |
| 851 | N | TYR | A | 311 | 21.478 | 34.751 | 15.174 | 1.00 | 16.44 |
| 852 | CA | TYR | A | 311 | 22.788 | 34.459 | 14.601 | 1.00 | 19.84 |
| 853 | CB | TYR | A | 311 | 23.784 | 34.136 | 15.719 | 1.00 | 23.67 |
| 854 | CG | TYR | A | 311 | 24.144 | 35.333 | 16.568 | 1.00 | 27.71 |
| 855 | CD1 | TYR | A | 311 | 24.994 | 36.325 | 16.083 | 1.00 | 30.38 |
| 856 | CE1 | TYR | A | 311 | 25.315 | 37.439 | 16.856 | 1.00 | 32.19 |
| 857 | CD2 | TYR | A | 311 | 23.620 | 35.484 | 17.850 | 1.00 | 30.97 |
| 858 | CE2 | TYR | A | 311 | 23.933 | 36.595 | 18.631 | 1.00 | 33.09 |
| 859 | CZ | TYR | A | 311 | 24.782 | 37.566 | 18.127 | 1.00 | 33.75 |
| 860 | OH | TYR | A | 311 | 25.107 | 38.659 | 18.895 | 1.00 | 36.85 |
| 861 | C | TYR | A | 311 | 22.803 | 33.336 | 13.573 | 1.00 | 17.92 |
| 862 | O | TYR | A | 311 | 23.712 | 33.261 | 12.743 | 1.00 | 20.86 |
| 863 | N | GLY | A | 312 | 21.799 | 32.469 | 13.610 | 1.00 | 16.75 |
| 864 | CA | GLY | A | 312 | 21.784 | 31.364 | 12.669 | 1.00 | 15.82 |
| 865 | C | GLY | A | 312 | 20.979 | 31.509 | 11.390 | 1.00 | 16.03 |
| 866 | O | GLY | A | 312 | 21.278 | 30.834 | 10.403 | 1.00 | 16.07 |
| 867 | N | VAL | A | 313 | 19.971 | 32.377 | 11.372 | 1.00 | 15.36 |
| 868 | CA | VAL | A | 313 | 19.154 | 32.494 | 10.170 | 1.00 | 16.30 |
| 869 | CB | VAL | A | 313 | 17.975 | 33.506 | 10.353 | 1.00 | 17.29 |
| 870 | CG1 | VAL | A | 313 | 18.485 | 34.904 | 10.609 | 1.00 | 16.70 |
| 871 | CG2 | VAL | A | 313 | 17.093 | 33.486 | 9.124 | 1.00 | 23.41 |
| 872 | C | VAL | A | 313 | 19.901 | 32.811 | 8.878 | 1.00 | 14.96 |
| 873 | O | VAL | A | 313 | 19.631 | 32.198 | 7.846 | 1.00 | 12.97 |
| 874 | N | TYR | A | 314 | 20.852 | 33.738 | 8.902 | 1.00 | 13.63 |
| 875 | CA | TYR | A | 314 | 21.539 | 34.047 | 7.654 | 1.00 | 14.50 |
| 876 | CB | TYR | A | 314 | 22.206 | 35.429 | 7.729 | 1.00 | 15.24 |
| 877 | CG | TYR | A | 314 | 21.201 | 36.518 | 7.423 | 1.00 | 15.95 |
| 878 | CD1 | TYR | A | 314 | 20.785 | 36.754 | 6.115 | 1.00 | 16.47 |
| 879 | CE1 | TYR | A | 314 | 19.764 | 37.664 | 5.838 | 1.00 | 17.10 |
| 880 | CD2 | TYR | A | 314 | 20.578 | 37.230 | 8.452 | 1.00 | 15.91 |
| 881 | CE2 | TYR | A | 314 | 19.563 | 38.137 | 8.190 | 1.00 | 15.61 |
| 882 | CZ | TYR | A | 314 | 19.156 | 38.349 | 6.883 | 1.00 | 16.58 |
| 883 | OH | TYR | A | 314 | 18.128 | 39.229 | 6.619 | 1.00 | 17.94 |
| 884 | C | TYR | A | 314 | 22.514 | 32.968 | 7.212 | 1.00 | 14.15 |
| 885 | O | TYR | A | 314 | 22.786 | 32.827 | 6.017 | 1.00 | 14.15 |
| 886 | N | GLU | A | 315 | 23.041 | 32.198 | 8.159 | 1.00 | 12.79 |
| 887 | CA | GLU | A | 315 | 23.939 | 31.113 | 7.776 | 1.00 | 13.63 |
| 888 | CB | GLU | A | 315 | 24.581 | 30.471 | 9.012 | 1.00 | 15.05 |
| 889 | CG | GLU | A | 315 | 25.608 | 31.379 | 9.701 | 1.00 | 15.42 |
| 890 | CD | GLU | A | 315 | 26.281 | 30.734 | 10.900 | 1.00 | 16.70 |
| 891 | OE1 | GLU | A | 315 | 26.093 | 29.520 | 11.125 | 1.00 | 17.95 |
| 892 | OE2 | GLU | A | 315 | 27.008 | 31.446 | 11.617 | 1.00 | 16.57 |
| 893 | C | GLU | A | 315 | 23.055 | 30.102 | 7.034 | 1.00 | 14.04 |
| 894 | O | GLU | A | 315 | 23.449 | 29.549 | 6.007 | 1.00 | 12.76 |
| 895 | N | ALA | A | 316 | 21.842 | 29.895 | 7.545 | 1.00 | 13.17 |
| 896 | CA | ALA | A | 316 | 20.906 | 28.953 | 6.930 | 1.00 | 13.03 |
| 897 | CB | ALA | A | 316 | 19.701 | 28.721 | 7.846 | 1.00 | 13.75 |
| 898 | C | ALA | A | 316 | 20.435 | 29.488 | 5.589 | 1.00 | 13.24 |
| 899 | O | ALA | A | 316 | 20.327 | 28.748 | 4.612 | 1.00 | 12.79 |
| 900 | N | ILE | A | 317 | 20.146 | 30.784 | 5.544 | 1.00 | 12.44 |
| 901 | CA | ILE | A | 317 | 19.693 | 31.399 | 4.306 | 1.00 | 11.19 |
| 902 | CB | ILE | A | 317 | 19.360 | 32.896 | 4.534 | 1.00 | 10.99 |
| 903 | CG2 | ILE | A | 317 | 19.215 | 33.639 | 3.193 | 1.00 | 11.43 |
| 904 | CG1 | ILE | A | 317 | 18.053 | 32.999 | 5.325 | 1.00 | 12.81 |
| 905 | CD1 | ILE | A | 317 | 17.711 | 34.415 | 5.775 | 1.00 | 13.13 |
| 906 | C | ILE | A | 317 | 20.720 | 31.242 | 3.184 | 1.00 | 12.02 |
| 907 | O | ILE | A | 317 | 20.377 | 30.815 | 2.082 | 1.00 | 12.18 |
| 908 | N | PHE | A | 318 | 21.980 | 31.566 | 3.450 | 1.00 | 13.30 |
| 909 | CA | PHE | A | 318 | 22.982 | 31.429 | 2.393 | 1.00 | 13.60 |
| 910 | CB | PHE | A | 318 | 24.275 | 32.137 | 2.797 | 1.00 | 14.04 |
| 911 | CG | PHE | A | 318 | 24.095 | 33.610 | 3.056 | 1.00 | 13.96 |
| 912 | CD1 | PHE | A | 318 | 23.204 | 34.355 | 2.287 | 1.00 | 16.84 |
| 913 | CD2 | PHE | A | 318 | 24.815 | 34.252 | 4.056 | 1.00 | 15.29 |
| 914 | CE1 | PHE | A | 318 | 23.030 | 35.722 | 2.509 | 1.00 | 17.71 |
| 915 | CE2 | PHE | A | 318 | 24.649 | 35.616 | 4.285 | 1.00 | 18.04 |
| 916 | CZ | PHE | A | 318 | 23.750 | 36.350 | 3.506 | 1.00 | 15.92 |
| 917 | C | PHE | A | 318 | 23.233 | 29.959 | 2.024 | 1.00 | 13.74 |
| 918 | O | PHE | A | 318 | 23.540 | 29.647 | 0.875 | 1.00 | 13.80 |
| 919 | N | ALA | A | 319 | 23.103 | 29.054 | 2.989 | 1.00 | 13.03 |
| 920 | CA | ALA | A | 319 | 23.275 | 27.631 | 2.698 | 1.00 | 12.43 |
| 921 | CB | ALA | A | 319 | 23.257 | 26.807 | 4.001 | 1.00 | 11.53 |
| 922 | C | ALA | A | 319 | 22.127 | 27.186 | 1.781 | 1.00 | 13.70 |
| 923 | O | ALA | A | 319 | 22.340 | 26.474 | 0.792 | 1.00 | 13.68 |
| 924 | N | MET | A | 320 | 20.906 | 27.612 | 2.102 | 1.00 | 12.83 |
| 925 | CA | MET | A | 320 | 19.754 | 27.229 | 1.291 | 1.00 | 11.87 |
| 926 | CB | MET | A | 320 | 18.446 | 27.436 | 2.071 | 1.00 | 14.42 |
| 927 | CG | MET | A | 320 | 18.375 | 26.587 | 3.347 | 1.00 | 16.28 |
| 928 | SD | MET | A | 320 | 16.760 | 26.610 | 4.149 | 1.00 | 18.54 |
| 929 | CE | MET | A | 320 | 16.612 | 28.349 | 4.548 | 1.00 | 20.41 |
| 930 | C | MET | A | 320 | 19.690 | 27.941 | −0.060 | 1.00 | 13.87 |
| 931 | O | MET | A | 320 | 19.023 | 27.464 | −0.976 | 1.00 | 12.13 |
| 932 | N | LEU | A | 321 | 20.376 | 29.077 | −0.197 | 1.00 | 13.97 |
| 933 | CA | LEU | A | 321 | 20.371 | 29.775 | −1.485 | 1.00 | 15.41 |
| 934 | CB | LEU | A | 321 | 21.223 | 31.051 | −1.424 | 1.00 | 15.37 |
| 935 | CG | LEU | A | 321 | 20.547 | 32.292 | −0.829 | 1.00 | 18.20 |
| 936 | CD1 | LEU | A | 321 | 21.490 | 33.490 | −0.942 | 1.00 | 19.32 |
| 937 | CD2 | LEU | A | 321 | 19.231 | 32.569 | −1.561 | 1.00 | 18.93 |
| 938 | C | LEU | A | 321 | 20.935 | 28.854 | −2.560 | 1.00 | 13.60 |
| 939 | O | LEU | A | 321 | 20.499 | 28.874 | −3.712 | 1.00 | 15.79 |
| 940 | N | SER | A | 322 | 21.919 | 28.052 | −2.168 | 1.00 | 12.99 |
| 941 | CA | SER | A | 322 | 22.575 | 27.121 | −3.072 | 1.00 | 14.61 |
| 942 | CB | SER | A | 322 | 23.541 | 26.240 | −2.276 | 1.00 | 14.55 |
| 943 | OG | SER | A | 322 | 24.372 | 27.042 | −1.457 | 1.00 | 15.05 |
| 944 | C | SER | A | 322 | 21.552 | 26.244 | −3.797 | 1.00 | 15.16 |
| 945 | O | SER | A | 322 | 21.734 | 25.892 | −4.963 | 1.00 | 16.58 |
| 946 | N | SER | A | 323 | 20.476 | 25.899 | −3.099 | 1.00 | 14.05 |
| 947 | CA | SER | A | 323 | 19.439 | 25.051 | −3.675 | 1.00 | 14.37 |
| 948 | CB | SER | A | 323 | 18.390 | 24.708 | −2.615 | 1.00 | 16.17 |
| 949 | OG | SER | A | 323 | 18.989 | 24.036 | −1.524 | 1.00 | 14.97 |
| 950 | C | SER | A | 323 | 18.741 | 25.667 | −4.883 | 1.00 | 15.39 |
| 951 | O | SER | A | 323 | 18.260 | 24.947 | −5.759 | 1.00 | 15.48 |
| 952 | N | VAL | A | 324 | 18.672 | 26.995 | −4.927 | 1.00 | 16.08 |
| 953 | CA | VAL | A | 324 | 17.999 | 27.667 | −6.029 | 1.00 | 16.93 |
| 954 | CB | VAL | A | 324 | 16.974 | 28.713 | −5.516 | 1.00 | 18.06 |
| 955 | CG1 | VAL | A | 324 | 15.878 | 28.020 | −4.716 | 1.00 | 20.27 |
| 956 | CG2 | VAL | A | 324 | 17.674 | 29.766 | −4.667 | 1.00 | 19.05 |
| 957 | C | VAL | A | 324 | 18.967 | 28.358 | −6.973 | 1.00 | 16.26 |
| 958 | O | VAL | A | 324 | 18.551 | 29.150 | −7.816 | 1.00 | 17.98 |
| 959 | N | MET | A | 325 | 20.251 | 28.034 | −6.853 | 1.00 | 16.16 |
| 960 | CA | MET | A | 325 | 21.276 | 28.648 | −7.700 | 1.00 | 17.20 |
| 961 | CB | MET | A | 325 | 22.355 | 29.329 | −6.846 | 1.00 | 16.55 |
| 962 | CG | MET | A | 325 | 21.941 | 30.527 | −6.005 | 1.00 | 16.51 |
| 963 | SD | MET | A | 325 | 23.361 | 31.079 | −4.984 | 1.00 | 17.83 |
| 964 | CE | MET | A | 325 | 22.982 | 32.821 | −4.800 | 1.00 | 19.15 |
| 965 | C | MET | A | 325 | 22.021 | 27.664 | −8.596 | 1.00 | 19.13 |
| 966 | O | MET | A | 325 | 22.098 | 26.470 | −8.308 | 1.00 | 20.24 |
| 967 | N | ASN | A | 326 | 22.557 | 28.188 | −9.694 | 1.00 | 19.31 |
| 968 | CA | ASN | A | 326 | 23.425 | 27.418 | −10.572 | 1.00 | 20.10 |
| 969 | CB | ASN | A | 326 | 22.716 | 26.858 | −11.821 | 1.00 | 21.23 |
| 970 | CG | ASN | A | 326 | 22.149 | 27.919 | −12.730 | 1.00 | 20.10 |
| 971 | OD1 | ASN | A | 326 | 22.672 | 29.028 | −12.837 | 1.00 | 21.27 |
| 972 | ND2 | ASN | A | 326 | 21.076 | 27.559 | −13.434 | 1.00 | 23.09 |
| 973 | C | ASN | A | 326 | 24.498 | 28.453 | −10.909 | 1.00 | 21.45 |
| 974 | O | ASN | A | 326 | 24.426 | 29.580 | −10.423 | 1.00 | 19.65 |
| 975 | N | LYS | A | 327 | 25.488 | 28.098 | −11.717 | 1.00 | 21.83 |
| 976 | CA | LYS | A | 327 | 26.560 | 29.043 | −12.011 | 1.00 | 23.64 |
| 977 | CB | LYS | A | 327 | 27.655 | 28.364 | −12.841 | 1.00 | 26.31 |
| 978 | CG | LYS | A | 327 | 27.285 | 28.119 | −14.297 | 1.00 | 30.33 |
| 979 | CD | LYS | A | 327 | 28.506 | 27.647 | −15.083 | 1.00 | 34.09 |
| 980 | CE | LYS | A | 327 | 28.258 | 27.640 | −16.589 | 1.00 | 36.35 |
| 981 | NZ | LYS | A | 327 | 27.208 | 26.665 | −16.992 | 1.00 | 39.18 |
| 982 | C | LYS | A | 327 | 26.158 | 30.342 | −12.704 | 1.00 | 22.90 |
| 983 | O | LYS | A | 327 | 26.924 | 31.308 | −12.685 | 1.00 | 22.33 |
| 984 | N | ASP | A | 328 | 24.965 | 30.387 | −13.290 | 1.00 | 21.50 |
| 985 | CA | ASP | A | 328 | 24.544 | 31.582 | −14.020 | 1.00 | 21.46 |
| 986 | CB | ASP | A | 328 | 24.110 | 31.185 | −15.433 | 1.00 | 22.79 |
| 987 | CG | ASP | A | 328 | 25.228 | 30.535 | −16.219 | 1.00 | 24.81 |
| 988 | OD1 | ASP | A | 328 | 26.327 | 31.124 | −16.295 | 1.00 | 28.16 |
| 989 | OD2 | ASP | A | 328 | 25.010 | 29.440 | −16.760 | 1.00 | 26.38 |
| 990 | C | ASP | A | 328 | 23.462 | 32.463 | −13.404 | 1.00 | 19.81 |
| 991 | O | ASP | A | 328 | 23.123 | 33.505 | −13.967 | 1.00 | 18.99 |
| 992 | N | GLY | A | 329 | 22.915 | 32.061 | −12.263 | 1.00 | 18.40 |
| 993 | CA | GLY | A | 329 | 21.879 | 32.872 | −11.648 | 1.00 | 17.35 |
| 994 | C | GLY | A | 329 | 21.093 | 32.134 | −10.584 | 1.00 | 16.28 |
| 995 | O | GLY | A | 329 | 21.466 | 31.030 | −10.186 | 1.00 | 15.47 |
| 996 | N | MET | A | 330 | 20.002 | 32.739 | −10.120 | 1.00 | 17.05 |
| 997 | CA | MET | A | 330 | 19.183 | 32.109 | −9.089 | 1.00 | 17.55 |
| 998 | CB | MET | A | 330 | 19.563 | 32.642 | −7.701 | 1.00 | 19.90 |
| 999 | CG | MET | A | 330 | 19.221 | 34.098 | −7.438 | 1.00 | 22.04 |
| 1000 | SD | MET | A | 330 | 19.415 | 34.525 | −5.667 | 1.00 | 24.36 |
| 1001 | CE | MET | A | 330 | 17.856 | 34.142 | −5.014 | 1.00 | 23.38 |
| 1002 | C | MET | A | 330 | 17.689 | 32.295 | −9.308 | 1.00 | 17.11 |
| 1003 | O | MET | A | 330 | 17.249 | 33.259 | −9.930 | 1.00 | 17.45 |
| 1004 | N | LEU | A | 331 | 16.908 | 31.350 | −8.799 | 1.00 | 16.64 |
| 1005 | CA | LEU | A | 331 | 15.460 | 31.411 | −8.912 | 1.00 | 16.30 |
| 1006 | CB | LEU | A | 331 | 14.843 | 30.051 | −8.595 | 1.00 | 17.42 |
| 1007 | CG | LEU | A | 331 | 15.026 | 28.943 | −9.620 | 1.00 | 19.04 |
| 1008 | CD1 | LEU | A | 331 | 14.408 | 27.650 | −9.079 | 1.00 | 18.84 |
| 1009 | CD2 | LEU | A | 331 | 14.363 | 29.359 | −10.925 | 1.00 | 17.82 |
| 1010 | C | LEU | A | 331 | 14.930 | 32.414 | −7.903 | 1.00 | 16.49 |
| 1011 | O | LEU | A | 331 | 15.415 | 32.465 | −6.774 | 1.00 | 17.61 |
| 1012 | N | VAL | A | 332 | 13.932 | 33.194 | −8.305 | 1.00 | 14.86 |
| 1013 | CA | VAL | A | 332 | 13.331 | 34.173 | −7.409 | 1.00 | 15.11 |
| 1014 | CB | VAL | A | 332 | 13.880 | 35.603 | −7.664 | 1.00 | 14.89 |
| 1015 | CG1 | VAL | A | 332 | 15.406 | 35.600 | −7.537 | 1.00 | 17.85 |
| 1016 | CG2 | VAL | A | 332 | 13.453 | 36.099 | −9.040 | 1.00 | 16.47 |
| 1017 | C | VAL | A | 332 | 11.821 | 34.193 | −7.589 | 1.00 | 14.36 |
| 1018 | O | VAL | A | 332 | 11.283 | 33.559 | −8.501 | 1.00 | 15.87 |
| 1019 | N | ALA | A | 333 | 11.148 | 34.928 | −6.713 | 1.00 | 13.97 |
| 1020 | CA | ALA | A | 333 | 9.698 | 35.071 | −6.757 | 1.00 | 14.91 |
| 1021 | CB | ALA | A | 333 | 9.294 | 35.898 | −7.977 | 1.00 | 15.58 |
| 1022 | C | ALA | A | 333 | 8.978 | 33.724 | −6.768 | 1.00 | 14.63 |
| 1023 | O | ALA | A | 333 | 8.168 | 33.441 | −7.660 | 1.00 | 14.60 |
| 1024 | N | TYR | A | 334 | 9.285 | 32.899 | −5.772 | 1.00 | 14.26 |
| 1025 | CA | TYR | A | 334 | 8.660 | 31.589 | −5.630 | 1.00 | 15.27 |
| 1026 | CB | TYR | A | 334 | 7.195 | 31.781 | −5.230 | 1.00 | 15.76 |
| 1027 | CG | TYR | A | 334 | 7.086 | 32.362 | −3.840 | 1.00 | 18.04 |
| 1028 | CD1 | TYR | A | 334 | 7.025 | 31.532 | −2.722 | 1.00 | 19.51 |
| 1029 | CE1 | TYR | A | 334 | 7.073 | 32.055 | −1.432 | 1.00 | 20.29 |
| 1030 | CD2 | TYR | A | 334 | 7.182 | 33.737 | −3.634 | 1.00 | 19.02 |
| 1031 | CE2 | TYR | A | 334 | 7.237 | 34.272 | −2.348 | 1.00 | 20.93 |
| 1032 | CZ | TYR | A | 334 | 7.186 | 33.427 | −1.253 | 1.00 | 20.76 |
| 1033 | OH | TYR | A | 334 | 7.284 | 33.950 | 0.019 | 1.00 | 21.61 |
| 1034 | C | TYR | A | 334 | 8.797 | 30.715 | −6.871 | 1.00 | 14.54 |
| 1035 | O | TYR | A | 334 | 7.854 | 30.038 | −7.290 | 1.00 | 15.22 |
| 1036 | N | GLY | A | 335 | 9.996 | 30.744 | −7.441 | 1.00 | 15.09 |
| 1037 | CA | GLY | A | 335 | 10.306 | 29.944 | −8.612 | 1.00 | 16.73 |
| 1038 | C | GLY | A | 335 | 9.795 | 30.441 | −9.946 | 1.00 | 18.37 |
| 1039 | O | GLY | A | 335 | 9.965 | 29.761 | −10.958 | 1.00 | 18.69 |
| 1040 | N | ASN | A | 336 | 9.188 | 31.623 | −9.972 | 1.00 | 18.57 |
| 1041 | CA | ASN | A | 336 | 8.654 | 32.133 | −11.227 | 1.00 | 18.81 |
| 1042 | CB | ASN | A | 336 | 7.322 | 32.835 | −10.988 | 1.00 | 21.87 |
| 1043 | CG | ASN | A | 336 | 6.149 | 31.866 | −10.981 | 1.00 | 24.72 |
| 1044 | OD1 | ASN | A | 336 | 5.003 | 32.275 | −10.900 | 1.00 | 32.52 |
| 1045 | ND2 | ASN | A | 336 | 6.437 | 30.579 | −11.071 | 1.00 | 29.15 |
| 1046 | C | ASN | A | 336 | 9.595 | 33.053 | −11.993 | 1.00 | 18.54 |
| 1047 | O | ASN | A | 336 | 9.277 | 33.492 | −13.096 | 1.00 | 16.73 |
| 1048 | N | GLY | A | 337 | 10.749 | 33.335 | −11.406 | 1.00 | 16.20 |
| 1049 | CA | GLY | A | 337 | 11.717 | 34.187 | −12.066 | 1.00 | 16.52 |
| 1050 | C | GLY | A | 337 | 13.114 | 33.638 | −11.886 | 1.00 | 16.47 |
| 1051 | O | GLY | A | 337 | 13.347 | 32.809 | −11.008 | 1.00 | 15.40 |
| 1052 | N | PHE | A | 338 | 14.041 | 34.090 | −12.728 | 1.00 | 15.82 |
| 1053 | CA | PHE | A | 338 | 15.436 | 33.672 | −12.654 | 1.00 | 15.40 |
| 1054 | CB | PHE | A | 338 | 15.722 | 32.543 | −13.651 | 1.00 | 16.65 |
| 1055 | CG | PHE | A | 338 | 17.156 | 32.066 | −13.641 | 1.00 | 20.70 |
| 1056 | CD1 | PHE | A | 338 | 18.113 | 32.664 | −14.464 | 1.00 | 19.82 |
| 1057 | CD2 | PHE | A | 338 | 17.548 | 31.021 | −12.808 | 1.00 | 19.54 |
| 1058 | CE1 | PHE | A | 338 | 19.433 | 32.225 | −14.460 | 1.00 | 22.34 |
| 1059 | CE2 | PHE | A | 338 | 18.873 | 30.570 | −12.794 | 1.00 | 22.09 |
| 1060 | CZ | PHE | A | 338 | 19.816 | 31.174 | −13.624 | 1.00 | 22.09 |
| 1061 | C | PHE | A | 338 | 16.265 | 34.904 | −12.994 | 1.00 | 16.41 |
| 1062 | O | PHE | A | 338 | 16.212 | 35.411 | −14.117 | 1.00 | 15.80 |
| 1063 | N | ILE | A | 339 | 17.014 | 35.397 | −12.015 | 1.00 | 14.27 |
| 1064 | CA | ILE | A | 339 | 17.828 | 36.581 | −12.229 | 1.00 | 15.49 |
| 1065 | CB | ILE | A | 339 | 17.689 | 37.550 | −11.028 | 1.00 | 15.76 |
| 1066 | CG2 | ILE | A | 339 | 18.192 | 36.892 | −9.760 | 1.00 | 17.30 |
| 1067 | CG1 | ILE | A | 339 | 18.434 | 38.853 | −11.311 | 1.00 | 15.24 |
| 1068 | CD1 | ILE | A | 339 | 17.955 | 39.999 | −10.429 | 1.00 | 16.30 |
| 1069 | C | ILE | A | 339 | 19.274 | 36.151 | −12.453 | 1.00 | 16.57 |
| 1070 | O | ILE | A | 339 | 19.835 | 35.379 | −11.676 | 1.00 | 16.99 |
| 1071 | N | THR | A | 340 | 19.884 | 36.644 | −13.526 | 1.00 | 15.69 |
| 1072 | CA | THR | A | 340 | 21.251 | 36.232 | −13.835 | 1.00 | 17.54 |
| 1073 | CB | THR | A | 340 | 21.625 | 36.501 | −15.321 | 1.00 | 18.08 |
| 1074 | OG1 | THR | A | 340 | 21.680 | 37.913 | −15.563 | 1.00 | 18.08 |
| 1075 | CG2 | THR | A | 340 | 20.611 | 35.876 | −16.239 | 1.00 | 19.40 |
| 1076 | C | THR | A | 340 | 22.337 | 36.840 | −12.973 | 1.00 | 17.79 |
| 1077 | O | THR | A | 340 | 22.272 | 38.001 | −12.551 | 1.00 | 17.03 |
| 1078 | N | ARG | A | 341 | 23.347 | 36.019 | −12.729 | 1.00 | 18.29 |
| 1079 | CA | ARG | A | 341 | 24.503 | 36.395 | −11.944 | 1.00 | 20.41 |
| 1080 | CB | ARG | A | 341 | 25.470 | 35.216 | −11.910 | 1.00 | 20.86 |
| 1081 | CG | ARG | A | 341 | 26.710 | 35.443 | −11.093 | 1.00 | 23.49 |
| 1082 | CD | ARG | A | 341 | 27.502 | 34.152 | −11.003 | 1.00 | 22.29 |
| 1083 | NE | ARG | A | 341 | 28.625 | 34.282 | −10.089 | 1.00 | 25.39 |
| 1084 | CZ | ARG | A | 341 | 29.432 | 33.283 | −9.755 | 1.00 | 25.74 |
| 1085 | NH1 | ARG | A | 341 | 29.239 | 32.074 | −10.264 | 1.00 | 25.82 |
| 1086 | NH2 | ARG | A | 341 | 30.427 | 33.495 | −8.908 | 1.00 | 25.63 |
| 1087 | C | ARG | A | 341 | 25.175 | 37.606 | −12.587 | 1.00 | 20.85 |
| 1088 | O | ARG | A | 341 | 25.630 | 38.515 | −11.900 | 1.00 | 20.39 |
| 1089 | N | GLU | A | 342 | 25.225 | 37.615 | −13.915 | 1.00 | 21.71 |
| 1090 | CA | GLU | A | 342 | 25.858 | 38.717 | −14.633 | 1.00 | 23.42 |
| 1091 | CB | GLU | A | 342 | 26.044 | 38.343 | −16.104 | 1.00 | 26.93 |
| 1092 | CG | GLU | A | 342 | 27.151 | 37.324 | −16.330 | 1.00 | 31.26 |
| 1093 | CD | GLU | A | 342 | 28.501 | 37.816 | −15.832 | 1.00 | 33.51 |
| 1094 | OE1 | GLU | A | 342 | 28.961 | 38.882 | −16.294 | 1.00 | 36.23 |
| 1095 | OE2 | GLU | A | 342 | 29.105 | 37.140 | −14.977 | 1.00 | 36.72 |
| 1096 | C | GLU | A | 342 | 25.101 | 40.037 | −14.510 | 1.00 | 21.78 |
| 1097 | O | GLU | A | 342 | 25.715 | 41.103 | −14.425 | 1.00 | 23.51 |
| 1098 | N | PUB | A | 343 | 23.774 | 39.974 | −14.496 | 1.00 | 20.79 |
| 1099 | CA | PUB | A | 343 | 22.982 | 41.188 | −14.358 | 1.00 | 20.13 |
| 1100 | CB | PHE | A | 343 | 21.491 | 40.880 | −14.534 | 1.00 | 21.21 |
| 1101 | CG | PHE | A | 343 | 20.598 | 42.065 | −14.304 | 1.00 | 22.31 |
| 1102 | CD1 | PHE | A | 343 | 20.800 | 43.255 | −15.003 | 1.00 | 22.58 |
| 1103 | CD2 | PHE | A | 343 | 19.556 | 41.998 | −13.385 | 1.00 | 22.12 |
| 1104 | CE1 | PUB | A | 343 | 19.978 | 44.356 | −14.784 | 1.00 | 22.50 |
| 1105 | CE2 | PHE | A | 343 | 18.729 | 43.095 | −13.159 | 1.00 | 21.07 |
| 1106 | CZ | PUB | A | 343 | 18.940 | 44.277 | −13.861 | 1.00 | 23.38 |
| 1107 | C | PHE | A | 343 | 23.237 | 41.787 | −12.973 | 1.00 | 20.06 |
| 1108 | O | PHE | A | 343 | 23.389 | 42.998 | −12.821 | 1.00 | 18.10 |
| 1109 | N | LEU | A | 344 | 23.291 | 40.929 | −11.959 | 1.00 | 19.09 |
| 1110 | CA | LEU | A | 344 | 23.535 | 41.390 | −10.599 | 1.00 | 19.98 |
| 1111 | CB | LEU | A | 344 | 23.434 | 40.211 | −9.629 | 1.00 | 18.81 |
| 1112 | CG | LEU | A | 344 | 22.003 | 39.699 | −9.449 | 1.00 | 17.36 |
| 1113 | CD1 | LEU | A | 344 | 22.030 | 38.298 | −8.865 | 1.00 | 17.66 |
| 1114 | CD2 | LEU | A | 344 | 21.222 | 40.668 | −8.541 | 1.00 | 18.64 |
| 1115 | C | LEU | A | 344 | 24.891 | 42.082 | −10.451 | 1.00 | 20.25 |
| 1116 | O | LEU | A | 344 | 25.000 | 43.113 | −9.785 | 1.00 | 20.17 |
| 1117 | N | LYS | A | 345 | 25.926 | 41.532 | −11.077 | 1.00 | 22.58 |
| 1118 | CA | LYS | A | 345 | 27.243 | 42.149 | −10.976 | 1.00 | 25.12 |
| 1119 | CB | LYS | A | 345 | 28.329 | 41.149 | −11.388 | 1.00 | 28.30 |
| 1120 | CG | LYS | A | 345 | 28.086 | 40.453 | −12.709 | 1.00 | 31.49 |
| 1121 | CD | LYS | A | 345 | 28.931 | 39.185 | −12.824 | 1.00 | 34.20 |
| 1122 | CE | LYS | A | 345 | 30.418 | 39.474 | −12.663 | 1.00 | 32.93 |
| 1123 | NZ | LYS | A | 345 | 31.242 | 38.267 | −12.935 | 1.00 | 33.54 |
| 1124 | C | LYS | A | 345 | 27.349 | 43.442 | −11.787 | 1.00 | 26.38 |
| 1125 | O | LYS | A | 345 | 28.246 | 44.259 | −11.551 | 1.00 | 28.05 |
| 1126 | N | SER | A | 346 | 26.411 | 43.644 | −12.713 | 1.00 | 25.99 |
| 1127 | CA | SER | A | 346 | 26.397 | 44.840 | −13.557 | 1.00 | 25.78 |
| 1128 | CB | SER | A | 346 | 25.596 | 44.590 | −14.834 | 1.00 | 27.38 |
| 1129 | OG | SER | A | 346 | 24.206 | 44.742 | −14.590 | 1.00 | 27.61 |
| 1130 | C | SER | A | 346 | 25.782 | 46.028 | −12.832 | 1.00 | 25.54 |
| 1131 | O | SER | A | 346 | 25.870 | 47.169 | −13.298 | 1.00 | 24.80 |
| 1132 | N | LEU | A | 347 | 25.151 | 45.762 | −11.694 | 1.00 | 21.74 |
| 1133 | CA | LEU | A | 347 | 24.529 | 46.826 | −10.925 | 1.00 | 21.47 |
| 1134 | CB | LEU | A | 347 | 23.686 | 46.242 | −9.790 | 1.00 | 20.68 |
| 1135 | CG | LEU | A | 347 | 22.544 | 45.312 | −10.207 | 1.00 | 17.94 |
| 1136 | CD1 | LEU | A | 347 | 21.932 | 44.695 | −8.952 | 1.00 | 17.43 |
| 1137 | CD2 | LEU | A | 347 | 21.497 | 46.094 | −10.998 | 1.00 | 18.91 |
| 1138 | C | LEU | A | 347 | 25.585 | 47.751 | −10.341 | 1.00 | 22.34 |
| 1139 | O | LEU | A | 347 | 26.773 | 47.431 | −10.306 | 1.00 | 21.32 |
| 1140 | N | ARG | A | 348 | 25.133 | 48.904 | −9.878 | 1.00 | 23.03 |
| 1141 | CA | ARG | A | 348 | 26.007 | 49.899 | −9.279 | 1.00 | 24.90 |
| 1142 | CB | ARG | A | 348 | 25.247 | 51.233 | −9.226 | 1.00 | 26.16 |
| 1143 | CG | ARG | A | 348 | 25.445 | 52.062 | −7.977 | 1.00 | 28.63 |
| 1144 | CD | ARG | A | 348 | 24.782 | 53.424 | −8.123 | 1.00 | 27.93 |
| 1145 | NE | ARG | A | 348 | 23.514 | 53.557 | −7.407 | 1.00 | 25.84 |
| 1146 | CZ | ARG | A | 348 | 23.385 | 53.485 | −6.086 | 1.00 | 25.53 |
| 1147 | NH1 | ARG | A | 348 | 24.446 | 53.264 | −5.318 | 1.00 | 26.92 |
| 1148 | NH2 | ARG | A | 348 | 22.201 | 53.676 | −5.526 | 1.00 | 26.63 |
| 1149 | C | ARG | A | 348 | 26.431 | 49.457 | −7.877 | 1.00 | 23.21 |
| 1150 | O | ARG | A | 348 | 25.666 | 48.796 | −7.177 | 1.00 | 22.61 |
| 1151 | N | LYS | A | 349 | 27.654 | 49.799 | −7.472 | 1.00 | 23.12 |
| 1152 | CA | LYS | A | 349 | 28.104 | 49.457 | −6.126 | 1.00 | 22.49 |
| 1153 | CB | LYS | A | 349 | 29.575 | 49.848 | −5.910 | 1.00 | 23.58 |
| 1154 | CG | LYS | A | 349 | 30.585 | 49.055 | −6.738 | 1.00 | 22.91 |
| 1155 | CD | LYS | A | 349 | 32.017 | 49.580 | −6.540 | 1.00 | 25.76 |
| 1156 | CE | LYS | A | 349 | 32.665 | 49.069 | −5.261 | 1.00 | 26.68 |
| 1157 | NZ | LYS | A | 349 | 33.144 | 47.662 | −5.407 | 1.00 | 28.29 |
| 1158 | C | LYS | A | 349 | 27.218 | 50.268 | −5.188 | 1.00 | 22.76 |
| 1159 | O | LYS | A | 349 | 26.765 | 51.361 | −5.545 | 1.00 | 24.51 |
| 1160 | N | PRO | A | 350 | 26.975 | 49.767 | −3.970 | 1.00 | 21.69 |
| 1161 | CD | PRO | A | 350 | 26.248 | 50.523 | −2.935 | 1.00 | 23.55 |
| 1162 | CA | PRO | A | 350 | 27.476 | 48.506 | −3.421 | 1.00 | 21.20 |
| 1163 | CB | PRO | A | 350 | 27.573 | 48.818 | −1.943 | 1.00 | 23.13 |
| 1164 | CG | PRO | A | 350 | 26.312 | 49.586 | −1.724 | 1.00 | 22.88 |
| 1165 | C | PRO | A | 350 | 26.544 | 47.322 | −3.685 | 1.00 | 20.65 |
| 1166 | O | PRO | A | 350 | 26.815 | 46.197 | −3.256 | 1.00 | 20.59 |
| 1167 | N | PHE | A | 351 | 25.447 | 47.576 | −4.387 | 1.00 | 19.16 |
| 1168 | CA | PHE | A | 351 | 24.475 | 46.525 | −4.666 | 1.00 | 18.85 |
| 1169 | CB | PHE | A | 351 | 23.252 | 47.140 | −5.349 | 1.00 | 20.28 |
| 1170 | CG | PHE | A | 351 | 22.539 | 48.141 | −4.487 | 1.00 | 19.94 |
| 1171 | CD1 | PHE | A | 351 | 21.737 | 47.720 | −3.427 | 1.00 | 18.19 |
| 1172 | CD2 | PHE | A | 351 | 22.720 | 49.507 | −4.690 | 1.00 | 18.89 |
| 1173 | CE1 | PHE | A | 351 | 21.125 | 48.645 | −2.576 | 1.00 | 19.37 |
| 1174 | CE2 | PHE | A | 351 | 22.115 | 50.436 | −3.848 | 1.00 | 19.54 |
| 1175 | CZ | PHE | A | 351 | 21.316 | 50.005 | −2.786 | 1.00 | 19.87 |
| 1176 | C | PHE | A | 351 | 25.040 | 45.361 | −5.469 | 1.00 | 19.99 |
| 1177 | O | PHE | A | 351 | 24.620 | 44.213 | −5.289 | 1.00 | 19.11 |
| 1178 | N | CYS | A | 352 | 26.011 | 45.642 | −6.334 | 1.00 | 17.57 |
| 1179 | CA | CYS | A | 352 | 26.622 | 44.589 | −7.138 | 1.00 | 18.30 |
| 1180 | CB | CYS | A | 352 | 27.358 | 45.197 | −8.335 | 1.00 | 18.99 |
| 1181 | SG | CYS | A | 352 | 28.659 | 46.372 | −7.868 | 1.00 | 20.67 |
| 1182 | C | CYS | A | 352 | 27.599 | 43.760 | −6.307 | 1.00 | 18.14 |
| 1183 | O | CYS | A | 352 | 28.107 | 42.743 | −6.772 | 1.00 | 17.82 |
| 1184 | N | ASP | A | 353 | 27.843 | 44.184 | −5.069 | 1.00 | 18.21 |
| 1185 | CA | ASP | A | 353 | 28.781 | 43.477 | −4.206 | 1.00 | 18.60 |
| 1186 | CB | ASP | A | 353 | 29.640 | 44.484 | −3.439 | 1.00 | 20.06 |
| 1187 | CG | ASP | A | 353 | 30.432 | 45.395 | −4.364 | 1.00 | 20.29 |
| 1188 | OD1 | ASP | A | 353 | 31.032 | 44.880 | −5.319 | 1.00 | 21.00 |
| 1189 | OD2 | ASP | A | 353 | 30.457 | 46.621 | −4.137 | 1.00 | 22.31 |
| 1190 | C | ASP | A | 353 | 28.109 | 42.533 | −3.223 | 1.00 | 19.14 |
| 1191 | O | ASP | A | 353 | 28.771 | 41.917 | −2.386 | 1.00 | 20.18 |
| 1192 | N | ILE | A | 354 | 26.794 | 42.410 | −3.340 | 1.00 | 17.15 |
| 1193 | CA | ILE | A | 354 | 26.023 | 41.562 | −2.445 | 1.00 | 18.64 |
| 1194 | CB | ILE | A | 354 | 24.539 | 42.001 | −2.422 | 1.00 | 19.28 |
| 1195 | CG2 | ILE | A | 354 | 23.727 | 41.075 | −1.521 | 1.00 | 17.16 |
| 1196 | CG1 | ILE | A | 354 | 24.431 | 43.451 | −1.940 | 1.00 | 19.43 |
| 1197 | CD1 | ILE | A | 354 | 23.014 | 43.997 | −1.956 | 1.00 | 19.79 |
| 1198 | C | ILE | A | 354 | 26.042 | 40.074 | −2.778 | 1.00 | 18.41 |
| 1199 | O | ILE | A | 354 | 26.427 | 39.245 | −1.951 | 1.00 | 17.58 |
| 1200 | N | MET | A | 355 | 25.630 | 39.743 | −3.996 | 1.00 | 20.04 |
| 1201 | CA | MET | A | 355 | 25.507 | 38.349 | −4.406 | 1.00 | 18.56 |
| 1202 | CB | MET | A | 355 | 24.429 | 38.241 | −5.490 | 1.00 | 19.49 |
| 1203 | CG | MET | A | 355 | 23.033 | 38.634 | −5.014 | 1.00 | 20.24 |
| 1204 | SD | MET | A | 355 | 22.487 | 37.687 | −3.565 | 1.00 | 20.20 |
| 1205 | CB | MET | A | 355 | 22.589 | 36.017 | −4.219 | 1.00 | 16.96 |
| 1206 | C | MET | A | 355 | 26.702 | 37.497 | −4.821 | 1.00 | 18.99 |
| 1207 | O | MET | A | 355 | 26.655 | 36.283 | −4.637 | 1.00 | 17.53 |
| 1208 | N | GLU | A | 356 | 27.764 | 38.084 | −5.373 | 1.00 | 19.55 |
| 1209 | CA | GLU | A | 356 | 28.904 | 37.265 | −5.800 | 1.00 | 19.92 |
| 1210 | CB | GLU | A | 356 | 30.072 | 38.135 | −6.292 | 1.00 | 21.97 |
| 1211 | CG | GLU | A | 356 | 30.017 | 38.502 | −7.762 | 1.00 | 26.26 |
| 1212 | CD | GLU | A | 356 | 29.835 | 37.292 | −8.663 | 1.00 | 27.30 |
| 1213 | OE1 | GLU | A | 356 | 28.697 | 37.049 | −9.107 | 1.00 | 26.68 |
| 1214 | OE2 | GLU | A | 356 | 30.826 | 36.575 | −8.920 | 1.00 | 29.91 |
| 1215 | C | GLU | A | 356 | 29.432 | 36.288 | −4.752 | 1.00 | 18.52 |
| 1216 | O | GLU | A | 356 | 29.624 | 35.112 | −5.046 | 1.00 | 18.23 |
| 1217 | N | PRO | A | 357 | 29.686 | 36.758 | −3.522 | 1.00 | 18.98 |
| 1218 | CD | PRO | A | 357 | 29.538 | 38.130 | −3.001 | 1.00 | 20.10 |
| 1219 | CA | PRO | A | 357 | 30.195 | 35.863 | −2.477 | 1.00 | 19.09 |
| 1220 | CB | PRO | A | 357 | 30.290 | 36.777 | −1.259 | 1.00 | 20.02 |
| 1221 | CG | PRO | A | 357 | 30.525 | 38.137 | −1.869 | 1.00 | 20.53 |
| 1222 | C | PRO | A | 357 | 29.273 | 34.664 | −2.217 | 1.00 | 17.84 |
| 1223 | O | PRO | A | 357 | 29.730 | 33.572 | −1.869 | 1.00 | 15.96 |
| 1224 | N | LYS | A | 358 | 27.973 | 34.883 | −2.379 | 1.00 | 17.85 |
| 1225 | CA | LYS | A | 358 | 26.984 | 33.832 | −2.161 | 1.00 | 16.69 |
| 1226 | CB | LYS | A | 358 | 25.594 | 34.448 | −1.963 | 1.00 | 17.55 |
| 1227 | CG | LYS | A | 358 | 25.399 | 35.180 | −0.625 | 1.00 | 16.13 |
| 1228 | CD | LYS | A | 358 | 26.255 | 36.439 | −0.520 | 1.00 | 18.57 |
| 1229 | CE | LYS | A | 358 | 25.812 | 37.337 | 0.632 | 1.00 | 16.06 |
| 1230 | NZ | LYS | A | 358 | 26.664 | 38.570 | 0.749 | 1.00 | 15.29 |
| 1231 | C | LYS | A | 358 | 26.961 | 32.848 | −3.327 | 1.00 | 17.68 |
| 1232 | O | LYS | A | 358 | 26.787 | 31.638 | −3.132 | 1.00 | 17.04 |
| 1233 | N | PHE | A | 359 | 27.122 | 33.359 | −4.544 | 1.00 | 17.39 |
| 1234 | CA | PHE | A | 359 | 27.153 | 32.478 | −5.709 | 1.00 | 16.62 |
| 1235 | CB | PHE | A | 359 | 27.167 | 33.290 | −7.012 | 1.00 | 16.38 |
| 1236 | CG | PHE | A | 359 | 25.795 | 33.651 | −7.523 | 1.00 | 18.08 |
| 1237 | GD1 | PHE | A | 359 | 24.944 | 32.671 | −8.037 | 1.00 | 17.39 |
| 1238 | CD2 | PHE | A | 359 | 25.355 | 34.970 | −7.497 | 1.00 | 16.89 |
| 1239 | CE1 | PHE | A | 359 | 23.679 | 33.004 | −8.514 | 1.00 | 17.41 |
| 1240 | CE2 | PHE | A | 359 | 24.093 | 35.313 | −7.970 | 1.00 | 16.42 |
| 1241 | CZ | PHE | A | 359 | 23.253 | 34.327 | −8.481 | 1.00 | 16.76 |
| 1242 | C | PHE | A | 359 | 28.421 | 31.633 | −5.598 | 1.00 | 16.15 |
| 1243 | O | PHE | A | 359 | 28.400 | 30.424 | −5.851 | 1.00 | 16.24 |
| 1244 | N | ASP | A | 360 | 29.526 | 32.263 | −5.201 | 1.00 | 18.27 |
| 1245 | CA | ASP | A | 360 | 30.777 | 31.519 | −5.050 | 1.00 | 18.69 |
| 1246 | CB | ASP | A | 360 | 31.948 | 32.453 | −4.710 | 1.00 | 20.51 |
| 1247 | CG | ASP | A | 360 | 32.445 | 33.229 | −5.922 | 1.00 | 22.65 |
| 1248 | OD1 | ASP | A | 360 | 32.334 | 32.705 | −7.048 | 1.00 | 23.09 |
| 1249 | OD2 | ASP | A | 360 | 32.958 | 34.354 | −5.752 | 1.00 | 27.87 |
| 1250 | C | ASP | A | 360 | 30.637 | 30.432 | −3.988 | 1.00 | 18.16 |
| 1251 | O | ASP | A | 360 | 31.115 | 29.311 | −4.177 | 1.00 | 17.43 |
| 1252 | N | PHE | A | 361 | 29.975 | 30.745 | −2.876 | 1.00 | 16.51 |
| 1253 | CA | PHE | A | 361 | 29.793 | 29.736 | −1.838 | 1.00 | 17.11 |
| 1254 | OB | PHE | A | 361 | 29.160 | 30.326 | −0.574 | 1.00 | 17.43 |
| 1255 | CG | PHE | A | 361 | 28.769 | 29.280 | 0.441 | 1.00 | 16.12 |
| 1256 | CD1 | PHE | A | 361 | 27.557 | 28.600 | 0.328 | 1.00 | 15.11 |
| 1257 | CD2 | PHE | A | 361 | 29.642 | 28.927 | 1.469 | 1.00 | 16.71 |
| 1258 | CE1 | PHE | A | 361 | 27.219 | 27.580 | 1.223 | 1.00 | 14.11 |
| 1259 | CE2 | PHE | A | 361 | 29.314 | 27.909 | 2.367 | 1.00 | 15.23 |
| 1260 | CZ | PHE | A | 361 | 28.101 | 27.235 | 2.242 | 1.00 | 11.27 |
| 1261 | C | PHE | A | 361 | 28.905 | 28.605 | −2.347 | 1.00 | 16.98 |
| 1262 | O | PHE | A | 361 | 29.211 | 27.431 | −2.147 | 1.00 | 16.87 |
| 1263 | N | ALA | A | 362 | 27.803 | 28.971 | −2.994 | 1.00 | 16.32 |
| 1264 | CA | ALA | A | 362 | 26.851 | 27.993 | −3.517 | 1.00 | 19.25 |
| 1265 | CB | ALA | A | 362 | 25.659 | 28.714 | −4.145 | 1.00 | 17.22 |
| 1266 | C | ALA | A | 362 | 27.460 | 27.021 | −4.528 | 1.00 | 19.60 |
| 1267 | O | ALA | A | 362 | 27.080 | 25.850 | −4.579 | 1.00 | 20.69 |
| 1268 | N | MET | A | 363 | 28.396 | 27.502 | −5.337 | 1.00 | 19.90 |
| 1269 | CA | MET | A | 363 | 29.021 | 26.649 | −6.343 | 1.00 | 23.22 |
| 1270 | CB | MET | A | 363 | 29.940 | 27.473 | −7.244 | 1.00 | 25.16 |
| 1271 | CG | MET | A | 363 | 29.601 | 27.362 | −8.717 | 1.00 | 31.86 |
| 1272 | SD | MET | A | 363 | 27.851 | 27.659 | −9.059 | 1.00 | 35.24 |
| 1273 | CE | MET | A | 363 | 27.233 | 25.996 | −9.144 | 1.00 | 37.40 |
| 1274 | C | MET | A | 363 | 29.808 | 25.533 | −5.672 | 1.00 | 22.48 |
| 1275 | O | MET | A | 363 | 29.718 | 24.373 | −6.069 | 1.00 | 22.07 |
| 1276 | N | LYS | A | 364 | 30.576 | 25.884 | −4.647 | 1.00 | 21.68 |
| 1277 | CA | LYS | A | 364 | 31.349 | 24.884 | −3.932 | 1.00 | 22.55 |
| 1278 | CB | LYS | A | 364 | 32.446 | 25.564 | −3.104 | 1.00 | 24.59 |
| 1279 | CG | LYS | A | 364 | 33.595 | 26.061 | −3.982 | 1.00 | 28.52 |
| 1280 | CD | LYS | A | 364 | 34.721 | 26.718 | −3.203 | 1.00 | 31.12 |
| 1281 | CE | LYS | A | 364 | 34.316 | 28.080 | −2.674 | 1.00 | 33.08 |
| 1282 | NZ | LYS | A | 364 | 35.512 | 28.842 | −2.220 | 1.00 | 34.91 |
| 1283 | C | LYS | A | 364 | 30.438 | 24.022 | −3.056 | 1.00 | 22.72 |
| 1284 | O | LYS | A | 364 | 30.699 | 22.834 | −2.861 | 1.00 | 22.63 |
| 1285 | N | PHE | A | 365 | 29.358 | 24.610 | −2.547 | 1.00 | 21.24 |
| 1286 | CA | PHE | A | 365 | 28.429 | 23.855 | −1.705 | 1.00 | 20.87 |
| 1287 | CB | PHE | A | 365 | 27.423 | 24.794 | −1.027 | 1.00 | 19.09 |
| 1288 | CG | PHE | A | 365 | 26.652 | 24.151 | 0.098 | 1.00 | 19.22 |
| 1289 | CD1 | PHE | A | 365 | 27.263 | 23.902 | 1.322 | 1.00 | 19.37 |
| 1290 | CD2 | PHE | A | 365 | 25.311 | 23.807 | −0.063 | 1.00 | 19.20 |
| 1291 | CE1 | PHE | A | 365 | 26.547 | 23.321 | 2.376 | 1.00 | 18.07 |
| 1292 | CE2 | PHE | A | 365 | 24.585 | 23.227 | 0.978 | 1.00 | 18.27 |
| 1293 | CZ | PHE | A | 365 | 25.204 | 22.984 | 2.202 | 1.00 | 18.45 |
| 1294 | C | PHE | A | 365 | 27.667 | 22.819 | −2.532 | 1.00 | 20.34 |
| 1295 | O | PHE | A | 365 | 27.492 | 21.672 | −2.106 | 1.00 | 19.46 |
| 1296 | N | ASN | A | 366 | 27.212 | 23.232 | −3.712 | 1.00 | 20.08 |
| 1297 | CA | ASN | A | 366 | 26.463 | 22.354 | −4.606 | 1.00 | 21.22 |
| 1298 | CB | ASN | A | 366 | 25.910 | 23.158 | −5.789 | 1.00 | 21.85 |
| 1299 | CG | ASN | A | 366 | 24.619 | 23.893 | −5.448 | 1.00 | 22.68 |
| 1300 | OD1 | ASN | A | 366 | 24.237 | 24.853 | −6.124 | 1.00 | 22.40 |
| 1301 | ND2 | ASN | A | 366 | 23.935 | 23.434 | −4.406 | 1.00 | 20.32 |
| 1302 | C | ASN | A | 366 | 27.324 | 21.202 | −5.120 | 1.00 | 21.97 |
| 1303 | O | ASN | A | 366 | 26.806 | 20.147 | −5.495 | 1.00 | 22.05 |
| 1304 | N | ALA | A | 367 | 28.636 | 21.409 | −5.132 | 1.00 | 20.80 |
| 1305 | CA | ALA | A | 367 | 29.564 | 20.381 | −5.587 | 1.00 | 22.52 |
| 1306 | CB | ALA | A | 367 | 30.977 | 20.942 | −5.660 | 1.00 | 22.39 |
| 1307 | C | ALA | A | 367 | 29.519 | 19.188 | −4.638 | 1.00 | 23.80 |
| 1308 | O | ALA | A | 367 | 30.003 | 18.102 | −4.969 | 1.00 | 24.70 |
| 1309 | N | LEU | A | 368 | 28.942 | 19.395 | −3.455 | 1.00 | 22.37 |
| 1310 | CA | LEU | A | 368 | 28.820 | 18.328 | −2.466 | 1.00 | 23.08 |
| 1311 | CB | LEU | A | 368 | 28.576 | 18.913 | −1.070 | 1.00 | 23.25 |
| 1312 | CG | LEU | A | 368 | 29.713 | 19.763 | −0.491 | 1.00 | 23.11 |
| 1313 | CD1 | LEU | A | 368 | 29.369 | 20.196 | 0.932 | 1.00 | 22.57 |
| 1314 | CD2 | LEU | A | 368 | 31.002 | 18.960 | −0.492 | 1.00 | 24.48 |
| 1315 | C | LEU | A | 368 | 27.681 | 17.382 | −2.834 | 1.00 | 22.79 |
| 1316 | O | LEU | A | 368 | 27.542 | 16.306 | −2.250 | 1.00 | 22.44 |
| 1317 | N | GLU | A | 369 | 26.862 | 17.796 | −3.796 | 1.00 | 22.82 |
| 1318 | CA | GLU | A | 369 | 25.744 | 16.983 | −4.260 | 1.00 | 24.48 |
| 1319 | CB | GLU | A | 369 | 26.279 | 15.731 | −4.955 | 1.00 | 26.99 |
| 1320 | CG | GLU | A | 369 | 26.173 | 15.757 | −6.461 | 1.00 | 33.00 |
| 1321 | CD | GLU | A | 369 | 26.960 | 14.633 | −7.104 | 1.00 | 35.36 |
| 1322 | OE1 | GLU | A | 369 | 26.965 | 13.513 | −6.547 | 1.00 | 37.48 |
| 1323 | OE2 | GLU | A | 369 | 27.568 | 14.870 | −8.166 | 1.00 | 36.92 |
| 1324 | C | GLU | A | 369 | 24.778 | 16.571 | −3.155 | 1.00 | 22.38 |
| 1325 | O | GLU | A | 369 | 24.286 | 15.445 | −3.144 | 1.00 | 23.39 |
| 1326 | N | LEU | A | 370 | 24.507 | 17.476 | −2.221 | 1.00 | 21.57 |
| 1327 | CA | LEU | A | 370 | 23.586 | 17.168 | −1.133 | 1.00 | 18.06 |
| 1328 | CB | LEU | A | 370 | 23.717 | 18.197 | −0.009 | 1.00 | 18.81 |
| 1329 | CG | LEU | A | 370 | 25.064 | 18.386 | 0.688 | 1.00 | 16.97 |
| 1330 | CD1 | LEU | A | 370 | 24.875 | 19.342 | 1.856 | 1.00 | 16.61 |
| 1331 | CD2 | LEU | A | 370 | 25.578 | 17.044 | 1.194 | 1.00 | 18.25 |
| 1332 | C | LEU | A | 370 | 22.148 | 17.199 | −1.635 | 1.00 | 18.75 |
| 1333 | O | LEU | A | 370 | 21.845 | 17.849 | −2.639 | 1.00 | 19.00 |
| 1334 | N | ASP | A | 371 | 21.272 | 16.476 | −0.947 | 1.00 | 19.17 |
| 1335 | CA | ASP | A | 371 | 19.860 | 16.477 | −1.292 | 1.00 | 18.60 |
| 1336 | CB | ASP | A | 371 | 19.336 | 15.060 | −1.583 | 1.00 | 19.63 |
| 1337 | CG | ASP | A | 371 | 19.486 | 14.118 | −0.411 | 1.00 | 21.42 |
| 1338 | OD1 | ASP | A | 371 | 19.258 | 14.547 | 0.738 | 1.00 | 18.99 |
| 1339 | OD2 | ASP | A | 371 | 19.813 | 12.934 | −0.647 | 1.00 | 24.09 |
| 1340 | C | ASP | A | 371 | 19.152 | 17.091 | −0.090 | 1.00 | 18.66 |
| 1341 | O | ASP | A | 371 | 19.789 | 17.388 | 0.919 | 1.00 | 19.31 |
| 1342 | N | ASP | A | 372 | 17.845 | 17.287 | −0.190 | 1.00 | 18.35 |
| 1343 | CA | ASP | A | 372 | 17.098 | 17.902 | 0.896 | 1.00 | 18.08 |
| 1344 | CB | ASP | A | 372 | 15.641 | 18.084 | 0.477 | 1.00 | 20.09 |
| 1345 | CG | ASP | A | 372 | 15.487 | 19.092 | −0.654 | 1.00 | 21.21 |
| 1346 | OD1 | ASP | A | 372 | 15.925 | 20.253 | −0.478 | 1.00 | 20.02 |
| 1347 | OD2 | ASP | A | 372 | 14.932 | 18.728 | −1.717 | 1.00 | 22.56 |
| 1348 | C | ASP | A | 372 | 17.189 | 17.174 | 2.236 | 1.00 | 17.57 |
| 1349 | O | ASP | A | 372 | 17.165 | 17.816 | 3.285 | 1.00 | 17.01 |
| 1350 | N | SER | A | 373 | 17.298 | 15.848 | 2.224 | 1.00 | 16.66 |
| 1351 | CA | SER | A | 373 | 17.406 | 15.132 | 3.493 | 1.00 | 17.74 |
| 1352 | CB | SER | A | 373 | 17.371 | 13.602 | 3.285 | 1.00 | 16.26 |
| 1353 | OG | SER | A | 373 | 18.514 | 13.111 | 2.609 | 1.00 | 18.96 |
| 1354 | C | SER | A | 373 | 18.697 | 15.552 | 4.201 | 1.00 | 17.27 |
| 1355 | O | SER | A | 373 | 18.723 | 15.696 | 5.423 | 1.00 | 19.27 |
| 1356 | N | ASP | A | 374 | 19.763 | 15.765 | 3.433 | 1.00 | 16.48 |
| 1357 | CA | ASP | A | 374 | 21.045 | 16.188 | 4.007 | 1.00 | 15.88 |
| 1358 | CB | ASP | A | 374 | 22.174 | 16.142 | 2.969 | 1.00 | 15.19 |
| 1359 | CG | ASP | A | 374 | 22.316 | 14.795 | 2.301 | 1.00 | 18.19 |
| 1360 | OD1 | ASP | A | 374 | 22.362 | 13.768 | 3.014 | 1.00 | 19.73 |
| 1361 | OD2 | ASP | A | 374 | 22.404 | 14.777 | 1.053 | 1.00 | 17.35 |
| 1362 | C | ASP | A | 374 | 20.947 | 17.631 | 4.494 | 1.00 | 15.34 |
| 1363 | O | ASP | A | 374 | 21.355 | 17.954 | 5.608 | 1.00 | 13.16 |
| 1364 | N | ILE | A | 375 | 20.418 | 18.492 | 3.629 | 1.00 | 14.16 |
| 1365 | CA | ILE | A | 375 | 20.283 | 19.912 | 3.924 | 1.00 | 14.77 |
| 1366 | CB | ILE | A | 375 | 19.667 | 20.661 | 2.713 | 1.00 | 14.32 |
| 1367 | CG2 | ILE | A | 375 | 19.457 | 22.126 | 3.054 | 1.00 | 13.42 |
| 1368 | CG1 | ILE | A | 375 | 20.598 | 20.528 | 1.506 | 1.00 | 12.81 |
| 1369 | CD1 | ILE | A | 375 | 19.942 | 20.836 | 0.159 | 1.00 | 10.35 |
| 1370 | C | ILE | A | 375 | 19.456 | 20.185 | 5.178 | 1.00 | 15.58 |
| 1371 | O | ILE | A | 375 | 19.812 | 21.039 | 5.993 | 1.00 | 15.62 |
| 1372 | N | SER | A | 376 | 18.363 | 19.449 | 5.347 | 1.00 | 15.94 |
| 1373 | CA | SER | A | 376 | 17.517 | 19.656 | 6.516 | 1.00 | 17.39 |
| 1374 | CR | SER | A | 376 | 16.329 | 18.691 | 6.509 | 1.00 | 18.46 |
| 1375 | OG | SER | A | 376 | 16.754 | 17.354 | 6.681 | 1.00 | 19.88 |
| 1376 | C | SER | A | 376 | 18.313 | 19.472 | 7.799 | 1.00 | 16.76 |
| 1377 | O | SER | A | 376 | 18.141 | 20.231 | 8.740 | 1.00 | 15.46 |
| 1378 | N | LEU | A | 377 | 19.179 | 18.462 | 7.839 | 1.00 | 17.17 |
| 1379 | CA | LEU | A | 377 | 19.985 | 18.213 | 9.036 | 1.00 | 18.15 |
| 1380 | CB | LEU | A | 377 | 20.649 | 16.825 | 8.966 | 1.00 | 18.31 |
| 1381 | CG | LEU | A | 377 | 19.691 | 15.629 | 8.897 | 1.00 | 20.80 |
| 1382 | CD1 | LEU | A | 377 | 20.471 | 14.320 | 8.902 | 1.00 | 22.15 |
| 1383 | CD2 | LEU | A | 377 | 18.737 | 15.675 | 10.085 | 1.00 | 22.01 |
| 1384 | C | LEU | A | 377 | 21.058 | 19.285 | 9.203 | 1.00 | 17.32 |
| 1385 | O | LEU | A | 377 | 21.344 | 19.720 | 10.317 | 1.00 | 17.55 |
| 1386 | N | PHE | A | 378 | 21.650 | 19.697 | 8.085 | 1.00 | 15.68 |
| 1387 | CA | PHE | A | 378 | 22.694 | 20.723 | 8.085 | 1.00 | 15.42 |
| 1388 | CB | PHE | A | 378 | 23.185 | 20.951 | 6.652 | 1.00 | 14.83 |
| 1389 | CG | PHE | A | 378 | 24.352 | 21.895 | 6.547 | 1.00 | 17.91 |
| 1390 | CD1 | PHE | A | 378 | 25.627 | 21.491 | 6.928 | 1.00 | 18.12 |
| 1391 | CD2 | PHE | A | 378 | 24.172 | 23.191 | 6.070 | 1.00 | 17.17 |
| 1392 | CE1 | PHE | A | 378 | 26.709 | 22.364 | 6.835 | 1.00 | 19.84 |
| 1393 | CE2 | PHE | A | 378 | 25.248 | 24.072 | 5.974 | 1.00 | 17.27 |
| 1394 | CZ | PHE | A | 378 | 26.519 | 23.657 | 6.357 | 1.00 | 18.98 |
| 1395 | C | PHE | A | 378 | 22.138 | 22.032 | 8.664 | 1.00 | 14.65 |
| 1396 | O | PHE | A | 378 | 22.778 | 22.688 | 9.489 | 1.00 | 14.80 |
| 1397 | N | VAL | A | 379 | 20.937 | 22.403 | 8.230 | 1.00 | 14.52 |
| 1398 | CA | VAL | A | 379 | 20.305 | 23.625 | 8.713 | 1.00 | 13.14 |
| 1399 | CB | VAL | A | 379 | 19.060 | 23.964 | 7.861 | 1.00 | 14.31 |
| 1400 | CG1 | VAL | A | 379 | 18.258 | 25.093 | 8.492 | 1.00 | 13.46 |
| 1401 | CG2 | VAL | A | 379 | 19.520 | 24.388 | 6.460 | 1.00 | 13.68 |
| 1402 | C | VAL | A | 379 | 19.945 | 23.536 | 10.200 | 1.00 | 14.97 |
| 1403 | O | VAL | A | 379 | 20.093 | 24.511 | 10.942 | 1.00 | 14.16 |
| 1404 | N | ALA | A | 380 | 19.479 | 22.372 | 10.642 | 1.00 | 14.60 |
| 1405 | CA | ALA | A | 380 | 19.139 | 22.212 | 12.055 | 1.00 | 15.75 |
| 1406 | CB | ALA | A | 380 | 18.541 | 20.830 | 12.313 | 1.00 | 14.49 |
| 1407 | C | ALA | A | 380 | 20.412 | 22.389 | 12.875 | 1.00 | 17.48 |
| 1408 | O | ALA | A | 380 | 20.388 | 22.955 | 13.967 | 1.00 | 18.76 |
| 1409 | N | ALA | A | 381 | 21.521 | 21.890 | 12.337 | 1.00 | 17.47 |
| 1410 | CA | ALA | A | 381 | 22.809 | 21.989 | 13.008 | 1.00 | 18.52 |
| 1411 | CB | ALA | A | 381 | 23.843 | 21.138 | 12.275 | 1.00 | 18.08 |
| 1412 | C | ALA | A | 381 | 23.295 | 23.440 | 13.116 | 1.00 | 18.95 |
| 1413 | O | ALA | A | 381 | 23.826 | 23.843 | 14.149 | 1.00 | 18.98 |
| 1414 | N | ILE | A | 382 | 23.120 | 24.237 | 12.065 | 1.00 | 18.97 |
| 1415 | CA | ILE | A | 382 | 23.582 | 25.621 | 12.157 | 1.00 | 19.61 |
| 1416 | CB | ILE | A | 382 | 23.589 | 26.346 | 10.767 | 1.00 | 22.32 |
| 1417 | CG2 | ILE | A | 382 | 24.054 | 25.408 | 9.685 | 1.00 | 22.00 |
| 1418 | CG1 | ILE | A | 382 | 22.217 | 26.918 | 10.442 | 1.00 | 25.81 |
| 1419 | CD1 | ILE | A | 382 | 21.988 | 28.285 | 11.051 | 1.00 | 26.92 |
| 1420 | C | ILE | A | 382 | 22.722 | 26.408 | 13.152 | 1.00 | 19.24 |
| 1421 | O | ILE | A | 382 | 23.223 | 27.278 | 13.869 | 1.00 | 18.21 |
| 1422 | N | ILE | A | 383 | 21.434 | 26.086 | 13.209 | 1.00 | 17.90 |
| 1423 | CA | ILE | A | 383 | 20.518 | 26.775 | 14.113 | 1.00 | 18.91 |
| 1424 | CB | ILE | A | 383 | 19.049 | 26.426 | 13.786 | 1.00 | 20.25 |
| 1425 | CG2 | ILE | A | 383 | 18.122 | 26.923 | 14.899 | 1.00 | 20.50 |
| 1426 | CG1 | ILE | A | 383 | 18.655 | 27.055 | 12.446 | 1.00 | 19.74 |
| 1427 | CD1 | ILE | A | 383 | 17.224 | 26.779 | 12.032 | 1.00 | 20.27 |
| 1428 | C | ILE | A | 383 | 20.788 | 26.438 | 15.578 | 1.00 | 19.83 |
| 1429 | O | ILE | A | 383 | 20.882 | 27.328 | 16.426 | 1.00 | 18.98 |
| 1430 | N | CYS | A | 384 | 20.930 | 25.154 | 15.876 | 1.00 | 21.71 |
| 1431 | CA | CYS | A | 384 | 21.169 | 24.735 | 17.253 | 1.00 | 24.63 |
| 1432 | CB | CYS | A | 384 | 20.583 | 23.343 | 17.466 | 1.00 | 26.05 |
| 1433 | SG | CYS | A | 384 | 18.829 | 23.300 | 17.088 | 1.00 | 27.14 |
| 1434 | C | CYS | A | 384 | 22.653 | 24.763 | 17.576 | 1.00 | 25.29 |
| 1435 | O | CYS | A | 384 | 23.277 | 23.732 | 17.827 | 1.00 | 26.58 |
| 1436 | N | CYS | A | 385 | 23.197 | 25.974 | 17.564 | 1.00 | 27.00 |
| 1437 | CA | CYS | A | 385 | 24.605 | 26.233 | 17.822 | 1.00 | 28.30 |
| 1438 | CB | CYS | A | 385 | 25.104 | 27.255 | 16.804 | 1.00 | 30.14 |
| 1439 | SG | CYS | A | 385 | 26.815 | 27.715 | 16.976 | 1.00 | 32.84 |
| 1440 | C | CYS | A | 385 | 24.816 | 26.764 | 19.243 | 1.00 | 27.82 |
| 1441 | O | CYS | A | 385 | 24.267 | 27.801 | 19.615 | 1.00 | 27.12 |
| 1442 | N | GLY | A | 386 | 25.624 | 26.055 | 20.026 | 1.00 | 28.23 |
| 1443 | CA | GLY | A | 386 | 25.872 | 26.464 | 21.398 | 1.00 | 28.09 |
| 1444 | C | GLY | A | 386 | 26.927 | 27.539 | 21.582 | 1.00 | 28.17 |
| 1445 | O | GLY | A | 386 | 27.159 | 27.987 | 22.702 | 1.00 | 30.05 |
| 1446 | N | ASP | A | 387 | 27.555 | 27.965 | 20.490 | 1.00 | 29.00 |
| 1447 | CA | ASP | A | 387 | 28.602 | 28.982 | 20.555 | 1.00 | 29.78 |
| 1448 | CB | ASP | A | 387 | 29.691 | 28.690 | 19.520 | 1.00 | 33.93 |
| 1449 | CG | ASP | A | 387 | 30.174 | 27.263 | 19.569 | 1.00 | 37.57 |
| 1450 | OD1 | ASP | A | 387 | 30.417 | 26.758 | 20.687 | 1.00 | 40.66 |
| 1451 | OD2 | ASP | A | 387 | 30.320 | 26.651 | 18.489 | 1.00 | 39.44 |
| 1452 | C | ASP | A | 387 | 28.107 | 30.404 | 20.330 | 1.00 | 27.38 |
| 1453 | O | ASP | A | 387 | 28.889 | 31.348 | 20.396 | 1.00 | 27.02 |
| 1454 | N | ARG | A | 388 | 26.818 | 30.563 | 20.057 | 1.00 | 25.37 |
| 1455 | CA | ARG | A | 388 | 26.271 | 31.889 | 19.807 | 1.00 | 24.60 |
| 1456 | CB | ARG | A | 388 | 24.785 | 31.791 | 19.462 | 1.00 | 23.34 |
| 1457 | CG | ARG | A | 388 | 24.471 | 30.855 | 18.309 | 1.00 | 19.60 |
| 1458 | CD | ARG | A | 388 | 25.262 | 31.214 | 17.061 | 1.00 | 20.49 |
| 1459 | NE | ARG | A | 388 | 24.765 | 30.481 | 15.900 | 1.00 | 14.79 |
| 1460 | CZ | ARG | A | 388 | 25.332 | 30.498 | 14.700 | 1.00 | 16.61 |
| 1461 | NH1 | ARG | A | 388 | 26.430 | 31.215 | 14.489 | 1.00 | 14.39 |
| 1462 | NH2 | ARG | A | 388 | 24.798 | 29.789 | 13.714 | 1.00 | 14.57 |
| 1463 | C | ARG | A | 388 | 26.457 | 32.840 | 20.987 | 1.00 | 26.30 |
| 1464 | O | ARG | A | 388 | 26.286 | 32.458 | 22.143 | 1.00 | 26.77 |
| 1465 | N | PRO | A | 389 | 26.821 | 34.098 | 20.704 | 1.00 | 27.02 |
| 1466 | CD | PRO | A | 389 | 27.215 | 34.629 | 19.388 | 1.00 | 26.91 |
| 1467 | CA | PRO | A | 389 | 27.025 | 35.101 | 21.752 | 1.00 | 27.46 |
| 1468 | CB | PRO | A | 389 | 27.454 | 36.338 | 20.966 | 1.00 | 28.18 |
| 1469 | CG | PRO | A | 389 | 28.135 | 35.762 | 19.770 | 1.00 | 28.30 |
| 1470 | C | PRO | A | 389 | 25.739 | 35.352 | 22.539 | 1.00 | 28.08 |
| 1471 | O | PRO | A | 389 | 24.643 | 35.288 | 21.982 | 1.00 | 28.60 |
| 1472 | N | GLY | A | 390 | 25.881 | 35.620 | 23.834 | 1.00 | 27.99 |
| 1473 | CA | GLY | A | 390 | 24.731 | 35.912 | 24.674 | 1.00 | 27.31 |
| 1474 | C | GLY | A | 390 | 23.805 | 34.779 | 25.077 | 1.00 | 26.48 |
| 1475 | O | GLY | A | 390 | 22.720 | 35.038 | 25.601 | 1.00 | 25.44 |
| 1476 | N | LEU | A | 391 | 24.207 | 33.534 | 24.841 | 1.00 | 25.96 |
| 1477 | CA | LEU | A | 391 | 23.369 | 32.396 | 25.211 | 1.00 | 26.11 |
| 1478 | CB | LEU | A | 391 | 23.808 | 31.132 | 24.474 | 1.00 | 26.09 |
| 1479 | CG | LEU | A | 391 | 23.410 | 30.999 | 23.004 | 1.00 | 25.05 |
| 1480 | OD1 | LEU | A | 391 | 24.037 | 29.734 | 22.431 | 1.00 | 23.61 |
| 1481 | CD2 | LEU | A | 391 | 21.895 | 30.941 | 22.884 | 1.00 | 23.97 |
| 1482 | C | LEU | A | 391 | 23.430 | 32.135 | 26.708 | 1.00 | 27.86 |
| 1483 | O | LEU | A | 391 | 24.468 | 32.340 | 27.341 | 1.00 | 26.88 |
| 1484 | N | LEU | A | 392 | 22.318 | 31.662 | 27.260 | 1.00 | 28.14 |
| 1485 | CA | LEU | A | 392 | 22.230 | 31.366 | 28.682 | 1.00 | 31.00 |
| 1486 | CB | LEU | A | 392 | 20.863 | 31.794 | 29.225 | 1.00 | 30.68 |
| 1487 | CG | LEU | A | 392 | 20.575 | 31.444 | 30.690 | 1.00 | 32.59 |
| 1488 | CD1 | LEU | A | 392 | 21.613 | 32.099 | 31.595 | 1.00 | 32.90 |
| 1489 | CD2 | LEU | A | 392 | 19.173 | 31.906 | 31.057 | 1.00 | 31.69 |
| 1490 | C | LEU | A | 392 | 22.428 | 29.883 | 28.943 | 1.00 | 31.60 |
| 1491 | O | LEU | A | 392 | 23.371 | 29.471 | 29.618 | 1.00 | 33.05 |
| 1492 | N | ASN | A | 393 | 21.527 | 29.084 | 28.388 | 1.00 | 31.62 |
| 1493 | CA | ASN | A | 393 | 21.555 | 27.642 | 28.565 | 1.00 | 32.32 |
| 1494 | CB | ASN | A | 393 | 20.136 | 27.109 | 28.403 | 1.00 | 34.00 |
| 1495 | CG | ASN | A | 393 | 19.940 | 25.769 | 29.056 | 1.00 | 36.18 |
| 1496 | OD1 | ASN | A | 393 | 18.821 | 25.265 | 29.121 | 1.00 | 38.80 |
| 1497 | ND2 | ASN | A | 393 | 21.025 | 25.178 | 29.547 | 1.00 | 37.30 |
| 1498 | C | ASN | A | 393 | 22.501 | 26.938 | 27.586 | 1.00 | 31.82 |
| 1499 | O | ASN | A | 393 | 22.112 | 25.986 | 26.913 | 1.00 | 30.71 |
| 1500 | N | VAL | A | 394 | 23.743 | 27.406 | 27.526 | 1.00 | 31.16 |
| 1501 | CA | VAL | A | 394 | 24.748 | 26.838 | 26.634 | 1.00 | 31.48 |
| 1502 | CB | VAL | A | 394 | 26.140 | 27.467 | 26.912 | 1.00 | 31.26 |
| 1503 | CG1 | VAL | A | 394 | 26.485 | 27.325 | 28.385 | 1.00 | 32.59 |
| 1504 | CG2 | VAL | A | 394 | 27.204 | 26.802 | 26.049 | 1.00 | 31.72 |
| 1505 | C | VAL | A | 394 | 24.843 | 25.311 | 26.739 | 1.00 | 31.80 |
| 1506 | O | VAL | A | 394 | 24.984 | 24.619 | 25.728 | 1.00 | 32.39 |
| 1507 | N | GLY | A | 395 | 24.753 | 24.789 | 27.958 | 1.00 | 30.80 |
| 1508 | CA | GLY | A | 395 | 24.834 | 23.351 | 28.150 | 1.00 | 30.87 |
| 1509 | C | GLY | A | 395 | 23.747 | 22.558 | 27.443 | 1.00 | 31.11 |
| 1510 | O | GLY | A | 395 | 24.037 | 21.612 | 26.707 | 1.00 | 31.28 |
| 1511 | N | HIS | A | 396 | 22.493 | 22.940 | 27.662 | 1.00 | 30.39 |
| 1512 | CA | HIS | A | 396 | 21.370 | 22.248 | 27.047 | 1.00 | 30.64 |
| 1513 | CB | HIS | A | 396 | 20.046 | 22.779 | 27.613 | 1.00 | 33.97 |
| 1514 | CG | HIS | A | 396 | 19.864 | 22.503 | 29.077 | 1.00 | 38.64 |
| 1515 | CD2 | HIS | A | 396 | 18.831 | 22.763 | 29.914 | 1.00 | 40.99 |
| 1516 | ND1 | HIS | A | 396 | 20.841 | 21.913 | 29.849 | 1.00 | 39.58 |
| 1517 | CE1 | HIS | A | 396 | 20.420 | 21.824 | 31.098 | 1.00 | 41.27 |
| 1518 | NE2 | HIS | A | 396 | 19.204 | 22.334 | 31.165 | 1.00 | 41.61 |
| 1519 | C | HIS | A | 396 | 21.386 | 22.376 | 25.526 | 1.00 | 29.36 |
| 1520 | O | HIS | A | 396 | 20.970 | 21.465 | 24.821 | 1.00 | 28.36 |
| 1521 | N | ILE | A | 397 | 21.873 | 23.504 | 25.022 | 1.00 | 27.89 |
| 1522 | CA | ILE | A | 397 | 21.935 | 23.707 | 23.579 | 1.00 | 27.60 |
| 1523 | CB | ILE | A | 397 | 22.208 | 25.194 | 23.240 | 1.00 | 25.64 |
| 1524 | CG2 | ILE | A | 397 | 22.465 | 25.366 | 21.743 | 1.00 | 25.46 |
| 1525 | CG1 | ILE | A | 397 | 20.998 | 26.036 | 23.649 | 1.00 | 23.43 |
| 1526 | CD1 | ILE | A | 397 | 21.234 | 27.535 | 23.573 | 1.00 | 23.81 |
| 1527 | C | ILE | A | 397 | 23.014 | 22.817 | 22.961 | 1.00 | 28.42 |
| 1528 | O | ILE | A | 397 | 22.837 | 22.292 | 21.863 | 1.00 | 28.52 |
| 1529 | N | GLU | A | 398 | 24.124 | 22.635 | 23.671 | 1.00 | 28.38 |
| 1530 | CA | GLU | A | 398 | 25.202 | 21.789 | 23.167 | 1.00 | 30.86 |
| 1531 | CB | GLU | A | 398 | 26.423 | 21.850 | 24.091 | 1.00 | 31.87 |
| 1532 | CG | GLU | A | 398 | 27.051 | 23.224 | 24.207 | 1.00 | 35.51 |
| 1533 | CD | GLU | A | 398 | 28.340 | 23.206 | 25.006 | 1.00 | 37.24 |
| 1534 | OE1 | GLU | A | 398 | 28.347 | 22.630 | 26.114 | 1.00 | 38.47 |
| 1535 | OE2 | GLU | A | 398 | 29.342 | 23.776 | 24.526 | 1.00 | 39.11 |
| 1536 | C | GLU | A | 398 | 24.743 | 20.339 | 23.037 | 1.00 | 30.96 |
| 1537 | O | GLU | A | 398 | 25.105 | 19.655 | 22.084 | 1.00 | 32.65 |
| 1538 | N | LYS | A | 399 | 23.950 | 19.870 | 23.996 | 1.00 | 31.74 |
| 1539 | CA | LYS | A | 399 | 23.457 | 18.495 | 23.966 | 1.00 | 32.12 |
| 1540 | CB | LYS | A | 399 | 22.721 | 18.166 | 25.272 | 1.00 | 34.57 |
| 1541 | CG | LYS | A | 399 | 21.269 | 18.617 | 25.317 | 1.00 | 37.40 |
| 1542 | CD | LYS | A | 399 | 20.332 | 17.512 | 24.854 | 1.00 | 39.89 |
| 1543 | CE | LYS | A | 399 | 19.038 | 18.078 | 24.284 | 1.00 | 40.38 |
| 1544 | NZ | LYS | A | 399 | 18.417 | 19.102 | 25.174 | 1.00 | 42.06 |
| 1545 | C | LYS | A | 399 | 22.522 | 18.335 | 22.770 | 1.00 | 30.88 |
| 1546 | O | LYS | A | 399 | 22.437 | 17.264 | 22.166 | 1.00 | 29.64 |
| 1547 | N | MET | A | 400 | 21.823 | 19.413 | 22.433 | 1.00 | 30.20 |
| 1548 | CA | MET | A | 400 | 20.909 | 19.408 | 21.298 | 1.00 | 30.04 |
| 1549 | CB | MET | A | 400 | 20.125 | 20.719 | 21.248 | 1.00 | 31.30 |
| 1550 | CG | MET | A | 400 | 18.626 | 20.562 | 21.377 | 1.00 | 34.42 |
| 1551 | SD | MET | A | 400 | 17.780 | 22.138 | 21.146 | 1.00 | 37.50 |
| 1552 | CE | MET | A | 400 | 17.589 | 22.649 | 22.799 | 1.00 | 35.32 |
| 1553 | C | MET | A | 400 | 21.705 | 19.251 | 20.006 | 1.00 | 28.14 |
| 1554 | O | MET | A | 400 | 21.406 | 18.386 | 19.177 | 1.00 | 27.06 |
| 1555 | N | GLN | A | 401 | 22.719 | 20.095 | 19.836 | 1.00 | 27.24 |
| 1556 | CA | GLN | A | 401 | 23.548 | 20.044 | 18.638 | 1.00 | 27.85 |
| 1557 | CB | GLN | A | 401 | 24.571 | 21.188 | 18.628 | 1.00 | 29.46 |
| 1558 | CG | GLN | A | 401 | 25.434 | 21.214 | 17.366 | 1.00 | 32.43 |
| 1559 | CD | GLN | A | 401 | 26.273 | 22.480 | 17.228 | 1.00 | 34.32 |
| 1560 | CE1 | GLN | A | 401 | 27.074 | 22.809 | 18.102 | 1.00 | 34.44 |
| 1561 | NE2 | GLN | A | 401 | 26.093 | 23.190 | 16.118 | 1.00 | 32.43 |
| 1562 | C | GLN | A | 401 | 24.269 | 18.705 | 18.539 | 1.00 | 27.48 |
| 1563 | O | GLN | A | 401 | 24.474 | 18.184 | 17.445 | 1.00 | 26.07 |
| 1564 | N | GLU | A | 402 | 24.641 | 18.149 | 19.687 | 1.00 | 27.34 |
| 1565 | CA | GLU | A | 402 | 25.334 | 16.865 | 19.729 | 1.00 | 28.25 |
| 1566 | CB | GLU | A | 402 | 25.617 | 16.464 | 21.184 | 1.00 | 30.37 |
| 1567 | CO | GLU | A | 402 | 26.263 | 15.089 | 21.339 | 1.00 | 35.80 |
| 1568 | CD | GLU | A | 402 | 26.573 | 14.744 | 22.787 | 1.00 | 38.57 |
| 1569 | CE1 | GLU | A | 402 | 27.527 | 15.326 | 23.347 | 1.00 | 39.33 |
| 1570 | OE2 | GLU | A | 402 | 25.856 | 13.895 | 23.365 | 1.00 | 39.79 |
| 1571 | C | GLU | A | 402 | 24.504 | 15.782 | 19.044 | 1.00 | 26.37 |
| 1572 | O | GLU | A | 402 | 25.017 | 15.030 | 18.216 | 1.00 | 26.12 |
| 1573 | N | GLY | A | 403 | 23.223 | 15.714 | 19.398 | 1.00 | 22.95 |
| 1574 | CA | GLY | A | 403 | 22.335 | 14.727 | 18.814 | 1.00 | 22.23 |
| 1575 | C | GLY | A | 403 | 22.119 | 14.941 | 17.326 | 1.00 | 22.14 |
| 1576 | O | GLY | A | 403 | 22.068 | 13.985 | 16.553 | 1.00 | 22.69 |
| 1577 | N | ILE | A | 404 | 21.991 | 16.199 | 16.918 | 1.00 | 20.27 |
| 1578 | CA | ILE | A | 404 | 21.790 | 16.528 | 15.508 | 1.00 | 20.28 |
| 1579 | CB | ILE | A | 404 | 21.501 | 18.047 | 15.333 | 1.00 | 18.92 |
| 1580 | CG2 | ILE | A | 404 | 21.570 | 18.439 | 13.853 | 1.00 | 18.08 |
| 1581 | CG1 | ILE | A | 404 | 20.130 | 18.370 | 15.931 | 1.00 | 20.64 |
| 1582 | CD1 | ILE | A | 404 | 19.754 | 19.853 | 15.927 | 1.00 | 21.12 |
| 1583 | C | ILE | A | 404 | 23.012 | 16.129 | 14.683 | 1.00 | 20.45 |
| 1584 | O | ILE | A | 404 | 22.885 | 15.494 | 13.634 | 1.00 | 19.39 |
| 1585 | N | VAL | A | 405 | 24.195 | 16.491 | 15.170 | 1.00 | 22.04 |
| 1586 | CA | VAL | A | 405 | 25.441 | 16.167 | 14.484 | 1.00 | 24.01 |
| 1587 | CB | VAL | A | 405 | 26.654 | 16.764 | 15.229 | 1.00 | 24.63 |
| 1588 | CG1 | VAL | A | 405 | 27.950 | 16.264 | 14.608 | 1.00 | 26.70 |
| 1589 | CG2 | VAL | A | 405 | 26.597 | 18.282 | 15.168 | 1.00 | 25.08 |
| 1590 | C | VAL | A | 405 | 25.623 | 14.656 | 14.376 | 1.00 | 24.35 |
| 1591 | O | VAL | A | 405 | 26.062 | 14.145 | 13.347 | 1.00 | 24.50 |
| 1592 | N | HIS | A | 406 | 25.286 | 13.951 | 15.451 | 1.00 | 24.73 |
| 1593 | CA | HIS | A | 406 | 25.393 | 12.499 | 15.494 | 1.00 | 25.40 |
| 1594 | CB | HIS | A | 406 | 24.870 | 11.980 | 16.834 | 1.00 | 27.91 |
| 1595 | CG | HIS | A | 406 | 24.719 | 10.492 | 16.890 | 1.00 | 29.97 |
| 1596 | CD2 | HIS | A | 406 | 23.623 | 9.704 | 16.779 | 1.00 | 31.72 |
| 1597 | ND1 | HIS | A | 406 | 25.790 | 9.638 | 17.041 | 1.00 | 32.64 |
| 1598 | CE1 | HIS | A | 406 | 25.362 | 8.388 | 17.020 | 1.00 | 31.24 |
| 1599 | NE2 | HIS | A | 406 | 24.051 | 8.400 | 16.861 | 1.00 | 32.29 |
| 1600 | C | HIS | A | 406 | 24.573 | 11.891 | 14.362 | 1.00 | 24.78 |
| 1601 | O | HIS | A | 406 | 25.073 | 11.085 | 13.573 | 1.00 | 23.70 |
| 1602 | N | VAL | A | 407 | 23.306 | 12.287 | 14.297 | 1.00 | 24.16 |
| 1603 | CA | VAL | A | 407 | 22.394 | 11.797 | 13.276 | 1.00 | 24.60 |
| 1604 | CB | VAL | A | 407 | 20.962 | 12.319 | 13.536 | 1.00 | 26.47 |
| 1605 | CG1 | VAL | A | 407 | 20.059 | 12.000 | 12.360 | 1.00 | 29.88 |
| 1606 | CG2 | VAL | A | 407 | 20.412 | 11.675 | 14.805 | 1.00 | 27.86 |
| 1607 | C | VAL | A | 407 | 22.872 | 12.218 | 11.885 | 1.00 | 23.32 |
| 1608 | O | VAL | A | 407 | 22.752 | 11.455 | 10.922 | 1.00 | 21.28 |
| 1609 | N | LEU | A | 408 | 23.430 | 13.422 | 11.785 | 1.00 | 21.40 |
| 1610 | CA | LEU | A | 408 | 23.939 | 13.914 | 10.510 | 1.00 | 20.88 |
| 1611 | CB | LEU | A | 408 | 24.388 | 15.378 | 10.636 | 1.00 | 20.25 |
| 1612 | CG | LEU | A | 408 | 25.166 | 15.976 | 9.458 | 1.00 | 19.60 |
| 1613 | CD1 | LEU | A | 408 | 24.324 | 15.912 | 8.188 | 1.00 | 18.77 |
| 1614 | CD2 | LEU | A | 408 | 25.545 | 17.412 | 9.770 | 1.00 | 20.66 |
| 1615 | C | LEU | A | 408 | 25.108 | 13.058 | 10.026 | 1.00 | 21.76 |
| 1616 | O | LEU | A | 408 | 25.145 | 12.653 | 8.867 | 1.00 | 21.23 |
| 1617 | N | ARG | A | 409 | 26.057 | 12.776 | 10.917 | 1.00 | 22.31 |
| 1618 | CA | ARG | A | 409 | 27.217 | 11.971 | 10.547 | 1.00 | 23.13 |
| 1619 | CB | ARG | A | 409 | 28.170 | 11.788 | 11.736 | 1.00 | 25.75 |
| 1620 | CG | ARG | A | 409 | 29.480 | 11.102 | 11.332 | 1.00 | 30.84 |
| 1621 | CD | ARG | A | 409 | 30.361 | 10.722 | 12.517 | 1.00 | 34.64 |
| 1622 | NE | ARG | A | 409 | 30.566 | 11.828 | 13.444 | 1.00 | 36.72 |
| 1623 | CZ | ARG | A | 409 | 29.936 | 11.949 | 14.607 | 1.00 | 38.12 |
| 1624 | NH1 | ARG | A | 409 | 29.063 | 11.027 | 14.986 | 1.00 | 40.03 |
| 1625 | NH2 | ARG | A | 409 | 30.175 | 12.993 | 15.390 | 1.00 | 40.43 |
| 1626 | C | ARG | A | 409 | 26.800 | 10.598 | 10.039 | 1.00 | 23.39 |
| 1627 | O | ARG | A | 409 | 27.345 | 10.093 | 9.056 | 1.00 | 22.61 |
| 1628 | N | LEU | A | 410 | 25.835 | 9.991 | 10.718 | 1.00 | 22.60 |
| 1629 | CA | LEU | A | 410 | 25.351 | 8.673 | 10.332 | 1.00 | 24.37 |
| 1630 | CB | LEU | A | 410 | 24.452 | 8.106 | 11.438 | 1.00 | 24.59 |
| 1631 | CC | LEU | A | 410 | 25.217 | 7.686 | 12.701 | 1.00 | 27.68 |
| 1632 | CD1 | LEU | A | 410 | 24.251 | 7.309 | 13.813 | 1.00 | 27.24 |
| 1633 | CD2 | LEU | A | 410 | 26.133 | 6.519 | 12.366 | 1.00 | 26.90 |
| 1634 | C | LEU | A | 410 | 24.595 | 8.737 | 9.007 | 1.00 | 23.58 |
| 1635 | O | LEU | A | 410 | 24.750 | 7.870 | 8.143 | 1.00 | 22.71 |
| 1636 | N | HIS | A | 411 | 23.786 | 9.776 | 8.844 | 1.00 | 22.55 |
| 1637 | CA | HIS | A | 411 | 23.015 | 9.944 | 7.618 | 1.00 | 22.81 |
| 1638 | CB | HIS | A | 411 | 22.097 | 11.168 | 7.741 | 1.00 | 22.24 |
| 1639 | CG | HIS | A | 411 | 21.156 | 11.337 | 6.590 | 1.00 | 22.11 |
| 1640 | CD2 | HIS | A | 411 | 19.946 | 10.782 | 6.342 | 1.00 | 23.05 |
| 1641 | ND1 | HIS | A | 411 | 21.437 | 12.140 | 5.505 | 1.00 | 23.65 |
| 1642 | CE1 | HIS | A | 411 | 20.441 | 12.074 | 4.639 | 1.00 | 20.84 |
| 1643 | NE2 | HIS | A | 411 | 19.524 | 11.256 | 5.122 | 1.00 | 24.45 |
| 1644 | C | HIS | A | 411 | 23.933 | 10.092 | 6.406 | 1.00 | 22.74 |
| 1645 | O | HIS | A | 411 | 23.689 | 9.497 | 5.351 | 1.00 | 21.42 |
| 1646 | N | LEU | A | 412 | 24.990 | 10.886 | 6.555 | 1.00 | 20.60 |
| 1647 | CA | LEU | A | 412 | 25.931 | 11.093 | 5.460 | 1.00 | 22.09 |
| 1648 | CB | LEU | A | 412 | 26.972 | 12.151 | 5.838 | 1.00 | 21.12 |
| 1649 | CG | LEU | A | 412 | 26.441 | 13.576 | 6.011 | 1.00 | 20.72 |
| 1650 | OD1 | LEU | A | 412 | 27.572 | 14.496 | 6.451 | 1.00 | 20.64 |
| 1651 | OD2 | LEU | A | 412 | 25.828 | 14.060 | 4.693 | 1.00 | 21.54 |
| 1652 | C | LEU | A | 412 | 26.640 | 9.806 | 5.063 | 1.00 | 22.50 |
| 1653 | O | LEU | A | 412 | 26.921 | 9.587 | 3.886 | 1.00 | 22.76 |
| 1654 | N | GLN | A | 413 | 26.944 | 8.960 | 6.041 | 1.00 | 24.45 |
| 1655 | CA | GLN | A | 413 | 27.622 | 7.702 | 5.748 | 1.00 | 26.41 |
| 1656 | CB | GLN | A | 413 | 28.021 | 6.987 | 7.043 | 1.00 | 28.87 |
| 1657 | OG | GLN | A | 413 | 29.245 | 7.579 | 7.714 | 1.00 | 32.50 |
| 1658 | CD | GLN | A | 413 | 30.154 | 6.519 | 8.306 | 1.00 | 35.70 |
| 1659 | OE1 | GLN | A | 413 | 29.809 | 5.862 | 9.286 | 1.00 | 36.19 |
| 1660 | NE2 | GLN | A | 413 | 31.326 | 6.343 | 7.702 | 1.00 | 38.34 |
| 1661 | C | GLN | A | 413 | 26.765 | 6.775 | 4.889 | 1.00 | 26.96 |
| 1662 | O | GLN | A | 413 | 27.271 | 6.120 | 3.976 | 1.00 | 26.69 |
| 1663 | N | SER | A | 414 | 25.467 | 6.729 | 5.173 | 1.00 | 26.98 |
| 1664 | CA | SER | A | 414 | 24.555 | 5.877 | 4.417 | 1.00 | 27.71 |
| 1665 | CB | SER | A | 414 | 23.380 | 5.452 | 5.300 | 1.00 | 29.19 |
| 1666 | OG | SER | A | 414 | 22.560 | 6.558 | 5.632 | 1.00 | 33.45 |
| 1667 | C | SER | A | 414 | 24.017 | 6.521 | 3.138 | 1.00 | 26.94 |
| 1668 | O | SER | A | 414 | 23.709 | 5.820 | 2.175 | 1.00 | 27.54 |
| 1669 | N | ASN | A | 415 | 23.901 | 7.848 | 3.122 | 1.00 | 25.54 |
| 1670 | CA | ASN | A | 415 | 23.377 | 8.552 | 1.947 | 1.00 | 24.76 |
| 1671 | CB | ASN | A | 415 | 22.679 | 9.853 | 2.377 | 1.00 | 24.73 |
| 1672 | CG | ASN | A | 415 | 21.714 | 10.390 | 1.317 | 1.00 | 26.54 |
| 1673 | OD1 | ASN | A | 415 | 21.324 | 11.562 | 1.350 | 1.00 | 25.37 |
| 1674 | ND2 | ASN | A | 415 | 21.310 | 9.529 | 0.386 | 1.00 | 24.83 |
| 1675 | C | ASN | A | 415 | 24.472 | 8.870 | 0.927 | 1.00 | 24.23 |
| 1676 | O | ASN | A | 415 | 24.202 | 8.951 | −0.271 | 1.00 | 23.51 |
| 1677 | N | HIS | A | 416 | 25.704 | 9.053 | 1.403 | 1.00 | 23.19 |
| 1678 | CA | HIS | A | 416 | 26.837 | 9.354 | 0.526 | 1.00 | 24.23 |
| 1679 | CB | HIS | A | 416 | 27.273 | 10.820 | 0.674 | 1.00 | 22.51 |
| 1680 | CG | HIS | A | 416 | 26.236 | 11.811 | 0.239 | 1.00 | 21.91 |
| 1681 | CD2 | HIS | A | 416 | 25.999 | 12.381 | −0.967 | 1.00 | 19.28 |
| 1682 | ND1 | HIS | A | 416 | 25.279 | 12.314 | 1.094 | 1.00 | 22.80 |
| 1683 | CE1 | HIS | A | 416 | 24.497 | 13.152 | 0.434 | 1.00 | 17.33 |
| 1684 | NE2 | HIS | A | 416 | 24.912 | 13.210 | −0.819 | 1.00 | 20.76 |
| 1685 | C | HIS | A | 416 | 28.019 | 8.445 | 0.859 | 1.00 | 26.11 |
| 1686 | O | HIS | A | 416 | 29.076 | 8.912 | 1.285 | 1.00 | 24.94 |
| 1687 | N | PRO | A | 417 | 27.859 | 7.131 | 0.647 | 1.00 | 29.11 |
| 1688 | CD | PRO | A | 417 | 26.708 | 6.470 | 0.007 | 1.00 | 29.49 |
| 1689 | CA | PRO | A | 417 | 28.920 | 6.163 | 0.935 | 1.00 | 30.85 |
| 1690 | CB | PRO | A | 417 | 28.241 | 4.824 | 0.656 | 1.00 | 31.26 |
| 1691 | CG | PRO | A | 417 | 27.314 | 5.162 | −0.472 | 1.00 | 30.77 |
| 1692 | C | PRO | A | 417 | 30.193 | 6.350 | 0.118 | 1.00 | 33.62 |
| 1693 | O | PRO | A | 417 | 31.264 | 5.895 | 0.524 | 1.00 | 35.12 |
| 1694 | N | ASP | A | 418 | 30.084 | 7.019 | −1.025 | 1.00 | 34.60 |
| 1695 | CA | ASP | A | 418 | 31.247 | 7.230 | −1.874 | 1.00 | 36.67 |
| 1696 | CB | ASP | A | 418 | 30.832 | 7.265 | −3.349 | 1.00 | 39.63 |
| 1697 | CG | ASP | A | 418 | 30.147 | 5.981 | −3.794 | 1.00 | 41.06 |
| 1698 | OD1 | ASP | A | 418 | 30.576 | 4.893 | −3.352 | 1.00 | 42.57 |
| 1699 | OD2 | ASP | A | 418 | 29.188 | 6.058 | −4.592 | 1.00 | 43.11 |
| 1700 | C | ASP | A | 418 | 32.048 | 8.483 | −1.541 | 1.00 | 37.42 |
| 1701 | O | ASP | A | 418 | 33.252 | 8.534 | −1.802 | 1.00 | 37.62 |
| 1702 | N | ASP | A | 419 | 31.399 | 9.492 | −0.966 | 1.00 | 37.02 |
| 1703 | CA | ASP | A | 419 | 32.118 | 10.715 | −0.632 | 1.00 | 37.69 |
| 1704 | CB | ASP | A | 419 | 31.185 | 11.909 | −0.446 | 1.00 | 36.87 |
| 1705 | CG | ASP | A | 419 | 31.953 | 13.225 | −0.346 | 1.00 | 37.47 |
| 1706 | OD1 | ASP | A | 419 | 33.059 | 13.232 | 0.238 | 1.00 | 36.42 |
| 1707 | OD2 | ASP | A | 419 | 31.455 | 14.253 | −0.845 | 1.00 | 37.98 |
| 1708 | C | ASP | A | 419 | 32.953 | 10.566 | 0.620 | 1.00 | 38.20 |
| 1709 | O | ASP | A | 419 | 32.444 | 10.440 | 1.736 | 1.00 | 37.99 |
| 1710 | N | ILE | A | 420 | 34.255 | 10.612 | 0.399 | 1.00 | 39.15 |
| 1711 | CA | ILE | A | 420 | 35.263 | 10.494 | 1.429 | 1.00 | 37.95 |
| 1712 | CB | ILE | A | 420 | 36.621 | 10.896 | 0.829 | 1.00 | 39.91 |
| 1713 | CG2 | ILE | A | 420 | 37.041 | 9.869 | −0.219 | 1.00 | 40.60 |
| 1714 | CG1 | ILE | A | 420 | 36.499 | 12.270 | 0.150 | 1.00 | 41.10 |
| 1715 | CD1 | ILE | A | 420 | 37.772 | 12.762 | −0.510 | 1.00 | 42.96 |
| 1716 | C | ILE | A | 420 | 35.022 | 11.292 | 2.716 | 1.00 | 35.07 |
| 1717 | O | ILE | A | 420 | 34.524 | 10.764 | 3.711 | 1.00 | 35.43 |
| 1718 | N | PHE | A | 421 | 35.380 | 12.568 | 2.685 | 1.00 | 31.63 |
| 1719 | CA | PHE | A | 421 | 35.264 | 13.435 | 3.846 | 1.00 | 26.48 |
| 1720 | CB | PHE | A | 421 | 36.496 | 14.341 | 3.943 | 1.00 | 29.04 |
| 1721 | CG | PHE | A | 421 | 37.809 | 13.614 | 3.925 | 1.00 | 30.93 |
| 1722 | OD1 | PHE | A | 421 | 38.353 | 13.160 | 2.733 | 1.00 | 33.42 |
| 1723 | CD2 | PHE | A | 421 | 38.524 | 13.423 | 5.100 | 1.00 | 31.75 |
| 1724 | CE1 | PHE | A | 421 | 39.599 | 12.529 | 2.709 | 1.00 | 34.29 |
| 1725 | CE2 | PHE | A | 421 | 39.768 | 12.794 | 5.089 | 1.00 | 31.88 |
| 1726 | CZ | PHE | A | 421 | 40.306 | 12.348 | 3.892 | 1.00 | 33.09 |
| 1727 | C | PHE | A | 421 | 34.039 | 14.339 | 3.827 | 1.00 | 23.47 |
| 1728 | O | PHE | A | 421 | 34.156 | 15.505 | 4.176 | 1.00 | 21.09 |
| 1729 | N | LEU | A | 422 | 32.869 | 13.832 | 3.449 | 1.00 | 20.27 |
| 1730 | CA | LEU | A | 422 | 31.704 | 14.715 | 3.403 | 1.00 | 17.44 |
| 1731 | CB | LEU | A | 422 | 30.476 | 13.972 | 2.851 | 1.00 | 15.99 |
| 1732 | CG | LEU | A | 422 | 29.237 | 14.847 | 2.598 | 1.00 | 14.82 |
| 1733 | CD1 | LEU | A | 422 | 29.607 | 16.092 | 1.808 | 1.00 | 15.78 |
| 1734 | CD2 | LEU | A | 422 | 28.190 | 14.039 | 1.867 | 1.00 | 16.91 |
| 1735 | C | LEU | A | 422 | 31.376 | 15.381 | 4.745 | 1.00 | 17.17 |
| 1736 | O | LEU | A | 422 | 30.989 | 16.561 | 4.784 | 1.00 | 15.16 |
| 1737 | N | PHE | A | 423 | 31.537 | 14.656 | 5.850 | 1.00 | 15.85 |
| 1738 | CA | PHE | A | 423 | 31.242 | 15.247 | 7.154 | 1.00 | 15.61 |
| 1739 | CB | PHE | A | 423 | 31.333 | 14.189 | 8.264 | 1.00 | 17.14 |
| 1740 | CG | PHE | A | 423 | 30.974 | 14.708 | 9.626 | 1.00 | 18.64 |
| 1741 | CD1 | PHE | A | 423 | 29.698 | 15.202 | 9.888 | 1.00 | 20.33 |
| 1742 | CD2 | PHE | A | 423 | 31.910 | 14.705 | 10.651 | 1.00 | 20.31 |
| 1743 | CE1 | PHE | A | 423 | 29.360 | 15.685 | 11.157 | 1.00 | 20.29 |
| 1744 | CE2 | PHE | A | 423 | 31.583 | 15.186 | 11.924 | 1.00 | 21.44 |
| 1745 | GZ | PHE | A | 423 | 30.305 | 15.676 | 12.175 | 1.00 | 22.31 |
| 1746 | C | PHE | A | 423 | 32.194 | 16.416 | 7.451 | 1.00 | 16.28 |
| 1747 | O | PI1E | A | 423 | 31.750 | 17.537 | 7.727 | 1.00 | 14.24 |
| 1748 | N | PRO | A | 424 | 33.519 | 16.174 | 7.416 | 1.00 | 17.45 |
| 1749 | CD | PRO | A | 424 | 34.280 | 14.915 | 7.331 | 1.00 | 17.91 |
| 1750 | CA | PRO | A | 424 | 34.397 | 17.313 | 7.697 | 1.00 | 16.84 |
| 1751 | CB | PRO | A | 424 | 35.797 | 16.680 | 7.749 | 1.00 | 19.13 |
| 1752 | CG | PRO | A | 424 | 35.657 | 15.406 | 6.971 | 1.00 | 18.96 |
| 1753 | C | PRO | A | 424 | 34.255 | 18.437 | 6.657 | 1.00 | 15.92 |
| 1754 | O | PRO | A | 424 | 34.467 | 19.610 | 6.966 | 1.00 | 15.44 |
| 1755 | N | LYS | A | 425 | 33.882 | 18.086 | 5.428 | 1.00 | 16.05 |
| 1756 | CA | LYS | A | 425 | 33.680 | 19.100 | 4.396 | 1.00 | 15.22 |
| 1757 | CB | LYS | A | 425 | 33.268 | 18.456 | 3.068 | 1.00 | 15.46 |
| 1758 | CG | LYS | A | 425 | 34.373 | 17.716 | 2.332 | 1.00 | 16.19 |
| 1759 | CD | LYS | A | 425 | 33.824 | 17.126 | 1.044 | 1.00 | 17.44 |
| 1760 | CE | LYS | A | 425 | 34.866 | 16.327 | 0.278 | 1.00 | 19.89 |
| 1761 | NZ | LYS | A | 425 | 34.278 | 15.771 | −0.982 | 1.00 | 18.37 |
| 1762 | C | LYS | A | 425 | 32.561 | 20.034 | 4.850 | 1.00 | 14.66 |
| 1763 | O | LYS | A | 425 | 32.654 | 21.259 | 4.712 | 1.00 | 13.26 |
| 1764 | N | LEU | A | 426 | 31.497 | 19.445 | 5.394 | 1.00 | 13.97 |
| 1765 | CA | LEU | A | 426 | 30.351 | 20.219 | 5.864 | 1.00 | 14.01 |
| 1766 | CB | LEU | A | 426 | 29.155 | 19.301 | 6.136 | 1.00 | 14.60 |
| 1767 | CG | LEU | A | 426 | 28.490 | 18.720 | 4.887 | 1.00 | 18.11 |
| 1768 | CD1 | LEU | A | 426 | 27.366 | 17.777 | 5.289 | 1.00 | 19.14 |
| 1769 | CD2 | LEU | A | 426 | 27.948 | 19.859 | 4.027 | 1.00 | 19.11 |
| 1770 | C | LEU | A | 426 | 30.694 | 21.025 | 7.110 | 1.00 | 15.51 |
| 1771 | O | LEU | A | 426 | 30.200 | 22.138 | 7.290 | 1.00 | 16.69 |
| 1772 | N | LEU | A | 427 | 31.546 | 20.477 | 7.971 | 1.00 | 16.13 |
| 1773 | CA | LEU | A | 427 | 31.948 | 21.221 | 9.159 | 1.00 | 17.20 |
| 1774 | CB | LEU | A | 427 | 32.875 | 20.386 | 10.048 | 1.00 | 19.20 |
| 1775 | CG | LEU | A | 427 | 32.221 | 19.176 | 10.725 | 1.00 | 21.00 |
| 1776 | CD1 | LEU | A | 427 | 33.248 | 18.435 | 11.574 | 1.00 | 22.86 |
| 1777 | CD2 | LEU | A | 427 | 31.055 | 19.637 | 11.584 | 1.00 | 22.35 |
| 1778 | C | LEU | A | 427 | 32.669 | 22.480 | 8.691 | 1.00 | 17.44 |
| 1779 | O | LEU | A | 427 | 32.495 | 23.556 | 9.266 | 1.00 | 15.95 |
| 1780 | N | GLN | A | 428 | 33.480 | 22.357 | 7.643 | 1.00 | 16.16 |
| 1781 | CA | GLN | A | 428 | 34.183 | 23.532 | 7.134 | 1.00 | 16.84 |
| 1782 | CB | GLN | A | 428 | 35.235 | 23.150 | 6.087 | 1.00 | 17.05 |
| 1783 | CG | GLN | A | 428 | 36.001 | 24.372 | 5.560 | 1.00 | 20.71 |
| 1784 | CD | GLN | A | 428 | 36.981 | 24.039 | 4.453 | 1.00 | 22.77 |
| 1785 | OE1 | GLN | A | 428 | 36.625 | 23.398 | 3.468 | 1.00 | 25.06 |
| 1786 | NE2 | GLN | A | 428 | 38.224 | 24.488 | 4.606 | 1.00 | 26.03 |
| 1787 | C | GLN | A | 428 | 33.179 | 24.512 | 6.525 | 1.00 | 17.18 |
| 1788 | O | GLN | A | 428 | 33.333 | 25.728 | 6.656 | 1.00 | 17.33 |
| 1789 | N | LYS | A | 429 | 32.147 | 23.995 | 5.859 | 1.00 | 17.54 |
| 1790 | CA | LYS | A | 429 | 31.132 | 24.871 | 5.261 | 1.00 | 17.07 |
| 1791 | CB | LYS | A | 429 | 30.077 | 24.061 | 4.505 | 1.00 | 17.70 |
| 1792 | CG | LYS | A | 429 | 30.611 | 23.303 | 3.312 | 1.00 | 19.51 |
| 1793 | CD | LYS | A | 429 | 31.331 | 24.217 | 2.336 | 1.00 | 22.06 |
| 1794 | CE | LYS | A | 429 | 31.888 | 23.424 | 1.151 | 1.00 | 22.64 |
| 1795 | NZ | LYS | A | 429 | 32.800 | 24.262 | 0.326 | 1.00 | 21.64 |
| 1796 | C | LYS | A | 429 | 30.441 | 25.712 | 6.327 | 1.00 | 18.54 |
| 1797 | O | LYS | A | 429 | 30.070 | 26.865 | 6.079 | 1.00 | 16.97 |
| 1798 | N | MET | A | 430 | 30.258 | 25.136 | 7.513 | 1.00 | 18.74 |
| 1799 | CA | MET | A | 430 | 29.621 | 25.868 | 8.599 | 1.00 | 19.35 |
| 1800 | CB | MET | A | 430 | 29.418 | 24.957 | 9.810 | 1.00 | 22.86 |
| 1801 | CO | MET | A | 430 | 28.556 | 23.742 | 9.505 | 1.00 | 25.60 |
| 1802 | SD | MET | A | 430 | 28.185 | 22.755 | 10.953 | 1.00 | 29.86 |
| 1803 | CE | MET | A | 430 | 26.648 | 23.513 | 11.472 | 1.00 | 30.44 |
| 1804 | C | MET | A | 430 | 30.509 | 27.050 | 8.977 | 1.00 | 19.48 |
| 1805 | O | MET | A | 430 | 30.017 | 28.142 | 9.238 | 1.00 | 17.93 |
| 1806 | N | ALA | A | 431 | 31.819 | 26.824 | 9.010 | 1.00 | 19.56 |
| 1807 | CA | ALA | A | 431 | 32.768 | 27.883 | 9.345 | 1.00 | 18.57 |
| 1808 | CB | ALA | A | 431 | 34.160 | 27.293 | 9.551 | 1.00 | 20.87 |
| 1809 | C | ALA | A | 431 | 32.800 | 28.931 | 8.236 | 1.00 | 19.19 |
| 1810 | O | ALA | A | 431 | 32.910 | 30.136 | 8.498 | 1.00 | 18.72 |
| 1811 | N | ASP | A | 432 | 32.711 | 28.471 | 6.992 | 1.00 | 17.33 |
| 1812 | CA | ASP | A | 432 | 32.719 | 29.382 | 5.854 | 1.00 | 17.47 |
| 1813 | CB | ASP | A | 432 | 32.751 | 28.608 | 4.537 | 1.00 | 18.81 |
| 1814 | CG | ASP | A | 432 | 34.086 | 27.928 | 4.285 | 1.00 | 22.11 |
| 1815 | OD1 | ASP | A | 432 | 35.085 | 28.306 | 4.925 | 1.00 | 23.74 |
| 1816 | OD2 | ASP | A | 432 | 34.136 | 27.026 | 3.426 | 1.00 | 23.76 |
| 1817 | C | ASP | A | 432 | 31.477 | 30.268 | 5.890 | 1.00 | 17.16 |
| 1818 | O | ASP | A | 432 | 31.542 | 31.454 | 5.563 | 1.00 | 16.05 |
| 1819 | N | LEU | A | 433 | 30.349 | 29.691 | 6.296 | 1.00 | 15.92 |
| 1820 | CA | LEU | A | 433 | 29.097 | 30.442 | 6.367 | 1.00 | 15.51 |
| 1821 | CB | LEU | A | 433 | 27.919 | 29.500 | 6.645 | 1.00 | 14.97 |
| 1822 | CG | LEU | A | 433 | 27.457 | 28.645 | 5.461 | 1.00 | 16.25 |
| 1823 | OD1 | LBU | A | 433 | 26.435 | 27.633 | 5.928 | 1.00 | 15.43 |
| 1824 | CD2 | LEU | A | 433 | 26.852 | 29.532 | 4.379 | 1.00 | 16.30 |
| 1825 | C | LEU | A | 433 | 29.148 | 31.523 | 7.436 | 1.00 | 16.51 |
| 1826 | O | LEU | A | 433 | 28.643 | 32.623 | 7.240 | 1.00 | 15.91 |
| 1827 | N | ARG | A | 434 | 29.756 | 31.207 | 8.573 | 1.00 | 17.51 |
| 1828 | CA | ARG | A | 434 | 29.857 | 32.181 | 9.651 | 1.00 | 19.49 |
| 1829 | CB | ARG | A | 434 | 30.546 | 31.554 | 10.863 | 1.00 | 20.19 |
| 1830 | CG | ARG | A | 434 | 30.526 | 32.444 | 12.094 | 1.00 | 24.32 |
| 1831 | CD | ARG | A | 434 | 30.936 | 31.678 | 13.331 | 1.00 | 27.77 |
| 1832 | NE | ARG | A | 434 | 29.962 | 30.650 | 13.689 | 1.00 | 30.42 |
| 1833 | CZ | ARG | A | 434 | 30.013 | 29.949 | 14.816 | 1.00 | 32.49 |
| 1834 | NH1 | ARG | A | 434 | 30.992 | 30.171 | 15.682 | 1.00 | 33.93 |
| 1835 | NH2 | ARG | A | 434 | 29.086 | 29.037 | 15.086 | 1.00 | 32.62 |
| 1836 | C | ARG | A | 434 | 30.644 | 33.397 | 9.162 | 1.00 | 19.24 |
| 1837 | O | ARG | A | 434 | 30.275 | 34.539 | 9.432 | 1.00 | 20.11 |
| 1838 | N | GLN | A | 435 | 31.730 | 33.145 | 8.441 | 1.00 | 20.35 |
| 1839 | CA | GLN | A | 435 | 32.554 | 34.224 | 7.905 | 1.00 | 21.41 |
| 1840 | CB | GLN | A | 435 | 33.828 | 33.656 | 7.272 | 1.00 | 23.62 |
| 1841 | CG | GLN | A | 435 | 34.616 | 34.659 | 6.435 | 1.00 | 28.57 |
| 1842 | CD | GLN | A | 435 | 35.501 | 35.574 | 7.260 | 1.00 | 33.29 |
| 1843 | OE1 | GLN | A | 435 | 35.091 | 36.091 | 8.301 | 1.00 | 36.07 |
| 1844 | NE2 | GLN | A | 435 | 36.725 | 35.789 | 6.788 | 1.00 | 36.08 |
| 1845 | C | GLN | A | 435 | 31.757 | 34.987 | 6.852 | 1.00 | 20.82 |
| 1846 | O | GLN | A | 435 | 31.773 | 36.217 | 6.814 | 1.00 | 19.43 |
| 1847 | N | LEU | A | 436 | 31.058 | 34.244 | 5.999 | 1.00 | 19.72 |
| 1848 | CA | LEU | A | 436 | 30.251 | 34.842 | 4.945 | 1.00 | 18.86 |
| 1849 | CB | LEU | A | 436 | 29.571 | 33.742 | 4.121 | 1.00 | 18.95 |
| 1850 | CG | LEU | A | 436 | 28.865 | 34.160 | 2.829 | 1.00 | 20.08 |
| 1851 | CD1 | LEU | A | 436 | 29.911 | 34.595 | 1.800 | 1.00 | 20.86 |
| 1852 | CD2 | LEU | A | 436 | 28.053 | 32.982 | 2.281 | 1.00 | 21.90 |
| 1853 | C | LBU | A | 436 | 29.187 | 35.783 | 5.525 | 1.00 | 18.74 |
| 1854 | O | LEU | A | 436 | 28.905 | 36.838 | 4.951 | 1.00 | 17.29 |
| 1855 | N | VAL | A | 437 | 28.592 | 35.401 | 6.655 | 1.00 | 17.24 |
| 1856 | CA | VAL | A | 437 | 27.561 | 36.235 | 7.283 | 1.00 | 17.47 |
| 1857 | CB | VAL | A | 437 | 26.774 | 35.456 | 8.361 | 1.00 | 17.76 |
| 1858 | CG1 | VAL | A | 437 | 25.846 | 36.405 | 9.120 | 1.00 | 18.67 |
| 1859 | CG2 | VAL | A | 437 | 25.945 | 34.348 | 7.700 | 1.00 | 14.52 |
| 1860 | C | VAL | A | 437 | 28.170 | 37.478 | 7.928 | 1.00 | 18.46 |
| 1861 | O | VAL | A | 437 | 27.615 | 38.576 | 7.834 | 1.00 | 18.68 |
| 1862 | N | THR | A | 438 | 29.309 | 37.301 | 8.588 | 1.00 | 18.45 |
| 1863 | CA | THR | A | 438 | 29.984 | 38.418 | 9.237 | 1.00 | 19.82 |
| 1864 | CB | THR | A | 438 | 31.316 | 37.969 | 9.877 | 1.00 | 20.76 |
| 1865 | OG1 | THR | A | 438 | 31.058 | 36.955 | 10.856 | 1.00 | 22.73 |
| 1866 | CG2 | THR | A | 438 | 32.006 | 39.146 | 10.551 | 1.00 | 23.28 |
| 1867 | C | THR | A | 438 | 30.271 | 39.498 | 8.204 | 1.00 | 20.08 |
| 1868 | O | THR | A | 438 | 30.034 | 40.685 | 8.440 | 1.00 | 20.49 |
| 1869 | N | GLU | A | 439 | 30.778 | 39.071 | 7.052 | 1.00 | 18.53 |
| 1870 | CA | GLU | A | 439 | 31.104 | 39.983 | 5.972 | 1.00 | 19.18 |
| 1871 | CB | GLU | A | 439 | 31.900 | 39.236 | 4.897 | 1.00 | 19.51 |
| 1872 | CG | GLU | A | 439 | 33.061 | 38.440 | 5.491 | 1.00 | 23.78 |
| 1873 | CD | GLU | A | 439 | 33.912 | 37.735 | 4.455 | 1.00 | 25.56 |
| 1874 | OE1 | GLU | A | 439 | 33.347 | 37.165 | 3.502 | 1.00 | 27.66 |
| 1875 | OE2 | GLU | A | 439 | 35.152 | 37.738 | 4.607 | 1.00 | 27.05 |
| 1876 | C | GLU | A | 439 | 29.852 | 40.617 | 5.369 | 1.00 | 17.55 |
| 1877 | O | GLU | A | 439 | 29.862 | 41.786 | 4.988 | 1.00 | 18.18 |
| 1878 | N | HIS | A | 440 | 28.774 | 39.843 | 5.279 | 1.00 | 16.47 |
| 1879 | CA | HIS | A | 440 | 27.527 | 40.353 | 4.725 | 1.00 | 15.35 |
| 1880 | CB | HIS | A | 440 | 26.512 | 39.217 | 4.561 | 1.00 | 13.45 |
| 1881 | CG | HIS | A | 440 | 25.169 | 39.663 | 4.063 | 1.00 | 13.44 |
| 1882 | CD2 | HIS | A | 440 | 24.005 | 39.879 | 4.719 | 1.00 | 14.46 |
| 1883 | ND1 | HIS | A | 440 | 24.913 | 39.927 | 2.734 | 1.00 | 14.54 |
| 1884 | CE1 | HIS | A | 440 | 23.649 | 40.284 | 2.593 | 1.00 | 14.39 |
| 1885 | NE2 | HIS | A | 440 | 23.076 | 40.265 | 3.783 | 1.00 | 14.22 |
| 1886 | C | HIS | A | 440 | 26.947 | 41.444 | 5.627 | 1.00 | 15.34 |
| 1887 | O | HIS | A | 440 | 26.494 | 42.474 | 5.139 | 1.00 | 16.88 |
| 1888 | N | ALA | A | 441 | 26.956 | 41.216 | 6.936 | 1.00 | 16.12 |
| 1889 | CA | ALA | A | 441 | 26.425 | 42.197 | 7.879 | 1.00 | 17.96 |
| 1890 | CB | ALA | A | 441 | 26.513 | 41.661 | 9.305 | 1.00 | 17.75 |
| 1891 | C | ALA | A | 441 | 27.190 | 43.518 | 7.773 | 1.00 | 19.46 |
| 1892 | O | ALA | A | 441 | 26.619 | 44.594 | 7.956 | 1.00 | 18.94 |
| 1893 | N | GLN | A | 442 | 28.483 | 43.431 | 7.479 | 1.00 | 21.32 |
| 1894 | CA | GLN | A | 442 | 29.309 | 44.630 | 7.348 | 1.00 | 22.42 |
| 1895 | CB | GLN | A | 442 | 30.781 | 44.255 | 7.158 | 1.00 | 26.37 |
| 1896 | CG | GLN | A | 442 | 31.717 | 45.455 | 7.074 | 1.00 | 31.72 |
| 1897 | CD | GLN | A | 442 | 33.178 | 45.053 | 6.974 | 1.00 | 34.48 |
| 1898 | OE1 | GLN | A | 442 | 33.675 | 44.272 | 7.786 | 1.00 | 37.93 |
| 1899 | NE2 | GLN | A | 442 | 33.874 | 45.588 | 5.979 | 1.00 | 35.81 |
| 1900 | C | GLN | A | 442 | 28.831 | 45.449 | 6.157 | 1.00 | 21.76 |
| 1901 | O | GLN | A | 442 | 28.707 | 46.670 | 6.240 | 1.00 | 20.34 |
| 1902 | N | LEU | A | 443 | 28.557 | 44.771 | 5.047 | 1.00 | 19.43 |
| 1903 | CA | LEU | A | 443 | 28.087 | 45.452 | 3.851 | 1.00 | 20.58 |
| 1904 | CB | LEU | A | 443 | 28.060 | 44.491 | 2.661 | 1.00 | 21.26 |
| 1905 | CG | LEU | A | 443 | 27.571 | 45.092 | 1.339 | 1.00 | 23.72 |
| 1906 | CD1 | LEU | A | 443 | 28.492 | 46.244 | 0.924 | 1.00 | 23.27 |
| 1907 | CD2 | LEU | A | 443 | 27.542 | 44.016 | 0.268 | 1.00 | 23.43 |
| 1908 | C | LEU | A | 443 | 26.690 | 46.012 | 4.100 | 1.00 | 19.34 |
| 1909 | O | LEU | A | 443 | 26.373 | 47.114 | 3.663 | 1.00 | 20.88 |
| 1910 | N | VAL | A | 444 | 25.859 | 45.250 | 4.808 | 1.00 | 19.86 |
| 1911 | CA | VAL | A | 444 | 24.504 | 45.693 | 5.119 | 1.00 | 19.89 |
| 1912 | CB | VAL | A | 444 | 23.723 | 44.615 | 5.918 | 1.00 | 21.23 |
| 1913 | CG1 | VAL | A | 444 | 22.357 | 45.155 | 6.342 | 1.00 | 22.52 |
| 1914 | CG2 | VAL | A | 444 | 23.536 | 43.370 | 5.058 | 1.00 | 20.41 |
| 1915 | C | VAL | A | 444 | 24.557 | 46.992 | 5.928 | 1.00 | 21.15 |
| 1916 | O | VAL | A | 444 | 23.755 | 47.897 | 5.710 | 1.00 | 20.95 |
| 1917 | N | GLN | A | 445 | 25.512 | 47.083 | 6.849 | 1.00 | 21.46 |
| 1918 | CA | GLN | A | 445 | 25.663 | 48.278 | 7.672 | 1.00 | 23.89 |
| 1919 | CB | GLN | A | 445 | 26.722 | 48.047 | 8.752 | 1.00 | 27.62 |
| 1920 | CG | GLN | A | 445 | 26.863 | 49.191 | 9.753 | 1.00 | 32.28 |
| 1921 | CD | GLN | A | 445 | 25.636 | 49.366 | 10.638 | 1.00 | 35.43 |
| 1922 | OE1 | GLN | A | 445 | 25.623 | 50.212 | 11.533 | 1.00 | 38.17 |
| 1923 | NE2 | GLN | A | 445 | 24.602 | 48.567 | 10.395 | 1.00 | 37.92 |
| 1924 | C | GLN | A | 445 | 26.059 | 49.469 | 6.797 | 1.00 | 24.56 |
| 1925 | O | GLN | A | 445 | 25.586 | 50.588 | 7.003 | 1.00 | 22.97 |
| 1926 | N | ILE | A | 446 | 26.931 | 49.222 | 5.823 | 1.00 | 23.13 |
| 1927 | CA | ILE | A | 446 | 27.378 | 50.271 | 4.915 | 1.00 | 24.75 |
| 1928 | CB | ILE | A | 446 | 28.475 | 49.747 | 3.958 | 1.00 | 24.82 |
| 1929 | CG2 | ILE | A | 446 | 28.747 | 50.759 | 2.855 | 1.00 | 24.01 |
| 1930 | CG1 | ILE | A | 446 | 29.753 | 49.461 | 4.753 | 1.00 | 25.40 |
| 1931 | CD1 | ILE | A | 446 | 30.852 | 48.807 | 3.946 | 1.00 | 25.53 |
| 1932 | C | ILE | A | 446 | 26.194 | 50.780 | 4.099 | 1.00 | 25.90 |
| 1933 | O | ILE | A | 446 | 25.990 | 51.989 | 3.968 | 1.00 | 26.44 |
| 1934 | N | ILE | A | 447 | 25.413 | 49.850 | 3.552 | 1.00 | 26.77 |
| 1935 | CA | ILE | A | 447 | 24.236 | 50.201 | 2.766 | 1.00 | 29.88 |
| 1936 | CB | ILE | A | 447 | 23.539 | 48.937 | 2.213 | 1.00 | 30.75 |
| 1937 | CG2 | ILE | A | 447 | 22.150 | 49.299 | 1.663 | 1.00 | 33.39 |
| 1938 | CG1 | ILE | A | 447 | 24.407 | 48.307 | 1.121 | 1.00 | 31.69 |
| 1939 | CD1 | ILE | A | 447 | 23.811 | 47.036 | 0.510 | 1.00 | 34.09 |
| 1940 | C | ILE | A | 447 | 23.248 | 50.979 | 3.634 | 1.00 | 30.49 |
| 1941 | O | ILE | A | 447 | 22.670 | 51.979 | 3.206 | 1.00 | 30.43 |
| 1942 | N | LYS | A | 448 | 23.095 | 50.538 | 4.873 | 1.00 | 32.72 |
| 1943 | CA | LYS | A | 448 | 22.175 | 51.166 | 5.807 | 1.00 | 36.04 |
| 1944 | CB | LYS | A | 448 | 22.169 | 50.377 | 7.121 | 1.00 | 37.66 |
| 1945 | CG | LYS | A | 448 | 21.205 | 50.919 | 8.162 | 1.00 | 40.69 |
| 1946 | CD | LYS | A | 448 | 20.930 | 49.888 | 9.229 | 1.00 | 41.72 |
| 1947 | CE | LYS | A | 448 | 19.933 | 50.405 | 10.241 | 1.00 | 44.25 |
| 1948 | NZ | LYS | A | 448 | 20.486 | 51.487 | 11.103 | 1.00 | 45.65 |
| 1949 | C | LYS | A | 448 | 22.480 | 52.634 | 6.093 | 1.00 | 36.68 |
| 1950 | O | LYS | A | 448 | 21.566 | 53.450 | 6.206 | 1.00 | 36.26 |
| 1951 | N | LYS | A | 449 | 23.759 | 52.977 | 6.203 | 1.00 | 37.99 |
| 1952 | CA | LYS | A | 449 | 24.129 | 54.358 | 6.495 | 1.00 | 39.24 |
| 1953 | CB | LYS | A | 449 | 25.330 | 54.395 | 7.450 | 1.00 | 41.14 |
| 1954 | CG | LYS | A | 449 | 26.685 | 54.357 | 6.757 | 1.00 | 43.28 |
| 1955 | CD | LYS | A | 449 | 27.829 | 54.400 | 7.765 | 1.00 | 44.36 |
| 1956 | CE | LYS | A | 449 | 27.939 | 53.086 | 8.518 | 1.00 | 45.26 |
| 1957 | NZ | LYS | A | 449 | 28.212 | 51.958 | 7.586 | 1.00 | 44.67 |
| 1958 | C | LYS | A | 449 | 24.449 | 55.179 | 5.246 | 1.00 | 39.55 |
| 1959 | O | LYS | A | 449 | 24.523 | 56.408 | 5.307 | 1.00 | 39.79 |
| 1960 | N | THR | A | 450 | 24.630 | 54.502 | 4.117 | 1.00 | 39.02 |
| 1961 | CA | THR | A | 450 | 24.962 | 55.175 | 2.865 | 1.00 | 39.78 |
| 1962 | CB | THR | A | 450 | 26.150 | 54.462 | 2.168 | 1.00 | 40.83 |
| 1963 | OG1 | THR | A | 450 | 27.381 | 54.930 | 2.736 | 1.00 | 41.96 |
| 1964 | CG2 | THR | A | 450 | 26.149 | 54.722 | 0.671 | 1.00 | 42.44 |
| 1965 | C | THR | A | 450 | 23.802 | 55.298 | 1.879 | 1.00 | 39.33 |
| 1966 | O | THR | A | 450 | 23.724 | 56.263 | 1.120 | 1.00 | 39.15 |
| 1967 | N | GLU | A | 451 | 22.903 | 54.323 | 1.888 | 1.00 | 38.93 |
| 1968 | CA | GLU | A | 451 | 21.765 | 54.341 | 0.979 | 1.00 | 39.36 |
| 1969 | CB | GLU | A | 451 | 21.601 | 52.971 | 0.309 | 1.00 | 37.13 |
| 1970 | CG | GLU | A | 451 | 22.786 | 52.508 | −0.542 | 1.00 | 33.20 |
| 1971 | CD | GLU | A | 451 | 22.987 | 53.336 | −1.801 | 1.00 | 31.38 |
| 1972 | OE1 | GLU | A | 451 | 21.993 | 53.869 | −2.338 | 1.00 | 31.23 |
| 1973 | OE2 | GLU | A | 451 | 24.140 | 53.436 | −2.269 | 1.00 | 27.05 |
| 1974 | C | GLU | A | 451 | 20.480 | 54.701 | 1.718 | 1.00 | 41.61 |
| 1975 | O | GLU | A | 451 | 19.787 | 53.825 | 2.235 | 1.00 | 41.95 |
| 1976 | N | SER | A | 452 | 20.166 | 55.991 | 1.771 | 1.00 | 44.12 |
| 1977 | CA | SER | A | 452 | 18.955 | 56.445 | 2.444 | 1.00 | 46.91 |
| 1978 | CB | SER | A | 452 | 19.066 | 57.928 | 2.804 | 1.00 | 47.79 |
| 1979 | OG | SER | A | 452 | 20.074 | 58.140 | 3.781 | 1.00 | 49.02 |
| 1980 | C | SER | A | 452 | 17.767 | 56.216 | 1.521 | 1.00 | 48.12 |
| 1981 | O | SER | A | 452 | 16.611 | 56.306 | 1.933 | 1.00 | 48.49 |
| 1982 | N | ASP | A | 453 | 18.077 | 55.920 | 0.264 | 1.00 | 49.25 |
| 1983 | CA | ASP | A | 453 | 17.068 | 55.654 | −0.750 | 1.00 | 50.01 |
| 1984 | CB | ASP | A | 453 | 17.754 | 55.278 | −2.063 | 1.00 | 51.19 |
| 1985 | CG | ASP | A | 453 | 19.029 | 54.482 | −1.845 | 1.00 | 51.21 |
| 1986 | OD1 | ASP | A | 453 | 19.964 | 55.027 | −1.223 | 1.00 | 52.21 |
| 1987 | OD2 | ASP | A | 453 | 19.100 | 53.317 | −2.288 | 1.00 | 52.20 |
| 1988 | C | ASP | A | 453 | 16.174 | 54.516 | −0.287 | 1.00 | 49.54 |
| 1989 | O | ASP | A | 453 | 14.965 | 54.524 | −0.513 | 1.00 | 50.36 |
| 1990 | N | ALA | A | 454 | 16.786 | 53.537 | 0.367 | 1.00 | 49.32 |
| 1991 | CA | ALA | A | 454 | 16.069 | 52.382 | 0.877 | 1.00 | 47.69 |
| 1992 | CB | ALA | A | 454 | 16.179 | 51.241 | −0.102 | 1.00 | 48.66 |
| 1993 | C | ALA | A | 454 | 16.668 | 51.988 | 2.222 | 1.00 | 46.49 |
| 1994 | O | ALA | A | 454 | 17.885 | 51.940 | 2.373 | 1.00 | 47.87 |
| 1995 | N | ALA | A | 455 | 15.807 | 51.707 | 3.195 | 1.00 | 43.89 |
| 1996 | CA | ALA | A | 455 | 16.250 | 51.340 | 4.533 | 1.00 | 41.22 |
| 1997 | CB | ALA | A | 455 | 15.306 | 51.944 | 5.568 | 1.00 | 41.01 |
| 1998 | C | ALA | A | 455 | 16.341 | 49.829 | 4.724 | 1.00 | 39.82 |
| 1999 | O | ALA | A | 455 | 17.060 | 49.146 | 3.990 | 1.00 | 41.52 |
| 2000 | N | LEU | A | 456 | 15.625 | 49.329 | 5.728 | 1.00 | 36.01 |
| 2001 | CA | LEU | A | 456 | 15.580 | 47.904 | 6.063 | 1.00 | 32.30 |
| 2002 | CB | LEU | A | 456 | 16.744 | 47.508 | 6.981 | 1.00 | 33.09 |
| 2003 | CG | LEU | A | 456 | 18.083 | 47.052 | 6.390 | 1.00 | 33.05 |
| 2004 | CD1 | LEU | A | 456 | 18.977 | 46.576 | 7.525 | 1.00 | 32.55 |
| 2005 | CD2 | LEU | A | 456 | 17.870 | 45.925 | 5.391 | 1.00 | 31.55 |
| 2006 | C | LEU | A | 456 | 14.272 | 47.560 | 6.769 | 1.00 | 29.76 |
| 2007 | O | LEU | A | 456 | 13.758 | 48.345 | 7.574 | 1.00 | 26.72 |
| 2008 | N | HIS | A | 457 | 13.745 | 46.378 | 6.465 | 1.00 | 26.87 |
| 2009 | CA | HIS | A | 457 | 12.505 | 45.897 | 7.061 | 1.00 | 25.80 |
| 2010 | CB | HIS | A | 457 | 12.113 | 44.558 | 6.421 | 1.00 | 24.73 |
| 2011 | CG | HIS | A | 457 | 10.846 | 43.968 | 6.963 | 1.00 | 25.28 |
| 2012 | CD2 | HIS | A | 457 | 10.567 | 43.401 | 8.160 | 1.00 | 24.58 |
| 2013 | ND1 | HIS | A | 457 | 9.680 | 43.901 | 6.229 | 1.00 | 26.52 |
| 2014 | CE1 | HIS | A | 457 | 8.740 | 43.316 | 6.950 | 1.00 | 25.66 |
| 2015 | NE2 | HIS | A | 457 | 9.253 | 43.003 | 8.127 | 1.00 | 26.42 |
| 2016 | C | HIS | A | 457 | 12.711 | 45.716 | 8.567 | 1.00 | 25.90 |
| 2017 | O | HIS | A | 457 | 13.794 | 45.331 | 9.012 | 1.00 | 25.20 |
| 2018 | N | PRO | A | 458 | 11.669 | 45.995 | 9.368 | 1.00 | 25.60 |
| 2019 | CD | PRO | A | 458 | 10.368 | 46.543 | 8.941 | 1.00 | 26.08 |
| 2020 | CA | PRO | A | 458 | 11.719 | 45.868 | 10.829 | 1.00 | 25.42 |
| 2021 | CB | PRO | A | 458 | 10.257 | 46.032 | 11.229 | 1.00 | 25.91 |
| 2022 | CG | PRO | A | 458 | 9.777 | 47.043 | 10.243 | 1.00 | 27.38 |
| 2023 | C | PRO | A | 458 | 12.314 | 44.557 | 11.342 | 1.00 | 25.07 |
| 2024 | O | PRO | A | 458 | 13.151 | 44.559 | 12.246 | 1.00 | 25.29 |
| 2025 | N | LEU | A | 459 | 11.878 | 43.436 | 10.775 | 1.00 | 23.71 |
| 2026 | CA | LEU | A | 459 | 12.383 | 42.141 | 11.214 | 1.00 | 22.76 |
| 2027 | CB | LEU | A | 459 | 11.618 | 41.002 | 10.530 | 1.00 | 22.31 |
| 2028 | CG | LEU | A | 459 | 12.098 | 39.582 | 10.868 | 1.00 | 22.34 |
| 2029 | CD1 | LEU | A | 459 | 11.955 | 39.318 | 12.368 | 1.00 | 23.76 |
| 2030 | CD2 | LEU | A | 459 | 11.284 | 38.567 | 10.076 | 1.00 | 22.76 |
| 2031 | C | LEU | A | 459 | 13.872 | 41.998 | 10.930 | 1.00 | 22.18 |
| 2032 | O | LEU | A | 459 | 14.623 | 41.493 | 11.761 | 1.00 | 21.97 |
| 2033 | N | LEU | A | 460 | 14.301 | 42.438 | 9.753 | 1.00 | 21.66 |
| 2034 | CA | LEU | A | 460 | 15.710 | 42.337 | 9.397 | 1.00 | 21.60 |
| 2035 | CB | LEU | A | 460 | 15.892 | 42.619 | 7.902 | 1.00 | 20.70 |
| 2036 | CG | LEU | A | 460 | 14.974 | 41.760 | 7.022 | 1.00 | 19.18 |
| 2037 | CD1 | LEU | A | 460 | 15.343 | 41.949 | 5.560 | 1.00 | 20.00 |
| 2038 | CD2 | LEU | A | 460 | 15.098 | 40.285 | 7.418 | 1.00 | 16.85 |
| 2039 | C | LEU | A | 460 | 16.539 | 43.305 | 10.232 | 1.00 | 23.25 |
| 2040 | O | LEU | A | 460 | 17.679 | 43.015 | 10.606 | 1.00 | 21.71 |
| 2041 | N | GLN | A | 461 | 15.952 | 44.453 | 10.541 | 1.00 | 23.59 |
| 2042 | CA | GLN | A | 461 | 16.645 | 45.444 | 11.339 | 1.00 | 26.43 |
| 2043 | CB | GLN | A | 461 | 15.808 | 46.720 | 11.444 | 1.00 | 28.61 |
| 2044 | CG | GLN | A | 461 | 16.491 | 47.837 | 12.219 | 1.00 | 34.25 |
| 2045 | CD | GLN | A | 461 | 17.776 | 48.316 | 11.564 | 1.00 | 36.90 |
| 2046 | OE1 | GLN | A | 461 | 18.513 | 49.110 | 12.145 | 1.00 | 40.50 |
| 2047 | NE2 | GLN | A | 461 | 18.045 | 47.843 | 10.353 | 1.00 | 38.17 |
| 2048 | C | GLN | A | 461 | 16.950 | 44.899 | 12.734 | 1.00 | 25.75 |
| 2049 | O | GLN | A | 461 | 18.057 | 45.078 | 13.235 | 1.00 | 27.57 |
| 2050 | N | GLU | A | 462 | 15.989 | 44.224 | 13.361 | 1.00 | 25.39 |
| 2051 | CA | GLU | A | 462 | 16.241 | 43.696 | 14.699 | 1.00 | 26.81 |
| 2052 | CB | GLU | A | 462 | 14.930 | 43.290 | 15.401 | 1.00 | 28.93 |
| 2053 | CG | GLU | A | 462 | 14.134 | 42.155 | 14.784 | 1.00 | 30.21 |
| 2054 | CD | GLU | A | 462 | 12.838 | 41.880 | 15.553 | 1.00 | 32.16 |
| 2055 | OE1 | GLU | A | 462 | 11.991 | 42.795 | 15.644 | 1.00 | 32.22 |
| 2056 | OE2 | GLU | A | 462 | 12.665 | 40.755 | 16.071 | 1.00 | 30.73 |
| 2057 | C | GLU | A | 462 | 17.240 | 42.541 | 14.688 | 1.00 | 25.84 |
| 2058 | O | GLU | A | 462 | 17.970 | 42.332 | 15.659 | 1.00 | 25.91 |
| 2059 | N | ILE | A | 463 | 17.291 | 41.797 | 13.589 | 1.00 | 23.23 |
| 2060 | CA | ILE | A | 463 | 18.239 | 40.699 | 13.497 | 1.00 | 22.62 |
| 2061 | CB | ILE | A | 463 | 17.942 | 39.793 | 12.273 | 1.00 | 23.65 |
| 2062 | CG2 | ILE | A | 463 | 19.115 | 38.833 | 12.020 | 1.00 | 21.96 |
| 2063 | CG1 | ILE | A | 463 | 16.650 | 39.007 | 12.522 | 1.00 | 22.25 |
| 2064 | CD1 | ILE | A | 463 | 16.196 | 38.160 | 11.338 | 1.00 | 24.20 |
| 2065 | C | ILE | A | 463 | 19.658 | 41.263 | 13.396 | 1.00 | 23.04 |
| 2066 | O | ILE | A | 463 | 20.568 | 40.782 | 14.062 | 1.00 | 20.73 |
| 2067 | N | TYR | A | 464 | 19.839 | 42.298 | 12.579 | 1.00 | 23.82 |
| 2068 | CA | TYR | A | 464 | 21.160 | 42.899 | 12.400 | 1.00 | 25.85 |
| 2069 | CB | TYR | A | 464 | 21.226 | 43.636 | 11.063 | 1.00 | 24.50 |
| 2070 | CG | TYR | A | 464 | 21.403 | 42.697 | 9.892 | 1.00 | 22.75 |
| 2071 | CD1 | TYR | A | 464 | 22.563 | 41.933 | 9.758 | 1.00 | 23.07 |
| 2072 | CE1 | TYR | A | 464 | 22.712 | 41.029 | 8.703 | 1.00 | 22.58 |
| 2073 | CD2 | TYR | A | 464 | 20.397 | 42.538 | 8.943 | 1.00 | 23.44 |
| 2074 | CE2 | TYR | A | 464 | 20.537 | 41.644 | 7.893 | 1.00 | 20.95 |
| 2075 | CZ | TYR | A | 464 | 21.692 | 40.894 | 7.779 | 1.00 | 22.05 |
| 2076 | OH | TYR | A | 464 | 21.819 | 40.001 | 6.744 | 1.00 | 21.31 |
| 2077 | C | TYR | A | 464 | 21.608 | 43.827 | 13.523 | 1.00 | 27.80 |
| 2078 | O | TYR | A | 464 | 22.803 | 44.064 | 13.685 | 1.00 | 28.04 |
| 2079 | N | ARG | A | 465 | 20.661 | 44.350 | 14.294 | 1.00 | 29.91 |
| 2080 | CA | ARG | A | 465 | 21.002 | 45.238 | 15.403 | 1.00 | 33.29 |
| 2081 | CB | ARG | A | 465 | 19.731 | 45.764 | 16.074 | 1.00 | 35.76 |
| 2082 | CG | ARG | A | 465 | 19.978 | 46.538 | 17.368 | 1.00 | 38.99 |
| 2083 | CD | ARG | A | 465 | 18.669 | 47.023 | 17.976 | 1.00 | 42.46 |
| 2084 | NE | ARG | A | 465 | 18.868 | 47.706 | 19.252 | 1.00 | 44.44 |
| 2085 | CZ | ARG | A | 465 | 17.897 | 48.297 | 19.943 | 1.00 | 46.85 |
| 2086 | NH1 | ARG | A | 465 | 16.652 | 48.294 | 19.482 | 1.00 | 47.83 |
| 2087 | NH2 | ARG | A | 465 | 18.167 | 48.890 | 21.097 | 1.00 | 47.38 |
| 2088 | C | ARG | A | 465 | 21.858 | 44.503 | 16.436 | 1.00 | 34.75 |
| 2089 | O | ARG | A | 465 | 21.406 | 43.540 | 17.055 | 1.00 | 34.47 |
| 2090 | N | ASP | A | 466 | 23.095 | 44.961 | 16.608 | 1.00 | 36.62 |
| 2091 | CA | ASP | A | 466 | 24.026 | 44.372 | 17.569 | 1.00 | 38.83 |
| 2092 | CB | ASP | A | 466 | 23.456 | 44.470 | 18.988 | 1.00 | 39.46 |
| 2093 | CG | ASP | A | 466 | 23.231 | 45.902 | 19.429 | 1.00 | 40.61 |
| 2094 | OD1 | ASP | A | 466 | 24.151 | 46.729 | 19.242 | 1.00 | 40.51 |
| 2095 | OD2 | ASP | A | 466 | 22.141 | 46.198 | 19.968 | 1.00 | 40.94 |
| 2096 | C | ASP | A | 466 | 24.407 | 42.921 | 17.288 | 1.00 | 39.96 |
| 2097 | O | ASP | A | 466 | 24.783 | 42.187 | 18.203 | 1.00 | 39.85 |
| 2098 | N | MET | A | 467 | 24.311 | 42.500 | 16.031 | 1.00 | 41.66 |
| 2099 | CA | MET | A | 467 | 24.674 | 41.132 | 15.678 | 1.00 | 43.69 |
| 2100 | CB | MET | A | 467 | 24.328 | 40.841 | 14.217 | 1.00 | 43.01 |
| 2101 | CG | MET | A | 467 | 24.631 | 39.416 | 13.797 | 1.00 | 43.53 |
| 2102 | SD | MET | A | 467 | 24.328 | 39.125 | 12.053 | 1.00 | 45.18 |
| 2103 | CE | MET | A | 467 | 22.544 | 38.865 | 12.066 | 1.00 | 44.23 |
| 2104 | C | MET | A | 467 | 26.175 | 40.966 | 15.889 | 1.00 | 45.56 |
| 2105 | O | MET | A | 467 | 26.619 | 40.175 | 16.722 | 1.00 | 46.26 |
| 2106 | N | TYR | A | 468 | 26.950 | 41.724 | 15.122 | 1.00 | 46.99 |
| 2107 | CA | TYR | A | 468 | 28.403 | 41.691 | 15.215 | 1.00 | 48.84 |
| 2108 | CB | TYR | A | 468 | 29.016 | 41.118 | 13.930 | 1.00 | 48.71 |
| 2109 | CG | TYR | A | 468 | 28.731 | 39.646 | 13.712 | 1.00 | 48.95 |
| 2110 | CD1 | TYR | A | 468 | 27.913 | 39.218 | 12.665 | 1.00 | 49.23 |
| 2111 | CE1 | TYR | A | 468 | 27.644 | 37.861 | 12.468 | 1.00 | 49.07 |
| 2112 | CD2 | TYR | A | 468 | 29.273 | 38.680 | 14.560 | 1.00 | 49.57 |
| 2113 | CE2 | TYR | A | 468 | 29.011 | 37.324 | 14.374 | 1.00 | 49.37 |
| 2114 | CZ | TYR | A | 468 | 28.197 | 36.921 | 13.328 | 1.00 | 49.98 |
| 2115 | OH | TYR | A | 468 | 27.940 | 35.580 | 13.147 | 1.00 | 49.98 |
| 2116 | C | TYR | A | 468 | 28.928 | 43.101 | 15.462 | 1.00 | 49.62 |
| 2117 | O | TYR | A | 468 | 29.141 | 43.829 | 14.472 | 1.00 | 50.21 |
| 2118 | OT | TYR | A | 468 | 29.091 | 43.471 | 16.646 | 1.00 | 50.66 |
| 2119 | CB | GLU | B | 685 | 18.548 | 43.302 | 21.979 | 1.00 | 63.65 |
| 2120 | CG | GLU | B | 685 | 18.317 | 43.165 | 23.475 | 1.00 | 63.87 |
| 2121 | CD | GLU | B | 685 | 18.479 | 41.737 | 23.956 | 1.00 | 64.23 |
| 2122 | OE1 | GLU | B | 685 | 17.606 | 40.903 | 23.635 | 1.00 | 64.28 |
| 2123 | OE2 | GLU | B | 685 | 19.478 | 41.445 | 24.647 | 1.00 | 64.53 |
| 2124 | C | GLU | B | 685 | 16.486 | 44.447 | 21.144 | 1.00 | 61.94 |
| 2125 | O | GLU | B | 685 | 15.986 | 44.713 | 20.050 | 1.00 | 62.27 |
| 2126 | N | GLU | B | 685 | 18.323 | 45.755 | 22.227 | 1.00 | 62.90 |
| 2127 | CA | GLU | B | 685 | 17.990 | 44.588 | 21.363 | 1.00 | 62.73 |
| 2128 | N | ARG | B | 686 | 15.773 | 44.020 | 22.185 | 1.00 | 60.58 |
| 2129 | CA | ARG | B | 686 | 14.324 | 43.847 | 22.118 | 1.00 | 59.13 |
| 2130 | CB | ARG | B | 686 | 13.677 | 45.103 | 21.527 | 1.00 | 60.07 |
| 2131 | CG | ARG | B | 686 | 12.155 | 45.100 | 21.495 | 1.00 | 61.27 |
| 2132 | CD | ARG | B | 686 | 11.558 | 45.589 | 22.807 | 1.00 | 62.16 |
| 2133 | NE | ARG | B | 686 | 10.176 | 46.030 | 22.634 | 1.00 | 63.43 |
| 2134 | CZ | ARG | B | 686 | 9.159 | 45.223 | 22.350 | 1.00 | 64.44 |
| 2135 | NH1 | ARG | B | 686 | 9.361 | 43.920 | 22.210 | 1.00 | 64.89 |
| 2136 | NH2 | ARG | B | 686 | 7.940 | 45.722 | 22.188 | 1.00 | 65.34 |
| 2137 | C | ARG | B | 686 | 13.919 | 42.623 | 21.291 | 1.00 | 57.39 |
| 2138 | O | ARG | B | 686 | 13.657 | 41.552 | 21.841 | 1.00 | 58.48 |
| 2139 | N | HIS | B | 687 | 13.874 | 42.792 | 19.972 | 1.00 | 54.71 |
| 2140 | CA | HIS | B | 687 | 13.488 | 41.724 | 19.049 | 1.00 | 51.16 |
| 2141 | CB | HIS | B | 687 | 14.325 | 40.462 | 19.290 | 1.00 | 51.72 |
| 2142 | CG | HIS | B | 687 | 15.785 | 40.639 | 19.009 | 1.00 | 51.47 |
| 2143 | CD2 | HIS | B | 687 | 16.566 | 40.153 | 18.016 | 1.00 | 51.61 |
| 2144 | ND1 | HIS | B | 687 | 16.608 | 41.410 | 19.801 | 1.00 | 52.04 |
| 2145 | CE1 | HIS | B | 687 | 17.834 | 41.390 | 19.308 | 1.00 | 51.83 |
| 2146 | NE2 | HIS | B | 687 | 17.835 | 40.635 | 18.225 | 1.00 | 51.34 |
| 2147 | C | HIS | B | 687 | 12.008 | 41.392 | 19.204 | 1.00 | 48.80 |
| 2148 | O | HIS | B | 687 | 11.637 | 40.237 | 19.403 | 1.00 | 48.44 |
| 2149 | N | ALA | B | 688 | 11.168 | 42.417 | 19.100 | 1.00 | 45.79 |
| 2150 | CA | ALA | B | 688 | 9.725 | 42.258 | 19.240 | 1.00 | 43.75 |
| 2151 | CB | ALA | B | 688 | 9.041 | 43.614 | 19.092 | 1.00 | 43.70 |
| 2152 | C | ALA | B | 688 | 9.115 | 41.263 | 18.255 | 1.00 | 41.94 |
| 2153 | O | ALA | B | 688 | 8.490 | 40.284 | 18.661 | 1.00 | 40.94 |
| 2154 | N | ILE | B | 689 | 9.295 | 41.517 | 16.963 | 1.00 | 40.75 |
| 2155 | CA | ILE | B | 689 | 8.740 | 40.649 | 15.931 | 1.00 | 39.42 |
| 2156 | CB | ILE | B | 689 | 9.107 | 41.161 | 14.523 | 1.00 | 39.12 |
| 2157 | CG2 | ILE | B | 689 | 8.638 | 40.169 | 13.463 | 1.00 | 38.63 |
| 2158 | OG1 | ILE | B | 689 | 8.460 | 42.530 | 14.296 | 1.00 | 38.90 |
| 2159 | OD1 | ILE | B | 689 | 8.741 | 43.134 | 12.937 | 1.00 | 39.20 |
| 2160 | C | ILE | B | 689 | 9.189 | 39.200 | 16.077 | 1.00 | 38.83 |
| 2161 | O | ILE | B | 689 | 8.367 | 38.282 | 16.048 | 1.00 | 37.60 |
| 2162 | N | LEU | B | 690 | 10.491 | 38.998 | 16.238 | 1.00 | 38.40 |
| 2163 | CA | LEU | B | 690 | 11.033 | 37.656 | 16.393 | 1.00 | 38.63 |
| 2164 | CB | LEU | B | 690 | 12.548 | 37.730 | 16.597 | 1.00 | 39.36 |
| 2165 | CG | LEU | B | 690 | 13.382 | 36.561 | 16.069 | 1.00 | 40.30 |
| 2166 | CD1 | LEU | B | 690 | 14.860 | 36.928 | 16.113 | 1.00 | 39.81 |
| 2167 | CD2 | LEU | B | 690 | 13.108 | 35.314 | 16.889 | 1.00 | 41.50 |
| 2168 | C | LEU | B | 690 | 10.358 | 36.989 | 17.593 | 1.00 | 39.78 |
| 2169 | O | LEU | B | 690 | 9.895 | 35.850 | 17.505 | 1.00 | 38.83 |
| 2170 | N | HIS | B | 691 | 10.294 | 37.706 | 18.713 | 1.00 | 40.27 |
| 2171 | CA | HIS | B | 691 | 9.647 | 37.176 | 19.911 | 1.00 | 41.72 |
| 2172 | CB | HIS | B | 691 | 9.668 | 38.211 | 21.036 | 1.00 | 43.49 |
| 2173 | CG | HIS | B | 691 | 10.897 | 38.155 | 21.887 | 1.00 | 45.34 |
| 2174 | CD2 | HIS | B | 691 | 11.909 | 39.040 | 22.053 | 1.00 | 46.24 |
| 2175 | ND1 | HIS | B | 691 | 11.186 | 37.085 | 22.705 | 1.00 | 46.60 |
| 2176 | CE1 | HIS | B | 691 | 12.322 | 37.313 | 23.339 | 1.00 | 46.83 |
| 2177 | NE2 | HIS | B | 691 | 12.781 | 38.492 | 22.962 | 1.00 | 47.22 |
| 2178 | C | HIS | B | 691 | 8.205 | 36.799 | 19.596 | 1.00 | 41.36 |
| 2179 | O | HIS | B | 691 | 7.741 | 35.717 | 19.959 | 1.00 | 41.77 |
| 2180 | N | ARG | B | 692 | 7.502 | 37.698 | 18.915 | 1.00 | 40.51 |
| 2181 | CA | ARG | B | 692 | 6.112 | 37.459 | 18.543 | 1.00 | 40.58 |
| 2182 | CB | ARG | B | 692 | 5.588 | 38.617 | 17.692 | 1.00 | 41.97 |
| 2183 | CG | ARG | B | 692 | 4.108 | 38.532 | 17.339 | 1.00 | 44.31 |
| 2184 | CD | ARG | B | 692 | 3.755 | 39.573 | 16.287 | 1.00 | 46.39 |
| 2185 | NE | ARG | B | 692 | 4.450 | 39.308 | 15.029 | 1.00 | 48.87 |
| 2186 | GZ | ARG | B | 692 | 4.516 | 40.159 | 14.010 | 1.00 | 49.52 |
| 2187 | NH1 | ARG | B | 692 | 3.929 | 41.346 | 14.093 | 1.00 | 50.67 |
| 2188 | NH2 | ARG | B | 692 | 5.172 | 39.822 | 12.907 | 1.00 | 49.63 |
| 2189 | C | ARG | B | 692 | 5.998 | 36.152 | 17.760 | 1.00 | 39.77 |
| 2190 | O | ARG | B | 692 | 5.250 | 35.252 | 18.148 | 1.00 | 38.70 |
| 2191 | N | LEU | B | 693 | 6.746 | 36.052 | 16.663 | 1.00 | 38.99 |
| 2192 | CA | LEU | B | 693 | 6.730 | 34.854 | 15.825 | 1.00 | 39.44 |
| 2193 | CB | LEU | B | 693 | 7.812 | 34.932 | 14.741 | 1.00 | 38.36 |
| 2194 | CG | LEU | B | 693 | 7.643 | 35.944 | 13.604 | 1.00 | 38.71 |
| 2195 | CD1 | LEU | B | 693 | 8.848 | 35.865 | 12.684 | 1.00 | 37.17 |
| 2196 | CD2 | LEU | B | 693 | 6.365 | 35.657 | 12.828 | 1.00 | 37.74 |
| 2197 | C | LEU | B | 693 | 6.936 | 33.587 | 16.641 | 1.00 | 40.12 |
| 2198 | O | LEU | B | 693 | 6.290 | 32.569 | 16.399 | 1.00 | 40.20 |
| 2199 | N | LEU | B | 694 | 7.842 | 33.651 | 17.609 | 1.00 | 41.30 |
| 2200 | CA | LEU | B | 694 | 8.125 | 32.498 | 18.450 | 1.00 | 43.03 |
| 2201 | CB | LEU | B | 694 | 9.406 | 32.740 | 19.250 | 1.00 | 41.27 |
| 2202 | CG | LEU | B | 694 | 10.694 | 32.762 | 18.421 | 1.00 | 40.09 |
| 2203 | CD1 | LEU | B | 694 | 11.853 | 33.242 | 19.269 | 1.00 | 39.04 |
| 2204 | CD2 | LEU | B | 694 | 10.964 | 31.366 | 17.874 | 1.00 | 39.24 |
| 2205 | C | LEU | B | 694 | 6.974 | 32.178 | 19.399 | 1.00 | 45.64 |
| 2206 | O | LEU | B | 694 | 6.877 | 31.061 | 19.905 | 1.00 | 44.78 |
| 2207 | N | GLN | B | 695 | 6.099 | 33.153 | 19.628 | 1.00 | 49.29 |
| 2208 | CA | GLN | B | 695 | 4.967 | 32.964 | 20.532 | 1.00 | 53.51 |
| 2209 | CB | GLN | B | 695 | 4.350 | 34.316 | 20.912 | 1.00 | 53.94 |
| 2210 | CG | GLN | B | 695 | 5.336 | 35.354 | 21.441 | 1.00 | 54.96 |
| 2211 | CD | GLN | B | 695 | 6.069 | 34.916 | 22.699 | 1.00 | 55.61 |
| 2212 | OE1 | GLN | B | 695 | 6.860 | 35.677 | 23.263 | 1.00 | 55.52 |
| 2213 | NE2 | GLN | B | 695 | 5.814 | 33.690 | 23.144 | 1.00 | 55.62 |
| 2214 | C | GLN | B | 695 | 3.873 | 32.061 | 19.959 | 1.00 | 55.94 |
| 2215 | O | GLN | B | 695 | 3.035 | 31.553 | 20.702 | 1.00 | 55.76 |
| 2216 | N | GLIJ | B | 696 | 3.880 | 31.866 | 18.644 | 1.00 | 58.97 |
| 2217 | CA | GLU | B | 696 | 2.882 | 31.025 | 17.984 | 1.00 | 62.61 |
| 2218 | CB | GLU | B | 696 | 1.467 | 31.515 | 18.320 | 1.00 | 63.02 |
| 2219 | CG | GLU | B | 696 | 1.294 | 33.044 | 18.453 | 1.00 | 63.82 |
| 2220 | CD | GLU | B | 696 | 1.649 | 33.832 | 17.202 | 1.00 | 64.13 |
| 2221 | OE1 | GLU | B | 696 | 1.115 | 33.519 | 16.115 | 1.00 | 64.51 |
| 2222 | OE2 | GLU | B | 696 | 2.454 | 34.777 | 17.309 | 1.00 | 64.16 |
| 2223 | C | GLU | B | 696 | 3.055 | 31.054 | 16.473 | 1.00 | 64.98 |
| 2224 | O | GLU | B | 696 | 2.671 | 30.129 | 15.756 | 1.00 | 65.58 |
| 2225 | N | GLY | B | 697 | 3.643 | 32.150 | 16.021 | 1.00 | 67.35 |
| 2226 | CA | GLY | B | 697 | 3.875 | 32.400 | 14.618 | 1.00 | 70.03 |
| 2227 | C | GLY | B | 697 | 3.612 | 33.876 | 14.561 | 1.00 | 71.80 |
| 2228 | O | GLY | B | 697 | 3.004 | 34.366 | 13.584 | 1.00 | 71.76 |
| 2229 | OT | GLY | B | 697 | 4.003 | 34.560 | 15.534 | 1.00 | 71.76 |
| 2230 | O | HOH | S | 1 | 23.021 | 22.410 | −2.190 | 1.00 | 19.60 |
| 2231 | O | HOH | S | 2 | 16.829 | 36.197 | 0.666 | 1.00 | 18.07 |
| 2232 | O | HOH | S | 3 | 22.165 | 35.434 | 11.145 | 1.00 | 15.63 |
| 2233 | O | HOH | S | 4 | 24.975 | 20.256 | −2.262 | 1.00 | 20.14 |
| 2234 | O | HOH | S | 5 | 22.159 | 29.605 | 15.803 | 1.00 | 19.70 |
| 2235 | O | HOH | S | 6 | 24.989 | 31.512 | −0.644 | 1.00 | 16.40 |
| 2236 | O | HOH | S | 7 | 10.834 | 33.694 | −3.352 | 1.00 | 17.23 |
| 2237 | O | HOH | S | 8 | 22.460 | 38.715 | −17.965 | 1.00 | 31.92 |
| 2238 | O | HOH | S | 9 | 17.528 | 31.039 | 1.273 | 1.00 | 14.10 |
| 2239 | O | HOH | S | 10 | 29.026 | 37.829 | 2.231 | 1.00 | 16.59 |
| 2240 | O | HOH | S | 11 | 16.137 | 33.624 | 0.850 | 1.00 | 17.71 |
| 2241 | O | HOH | S | 12 | 21.555 | 23.965 | −0.430 | 1.00 | 13.97 |
| 2242 | O | HOH | S | 13 | 24.577 | 41.484 | −5.877 | 1.00 | 19.10 |
| 2243 | O | HOH | S | 14 | 34.344 | 22.171 | 2.655 | 1.00 | 23.28 |
| 2244 | O | HOH | S | 15 | 23.968 | 19.698 | −6.030 | 1.00 | 21.97 |
| 2245 | O | HOH | 5 | 16 | 12.341 | 31.084 | −5.780 | 1.00 | 18.26 |
| 2246 | O | HOH | S | 17 | 17.291 | 24.202 | 0.490 | 1.00 | 21.48 |
| 2247 | O | HOH | S | 18 | 12.775 | 35.658 | −4.277 | 1.00 | 15.87 |
| 2248 | O | HOH | S | 19 | 32.945 | 42.649 | 9.747 | 1.00 | 47.61 |
| 2249 | O | HOH | S | 20 | 6.636 | 45.304 | 0.256 | 1.00 | 32.40 |
| 2250 | O | HOH | S | 21 | 11.642 | 46.359 | 3.122 | 1.00 | 47.12 |
| 2251 | O | HOH | S | 22 | 15.366 | 32.455 | −1.716 | 1.00 | 24.92 |
| 2252 | O | HOH | S | 23 | 15.186 | 49.092 | −1.355 | 1.00 | 26.79 |
| 2253 | O | HOH | S | 24 | 17.321 | 27.755 | 27.447 | 1.00 | 23.87 |
| 2254 | O | HOH | S | 25 | 32.119 | 32.883 | −0.957 | 1.00 | 24.24 |
| 2255 | O | HOH | S | 26 | 17.226 | 13.378 | 7.046 | 1.00 | 30.98 |
| 2256 | O | HOH | S | 27 | 17.607 | 52.477 | −12.562 | 1.00 | 30.41 |
| 2257 | O | HOH | S | 28 | 11.582 | 28.462 | 0.833 | 1.00 | 27.74 |
| 2258 | O | HOH | S | 29 | 29.336 | 58.175 | 3.211 | 1.00 | 24.47 |
| 2259 | O | 110H | S | 30 | 20.328 | 34.102 | 24.740 | 1.00 | 27.25 |
| 2260 | O | HOH | S | 31 | 11.297 | 30.724 | −3.163 | 1.00 | 29.15 |
| 2261 | O | HOH | S | 32 | 14.539 | 17.647 | −8.907 | 1.00 | 23.07 |
| 2262 | O | HOH | S | 33 | 12.598 | 30.559 | 30.560 | 1.00 | 24.86 |
| 2263 | O | HOH | S | 34 | 5.025 | 18.187 | 3.778 | 1.00 | 28.69 |
| 2264 | O | HOH | S | 35 | 14.927 | 26.075 | 0.820 | 1.00 | 31.48 |
| 2265 | O | HOH | S | 36 | 26.367 | 38.413 | −9.182 | 1.00 | 23.21 |
| 2266 | O | HOH | S | 37 | 30.494 | 11.197 | 5.944 | 1.00 | 32.46 |
| 2267 | O | HOH | S | 38 | 11.293 | 49.824 | 6.074 | 1.00 | 48.93 |
| 2268 | O | HOH | S | 39 | 20.529 | 16.901 | −4.931 | 1.00 | 32.28 |
| 2269 | O | HOH | S | 40 | 24.843 | 35.083 | −15.519 | 1.00 | 21.88 |
| 2270 | O | HOH | S | 41 | 7.674 | 40.665 | 3.297 | 1.00 | 37.33 |
| 2271 | O | HOH | S | 42 | 27.523 | 46.282 | −18.345 | 1.00 | 37.96 |
| 2272 | O | HOH | S | 43 | 22.709 | 13.774 | −4.567 | 1.00 | 49.60 |
| 2273 | O | HOH | S | 44 | 7.423 | 32.242 | 11.910 | 1.00 | 23.05 |
| 2274 | O | HOH | S | 45 | 31.276 | 40.964 | 1.308 | 1.00 | 31.68 |
| 2275 | O | HOH | S | 46 | 33.265 | 32.328 | 3.517 | 1.00 | 19.13 |
| 2276 | O | HOH | S | 47 | 6.555 | 16.927 | 5.904 | 1.00 | 23.85 |
| 2277 | O | HOH | S | 48 | 17.606 | 50.658 | 7.865 | 1.00 | 28.11 |
| 2278 | O | HOH | S | 49 | 20.830 | 9.388 | 10.713 | 1.00 | 28.29 |
| 2279 | O | HOH | S | 50 | 13.364 | 37.050 | 29.629 | 1.00 | 23.94 |
| 2280 | O | HOH | S | 51 | 9.192 | 18.836 | −1.960 | 1.00 | 33.23 |
| 2281 | O | HOH | S | 52 | 23.567 | 48.701 | −14.763 | 1.00 | 55.81 |
| 2282 | O | HOH | S | 53 | 33.968 | 34.742 | 2.852 | 1.00 | 26.86 |
| 2283 | O | HOH | S | 54 | 29.820 | 10.990 | 8.338 | 1.00 | 28.51 |
| 2284 | O | HOH | S | 55 | 11.748 | 42.238 | 23.648 | 1.00 | 42.57 |
| 2285 | O | HOH | S | 56 | 14.767 | 33.631 | −4.195 | 1.00 | 32.75 |
| 2286 | O | HOH | S | 57 | 12.500 | 34.343 | 29.890 | 1.00 | 22.65 |
| 2287 | O | HOH | S | 58 | 28.117 | 41.866 | −15.415 | 1.00 | 31.74 |
| 2288 | O | HOH | S | 59 | 26.356 | 40.551 | −7.512 | 1.00 | 22.73 |
| 2289 | O | HOH | S | 60 | 22.268 | 46.641 | −15.890 | 1.00 | 50.83 |
| 2290 | O | HOH | S | 61 | 7.181 | 15.455 | 0.062 | 1.00 | 44.46 |
| 2291 | O | HOH | S | 62 | 3.620 | 30.584 | −0.194 | 1.00 | 36.39 |
| 2292 | O | HOH | S | 63 | 6.128 | 27.849 | 3.787 | 1.00 | 52.56 |
| 2293 | O | HOH | S | 64 | 31.099 | 55.471 | 8.227 | 1.00 | 30.35 |
| 2294 | O | HOH | S | 65 | 18.603 | 58.163 | −1.009 | 1.00 | 47.14 |
| 2295 | O | HOH | S | 66 | 8.356 | 23.629 | 26.974 | 1.00 | 32.72 |
| 2296 | O | HOH | S | 67 | 19.654 | 57.594 | −3.383 | 1.00 | 43.61 |
| 2297 | O | HOH | S | 68 | 20.930 | 28.296 | 33.352 | 1.00 | 40.59 |
| 2298 | O | HOH | S | 69 | 21.652 | 36.732 | 27.720 | 1.00 | 35.00 |
| 2299 | O | HOH | S | 70 | 31.556 | 42.782 | 3.272 | 1.00 | 30.88 |
| 2300 | O | HOH | S | 71 | 13.327 | 50.293 | 2.487 | 1.00 | 38.75 |
| 2301 | O | HOH | S | 72 | 19.141 | 53.232 | 5.426 | 1.00 | 37.22 |
| 2302 | O | HOH | S | 73 | 10.869 | 22.357 | 19.472 | 1.00 | 19.26 |
| 2303 | O | HOH | S | 74 | 4.005 | 20.489 | 2.211 | 1.00 | 37.71 |
| 2304 | O | HOH | S | 75 | 32.792 | 10.675 | 9.182 | 1.00 | 22.60 |
| 2305 | O | HOH | S | 76 | 15.967 | 27.450 | −1.311 | 1.00 | 30.63 |
| 2306 | O | HOH | S | 77 | 6.796 | 41.276 | 9.965 | 1.00 | 45.09 |
| 2307 | O | HOH | S | 78 | 6.261 | 30.440 | 7.929 | 1.00 | 33.02 |
| 2308 | O | HOH | S | 79 | 19.308 | 14.907 | 21.667 | 1.00 | 42.45 |
| 2309 | O | HOH | S | 80 | 2.565 | 29.418 | 1.814 | 1.00 | 40.89 |
| 2310 | O | HOH | S | 81 | 2.180 | 49.135 | −16.647 | 1.00 | 53.37 |
| 2311 | O | HOH | S | 82 | 36.343 | 8.679 | 3.325 | 1.00 | 40.82 |
| 2312 | O | HOH | S | 83 | 22.711 | 19.517 | 29.686 | 1.00 | 47.69 |
| 2313 | O | HOH | S | 84 | 33.973 | 34.630 | 0.258 | 1.00 | 31.56 |
| 2314 | O | HOH | S | 85 | 31.745 | 38.264 | 1.536 | 1.00 | 22.56 |
| 2315 | O | HOH | S | 86 | 5.827 | 37.873 | 6.417 | 1.00 | 48.05 |
| 2316 | O | HOH | S | 87 | 19.499 | 17.423 | −8.991 | 1.00 | 38.26 |
| 2317 | O | HOH | S | 88 | 40.418 | 35.587 | 7.944 | 1.00 | 35.76 |
| 2318 | O | HOH | S | 89 | 15.390 | 46.880 | 15.780 | 1.00 | 39.90 |
| 2319 | O | HOH | S | 90 | 6.878 | 29.542 | −13.424 | 1.00 | 40.19 |
| 2320 | O | HOH | S | 91 | 8.647 | 28.612 | 20.656 | 1.00 | 29.73 |
| 2321 | O | HOH | S | 92 | 22.042 | 37.435 | 22.086 | 1.00 | 37.50 |
| 2322 | O | HOH | S | 93 | 27.585 | 30.929 | 23.977 | 1.00 | 32.74 |
| 2323 | O | HOH | S | 94 | 10.974 | 31.175 | 27.894 | 1.00 | 24.96 |
| 2324 | O | HOH | S | 95 | 26.202 | 25.474 | −11.896 | 1.00 | 41.41 |
| 2325 | O | HOH | S | 96 | 6.726 | 33.443 | −14.585 | 1.00 | 36.10 |
| 2326 | O | HOH | S | 97 | 15.550 | 8.924 | 10.717 | 1.00 | 33.88 |
| 2327 | O | HOH | S | 98 | 16.476 | 16.841 | −2.591 | 1.00 | 26.55 |
| 2328 | O | HOH | S | 99 | 28.319 | 28.274 | 12.336 | 1.00 | 43.77 |
| 2329 | O | HOH | S | 100 | 27.769 | 3.457 | 13.155 | 1.00 | 48.23 |
| 2330 | O | HOH | S | 101 | 24.882 | 39.541 | −19.201 | 1.00 | 53.25 |
| 2331 | O | HOH | S | 102 | 12.968 | 44.583 | −8.525 | 1.00 | 25.37 |
| 2332 | O | HOH | S | 103 | 20.637 | 61.548 | 2.038 | 1.00 | 57.43 |
| 2333 | O | HOH | S | 104 | 18.394 | 21.172 | −12.670 | 1.00 | 30.69 |
| 2334 | O | HOH | S | 105 | 21.685 | 40.720 | 28.499 | 1.00 | 31.23 |
| 2335 | O | HOH | S | 106 | 37.452 | 24.342 | 8.575 | 1.00 | 43.15 |
| 2336 | O | HOH | S | 107 | 28.072 | 12.030 | −2.850 | 1.00 | 35.13 |
| 2337 | O | HOH | S | 108 | 13.643 | 6.793 | 9.813 | 1.00 | 59.43 |
| 2338 | O | HOH | S | 109 | 29.194 | 47.369 | −11.513 | 1.00 | 33.93 |
| 2339 | O | HOR | S | 110 | 37.211 | 25.400 | 11.266 | 1.00 | 49.29 |
| 2340 | O | HOH | S | 111 | 32.452 | 24.117 | 12.117 | 1.00 | 37.91 |
| 2341 | O | HOH | S | 112 | 20.047 | 18.433 | −15.782 | 1.00 | 52.69 |
| 2342 | O | HOH | S | 113 | 37.297 | 36.567 | 3.622 | 1.00 | 50.95 |
| 2343 | O | HOH | S | 114 | 26.638 | 37.932 | 27.578 | 1.00 | 60.19 |
| 2344 | O | HOH | S | 115 | 40.067 | 37.141 | 6.034 | 1.00 | 62.83 |
| 2345 | O | HOH | S | 116 | 16.702 | 28.787 | 32.709 | 1.00 | 45.22 |
| 2346 | O | HOH | S | 117 | 15.614 | 14.400 | −2.970 | 1.00 | 32.52 |
| 2347 | O | HOH | S | 118 | 25.304 | 43.389 | 12.324 | 1.00 | 46.21 |
| 2348 | O | HOH | S | 119 | 35.986 | 27.446 | −0.047 | 1.00 | 39.51 |
| 2349 | O | HOH | S | 120 | 33.667 | 38.724 | −6.920 | 1.00 | 55.40 |
| 2350 | O | HOH | S | 121 | 2.653 | 22.799 | 6.024 | 1.00 | 32.62 |
| 2351 | O | HOH | S | 122 | 21.427 | 16.319 | 32.924 | 1.00 | 56.84 |
| 2352 | O | HOH | S | 123 | 2.535 | 25.653 | 7.482 | 1.00 | 49.14 |
| 2353 | O | HOH | S | 124 | 38.296 | 26.623 | 7.497 | 1.00 | 36.28 |
| 2354 | O | HOH | S | 125 | 24.325 | 56.054 | −3.208 | 1.00 | 42.51 |
| 2355 | O | HOH | S | 126 | 31.374 | 39.772 | −15.850 | 1.00 | 43.44 |
| 2356 | O | HOH | S | 127 | 14.293 | 49.969 | 9.738 | 1.00 | 46.86 |
| 2357 | O | HOH | S | 128 | 29.446 | 46.358 | −15.130 | 1.00 | 56.58 |
| 2358 | O | HOH | S | 129 | 13.234 | 46.587 | 14.294 | 1.00 | 30.39 |
| 2359 | O | HOH | S | 130 | 28.696 | 21.460 | 14.541 | 1.00 | 43.43 |
| 2360 | O | HOH | S | 131 | 29.833 | 19.823 | 16.133 | 1.00 | 53.77 |
| 2361 | O | HOH | S | 132 | 28.668 | 40.610 | 0.027 | 1.00 | 27.60 |
| 2362 | O | HOH | S | 133 | 31.999 | 29.578 | 17.949 | 1.00 | 52.12 |
| 2363 | O | HOH | S | 134 | 32.331 | 41.291 | −7.181 | 1.00 | 48.91 |
| 2364 | O | HOH | S | 135 | 18.143 | 37.552 | 22.925 | 1.00 | 31.27 |
| 2365 | O | HOH | S | 136 | 16.874 | 57.740 | −7.181 | 1.00 | 36.07 |
| 2366 | O | HOH | S | 137 | 3.614 | 38.629 | 3.896 | 1.00 | 63.76 |
| 2367 | O | HOH | S | 138 | 18.541 | 13.341 | −5.104 | 1.00 | 59.11 |
| 2368 | O | HOH | S | 139 | 6.118 | 51.002 | −15.654 | 1.00 | 38.96 |
| 2369 | O | HOH | S | 140 | 14.730 | 25.982 | 29.459 | 1.00 | 32.23 |
| 2370 | O | HOH | S | 141 | 21.736 | 19.325 | −8.319 | 1.00 | 36.70 |
| 2371 | O | HOH | S | 142 | 8.176 | 16.537 | 17.699 | 1.00 | 58.27 |
| 2372 | O | HOH | S | 143 | 11.696 | 32.975 | 0.495 | 1.00 | 36.22 |
| 2373 | O | HOH | S | 144 | 13.563 | 48.324 | 0.595 | 1.00 | 37.61 |
| 2374 | O | HOH | S | 145 | 12.379 | 12.447 | 17.047 | 1.00 | 62.89 |
| 2375 | O | HOH | S | 146 | 9.604 | 31.872 | 1.213 | 1.00 | 68.72 |
| 2376 | O | HOH | S | 147 | 28.144 | 19.163 | 18.746 | 1.00 | 42.30 |
| 2377 | O | HOH | S | 148 | 32.515 | 45.933 | −7.466 | 1.00 | 37.61 |
| 2378 | O | HOH | S | 149 | 24.759 | 0.889 | 18.947 | 1.00 | 66.75 |
| 2379 | O | HOH | S | 150 | 39.683 | 22.653 | 2.230 | 1.00 | 35.42 |
| 2380 | O | HOH | S | 151 | 13.554 | 52.853 | 2.352 | 1.00 | 37.57 |
| 2381 | O | HOH | S | 152 | 8.318 | 48.977 | 2.507 | 1.00 | 73.06 |
| 2382 | O | HOH | S | 153 | 8.633 | 28.003 | −12.609 | 1.00 | 33.91 |
| 2383 | O | HOH | S | 154 | 2.882 | 30.152 | 4.149 | 1.00 | 62.49 |
| 2384 | O | HOH | S | 155 | 31.535 | 7.386 | 4.692 | 1.00 | 35.95 |
| 2385 | O | HOH | S | 156 | 5.852 | 26.333 | 22.676 | 1.00 | 39.01 |
| 2386 | O | HOH | S | 157 | 7.617 | 46.335 | −19.650 | 1.00 | 50.76 |
| 2387 | O | HOH | S | 158 | 23.004 | 9.857 | −2.493 | 1.00 | 64.92 |
| 2388 | O | HOH | S | 159 | 6.680 | 37.243 | 8.927 | 1.00 | 35.74 |
| 2389 | O | HOH | S | 160 | 1.655 | 40.656 | 10.739 | 1.00 | 68.96 |
| 2390 | O | HOH | S | 161 | 37.861 | 28.017 | 11.079 | 1.00 | 29.28 |
| 2391 | O | HOH | S | 162 | 2.363 | 30.081 | 11.152 | 1.00 | 47.90 |
| 2392 | O | HOH | S | 163 | 22.816 | 6.883 | 24.251 | 1.00 | 50.27 |
| 2393 | O | HOH | 5 | 164 | 25.232 | 24.018 | −9.509 | 1.00 | 56.25 |
| 2394 | O | HOH | S | 165 | 31.917 | 40.723 | −4.749 | 1.00 | 43.99 |
| 2395 | O | HOH | S | 166 | 29.060 | 32.100 | −14.160 | 1.00 | 48.37 |
| 2396 | O | HOH | S | 167 | 6.850 | 24.422 | 13.622 | 1.00 | 30.86 |
| 2397 | O | HOH | S | 168 | 31.128 | 26.378 | −0.266 | 1.00 | 26.16 |
| 2398 | O | HOH | S | 169 | 26.393 | 4.775 | 16.316 | 1.00 | 46.62 |
| 2399 | O | HOH | S | 170 | 30.226 | 52.800 | −0.982 | 1.00 | 62.86 |
| 2400 | O | HOH | S | 171 | 11.260 | 25.311 | −15.954 | 1.00 | 30.67 |
| 2401 | O | HOH | S | 172 | 8.236 | 22.803 | −8.257 | 1.00 | 36.47 |
| 2402 | O | HOH | S | 173 | 32.734 | 30.835 | 1.127 | 1.00 | 29.48 |
| 2403 | O | HOH | S | 174 | 12.162 | 13.528 | −6.989 | 1.00 | 54.81 |
| 2404 | O | HOH | S | 175 | 10.004 | 18.589 | −15.661 | 1.00 | 52.49 |
| 2405 | O | HOH | S | 176 | 2.597 | 15.841 | 3.847 | 1.00 | 36.47 |
| 2406 | O | HOH | S | 177 | 16.049 | 21.664 | 25.082 | 1.00 | 36.38 |
| 2407 | O | HOH | S | 178 | 9.566 | 53.021 | 5.395 | 1.00 | 51.65 |
| 2408 | O | HOH | S | 179 | 9.844 | 33.557 | 29.120 | 1.00 | 41.08 |
| 2409 | O | HOH | S | 180 | 28.907 | 9.505 | −2.595 | 1.00 | 43.51 |
| 2410 | O | HOH | S | 181 | 29.014 | 14.102 | −1.746 | 1.00 | 52.94 |
| 2411 | O | HOH | S | 182 | 8.615 | 16.558 | 11.985 | 1.00 | 34.51 |
| 2412 | O | HOH | S | 183 | 12.854 | 16.652 | −1.111 | 1.00 | 38.29 |
| 2413 | O | HOH | S | 184 | 28.378 | 56.857 | −0.308 | 1.00 | 70.56 |
| 2414 | O | HOH | S | 185 | 21.366 | 14.621 | 28.348 | 1.00 | 75.81 |
| 2415 | O | HOH | S | 186 | 29.539 | 7.952 | 13.215 | 1.00 | 47.28 |
| 2416 | O | HOH | S | 187 | 32.951 | 32.466 | 15.334 | 1.00 | 53.06 |
| 2417 | O | HOH | S | 188 | 6.055 | 23.083 | 15.885 | 1.00 | 44.59 |
| 2418 | O | HOH | S | 189 | 32.033 | 9.844 | 17.068 | 1.00 | 61.54 |
| 2419 | O | HOH | S | 190 | 31.234 | 15.535 | 16.608 | 1.00 | 47.53 |
| 2420 | O | HOH | 5 | 191 | 25.418 | 9.801 | 20.811 | 1.00 | 55.35 |
| 2421 | O | HOH | S | 192 | 17.915 | 17.399 | −4.929 | 1.00 | 38.99 |
| 2422 | O | HOH | S | 193 | 20.092 | 56.578 | −12.068 | 1.00 | 34.77 |
| 2423 | O | HOH | S | 194 | 5.198 | 29.909 | 5.015 | 1.00 | 54.91 |
| 2424 | O | HOH | S | 195 | 14.259 | 59.116 | −6.790 | 1.00 | 57.78 |
| 2425 | O | HOH | S | 196 | 2.335 | 36.084 | 19.359 | 1.00 | 71.38 |
| 2426 | O | HOH | S | 197 | 25.907 | 29.027 | 30.690 | 1.00 | 45.68 |
| 2427 | O | HOH | S | 198 | 7.004 | 27.666 | 7.371 | 1.00 | 33.37 |
| 2428 | O | HOH | S | 199 | 29.917 | 10.266 | 3.633 | 1.00 | 28.69 |
| 2429 | O | HOH | S | 200 | 3.229 | 39.595 | 8.996 | 1.00 | 64.29 |
| 2430 | O | HOH | S | 201 | 11.658 | 26.014 | 31.457 | 1.00 | 30.92 |
| 2431 | O | HOH | S | 202 | 30.585 | 43.900 | −10.316 | 1.00 | 35.47 |
| 2432 | O | HOH | S | 203 | 5.657 | 20.000 | 12.450 | 1.00 | 36.93 |
| 2433 | O | HOH | S | 204 | 9.001 | 8.083 | 12.525 | 1.00 | 44.27 |
| 2434 | O | HOH | S | 205 | 18.577 | 1.267 | 23.323 | 1.00 | 53.87 |
| 2435 | O | HOH | S | 206 | 14.129 | 50.521 | −14.443 | 1.00 | 54.64 |
| 2436 | O | HOH | S | 207 | 18.959 | 15.493 | 28.375 | 1.00 | 71.59 |
| 2437 | O | HOH | S | 208 | 0.669 | 37.375 | 17.680 | 1.00 | 49.95 |
| 2438 | O | HOH | S | 209 | 26.872 | 55.578 | −2.201 | 1.00 | 46.16 |
| 2439 | O | HOH | S | 210 | 6.168 | 22.883 | 11.718 | 1.00 | 31.87 |
| 2440 | O | HOH | S | 211 | 9.897 | 16.355 | −1.729 | 1.00 | 36.68 |
| 2441 | O | HOH | S | 212 | 30.265 | 27.848 | 23.670 | 1.00 | 53.45 |
| 2442 | O | HOH | S | 213 | 30.762 | 47.558 | −1.627 | 1.00 | 42.34 |
| 2443 | O | HOH | S | 214 | 32.189 | 35.014 | 15.024 | 1.00 | 44.29 |
| 2444 | O | HOH | S | 215 | 10.272 | 34.635 | −0.779 | 1.00 | 25.15 |
| 2445 | O | HOH | S | 216 | 4.890 | 17.836 | 16.021 | 1.00 | 58.71 |
| 2446 | O | HOH | S | 217 | 5.733 | 41.605 | 7.246 | 1.00 | 41.48 |
| 2447 | O | HOH | S | 218 | 24.986 | 27.076 | −7.629 | 1.00 | 61.37 |
| 2448 | O | HOH | S | 219 | 26.433 | 10.728 | 26.102 | 1.00 | 47.70 |
| 2449 | O | HOH | S | 220 | 9.403 | 26.411 | 28.194 | 1.00 | 51.51 |
| 2450 | O | HOH | S | 221 | 22.508 | 23.957 | −9.377 | 1.00 | 43.79 |
| 2451 | O | HOH | S | 222 | 36.297 | 31.156 | 7.134 | 1.00 | 49.35 |
| 2452 | O | HOH | S | 223 | 16.073 | 56.555 | 4.729 | 1.00 | 47.51 |
| 2453 | O | HOH | S | 224 | 9.102 | 45.988 | 15.199 | 1.00 | 51.54 |
| 2454 | O | HOH | S | 225 | 22.868 | 15.999 | −6.862 | 1.00 | 48.61 |
| 2455 | O | HOH | S | 226 | 4.554 | 37.721 | 10.138 | 1.00 | 63.61 |
| 2456 | O | HOH | S | 227 | 8.317 | 20.227 | −8.684 | 1.00 | 40.62 |
| 2457 | O | HOH | S | 228 | 26.553 | 17.081 | 26.527 | 1.00 | 60.48 |
| 2458 | O | HOH | S | 229 | 18.435 | 52.120 | −15.938 | 1.00 | 68.74 |
| 2459 | O | HOH | S | 230 | 15.731 | 11.256 | 6.138 | 1.00 | 40.27 |
| 2460 | O | HOH | S | 231 | −2.470 | 33.671 | 15.319 | 1.00 | 56.63 |
| 2461 | O | HOH | S | 232 | 9.235 | 37.391 | 27.202 | 1.00 | 50.70 |
| 2462 | O | HOH | S | 233 | 22.857 | 5.454 | −0.374 | 1.00 | 56.93 |
| 2463 | O | HOH | S | 234 | 1.412 | 28.065 | 19.093 | 1.00 | 54.97 |
| 2464 | O | HOH | S | 235 | 29.383 | 5.654 | 12.365 | 1.00 | 46.23 |
| 2465 | O | HOH | S | 236 | 21.394 | 2.024 | 21.053 | 1.00 | 54.78 |
| 2466 | O | HOH | S | 237 | 3.134 | 52.176 | −19.288 | 1.00 | 61.95 |
| 2467 | O | HOH | S | 238 | 22.005 | 57.555 | −0.605 | 1.00 | 48.17 |
| 2468 | O | HOH | S | 239 | 13.682 | 3.617 | 13.009 | 1.00 | 43.82 |
| 2469 | O | HOH | S | 240 | 19.176 | 16.596 | 19.466 | 1.00 | 31.39 |
| 2470 | O | HOH | S | 241 | 3.338 | 33.539 | 6.594 | 1.00 | 47.67 |
| 2471 | O | HOH | S | 242 | 10.760 | 2.995 | 19.481 | 1.00 | 70.14 |
| 2472 | O | HOH | S | 243 | 16.801 | 17.522 | 20.526 | 1.00 | 63.73 |
| 2473 | O | HOH | S | 244 | 2.600 | 44.637 | 16.700 | 1.00 | 56.53 |
| 2474 | O | HOH | S | 245 | 27.872 | 14.286 | 18.168 | 1.00 | 41.98 |
| 2475 | O | HOH | S | 246 | 31.396 | 45.519 | −13.517 | 1.00 | 53.59 |
| 2476 | O | HOH | S | 247 | 31.244 | 35.576 | −11.964 | 1.00 | 33.65 |
| 2477 | O | HOH | S | 248 | 21.693 | 4.459 | 26.360 | 1.00 | 52.63 |
| 2478 | O | HOH | S | 249 | 10.433 | 20.471 | 21.568 | 1.00 | 35.08 |
| 2479 | O | HOH | S | 250 | 17.785 | 18.041 | 18.016 | 1.00 | 49.80 |
| 2480 | O | HOH | S | 251 | 12.908 | 41.185 | 25.864 | 1.00 | 49.78 |
| 2481 | O | HOH | S | 252 | 11.825 | 38.262 | 27.686 | 1.00 | 41.47 |
| 2482 | O | HOH | S | 253 | 21.717 | 14.484 | 25.654 | 1.00 | 49.41 |
| 2483 | O | HOH | S | 254 | 3.264 | 19.434 | −21.232 | 1.00 | 58.93 |
| 2484 | O | HOH | S | 255 | 32.144 | 44.531 | −1.765 | 1.00 | 63.10 |
| 2485 | O | HOH | S | 256 | 30.528 | 19.733 | 26.321 | 1.00 | 56.72 |
| 2486 | O | HOH | S | 257 | 1.014 | 38.169 | 8.350 | 1.00 | 69.65 |
| 2487 | O | HOH | S | 258 | 15.650 | 56.451 | −11.110 | 1.00 | 45.85 |
| 2488 | O | HOH | S | 259 | 9.637 | 49.206 | −12.734 | 1.00 | 31.76 |
| 2489 | O | HOH | S | 260 | 26.271 | 33.962 | 11.972 | 1.00 | 28.74 |
| 2490 | O | HOH | S | 261 | 16.265 | 55.388 | −6.013 | 1.00 | 48.50 |
| 2491 | O | HOH | S | 262 | 35.225 | 25.151 | 1.974 | 1.00 | 37.37 |
| 2492 | O | HOH | S | 263 | 34.131 | 31.093 | 10.695 | 1.00 | 35.88 |
| 2493 | O | HOH | S | 264 | 9.808 | 12.096 | −10.102 | 1.00 | 42.96 |
| 2494 | O | HOH | S | 265 | 31.337 | 17.800 | 15.541 | 1.00 | 54.02 |
| 2495 | O | HOH | S | 266 | 6.034 | 17.605 | 1.265 | 1.00 | 32.48 |
| 2496 | O | HOH | S | 267 | 10.657 | 28.653 | 26.815 | 1.00 | 33.27 |
| 2497 | O | HOH | S | 268 | 33.123 | 28.220 | 0.778 | 1.00 | 32.26 |
| 2498 | O | HOH | S | 269 | 4.688 | 49.518 | −17.401 | 1.00 | 56.01 |
| 2499 | O | HOH | S | 270 | 19.934 | 38.276 | 25.088 | 1.00 | 52.01 |
| 2500 | O | HOH | S | 271 | 17.067 | 16.746 | −7.685 | 1.00 | 44.99 |
| 2501 | O | HOH | S | 272 | 8.184 | 32.997 | 31.099 | 1.00 | 38.13 |
| 2502 | O | HOH | S | 273 | 15.358 | 23.005 | 31.926 | 1.00 | 47.35 |
| 2503 | O | HOH | S | 274 | 37.594 | 33.163 | 10.831 | 1.00 | 50.51 |
| 2504 | O | HOH | S | 275 | 26.340 | 26.057 | 14.475 | 1.00 | 43.81 |
| 2505 | O | HOH | S | 276 | 9.314 | 11.770 | 3.305 | 1.00 | 52.95 |
| 2506 | O | HOH | S | 277 | 29.890 | 5.053 | 4.222 | 1.00 | 39.94 |
| 2507 | O | HOH | S | 278 | 21.596 | 63.668 | 5.719 | 1.00 | 47.51 |
| 2508 | O | HOH | S | 280 | 4.234 | 31.186 | 9.139 | 1.00 | 34.04 |
| 2509 | O | HOH | S | 281 | 20.211 | 52.099 | −12.173 | 1.00 | 32.14 |
| 2510 | O | HOH | S | 282 | 31.764 | 46.901 | −9.886 | 1.00 | 37.63 |
| 2511 | O | HOH | S | 283 | 29.356 | 16.377 | 18.834 | 1.00 | 47.88 |
| 2512 | O | HOH | S | 284 | 7.077 | 22.502 | 23.360 | 1.00 | 49.62 |
| 2513 | O | HOH | S | 285 | 10.457 | 17.845 | 24.918 | 1.00 | 45.38 |
| 2514 | O | HOH | S | 286 | 22.709 | 21.642 | −7.858 | 1.00 | 44.54 |
| 2515 | O | HOH | S | 287 | 41.743 | 38.399 | 4.312 | 1.00 | 51.42 |
| 2516 | O | HOH | S | 288 | 28.342 | 29.192 | 29.846 | 1.00 | 35.37 |
| 2517 | O | HOH | S | 289 | 19.871 | 8.666 | 8.338 | 1.00 | 36.88 |
| 2518 | O | HOH | S | 290 | 30.239 | 47.828 | 14.437 | 1.00 | 52.48 |
| 2519 | O | HOH | S | 291 | 38.133 | 31.699 | −0.085 | 1.00 | 48.80 |
| 2520 | O | HOH | S | 292 | 29.451 | 19.522 | 21.988 | 1.00 | 48.53 |
| 2521 | O | HOH | S | 293 | 28.207 | 17.784 | 23.529 | 1.00 | 49.19 |
| 2522 | O | HOH | S | 294 | 25.099 | 25.880 | 30.696 | 1.00 | 50.04 |
| 2523 | O | HOH | S | 295 | 20.984 | 24.305 | −13.635 | 1.00 | 35.02 |
| 2524 | O | HOH | S | 296 | 27.314 | 9.229 | 14.571 | 1.00 | 37.16 |
| 2525 | O | HOH | S | 297 | 5.590 | 44.198 | 15.517 | 1.00 | 46.00 |
| 2526 | O | HOH | S | 298 | 1.452 | 15.832 | 16.496 | 1.00 | 48.72 |
| 2527 | O | HOH | S | 299 | 1.895 | 52.120 | −9.294 | 1.00 | 36.65 |
| 2528 | O | HOH | S | 300 | 2.789 | 26.865 | 21.392 | 1.00 | 37.83 |
| 2529 | O | HOH | S | 301 | 10.822 | 21.853 | 1.469 | 1.00 | 38.42 |
| 2530 | O | HOH | S | 302 | 25.182 | 43.665 | 22.197 | 1.00 | 39.85 |
| 2531 | O | HOH | S | 303 | 10.013 | 44.307 | 3.131 | 1.00 | 61.53 |
| 2532 | O | HOH | S | 304 | 25.076 | 12.353 | −5.192 | 1.00 | 49.90 |
| 2533 | O | HOH | S | 305 | 24.684 | 26.059 | 34.224 | 1.00 | 51.37 |
| 2534 | O | HOH | S | 306 | 17.636 | 17.778 | −11.960 | 1.00 | 54.01 |
| 2535 | O | HOH | S | 307 | 21.679 | 9.218 | −4.605 | 1.00 | 50.75 |
| 2536 | O | HOH | S | 308 | 12.832 | 30.478 | −0.749 | 1.00 | 46.31 |
| 2537 | O | HOH | S | 309 | −0.832 | 31.716 | 16.046 | 1.00 | 49.15 |
| 2538 | O | HOH | S | 310 | 11.345 | 25.092 | 29.161 | 1.00 | 54.91 |
| 2539 | O | HOH | S | 311 | 38.438 | 33.351 | 4.404 | 1.00 | 47.87 |
| 2540 | O | HOH | S | 312 | 29.760 | 48.475 | −20.473 | 1.00 | 61.21 |
| 2541 | O | HOH | S | 313 | 19.889 | 22.967 | 34.487 | 1.00 | 53.69 |
| 2542 | O | HOH | S | 314 | 6.167 | 6.357 | 12.405 | 1.00 | 45.47 |
| 2543 | O | HOH | S | 315 | 24.800 | 24.028 | −13.646 | 1.00 | 41.36 |
| 2544 | O | HOH | S | 316 | 34.408 | 33.833 | 13.704 | 1.00 | 48.67 |
| 2545 | O | HOH | S | 317 | 28.499 | 24.877 | 15.179 | 1.00 | 42.44 |
| 2546 | O | HOH | S | 318 | 18.760 | 10.026 | −0.303 | 1.00 | 47.49 |
| 2547 | O | HOH | S | 319 | 27.889 | 33.343 | 15.965 | 1.00 | 46.60 |
| 2548 | O | HOH | S | 320 | 28.099 | 54.149 | −9.913 | 1.00 | 47.31 |
| 2549 | O | HOH | S | 321 | 6.669 | 20.044 | 17.321 | 1.00 | 66.41 |
| 2550 | O | HOH | S | 322 | 9.183 | 51.425 | −1.061 | 1.00 | 50.92 |
| 2551 | O | HOH | S | 323 | 26.136 | 49.957 | −13.069 | 1.00 | 44.73 |
| 2552 | O | HOH | S | 324 | 6.462 | 14.964 | 10.693 | 1.00 | 50.61 |
| 2553 | O | HOH | S | 325 | 27.434 | 21.342 | 29.941 | 1.00 | 46.70 |
| 2554 | O | HOH | S | 326 | −2.372 | 39.018 | 9.502 | 1.00 | 48.95 |
| 2555 | O | HOH | S | 327 | 39.676 | 34.723 | 11.054 | 1.00 | 54.77 |
| 2556 | O | HOH | S | 328 | 31.515 | 4.167 | 11.728 | 1.00 | 38.27 |
| 2557 | O | HOH | S | 329 | 30.273 | 43.414 | 12.468 | 1.00 | 56.22 |
| 2558 | O | HOH | S | 330 | 31.821 | 6.860 | 14.047 | 1.00 | 49.62 |
| 2559 | O | HOH | S | 331 | 34.176 | 36.382 | −11.484 | 1.00 | 54.81 |
| 2560 | O | HOH | S | 332 | 16.341 | 29.905 | −0.825 | 1.00 | 50.86 |
| 2561 | O | HOH | S | 333 | 16.034 | 14.050 | −0.096 | 1.00 | 26.81 |
| 2562 | O | HOH | S | 334 | 35.569 | 39.304 | 6.909 | 1.00 | 58.70 |
| 2563 | O | HOH | S | 335 | 7.222 | 18.767 | −13.380 | 1.00 | 48.09 |
| 2564 | O | HOH | S | 336 | 4.343 | 25.758 | 17.613 | 1.00 | 48.32 |
| 2565 | O | HOH | S | 337 | 23.980 | 49.898 | 20.317 | 1.00 | 45.46 |
| 2566 | O | HOH | S | 338 | −1.308 | 39.173 | 20.570 | 1.00 | 47.06 |
| 2567 | O | HOH | S | 339 | 29.402 | 35.057 | −23.568 | 1.00 | 51.53 |
| 2568 | O | HOH | S | 340 | 22.171 | 11.944 | −8.266 | 1.00 | 58.38 |
| 2569 | O | HOH | S | 341 | 28.556 | 23.420 | −8.393 | 1.00 | 38.50 |
| 2570 | O | HOH | S | 342 | 16.039 | 7.600 | 25.055 | 1.00 | 63.84 |
| 2571 | O | HOH | S | 343 | 2.335 | 34.042 | 2.843 | 1.00 | 52.94 |
| 2572 | O | HOH | S | 344 | 14.874 | 19.553 | 25.906 | 1.00 | 44.98 |
| 2573 | O | HOH | S | 345 | 33.947 | 47.332 | −12.606 | 1.00 | 52.85 |
| 2574 | O | HOH | S | 346 | 27.488 | 24.767 | 30.391 | 1.00 | 48.33 |
| 2575 | O | HOH | S | 347 | 20.467 | 43.261 | 26.119 | 1.00 | 52.94 |
| 2576 | O | HOH | S | 348 | 28.263 | 37.781 | 30.518 | 1.00 | 62.99 |
| 2577 | O | HOH | S | 349 | 27.485 | 34.119 | −15.359 | 1.00 | 50.71 |
| 2578 | O | HOH | S | 350 | 25.657 | 34.532 | 30.427 | 1.00 | 54.08 |
| 2579 | O | HOH | S | 351 | 29.768 | 1.154 | 1.312 | 1.00 | 59.34 |
| 2580 | O | HOH | S | 352 | 5.457 | 39.028 | 22.172 | 1.00 | 47.10 |
| 2581 | O | HOH | S | 353 | 5.067 | 16.762 | 20.070 | 1.00 | 60.26 |
| 2582 | O | HOH | S | 354 | 18.638 | 55.700 | −4.851 | 1.00 | ,55.62 |
| 2583 | O | HOH | S | 355 | 32.686 | 59.039 | 10.830 | 1.00 | 39.90 |
| 2584 | O | HOH | S | 356 | 28.255 | 51.067 | −11.879 | 1.00 | 52.57 |
| 2585 | O | HOH | S | 357 | 11.617 | 51.221 | −1.789 | 1.00 | 63.06 |
| 2586 | O | HOH | S | 358 | 2.982 | 19.431 | −24.499 | 1.00 | 45.57 |
| 2587 | O | HOH | S | 359 | 22.096 | 60.033 | −1.451 | 1.00 | 61.45 |
| 2588 | O | HOH | S | 360 | 22.637 | 43.238 | 31.026 | 1.00 | 47.28 |
| 2589 | O | HOH | S | 361 | 30.447 | 38.954 | 29.873 | 1.00 | 57.25 |
| 2590 | O | HOH | S | 362 | 35.833 | 38.025 | 1.980 | 1.00 | 61.81 |
| 2591 | O | HOH | S | 363 | 6.113 | 31.893 | −17.272 | 1.00 | 49.61 |
| 2592 | O | HOH | S | 364 | 6.724 | 15.601 | −22.408 | 1.00 | 59.42 |
| 2593 | O | HOH | S | 365 | 12.111 | 1.178 | 18.316 | 1.00 | 48.35 |
| 2594 | O | HOH | S | 366 | 4.240 | 32.098 | 1.799 | 1.00 | 44.18 |
| 2595 | O | HOH | S | 367 | 29.724 | 35.065 | 27.753 | 1.00 | 55.76 |
| 2596 | O | HOH | S | 368 | 26.360 | 7.462 | 25.028 | 1.00 | 51.90 |
| 2597 | O | HOH | S | 369 | 9.107 | 46.874 | 3.890 | 1.00 | 56.70 |
| 2598 | O | HOH | S | 370 | 10.650 | 46.430 | −20.344 | 1.00 | 37.21 |
| 2599 | O | HOH | S | 371 | 8.233 | 48.961 | 17.197 | 1.00 | 59.01 |
| 2600 | O | HOH | S | 372 | 20.322 | 15.007 | −8.818 | 1.00 | 55.36 |
| 2601 | O | HOH | S | 373 | 19.260 | −0.126 | 16.569 | 1.00 | 45.30 |
| 2602 | O | HOH | S | 374 | 7.343 | 49.778 | −0.668 | 1.00 | 55.02 |
| 2603 | O | HOH | S | 375 | 2.507 | 47.867 | 0.719 | 1.00 | 48.12 |
| 2604 | O | HOH | S | 376 | 13.136 | 54.591 | −2.145 | 1.00 | 53.44 |
| 2605 | O | HOH | S | 377 | 17.466 | 16.519 | 30.208 | 1.00 | 49.11 |
| 2606 | O | HOH | S | 378 | 20.118 | 10.769 | 23.252 | 1.00 | 55.34 |
| 2607 | O | HOH | S | 379 | 23.542 | 12.685 | 26.089 | 1.00 | 62.62 |
| 2608 | O | HOH | S | 380 | 20.375 | 47.120 | 12.159 | 1.00 | 51.92 |
| 2609 | O | HOH | S | 381 | 9.032 | 10.924 | 17.726 | 1.00 | 57.21 |
| 2610 | O | HOH | S | 382 | 15.077 | 15.226 | 30.470 | 1.00 | 56.74 |
| 2611 | O | HOH | S | 383 | 27.823 | 7.313 | −8.539 | 1.00 | 49.75 |
| 2612 | O | HOH | S | 384 | −1.210 | 31.371 | 18.699 | 1.00 | 53.14 |
| 2613 | O | HOH | S | 385 | 7.322 | 35.525 | 28.034 | 1.00 | 61.44 |
| 2614 | O | HOH | S | 386 | 14.217 | 16.674 | −13.221 | 1.00 | 42.98 |
| 2615 | O | HOH | S | 387 | 20.0785 | 9.930 | −2.925 | 1.00 | 54.66 |
| 2616 | O | HOH | S | 388 | 28.936 | 2.000 | 15.009 | 1.00 | 47.04 |
| 2617 | O | HOH | S | 389 | 14.166 | 28.692 | 31.577 | 1.00 | 59.65 |
| 2618 | O | HOH | S | 390 | 20.846 | 52.499 | 14.026 | 1.00 | 61.68 |
| 2619 | O | HOH | S | 391 | 13.294 | 5.368 | 25.444 | 1.00 | 52.09 |
| 2620 | O | HOH | S | 392 | 26.874 | 59.625 | −2.846 | 1.00 | 46.54 |
| 2621 | O | HOH | S | 393 | 11.393 | 5.392 | 16.760 | 1.00 | 45.29 |
| 2622 | O | HOH | S | 394 | 30.608 | 57.650 | 11.108 | 1.00 | 63.95 |
| 2623 | O | HOH | S | 395 | 33.858 | 39.183 | −13.250 | 1.00 | 54.28 |
| 2624 | O | HOH | S | 396 | 16.117 | 14.396 | −7.011 | 1.00 | 46.14 |
| 2625 | O | HOH | S | 397 | 37.725 | 38.263 | 6.165 | 1.00 | 64.28 |
| 2626 | O | HON | S | 398 | 6.555 | 42.050 | −19.474 | 1.00 | 48.56 |
| 2627 | C1A | 735 | C | 1 | 19.341 | 40.726 | 3.997 | 1.00 | 15.91 |
| 2628 | O1C | 735 | C | 1 | 18.234 | 40.214 | 4.328 | 1.00 | 17.26 |
| 2629 | O1B | 735 | C | 1 | 20.387 | 40.473 | 4.647 | 1.00 | 17.17 |
| 2630 | C1D | 735 | C | 1 | 19.458 | 41.690 | 2.760 | 1.00 | 14.73 |
| 2631 | C1X | 735 | C | 1 | 19.838 | 43.096 | 3.263 | 1.00 | 16.66 |
| 2632 | C1Y | 735 | C | 1 | 18.087 | 41.870 | 1.985 | 1.00 | 16.99 |
| 2633 | O1E | 735 | C | 1 | 20.617 | 41.213 | 1.907 | 1.00 | 15.62 |
| 2634 | C1F | 735 | C | 1 | 20.412 | 40.092 | 1.049 | 1.00 | 12.70 |
| 2635 | G1G | 735 | C | 1 | 20.433 | 40.315 | −0.334 | 1.00 | 16.64 |
| 2636 | G11 | 735 | C | 1 | 20.226 | 39.243 | −1.204 | 1.00 | 15.86 |
| 2637 | C1K | 735 | C | 1 | 19.985 | 37.892 | −0.696 | 1.00 | 15.12 |
| 2638 | C11 | 735 | C | 1 | 19.969 | 37.669 | 0.700 | 1.00 | 15.25 |
| 2639 | C1H | 735 | C | 1 | 20.181 | 38.761 | 1.576 | 1.00 | 16.34 |
| 2640 | C1L | 735 | C | 1 | 19.744 | 36.701 | −1.667 | 1.00 | 16.46 |
| 2641 | N1M | 735 | C | 1 | 19.050 | 37.255 | −2.868 | 1.00 | 16.48 |
| 2642 | C2A | 735 | C | 1 | 17.700 | 37.386 | −3.035 | 1.00 | 19.45 |
| 2643 | O2A | 735 | C | 1 | 16.905 | 37.016 | −2.143 | 1.00 | 20.40 |
| 2644 | S2C | 735 | C | 1 | 15.441 | 38.082 | −4.381 | 1.00 | 18.50 |
| 2645 | C2B | 735 | C | 1 | 17.156 | 37.942 | −4.212 | 1.00 | 18.76 |
| 2646 | C2D | 735 | C | 1 | 17.793 | 38.466 | −5.406 | 1.00 | 19.70 |
| 2647 | C2G | 735 | C | 1 | 19.306 | 38.558 | −5.720 | 1.00 | 20.26 |
| 2648 | N2E | 735 | C | 1 | 16.928 | 38.919 | −6.324 | 1.00 | 17.68 |
| 2649 | C2F | 735 | C | 1 | 15.614 | 38.789 | −5.945 | 1.00 | 19.94 |
| 2650 | C2H | 735 | C | 1 | 14.528 | 39.186 | −6.744 | 1.00 | 21.71 |
| 2651 | C2J | 735 | C | 1 | 13.173 | 39.004 | −6.286 | 1.00 | 20.47 |
| 2652 | C2L | 735 | C | 1 | 12.084 | 39.400 | −7.096 | 1.00 | 22.73 |
| 2653 | C2M | 735 | C | 1 | 12.305 | −39.993 | −8.391 | 1.00 | 20.47 |
| 2654 | C2K | 735 | C | 1 | 13.651 | 40.175 | −8.842 | 1.00 | 23.90 |
| 2655 | C2I | 735 | C | 1 | 14.735 | 39.782 | −8.042 | 1.00 | 22.13 |
| 2656 | C2N | 735 | C | 1 | 11.115 | 40.435 | −9.308 | 1.00 | 25.97 |
| 2657 | F2P | 735 | C | 1 | 10.952 | 39.499 | −10.296 | 1.00 | 31.88 |
| 2658 | F2Q | 735 | C | 1 | 9.934 | 40.532 | −8.627 | 1.00 | 31.88 |
| 2659 | F2O | 735 | C | 1 | 11.362 | 41.644 | −9.892 | 1.00 | 31.88 |

TABLE 3
ATOMIC STRUCTURE COORDINATE DATA OBTAINED FROM X-RAY
DIFFRACTION FROM THE LIGAND BINDING DOMAIN OF PPARc USED IN
MOLECULAR REPLACEMENT

| | ATOM | | PROTEIN | | | | | | |
|---|---|---|---|---|---|---|---|---|---|
| ATOM | TYPE | RESIDUE | # | # | X | Y | Z | OCC | B |
| 1 | CB | ASP | A | 211 | 14.51 | 5.574 | 19.848 | 1.00 | 7846 |
| 2 | CG | ASP | A | 211 | 14.259 | 4.095 | 20.068 | 1.00 | 79.53 |
| 3 | OD1 | ASP | A | 211 | 13.363 | 3.535 | 19.4 | 1.00 | 80.23 |
| 4 | OD2 | ASP | A | 211 | 14.961 | 3.492 | 20.908 | 1.00 | 80.14 |
| 5 | C | ASP | A | 211 | 15.241 | 7.375 | 18.272 | 1.00 | 76.33 |
| 6 | O | ASP | A | 211 | 14.371 | 8.176 | 17.929 | 1.00 | 76.50 |
| 7 | N | ASP | A | 211 | 16.106 | 5.066 | 18.029 | 1.00 | 77.36 |
| 8 | CA | ASP | A | 211 | 14.923 | 5.889 | 18.409 | 1.00 | 77.08 |
| 9 | N | LEU | A | 212 | 16.49 | 7.738 | 18.549 | 1.00 | 74.80 |
| 10 | CA | LEU | A | 212 | 16.926 | 9.125 | 18.438 | 1.00 | 72.96 |
| 11 | CB | LEU | A | 212 | 18.142 | 9.393 | 19.328 | 1.00 | 73.58 |
| 12 | CG | LEU | A | 212 | 18.809 | 10.76 | 19.098 | 1.00 | 73.64 |
| 13 | CD1 | LEU | A | 212 | 18.094 | 11.83 | 19.907 | 1.00 | 73.44 |
| 14 | CD2 | LEU | A | 212 | 20.274 | 10.68 | 19.492 | 1.00 | 73.65 |
| 15 | C | LEU | A | 212 | 17.315 | 9.398 | 16.997 | 1.00 | 71.55 |
| 16 | O | LEU | A | 212 | 17.304 | 10.54 | 16.548 | 1.00 | 71.54 |
| 17 | N | LYS | A | 213 | 17.672 | 8.346 | 16.274 | 1.00 | 69.51 |
| 18 | CA | LYS | A | 213 | 18.069 | 8.512 | 14.89 | 1.00 | 67.63 |
| 19 | CB | LYS | A | 213 | 18.837 | 7.279 | 14.416 | 1.00 | 68.39 |
| 20 | CG | LYS | A | 213 | 19.477 | 7.423 | 13.047 | 1.00 | 68.94 |
| 21 | CD | LYS | A | 213 | 20.908 | 6.9 | 13.051 | 1.00 | 69.41 |
| 22 | CE | LYS | A | 213 | 21.036 | 5.602 | 13.837 | 1.00 | 69.58 |
| 23 | NZ | LYS | A | 213 | 22.361 | 4.97 | 13.652 | 1.00 | 70.30 |
| 24 | C | LYS | A | 213 | 16.845 | 8.769 | 14.018 | 1.00 | 65.91 |
| 25 | O | LYS | A | 213 | 16.97 | 9.16 | 12.856 | 1.00 | 66.04 |
| 26 | N | SER | A | 214 | 15.661 | 8.558 | 14.587 | 1.00 | 62.82 |
| 27 | CA | SER | A | 214 | 14.425 | 8.817 | 13.86 | 1.00 | 59.48 |
| 28 | CB | SER | A | 214 | 13.216 | 8.267 | 14.624 | 1.00 | 59.65 |
| 29 | OG | SER | A | 214 | 12.983 | 9 | 15.815 | 1.00 | 59.47 |
| 30 | C | SER | A | 214 | 14.326 | 10.34 | 13.76 | 1.00 | 56.82 |
| 31 | O | SER | A | 214 | 13.501 | 10.87 | 13.019 | 1.00 | 56.27 |
| 32 | N | LEU | A | 215 | 15.183 | 11.01 | 14.524 | 1.00 | 53.62 |
| 33 | CA | LEU | A | 215 | 15.244 | 12.47 | 14.553 | 1.00 | 50.75 |
| 34 | CB | LEU | A | 215 | 16.368 | 12.93 | 15.49 | 1.00 | 50.59 |
| 35 | CG | LEU | A | 215 | 16.736 | 14.42 | 15.548 | 1.00 | 49.94 |
| 36 | CD1 | LEU | A | 215 | 15.58 | 15.23 | 16.111 | 1.00 | 49.18 |
| 37 | CD2 | LEU | A | 215 | 17.975 | 14.6 | 16.414 | 1.00 | 49.63 |
| 38 | C | LEU | A | 215 | 15.496 | 13.02 | 13.155 | 1.00 | 48.97 |
| 39 | O | LEU | A | 215 | 15.029 | 14.1 | 12.809 | 1.00 | 49.11 |
| 40 | N | ALA | A | 216 | 16.238 | 12.26 | 12.355 | 1.00 | 46.47 |
| 41 | CA | ALA | A | 216 | 16.553 | 12.66 | 10.994 | 1.00 | 45.03 |
| 42 | CB | ALA | A | 216 | 17.423 | 11.61 | 10.322 | 1.00 | 44.87 |
| 43 | C | ALA | A | 216 | 15.288 | 12.9 | 10.169 | 1.00 | 44.53 |
| 44 | O | ALA | A | 216 | 15.076 | 14 | 9.662 | 1.00 | 43.47 |
| 45 | N | LYS | A | 217 | 14.446 | 11.88 | 10.041 | 1.00 | 43.76 |
| 46 | CA | LYS | A | 217 | 13.219 | 12.02 | 9.264 | 1.00 | 42.20 |
| 47 | CB | LYS | A | 217 | 12.458 | 10.69 | 9.203 | 1.00 | 44.47 |
| 48 | CG | LYS | A | 217 | 11.537 | 10.58 | 7.988 | 1.00 | 45.43 |
| 49 | CD | LYS | A | 217 | 10.703 | 9.314 | 7.994 | 1.00 | 46.92 |
| 50 | CE | LYS | A | 217 | 9.639 | 9.354 | 9.08 | 1.00 | 48.09 |
| 51 | NZ | LYS | A | 217 | 8.771 | 8.147 | 9.043 | 1.00 | 49.24 |
| 52 | C | LYS | A | 217 | 12.325 | 13.09 | 9.866 | 1.00 | 41.19 |
| 53 | O | LYS | A | 217 | 11.653 | 13.83 | 9.145 | 1.00 | 40.18 |
| 54 | N | ARG | A | 218 | 12.325 | 13.18 | 11.193 | 1.00 | 38.94 |
| 55 | CA | ARG | A | 218 | 11.534 | 14.18 | 11.901 | 1.00 | 37.26 |
| 56 | CB | ARG | A | 218 | 11.748 | 14.03 | 13.411 | 1.00 | 39.88 |
| 57 | CG | ARG | A | 218 | 11.396 | 15.26 | 14.24 | 1.00 | 43.03 |
| 58 | CD | ARG | A | 218 | 9.903 | 15.54 | 14.26 | 1.00 | 45.71 |
| 59 | NE | ARG | A | 218 | 9.619 | 16.9 | 14.734 | 1.00 | 46.98 |
| 60 | CZ | ARG | A | 218 | 8.399 | 17.41 | 14.851 | 1.00 | 47.56 |
| 61 | NH1 | ARG | A | 218 | 7.335 | 16.68 | 14.531 | 1.00 | 48.63 |
| 62 | NH2 | ARG | A | 218 | 8.242 | 18.65 | 15.28 | 1.00 | 47.03 |
| 63 | C | ARG | A | 218 | 11.948 | 15.58 | 11.444 | 1.00 | 34.87 |
| 64 | O | ARG | A | 218 | 11.109 | 16.39 | 11.052 | 1.00 | 33.63 |
| 65 | N | ILE | A | 219 | 13.247 | 15.86 | 11.5 | 1.00 | 31.60 |
| 66 | CA | RE | A | 219 | 13.762 | 17.16 | 11.084 | 1.00 | 28.45 |
| 67 | CB | ILE | A | 219 | 15.285 | 17.27 | 11.356 | 1.00 | 27.35 |
| 68 | CG2 | RE | A | 219 | 15.874 | 18.46 | 10.625 | 1.00 | 26.86 |
| 69 | CG1 | RE | A | 219 | 15.53 | 17.39 | 12.863 | 1.00 | 27.32 |
| 70 | CD1 | RE | A | 219 | 16.997 | 17.3 | 13.258 | 1.00 | 26.22 |
| 71 | C | RE | A | 219 | 13.486 | 17.35 | 9.597 | 1.00 | 27.44 |
| 72 | O | RE | A | 219 | 13.153 | 18.45 | 9.153 | 1.00 | 26.71 |
| 73 | N | TYR | A | 220 | 13.619 | 16.28 | 8.834 | 1.00 | 25.68 |
| 74 | CA | TYR | A | 220 | 13.374 | 16.33 | 7.402 | 1.00 | 26.73 |
| 75 | CB | TYR | A | 220 | 13.786 | 15.01 | 6.748 | 1.00 | 25.17 |
| 76 | CG | TYR | A | 220 | 13.663 | 14.97 | 5.236 | 1.00 | 27.92 |
| 77 | CD1 | TYR | A | 220 | 13.856 | 16.12 | 4.465 | 1.00 | 27.10 |
| 78 | CE1 | TYR | A | 220 | 13.779 | 16.07 | 3.074 | 1.00 | 27.40 |
| 79 | CD2 | TYR | A | 220 | 13.391 | 13.77 | 4.572 | 1.00 | 28.20 |
| 80 | CE2 | TYR | A | 220 | 13.316 | 13.72 | 3.18 | 1.00 | 29.60 |
| 81 | CZ | TYR | A | 220 | 13.512 | 14.88 | 2.438 | 1.00 | 28.67 |
| 82 | OH | TYR | A | 220 | 13.453 | 14.83 | 1.062 | 1.00 | 30.22 |
| 83 | C | TYR | A | 220 | 11.895 | 16.64 | 7.155 | 1.00 | 26.35 |
| 84 | O | TYR | A | 220 | 11.554 | 17.41 | 6.263 | 1.00 | 22.94 |
| 85 | N | GLU | A | 221 | 11.013 | 16.04 | 7.948 | 1.00 | 27.41 |
| 86 | CA | GLU | A | 221 | 9.589 | 16.29 | 7.775 | 1.00 | 27.15 |
| 87 | CB | GLU | A | 221 | 8.76 | 15.39 | 8.694 | 1.00 | 29.37 |
| 88 | CG | GLU | A | 221 | 8.871 | 13.92 | 8.317 | 1.00 | 32.72 |
| 89 | CD | GLU | A | 221 | 7.949 | 13.01 | 9.119 | 1.00 | 34.56 |
| 90 | OE1 | GLU | A | 221 | 8.047 | 13 | 10.363 | 1.00 | 36.22 |
| 91 | OE2 | GLU | A | 221 | 7.127 | 12.31 | 8.498 | 1.00 | 36.44 |
| 92 | C | GLU | A | 221 | 9.301 | 17.77 | 8.056 | 1.00 | 25.89 |
| 93 | O | GLU | A | 221 | 8.54 | 18.4 | 7.329 | 1.00 | 25.26 |
| 94 | N | ALA | A | 222 | 9.929 | 18.3 | 9.099 | 1.00 | 23.75 |
| 95 | CA | ALA | A | 222 | 9.749 | 19.7 | 9.463 | 1.00 | 22.66 |
| 96 | CB | ALA | A | 222 | 10.526 | 20.02 | 10.733 | 1.00 | 24.94 |
| 97 | C | ALA | A | 222 | 10.225 | 20.6 | 8.325 | 1.00 | 21.68 |
| 98 | O | ALA | A | 222 | 9.625 | 21.64 | 8.041 | 1.00 | 20.08 |
| 99 | N | TYR | A | 223 | 11.311 | 20.2 | 7.678 | 1.00 | 20.59 |
| 100 | CA | TYR | A | 223 | 11.875 | 20.95 | 6.567 | 1.00 | 20.21 |
| 101 | CB | TYR | A | 223 | 13.226 | 20.34 | 6.205 | 1.00 | 21.59 |
| 102 | CG | TYR | A | 223 | 13.893 | 20.85 | 4.95 | 1.00 | 20.23 |
| 103 | CD1 | TYR | A | 223 | 13.563 | 20.31 | 3.702 | 1.00 | 21.49 |
| 104 | CE1 | TYR | A | 223 | 14.255 | 20.69 | 2.557 | 1.00 | 20.65 |
| 105 | CD2 | TYR | A | 223 | 14.93 | 21.77 | 5.017 | 1.00 | 19.83 |
| 106 | CE2 | TYR | A | 223 | 15.631 | 22.15 | 3.874 | 1.00 | 20.40 |
| 107 | CZ | TYR | A | 223 | 15.289 | 21.6 | 2.65 | 1.00 | 20.09 |
| 108 | OH | TYR | A | 223 | 16.005 | 21.93 | 1.52 | 1.00 | 22.28 |
| 109 | C | TYR | A | 223 | 10.919 | 20.96 | 5.371 | 1.00 | 20.36 |
| 110 | O | TYR | A | 223 | 10.657 | 22.01 | 4.78 | 1.00 | 18.08 |
| 111 | N | LEU | A | 224 | 10.374 | 19.8 | 5.03 | 1.00 | 19.65 |
| 112 | CA | LEU | A | 224 | 9.455 | 19.72 | 3.901 | 1.00 | 21.64 |
| 113 | CB | LEU | A | 224 | 9.163 | 18.25 | 3.565 | 1.00 | 22.15 |
| 114 | CG | LEU | A | 224 | 10.345 | 17.45 | 3.016 | 1.00 | 23.81 |
| 115 | CD1 | LEU | A | 224 | 9.94 | 15.99 | 2.85 | 1.00 | 24.83 |
| 116 | CD2 | LEU | A | 224 | 10.791 | 18.05 | 1.68 | 1.00 | 24.52 |
| 117 | C | LEU | A | 224 | 8.144 | 20.46 | 4.167 | 1.00 | 21.59 |
| 118 | O | LEU | A | 224 | 7.521 | 20.99 | 3.25 | 1.00 | 22.21 |
| 119 | N | LYS | A | 225 | 7.729 | 20.51 | 5.424 | 1.00 | 21.23 |
| 120 | CA | LYS | A | 225 | 6.482 | 21.17 | 5.776 | 1.00 | 24.07 |
| 121 | CB | LYS | A | 225 | 5.972 | 20.61 | 7.11 | 1.00 | 27.04 |
| 122 | CG | LYS | A | 225 | 4.766 | 21.32 | 7.725 | 1.00 | 32.48 |
| 123 | CD | LYS | A | 225 | 5.179 | 22.54 | 8.533 | 1.00 | 34.35 |
| 124 | CE | LYS | A | 225 | 3.985 | 23.17 | 9.244 | 1.00 | 36.46 |
| 125 | NZ | LYS | A | 225 | 4.381 | 24.35 | 10.071 | 1.00 | 35.63 |
| 126 | C | LYS | A | 225 | 6.571 | 22.69 | 5.862 | 1.00 | 23.63 |
| 127 | O | LYS | A | 225 | 5.582 | 23.39 | 5.616 | 1.00 | 22.64 |
| 128 | N | ASN | A | 226 | 7.757 | 23.21 | 6.177 | 1.00 | 21.43 |
| 129 | CA | ASN | A | 226 | 7.923 | 24.65 | 6.369 | 1.00 | 21.60 |
| 130 | CB | ASN | A | 226 | 8.631 | 24.88 | 7.704 | 1.00 | 21.30 |
| 131 | CG | ASN | A | 226 | 7.739 | 24.56 | 8.883 | 1.00 | 21.85 |
| 132 | OD1 | ASN | A | 226 | 6.769 | 25.28 | 9.142 | 1.00 | 21.90 |
| 133 | ND2 | ASN | A | 226 | 8.046 | 23.48 | 9.592 | 1.00 | 21.00 |
| 134 | C | ASN | A | 226 | 8.576 | 25.51 | 5.305 | 1.00 | 20.24 |
| 135 | O | ASN | A | 226 | 8.481 | 26.74 | 5.373 | 1.00 | 21.01 |
| 136 | N | PHE | A | 227 | 9.229 | 24.9 | 4.326 | 1.00 | 19.71 |
| 137 | CA | PHE | A | 227 | 9.883 | 25.69 | 3.279 | 1.00 | 20.43 |
| 138 | CD | PHE | A | 227 | 11.354 | 25.29 | 3.161 | 1.00 | 19.13 |
| 139 | CG | PHE | A | 227 | 12.162 | 25.6 | 4.392 | 1.00 | 18.17 |
| 140 | CD1 | PHE | A | 227 | 12.348 | 26.92 | 4.801 | 1.00 | 16.25 |
| 141 | CD2 | PHE | A | 227 | 12.736 | 24.58 | 5.136 | 1.00 | 16.60 |
| 142 | CE1 | PHE | A | 227 | 13.094 | 27.21 | 5.935 | 1.00 | 16.71 |
| 143 | CE2 | PHE | A | 227 | 13.488 | 24.86 | 6.275 | 1.00 | 17.69 |
| 144 | CZ | PHE | A | 227 | 13.668 | 26.18 | 6.678 | 1.00 | 18.05 |
| 145 | C | PHE | A | 227 | 9.185 | 25.5 | 1.941 | 1.00 | 21.87 |
| 146 | O | PHE | A | 227 | 9.147 | 24.41 | 1.401 | 1.00 | 23.21 |
| 147 | N | ASN | A | 228 | 8.644 | 26.59 | 1.407 | 1.00 | 22.66 |
| 148 | CA | ASN | A | 228 | 7.937 | 26.53 | 0.136 | 1.00 | 23.56 |
| 149 | CB | ASN | A | 228 | 7.251 | 27.87 | −0.135 | 1.00 | 26.95 |
| 150 | CG | ASN | A | 228 | 6.072 | 28.12 | 0.801 | 1.00 | 29.92 |
| 151 | OD1 | ASN | A | 228 | 5.14 | 27.31 | 0.867 | 1.00 | 33.43 |
| 152 | ND2 | ASN | A | 228 | 6.108 | 29.23 | 1.528 | 1.00 | 32.80 |
| 153 | C | ASN | A | 228 | 8.842 | 26.14 | −1.028 | 1.00 | 23.59 |
| 154 | O | ASN | A | 228 | 8.375 | 25.6 | −2.029 | 1.00 | 22.24 |
| 155 | N | MET | A | 229 | 10.136 | 26.41 | −0.891 | 1.00 | 22.41 |
| 156 | CA | MET | A | 229 | 11.09 | 26.05 | −1.931 | 1.00 | 23.18 |
| 157 | CB | MET | A | 229 | 11.724 | 27.31 | −2.537 | 1.00 | 23.66 |
| 158 | CG | MET | A | 229 | 12.717 | 27.05 | −3.673 | 1.00 | 25.31 |
| 159 | SD | MET | A | 229 | 11.966 | 26.31 | −5.134 | 1.00 | 26.48 |
| 160 | CE | MET | A | 229 | 10.916 | 27.66 | −5.681 | 1.00 | 24.83 |
| 161 | C | MET | A | 229 | 12.175 | 25.18 | −1.328 | 1.00 | 23.23 |
| 162 | O | MET | A | 229 | 12.629 | 25.42 | −0.206 | 1.00 | 23.23 |
| 163 | N | ASN | A | 230 | 12.56 | 24.14 | −2.067 | 1.00 | 21.71 |
| 164 | CA | ASN | A | 230 | 13.62 | 23.24 | −1.638 | 1.00 | 22.22 |
| 165 | CB | ASN | A | 230 | 13.057 | 22.03 | −0.889 | 1.00 | 23.15 |
| 166 | CG | ASN | A | 230 | 12.073 | 21.23 | −1.712 | 1.00 | 24.30 |
| 167 | OD1 | ASN | A | 230 | 12.321 | 20.93 | −2.874 | 1.00 | 25.96 |
| 168 | ND2 | ASN | A | 230 | 10.952 | 20.87 | −1.1 | 1.00 | 26.83 |
| 169 | C | ASN | A | 230 | 14.37 | 22.82 | −2.891 | 1.00 | 22.09 |
| 170 | O | ASN | A | 230 | 13.995 | 23.21 | −3.997 | 1.00 | 20.57 |
| 171 | N | LYS | A | 231 | 15.42 | 22.03 | −2.728 | 1.00 | 20.59 |
| 172 | CA | LYS | A | 231 | 16.235 | 21.61 | −3.861 | 1.00 | 20.89 |
| 173 | CB | LYS | A | 231 | 17.466 | 20.85 | −3.364 | 1.00 | 21.48 |
| 174 | CG | LYS | A | 231 | 18.656 | 20.95 | −4.295 | 1.00 | 20.77 |
| 175 | CD | LYS | A | 231 | 19.92 | 20.5 | −3.597 | 1.00 | 21.19 |
| 176 | CE | LYS | A | 231 | 21.125 | 20.63 | −4.496 | 1.00 | 21.09 |
| 177 | NZ | LYS | A | 231 | 22.355 | 20.17 | −3.814 | 1.00 | 19.55 |
| 178 | C | LYS | A | 231 | 15.503 | 20.79 | −4.916 | 1.00 | 20.69 |
| 179 | O | LYS | A | 231 | 15.676 | 21.02 | −6.11 | 1.00 | 19.10 |
| 180 | N | VAL | A | 232 | 14.694 | 19.82 | −4.494 | 1.00 | 20.29 |
| 181 | CA | VAL | A | 232 | 13.956 | 19.02 | −5.462 | 1.00 | 23.64 |
| 182 | CB | VAL | A | 232 | 13.006 | 18.01 | −4.772 | 1.00 | 25.30 |
| 183 | CG1 | VAL | A | 232 | 12.237 | 17.23 | −5.824 | 1.00 | 28.94 |
| 184 | CG2 | VAL | A | 232 | 13.796 | 17.07 | −3.894 | 1.00 | 28.35 |
| 185 | C | VAL | A | 232 | 13.125 | 19.93 | −6.371 | 1.00 | 22.27 |
| 186 | O | VAL | A | 232 | 13.222 | 19.84 | −7.596 | 1.00 | 22.87 |
| 187 | N | LYS | A | 233 | 12.32 | 20.8 | −5.762 | 1.00 | 22.90 |
| 188 | CA | LYS | A | 233 | 11.463 | 21.73 | −6.505 | 1.00 | 22.56 |
| 189 | CB | LYS | A | 233 | 10.644 | 22.6 | −5.544 | 1.00 | 25.17 |
| 190 | CG | LYS | A | 233 | 9.504 | 21.91 | −4.817 | 1.00 | 25.33 |
| 191 | CD | LYS | A | 233 | 8.687 | 22.94 | −4.048 | 1.00 | 27.14 |
| 192 | CE | LYS | A | 233 | 7.448 | 22.35 | −3.407 | 1.00 | 27.11 |
| 193 | NZ | LYS | A | 233 | 6.64 | 23.42 | −2.749 | 1.00 | 25.12 |
| 194 | C | LYS | A | 233 | 12.253 | 22.64 | −7.438 | 1.00 | 23.61 |
| 195 | O | LYS | A | 233 | 11.887 | 22.83 | −8.603 | 1.00 | 22.28 |
| 196 | N | ALA | A | 234 | 13.331 | 23.22 | −6.917 | 1.00 | 22.21 |
| 197 | CA | ALA | A | 234 | 14.173 | 24.12 | −7.696 | 1.00 | 21.88 |
| 198 | CB | ALA | A | 234 | 15.239 | 24.74 | −6.796 | 1.00 | 19.12 |
| 199 | C | ALA | A | 234 | 14.839 | 23.43 | −8.884 | 1.00 | 22.78 |
| 200 | O | ALA | A | 234 | 14.852 | 23.96 | −9.989 | 1.00 | 23.11 |
| 201 | N | ARG | A | 235 | 15.395 | 22.24 | −8.665 | 1.00 | 23.76 |
| 202 | CA | ARG | A | 235 | 16.064 | 21.52 | −9.745 | 1.00 | 26.20 |
| 203 | CB | ARG | A | 235 | 16.735 | 20.25 | −9.208 | 1.00 | 27.83 |
| 204 | CG | ARG | A | 235 | 18.065 | 20.54 | −8.523 | 1.00 | 28.58 |
| 205 | CD | ARG | A | 235 | 19.004 | 21.27 | −9.478 | 1.00 | 32.23 |
| 206 | NE | ARG | A | 235 | 20.002 | 22.08 | −8.784 | 1.00 | 34.36 |
| 207 | CZ | ARG | A | 235 | 19.701 | 23.04 | −7.919 | 1.00 | 37.04 |
| 208 | NH1 | ARG | A | 235 | 18.431 | 23.31 | −7.639 | 1.00 | 39.09 |
| 209 | NH2 | ARG | A | 235 | 20.662 | 23.76 | −7.352 | 1.00 | 35.24 |
| 210 | C | ARG | A | 235 | 15.153 | 21.19 | −10.92 | 1.00 | 27.67 |
| 211 | O | ARG | A | 235 | 15.6 | 21.18 | −12.07 | 1.00 | 29.64 |
| 212 | N | VAL | A | 236 | 13.881 | 20.92 | −10.64 | 1.00 | 27.53 |
| 213 | CA | VAL | A | 236 | 12.921 | 20.63 | −11.7 | 1.00 | 28.83 |
| 214 | CB | VAL | A | 236 | 11.55 | 20.23 | −11.12 | 1.00 | 30.09 |
| 215 | CG1 | VAL | A | 236 | 10.493 | 20.23 | −12.22 | 1.00 | 31.75 |
| 216 | CG2 | VAL | A | 236 | 11.641 | 18.85 | −10.49 | 1.00 | 31.01 |
| 217 | C | VAL | A | 236 | 12.734 | 21.88 | −12.55 | 1.00 | 28.74 |
| 218 | O | VAL | A | 236 | 12.763 | 21.83 | −13.78 | 1.00 | 27.81 |
| 219 | N | ILE | A | 237 | 12.543 | 23.01 | −11.87 | 1.00 | 26.31 |
| 220 | CA | ILE | A | 237 | 12.346 | 24.28 | −12.54 | 1.00 | 26.14 |
| 221 | CB | ILE | A | 237 | 12.041 | 25.39 | −11.51 | 1.00 | 24.59 |
| 222 | CG2 | ILE | A | 237 | 12.015 | 26.76 | −12.18 | 1.00 | 23.06 |
| 223 | CG1 | ILE | A | 237 | 10.7 | 25.1 | −10.83 | 1.00 | 23.57 |
| 224 | CD1 | ILE | A | 237 | 10.402 | 26 | −9.639 | 1.00 | 25.78 |
| 225 | C | ILE | A | 237 | 13.571 | 24.67 | −13.37 | 1.00 | 28.37 |
| 226 | O | ILE | A | 237 | 13.441 | 25.23 | −14.46 | 1.00 | 27.38 |
| 227 | N | LEU | A | 238 | 14.756 | 24.34 | −12.86 | 1.00 | 28.44 |
| 228 | CA | LEU | A | 238 | 16.006 | 24.67 | −13.54 | 1.00 | 31.74 |
| 229 | CB | LEU | A | 238 | 17.133 | 24.78 | −12.52 | 1.00 | 30.50 |
| 230 | CG | LEU | A | 238 | 17.026 | 25.98 | −11.57 | 1.00 | 30.22 |
| 231 | CD1 | LEU | A | 238 | 18.016 | 25.83 | −10.42 | 1.00 | 29.46 |
| 232 | CD2 | LEU | A | 238 | 17.283 | 27.26 | −12.35 | 1.00 | 29.52 |
| 233 | C | LEU | A | 238 | 16.415 | 23.68 | −14.62 | 1.00 | 34.68 |
| 234 | O | LEU | A | 238 | 17.29 | 23.96 | −15.44 | 1.00 | 35.11 |
| 235 | N | SER | A | 239 | 15.793 | 22.51 | −14.62 | 1.00 | 37.91 |
| 236 | CA | SER | A | 239 | 16.1 | 21.48 | −15.6 | 1.00 | 42.67 |
| 237 | CB | SER | A | 239 | 17.408 | 20.77 | −15.26 | 1.00 | 42.74 |
| 238 | OG | SER | A | 239 | 18.474 | 21.25 | −16.05 | 1.00 | 44.54 |
| 239 | C | SER | A | 239 | 14.991 | 20.46 | −15.67 | 1.00 | 45.44 |
| 240 | O | SER | A | 239 | 14.579 | 19.89 | −14.66 | 1.00 | 46.52 |
| 241 | N | GLY | A | 240 | 14.513 | 20.21 | −16.88 | 1.00 | 48.97 |
| 242 | CA | GLY | A | 240 | 13.452 | 19.25 | −17.04 | 1.00 | 52.46 |
| 243 | C | GLY | A | 240 | 12.655 | 19.58 | −18.27 | 1.00 | 55.01 |
| 244 | O | GLY | A | 240 | 12.16 | 18.68 | −18.93 | 1.00 | 55.62 |
| 245 | N | LYS | A | 241 | 12.554 | 20.88 | −18.57 | 1.00 | 57.05 |
| 246 | CA | LYS | A | 241 | 11.817 | 21.41 | −19.72 | 1.00 | 58.42 |
| 247 | CB | LYS | A | 241 | 12.707 | 21.39 | −20.96 | 1.00 | 59.37 |
| 248 | CG | LYS | A | 241 | 13.824 | 22.43 | −20.95 | 1.00 | 58.74 |
| 249 | CD | LYS | A | 241 | 14.872 | 22.09 | −21.99 | 1.00 | 59.57 |
| 250 | CE | LYS | A | 241 | 15.865 | 23.22 | −22.2 | 1.00 | 59.20 |
| 251 | NZ | LYS | A | 241 | 15.485 | 24.06 | −23.36 | 1.00 | 60.11 |
| 252 | C | LYS | A | 241 | 10.514 | 20.68 | −20.01 | 1.00 | 59.28 |
| 253 | O | LYS | A | 241 | 9.52 | 21.3 | −20.37 | 1.00 | 59.75 |
| 254 | N | ALA | A | 242 | 10.544 | 19.36 | −19.85 | 1.00 | 60.20 |
| 255 | CA | ALA | A | 242 | 9.408 | 18.47 | −20.04 | 1.00 | 60.65 |
| 256 | CB | ALA | A | 242 | 9.225 | 17.59 | −18.81 | 1.00 | 60.60 |
| 257 | C | ALA | A | 242 | 8.152 | 19.27 | −20.3 | 1.00 | 61.11 |
| 258 | O | ALA | A | 242 | 7.718 | 19.43 | −21.44 | 1.00 | 61.12 |
| 259 | N | SER | A | 243 | 7.581 | 19.79 | −19.21 | 1.00 | 61.07 |
| 260 | CA | SER | A | 243 | 6.382 | 20.59 | −19.3 | 1.00 | 61.06 |
| 261 | CB | SER | A | 243 | 5.726 | 20.72 | −17.92 | 1.00 | 61.00 |
| 262 | OG | SER | A | 243 | 4.64 | 21.63 | −17.95 | 1.00 | 59.89 |
| 263 | G | SER | A | 243 | 6.742 | 21.98 | −19.84 | 1.00 | 61.10 |
| 264 | O | SER | A | 243 | 7.434 | 22.11 | −20.85 | 1.00 | 61.74 |
| 265 | N | ASN | A | 244 | 6.279 | 23.01 | −19.14 | 1.00 | 60.20 |
| 266 | CA | ASN | A | 244 | 6.53 | 24.38 | −19.55 | 1.00 | 59.10 |
| 267 | CB | ASN | A | 244 | 5.91 | 24.6 | −20.95 | 1.00 | 58.65 |
| 268 | CG | ASN | A | 244 | 5.63 | 26.05 | −21.26 | 1.00 | 58.90 |
| 269 | OD1 | ASN | A | 244 | 4.647 | 26.62 | −20.78 | 1.00 | 59.73 |
| 270 | ND2 | ASN | A | 244 | 6.488 | 26.66 | −22.07 | 1.00 | 58.39 |
| 271 | C | ASN | A | 244 | 5.964 | 25.34 | −18.5 | 1.00 | 58.52 |
| 272 | O | ASN | A | 244 | 5.616 | 24.91 | −17.4 | 1.00 | 59.67 |
| 273 | N | ASN | A | 245 | 5.873 | 26.62 | −18.85 | 1.00 | 56.14 |
| 274 | CA | ASN | A | 245 | 5.406 | 27.68 | −17.94 | 1.00 | 51.26 |
| 275 | CB | ASN | A | 245 | 4.324 | 27.13 | −17.01 | 1.00 | 52.89 |
| 276 | CG | ASN | A | 245 | 4.047 | 28.05 | −15.84 | 1.00 | 52.48 |
| 277 | OD1 | ASN | A | 245 | 3.453 | 29.12 | −16 | 1.00 | 52.78 |
| 278 | ND2 | ASN | A | 245 | 4.489 | 27.64 | −14.66 | 1.00 | 52.90 |
| 279 | C | ASN | A | 245 | 6.685 | 28.03 | −17.18 | 1.00 | 47.02 |
| 280 | O | ASN | A | 245 | 6.689 | 28.2 | −15.96 | 1.00 | 47.14 |
| 281 | N | PRO | A | 246 | 7.789 | 28.19 | −17.93 | 1.00 | 42.04 |
| 282 | CD | PRO | A | 246 | 7.619 | 28.47 | −19.37 | 1.00 | 41.47 |
| 283 | CA | PRO | A | 246 | 9.161 | 28.51 | −17.54 | 1.00 | 38.16 |
| 284 | CB | PRO | A | 246 | 9.88 | 28.58 | −18.87 | 1.00 | 38.68 |
| 285 | CG | PRO | A | 246 | 8.86 | 29.26 | −19.71 | 1.00 | 39.97 |
| 286 | C | PRO | A | 246 | 9.379 | 29.77 | −16.74 | 1.00 | 34.87 |
| 287 | O | PRO | A | 246 | 8.668 | 30.76 | −16.9 | 1.00 | 34.21 |
| 288 | N | PRO | A | 247 | 10.379 | 29.75 | −15.85 | 1.00 | 32.12 |
| 289 | CD | PRO | A | 247 | 11.207 | 28.6 | −15.43 | 1.00 | 32.31 |
| 290 | CA | PRO | A | 247 | 10.669 | 30.93 | −15.04 | 1.00 | 29.30 |
| 291 | CB | PRO | A | 247 | 11.823 | 30.47 | −14.14 | 1.00 | 29.79 |
| 292 | CG | PRO | A | 247 | 12.418 | 29.28 | −14.88 | 1.00 | 32.45 |
| 293 | C | PRO | A | 247 | 11.053 | 32.07 | −15.97 | 1.00 | 27.36 |
| 294 | O | PRO | A | 247 | 11.692 | 31.86 | −17.01 | 1.00 | 25.06 |
| 295 | N | PHE | A | 248 | 10.639 | 33.28 | −15.62 | 1.00 | 24.30 |
| 296 | CA | PHE | A | 248 | 10.943 | 34.45 | −16.43 | 1.00 | 23.14 |
| 297 | CB | PHE | A | 248 | 10.004 | 35.6 | −16.07 | 1.00 | 25.05 |
| 298 | CG | PHE | A | 248 | 10.098 | 36.76 | −17.01 | 1.00 | 26.31 |
| 299 | CD1 | PHE | A | 248 | 9.46 | 36.72 | −18.25 | 1.00 | 27.99 |
| 300 | CD2 | PHE | A | 248 | 10.865 | 37.87 | −16.69 | 1.00 | 27.41 |
| 301 | CE1 | PHE | A | 248 | 9.585 | 37.77 | −19.15 | 1.00 | 28.47 |
| 302 | CE2 | PHE | A | 248 | 10.999 | 38.93 | −17.59 | 1.00 | 28.03 |
| 303 | CZ | PHE | A | 248 | 10.357 | 38.88 | −18.82 | 1.00 | 29.16 |
| 304 | C | PHE | A | 248 | 12.371 | 34.87 | −16.13 | 1.00 | 22.55 |
| 305 | O | PHE | A | 248 | 12.726 | 35.08 | −14.98 | 1.00 | 19.83 |
| 306 | N | VAL | A | 249 | 13.188 | 35 | −17.17 | 1.00 | 22.23 |
| 307 | CA | VAL | A | 249 | 14.578 | 35.38 | −16.97 | 1.00 | 21.89 |
| 308 | CB | VAL | A | 249 | 15.49 | 34.73 | −18.04 | 1.00 | 23.08 |
| 309 | CG1 | VAL | A | 249 | 16.917 | 35.26 | −17.91 | 1.00 | 22.71 |
| 310 | CG2 | VAL | A | 249 | 15.484 | 33.21 | −17.87 | 1.00 | 22.78 |
| 311 | C | VAL | A | 249 | 14.817 | 36.88 | −16.96 | 1.00 | 22.84 |
| 312 | O | VAL | A | 249 | 14.41 | 37.61 | −17.87 | 1.00 | 22.43 |
| 313 | N | ILE | A | 250 | 15.472 | 37.34 | −15.89 | 1.00 | 20.77 |
| 314 | CA | ILE | A | 250 | 15.814 | 38.74 | −15.73 | 1.00 | 20.91 |
| 315 | CB | ILE | A | 250 | 15.494 | 39.22 | −14.3 | 1.00 | 20.98 |
| 316 | CG2 | ILE | A | 250 | 15.899 | 40.69 | −14.14 | 1.00 | 19.06 |
| 317 | CG1 | ILE | A | 250 | 13.996 | 39.04 | −14.02 | 1.00 | 19.24 |
| 318 | CD1 | ILE | A | 250 | 13.602 | 39.29 | −12.57 | 1.00 | 21.68 |
| 319 | C | ILE | A | 250 | 17.314 | 38.83 | −16 | 1.00 | 21.63 |
| 320 | O | ILE | A | 250 | 18.134 | 38.4 | −15.18 | 1.00 | 19.53 |
| 321 | N | HIS | A | 251 | 17.668 | 39.37 | −17.17 | 1.00 | 20.99 |
| 322 | CA | HIS | A | 251 | 19.07 | 39.48 | −17.56 | 1.00 | 22.26 |
| 323 | CB | HIS | A | 251 | 19.343 | 38.57 | −18.76 | 1.00 | 24.28 |
| 324 | CG | HIS | A | 251 | 18.57 | 38.93 | −19.99 | 1.00 | 24.97 |
| 325 | CD2 | HIS | A | 251 | 17.449 | 38.4 | −20.53 | 1.00 | 25.77 |
| 326 | ND1 | HIS | A | 251 | 18.922 | 39.99 | −20.8 | 1.00 | 27.65 |
| 327 | CE1 | HIS | A | 251 | 18.049 | 40.09 | −21.79 | 1.00 | 25.99 |
| 328 | NE2 | HIS | A | 251 | 17.145 | 39.14 | −21.65 | 1.00 | 26.18 |
| 329 | C | HIS | A | 251 | 19.535 | 40.9 | −17.86 | 1.00 | 23.10 |
| 330 | O | HIS | A | 251 | 20.719 | 41.13 | −18.11 | 1.00 | 21.51 |
| 331 | N | ASP | A | 252 | 18.601 | 41.85 | −17.84 | 1.00 | 23.64 |
| 332 | CA | ASP | A | 252 | 18.912 | 43.26 | −18.07 | 1.00 | 26.61 |
| 333 | CB | ASP | A | 252 | 19.179 | 43.55 | −19.55 | 1.00 | 27.87 |
| 334 | CG | ASP | A | 252 | 17.985 | 43.26 | −20.44 | 1.00 | 29.61 |
| 335 | OD1 | ASP | A | 252 | 16.857 | 43.13 | −19.92 | 1.00 | 28.47 |
| 336 | OD2 | ASP | A | 252 | 18.181 | 43.18 | −21.68 | 1.00 | 31.76 |
| 337 | C | ASP | A | 252 | 17.791 | 44.15 | −17.55 | 1.00 | 27.23 |
| 338 | O | ASP | A | 252 | 16.797 | 43.67 | −17.01 | 1.00 | 25.64 |
| 339 | N | MET | A | 253 | 17.951 | 45.46 | −17.73 | 1.00 | 28.20 |
| 340 | CA | MET | A | 253 | 16.958 | 46.41 | −17.25 | 1.00 | 28.88 |
| 341 | CB | MET | A | 253 | 17.459 | 47.83 | −17.49 | 1.00 | 31.16 |
| 342 | CC | MET | A | 253 | 18.755 | 48.11 | −16.76 | 1.00 | 33.46 |
| 343 | SD | MET | A | 253 | 18.548 | 48.1 | −14.96 | 1.00 | 36.77 |
| 344 | CE | MET | A | 253 | 19.322 | 49.69 | −14.59 | 1.00 | 37.18 |
| 345 | C | MET | A | 253 | 15.566 | 46.24 | −17.84 | 1.00 | 28.34 |
| 346 | O | MET | A | 253 | 14.565 | 46.4 | −17.14 | 1.00 | 27.10 |
| 347 | N | GLU | A | 254 | 15.493 | 45.89 | −19.12 | 1.00 | 28.20 |
| 348 | CA | GLU | A | 254 | 14.199 | 45.71 | −19.76 | 1.00 | 27.38 |
| 349 | CB | GLU | A | 254 | 14.368 | 45.54 | −21.28 | 1.00 | 30.74 |
| 350 | CC | GLU | A | 254 | 13.066 | 45.25 | −22 | 1.00 | 34.46 |
| 351 | CD | GLU | A | 254 | 13.222 | 45.23 | −23.51 | 1.00 | 37.18 |
| 352 | OE1 | GLU | A | 254 | 14.097 | 44.5 | −24.02 | 1.00 | 38.99 |
| 353 | OE2 | GLU | A | 254 | 12.46 | 45.95 | −24.19 | 1.00 | 39.82 |
| 354 | C | GLU | A | 254 | 13.443 | 44.51 | −19.19 | 1.00 | 26.77 |
| 355 | O | GLU | A | 254 | 12.267 | 44.62 | −18.84 | 1.00 | 24.55 |
| 356 | N | THR | A | 255 | 14.116 | 43.37 | −19.09 | 1.00 | 25.14 |
| 357 | CA | THR | A | 255 | 13.476 | 42.17 | −18.55 | 1.00 | 22.92 |
| 358 | CB | THR | A | 255 | 14.335 | 40.91 | −18.81 | 1.00 | 22.22 |
| 359 | OG1 | THR | A | 255 | 15.677 | 41.13 | −18.36 | 1.00 | 20.92 |
| 360 | CG2 | THR | A | 255 | 14.349 | 40.58 | −20.3 | 1.00 | 22.08 |
| 361 | C | THR | A | 255 | 13.167 | 42.3 | −17.06 | 1.00 | 22.64 |
| 362 | O | THR | A | 255 | 12.277 | 41.62 | −16.54 | 1.00 | 21.75 |
| 363 | N | LEU | A | 256 | 13.895 | 43.17 | −16.36 | 1.00 | 21.77 |
| 364 | CA | LEU | A | 256 | 13.629 | 43.39 | −14.94 | 1.00 | 20.25 |
| 365 | CB | LEU | A | 256 | 14.7 | 44.28 | −14.29 | 1.00 | 20.20 |
| 366 | CG | LEU | A | 256 | 14.347 | 44.72 | −12.86 | 1.00 | 18.26 |
| 367 | CD1 | LEU | A | 256 | 14.292 | 43.5 | −11.94 | 1.00 | 19.55 |
| 368 | CD2 | LEU | A | 256 | 15.378 | 45.72 | −12.34 | 1.00 | 17.68 |
| 369 | C | LEU | A | 256 | 12.282 | 44.09 | −14.84 | 1.00 | 20.44 |
| 370 | O | LEU | A | 256 | 11.424 | 43.7 | −14.06 | 1.00 | 19.24 |
| 371 | N | GYS | A | 257 | 12.092 | 45.13 | −15.64 | 1.00 | 20.05 |
| 372 | CA | CYS | A | 257 | 10.828 | 45.85 | −15.61 | 1.00 | 22.13 |
| 373 | CB | GYS | A | 257 | 10.893 | 47.09 | −16.53 | 1.00 | 22.88 |
| 374 | SG | GYS | A | 257 | 12.084 | 48.36 | −16 | 1.00 | 28.89 |
| 375 | C | GYS | A | 257 | 9.675 | 44.94 | −16.02 | 1.00 | 22.46 |
| 376 | O | CYS | A | 257 | 8.588 | 45.02 | −15.46 | 1.00 | 23.21 |
| 377 | N | MET | A | 258 | 9.915 | 44.06 | −17 | 1.00 | 24.21 |
| 378 | CA | MET | A | 258 | 8.881 | 43.13 | −17.47 | 1.00 | 25.70 |
| 379 | CB | MET | A | 258 | 9.383 | 42.33 | −18.67 | 1.00 | 28.67 |
| 380 | CG | MET | A | 258 | 9.875 | 43.17 | −19.85 | 1.00 | 34.04 |
| 381 | SD | MET | A | 258 | 10.485 | 42.19 | −21.26 | 1.00 | 38.98 |
| 382 | CE | MET | A | 258 | 10.123 | 43.33 | −22.62 | 1.00 | 38.27 |
| 383 | C | MET | A | 258 | 8.496 | 42.18 | −16.34 | 1.00 | 25.87 |
| 384 | O | MET | A | 258 | 7.314 | 41.92 | −16.1 | 1.00 | 25.43 |
| 385 | N | ALA | A | 259 | 9.503 | 41.64 | −15.66 | 1.00 | 24.88 |
| 386 | CA | ALA | A | 259 | 9.262 | 40.72 | −14.56 | 1.00 | 23.76 |
| 387 | CB | ALA | A | 259 | 10.586 | 40.19 | −14.03 | 1.00 | 22.98 |
| 388 | C | ALA | A | 259 | 8.482 | 41.41 | −13.44 | 1.00 | 22.99 |
| 389 | O | ALA | A | 259 | 7.546 | 40.83 | −12.89 | 1.00 | 23.42 |
| 390 | N | GLU | A | 260 | 8.864 | 42.64 | −13.12 | 1.00 | 22.90 |
| 391 | CA | GLU | A | 260 | 8.178 | 43.38 | −12.07 | 1.00 | 23.30 |
| 392 | CB | GLU | A | 260 | 8.843 | 44.75 | −11.85 | 1.00 | 23.04 |
| 393 | CG | GLU | A | 260 | 10.269 | 44.67 | −11.31 | 1.00 | 23.77 |
| 394 | CD | GLU | A | 260 | 10.974 | 46.02 | −11.26 | 1.00 | 25.32 |
| 395 | OE1 | GLU | A | 260 | 10.929 | 46.75 | −12.27 | 1.00 | 26.09 |
| 396 | OE2 | GLU | A | 260 | 11.59 | 46.34 | −10.23 | 1.00 | 24.65 |
| 397 | C | GLU | A | 260 | 6.717 | 43.57 | −12.46 | 1.00 | 24.60 |
| 398 | O | GLU | A | 260 | 5.818 | 43.41 | −11.64 | 1.00 | 23.99 |
| 399 | N | LYS | A | 261 | 6.489 | 43.91 | −13.72 | 1.00 | 26.88 |
| 400 | CA | LYS | A | 261 | 5.138 | 44.13 | −14.22 | 1.00 | 29.38 |
| 401 | CB | LYS | A | 261 | 5.18 | 44.42 | −15.72 | 1.00 | 31.11 |
| 402 | CG | LYS | A | 261 | 3.818 | 44.72 | −16.31 | 1.00 | 34.55 |
| 403 | CD | LYS | A | 261 | 3.758 | 46.12 | −16.89 | 1.00 | 38.59 |
| 404 | CE | LYS | A | 261 | 4.149 | 47.18 | −15.87 | 1.00 | 40.59 |
| 405 | NZ | LYS | A | 261 | 5.627 | 47.33 | −15.75 | 1.00 | 42.89 |
| 406 | C | LYS | A | 261 | 4.233 | 42.92 | −13.96 | 1.00 | 30.43 |
| 407 | O | LYS | A | 261 | 3.05 | 43.07 | −13.65 | 1.00 | 30.61 |
| 408 | N | THR | A | 262 | 4.797 | 41.72 | −14.08 | 1.00 | 31.64 |
| 409 | CA | THR | A | 262 | 4.031 | 40.5 | −13.88 | 1.00 | 32.66 |
| 410 | CB | THR | A | 262 | 4.501 | 39.39 | −14.85 | 1.00 | 34.32 |
| 411 | OG1 | THR | A | 262 | 4.522 | 39.9 | −16.19 | 1.00 | 37.49 |
| 412 | CG2 | THR | A | 262 | 3.551 | 38.2 | −14.79 | 1.00 | 34.76 |
| 413 | C | THR | A | 262 | 4.078 | 39.92 | −12.47 | 1.00 | 32.57 |
| 414 | O | THR | A | 262 | 3.04 | 39.59 | −11.89 | 1.00 | 33.27 |
| 415 | N | LEU | A | 263 | 5.279 | 39.8 | −11.91 | 1.00 | 31.54 |
| 416 | CA | LEU | A | 263 | 5.453 | 39.21 | −10.58 | 1.00 | 31.84 |
| 417 | CB | LEU | A | 263 | 6.819 | 38.53 | −10.51 | 1.00 | 32.07 |
| 418 | CG | LEU | A | 263 | 6.945 | 37.14 | −11.14 | 1.00 | 33.07 |
| 419 | CD1 | LEU | A | 263 | 5.767 | 36.84 | −12.06 | 1.00 | 32.88 |
| 420 | CD2 | LEU | A | 263 | 8.261 | 37.06 | 41.88 | 1.00 | 32.53 |
| 421 | C | LEU | A | 263 | 5.275 | 40.13 | −9.371 | 1.00 | 32.52 |
| 422 | O | LEU | A | 263 | 4.808 | 39.68 | −8.319 | 1.00 | 31.50 |
| 423 | N | VAL | A | 264 | 5.662 | 41.39 | −9.504 | 1.00 | 32.08 |
| 424 | CA | VAL | A | 264 | 5.532 | 42.35 | −8.412 | 1.00 | 2.49 |
| 425 | CB | VAL | A | 264 | 6.897 | 42.61 | −7.727 | 1.00 | 32.30 |
| 426 | CG1 | VAL | A | 264 | 6.752 | 43.68 | −6.667 | 1.00 | 33.97 |
| 427 | CG2 | VAL | A | 264 | 7.403 | 41.32 | −7.079 | 1.00 | 33.63 |
| 428 | C | VAL | A | 264 | 4.992 | 43.64 | −9.009 | 1.00 | 33.56 |
| 429 | O | VAI | A | 264 | 5.659 | 44.68 | −9.012 | 1.00 | 32.17 |
| 430 | N | ALA | A | 265 | 3.768 | 43.55 | −9.518 | 1.00 | 34.97 |
| 431 | CA | ALA | A | 265 | 3.079 | 44.66 | −10.17 | 1.00 | 35.76 |
| 432 | CB | ALA | A | 265 | 1.623 | 44.28 | −10.4 | 1.00 | 37.47 |
| 433 | C | ALA | A | 265 | 3.148 | 46.04 | −9.512 | 1.00 | 37.14 |
| 434 | O | ALA | A | 265 | 3.094 | 47.06 | −10.21 | 1.00 | 35.98 |
| 435 | N | LYS | A | 266 | 3.271 | 46.1 | −8.189 | 1.00 | 37.87 |
| 436 | CA | LYS | A | 266 | 3.313 | 47.39 | −7.507 | 1.00 | 39.85 |
| 437 | CB | LYS | A | 266 | 3.159 | 47.21 | −5.989 | 1.00 | 39.30 |
| 438 | CG | LYS | A | 266 | 3.099 | 48.54 | −5.222 | 1.00 | 40.41 |
| 439 | CD | LYS | A | 266 | 3.065 | 48.35 | −3.709 | 1.00 | 41.21 |
| 440 | CE | LYS | A | 266 | 1.695 | 47.91 | −3.21 | 1.00 | 42.93 |
| 441 | NZ | LYS | A | 266 | 0.646 | 48.94 | −3.476 | 1.00 | 44.25 |
| 442 | C | LYS | A | 266 | 4.565 | 48.24 | −7.782 | 1.00 | 40.87 |
| 443 | O | LYS | A | 266 | 4.519 | 49.46 | −7.702 | 1.00 | 41.16 |
| 444 | N | LEU | A | 267 | 5.675 | 47.58 | −8.11 | 1.00 | 41.92 |
| 445 | CA | LEU | A | 267 | 6.922 | 48.3 | −8.358 | 1.00 | 43.90 |
| 446 | CB | LEU | A | 267 | 8.114 | 47.38 | −8.094 | 1.00 | 43.36 |
| 447 | CG | LEU | A | 267 | 8.078 | 46.63 | −6.758 | 1.00 | 42.94 |
| 448 | CD1 | LEU | A | 267 | 9.45 | 46.03 | −6.48 | 1.00 | 42.92 |
| 449 | CD2 | LEU | A | 267 | 7.673 | 47.57 | −5.63 | 1.00 | 43.54 |
| 450 | C | LEU | A | 267 | 7.054 | 48.92 | −9.748 | 1.00 | 45.44 |
| 451 | O | LEU | A | 267 | 8.128 | 49.41 | −10.12 | 1.00 | 45.30 |
| 452 | N | VAL | A | 268 | 5.967 | 48.92 | −10.51 | 1.00 | 47.08 |
| 453 | CA | VAL | A | 268 | 5.986 | 49.49 | −11.85 | 1.00 | 49.10 |
| 454 | CB | VAL | A | 268 | 5.831 | 48.4 | −12.93 | 1.00 | 49.12 |
| 455 | CG1 | VAL | A | 268 | 7.032 | 47.47 | −12.91 | 1.00 | 49.41 |
| 456 | CG2 | VAL | A | 268 | 4.547 | 47.62 | −12.69 | 1.00 | 49.20 |
| 457 | C | VAL | A | 268 | 4.876 | 50.52 | −12.07 | 1.00 | 50.30 |
| 458 | O | VAL | A | 268 | 4.885 | 51.25 | −13.06 | 1.00 | 50.13 |
| 459 | N | ALA | A | 269 | 3.929 | 50.57 | −11.13 | 1.00 | 51.85 |
| 460 | CA | ALA | A | 269 | 2.799 | 51.49 | −11.25 | 1.00 | 53.91 |
| 461 | CB | ALA | A | 269 | 1.491 | 50.71 | −11.16 | 1.00 | 53.84 |
| 462 | C | ALA | A | 269 | 2.779 | 52.65 | −10.26 | 1.00 | 55.04 |
| 463 | O | ALA | A | 269 | 1.788 | 53.37 | −10.16 | 1.00 | 55.68 |
| 464 | N | GLY | A | 270 | 3.863 | 52.82 | −9.508 | 1.00 | 55.70 |
| 465 | CA | GLY | A | 270 | 3.921 | 53.9 | −8.54 | 1.00 | 57.57 |
| 466 | C | GLY | A | 270 | 5.338 | 54.41 | −8.407 | 1.00 | 58.36 |
| 467 | O | GLY | A | 270 | 5.581 | 55.62 | −8.394 | 1.00 | 58.74 |
| 468 | N | ILE | A | 271 | 6.272 | 53.47 | −8.31 | 1.00 | 58.34 |
| 469 | CA | ILE | A | 271 | 7.695 | 53.76 | −8.188 | 1.00 | 57.76 |
| 470 | CB | ILE | A | 271 | 8.424 | 52.63 | −7.416 | 1.00 | 58.40 |
| 471 | CG2 | ILE | A | 271 | 9.919 | 52.65 | −7.72 | 1.00 | 58.51 |
| 472 | CG1 | ILE | A | 271 | 8.159 | 52.76 | −5.916 | 1.00 | 58.87 |
| 473 | CD1 | ILE | A | 271 | 8.831 | 51.68 | −5.081 | 1.00 | 59.43 |
| 474 | C | ILE | A | 271 | 8.307 | 53.86 | −9.58 | 1.00 | 57.27 |
| 475 | O | ILE | A | 271 | 8.638 | 54.95 | −10.06 | 1.00 | 57.93 |
| 476 | N | GLN | A | 272 | 8.435 | 52.7 | −10.21 | 1.00 | 55.75 |
| 477 | CA | GLN | A | 272 | 9.007 | 52.52 | −11.54 | 1.00 | 54.43 |
| 478 | CB | GLN | A | 272 | 7.969 | 52.82 | −12.64 | 1.00 | 54.62 |
| 479 | CG | GLN | A | 272 | 7.554 | 54.26 | −12.78 | 1.00 | 54.62 |
| 480 | CD | GLN | A | 272 | 6.052 | 54.43 | −12.71 | 1.00 | 54.37 |
| 481 | OE1 | GLN | A | 272 | 5.471 | 54.46 | −11.63 | 1.00 | 54.65 |
| 482 | NE2 | GLN | A | 272 | 5.411 | 54.52 | −13.87 | 1.00 | 53.99 |
| 483 | C | GLN | A | 272 | 10.318 | 53.24 | −11.84 | 1.00 | 53.23 |
| 484 | O | GLN | A | 272 | 11.049 | 52.83 | −12.74 | 1.00 | 53.33 |
| 485 | N | ASN | A | 274 | 10.634 | 54.31 | −11.11 | 1.00 | 51.79 |
| 486 | CA | ASN | A | 274 | 11.922 | 54.94 | −11.37 | 1.00 | 50.16 |
| 487 | CB | ASN | A | 274 | 11.843 | 56.1 | −12.34 | 1.00 | 52.29 |
| 488 | CG | ASN | A | 274 | 13.008 | 56.09 | −13.33 | 1.00 | 54.63 |
| 489 | OD1 | ASN | A | 274 | 13.481 | 57.13 | −13.78 | 1.00 | 56.51 |
| 490 | ND2 | ASN | A | 274 | 13.472 | 54.89 | −13.67 | 1.00 | 55.84 |
| 491 | C | ASN | A | 274 | 12.762 | 55.35 | −10.18 | 1.00 | 47.37 |
| 492 | O | ASN | A | 274 | 13.387 | 56.41 | −10.18 | 1.00 | 47.22 |
| 493 | N | LYS | A | 275 | 12.732 | 54.52 | −9.146 | 1.00 | 43.69 |
| 494 | CA | LYS | A | 275 | 13.627 | 54.71 | −8.026 | 1.00 | 38.70 |
| 495 | CB | LYS | A | 275 | 13.13 | 53.98 | −6.779 | 1.00 | 39.95 |
| 496 | CG | LYS | A | 275 | 11.997 | 54.69 | −6.051 | 1.00 | 41.04 |
| 497 | CD | LYS | A | 275 | 11.744 | 54.06 | −4.686 | 1.00 | 42.78 |
| 498 | CE | LYS | A | 275 | 10.677 | 54.82 | −3.905 | 1.00 | 43.67 |
| 499 | NZ | LYS | A | 275 | 10.448 | 54.23 | −2.551 | 1.00 | 44.37 |
| 500 | C | LYS | A | 275 | 14.632 | 53.86 | −8.794 | 1.00 | 35.85 |
| 501 | O | LYS | A | 275 | 14.203 | 53.01 | −9.578 | 1.00 | 32.72 |
| 502 | N | GLU | A | 276 | 15.934 | 54.07 | −8.645 | 1.00 | 33.11 |
| 503 | CA | GLU | A | 276 | 16.802 | 53.24 | −9.467 | 1.00 | 30.98 |
| 504 | CB | GLU | A | 276 | 18.279 | 53.64 | −9.331 | 1.00 | 34.18 |
| 505 | CG | GLU | A | 276 | 18.874 | 53.68 | −7.955 | 1.00 | 36.39 |
| 506 | CD | GLU | A | 276 | 20.194 | 54.43 | −7.958 | 1.00 | 36.02 |
| 507 | OE1 | GLU | A | 276 | 21.05 | 54.15 | −8.823 | 1.00 | 36.54 |
| 508 | OE2 | GLU | A | 276 | 20.376 | 55.31 | −7.097 | 1.00 | 38.61 |
| 509 | C | GLU | A | 276 | 16.587 | 51.76 | −9.234 | 1.00 | 29.49 |
| 510 | O | GLU | A | 276 | 16.175 | 51.32 | −8.157 | 1.00 | 26.80 |
| 511 | N | VAL | A | 277 | 16.834 | 50.98 | −10.28 | 1.00 | 26.80 |
| 512 | CA | VAL | A | 277 | 16.636 | 49.55 | −10.23 | 1.00 | 27.28 |
| 513 | CB | VAL | A | 277 | 17.141 | 48.89 | −11.52 | 1.00 | 26.70 |
| 514 | CG1 | VAL | A | 277 | 16.422 | 49.49 | −12.71 | 1.00 | 31.18 |
| 515 | CG2 | VAL | A | 277 | 18.627 | 49.07 | −11.64 | 1.00 | 31.22 |
| 516 | C | VAL | A | 277 | 17.268 | 48.84 | −9.038 | 1.00 | 24.93 |
| 517 | O | VAL | A | 277 | 16.65 | 47.95 | −8.457 | 1.00 | 23.64 |
| 518 | N | GLU | A | 278 | 18.487 | 49.23 | −8.662 | 1.00 | 22.59 |
| 519 | CA | GLU | A | 278 | 19.126 | 48.56 | −7.539 | 1.00 | 22.23 |
| 520 | CB | GLU | A | 278 | 20.549 | 49.1 | −7.281 | 1.00 | 23.67 |
| 521 | CG | GLU | A | 278 | 20.912 | 50.44 | −7.901 | 1.00 | 27.55 |
| 522 | CD | GLU | A | 278 | 21.043 | 50.38 | −9.41 | 1.00 | 24.59 |
| 523 | OE1 | GLU | A | 278 | 20.075 | 50.75 | −10.08 | 1.00 | 26.10 |
| 524 | OE2 | GLU | A | 278 | 22.104 | 49.96 | −9.927 | 1.00 | 26.97 |
| 525 | C | GLU | A | 278 | 18.3 | 48.65 | −6.26 | 1.00 | 22.57 |
| 526 | O | GLU | A | 278 | 18.329 | 47.74 | −5.441 | 1.00 | 21.72 |
| 527 | N | VAL | A | 279 | 17.551 | 49.74 | −6.094 | 1.00 | 21.59 |
| 528 | CA | VAL | A | 279 | 16.731 | 49.9 | −4.895 | 1.00 | 21.26 |
| 529 | CB | VAL | A | 279 | 16.303 | 51.37 | −4.721 | 1.00 | 23.06 |
| 530 | CG1 | VAL | A | 279 | 15.292 | 51.5 | −3.596 | 1.00 | 25.08 |
| 531 | CG2 | VAL | A | 279 | 17.53 | 52.22 | −4.415 | 1.00 | 25.52 |
| 532 | G | VAL | A | 279 | 15.5 | 49 | −4.947 | 1.00 | 19.49 |
| 533 | O | VAL | A | 279 | 15.058 | 48.47 | −3.918 | 1.00 | 18.78 |
| 534 | N | ARG | A | 280 | 14.956 | 48.81 | −6.146 | 1.00 | 18.65 |
| 535 | CA | ARG | A | 280 | 13.796 | 47.94 | −6.338 | 1.00 | 17.90 |
| 536 | CB | ARG | A | 280 | 13.243 | 48.1 | −7.762 | 1.00 | 18.81 |
| 537 | CG | ARG | A | 280 | 12.211 | 49.2 | −7.895 | 1.00 | 23.01 |
| 538 | CD | ARG | A | 280 | 12.321 | 49.93 | −9.216 | 1.00 | 23.63 |
| 539 | NE | ARG | A | 280 | 12.387 | 49.04 | −10.37 | 1.00 | 21.82 |
| 540 | CZ | ARG | A | 280 | 12.895 | 49.4 | −11.55 | 1.00 | 23.69 |
| 541 | NH1 | ARG | A | 280 | 13.376 | 50.63 | −11.7 | 1.00 | 25.06 |
| 542 | NH2 | ARG | A | 280 | 12.928 | 48.55 | −12.56 | 1.00 | 24.09 |
| 543 | C | ARG | A | 280 | 14.217 | 46.49 | −6.096 | 1.00 | 17.66 |
| 544 | O | ARG | A | 280 | 13.531 | 45.74 | −5.405 | 1.00 | 16.97 |
| 545 | N | ILE | A | 281 | 15.356 | 46.11 | −6.661 | 1.00 | 17.24 |
| 546 | CA | ILE | A | 281 | 15.877 | 44.76 | −6.496 | 1.00 | 16.58 |
| 547 | CB | ILE | A | 281 | 17.152 | 44.55 | −7.34 | 1.00 | 17.41 |
| 548 | CG2 | ILE | A | 281 | 17.846 | 43.24 | −6.943 | 1.00 | 17.49 |
| 549 | CG1 | ILE | A | 281 | 16.779 | 44.56 | −8.828 | 1.00 | 17.66 |
| 550 | CD1 | ILE | A | 281 | 17.964 | 44.53 | −9.781 | 1.00 | 19.17 |
| 551 | C | ILE | A | 281 | 16.192 | 44.5 | −5.024 | 1.00 | 16.90 |
| 552 | O | ILE | A | 281 | 15.898 | 43.43 | −4.497 | 1.00 | 16.85 |
| 553 | N | PHE | A | 282 | 16.776 | 45.5 | −4.359 | 1.00 | 14.57 |
| 554 | CA | PHE | A | 282 | 17.111 | 45.36 | −2.942 | 1.00 | 13.82 |
| 555 | CB | PHE | A | 282 | 17.886 | 46.59 | −2.455 | 1.00 | 14.33 |
| 556 | CG | PHE | A | 282 | 18.48 | 46.42 | −1.084 | 1.00 | 16.86 |
| 557 | CD1 | PHE | A | 282 | 19.595 | 45.62 | −0.894 | 1.00 | 17.16 |
| 558 | CD2 | PHE | A | 282 | 17.927 | 47.07 | 0.014 | 1.00 | 16.21 |
| 559 | CE1 | PHE | A | 282 | 20.157 | 45.46 | 0.364 | 1.00 | 18.94 |
| 560 | CE2 | PHE | A | 282 | 18.481 | 46.92 | 1.28 | 1.00 | 19.15 |
| 561 | CZ | PHE | A | 282 | 19.601 | 46.11 | 1.456 | 1.00 | 20.18 |
| 562 | C | PHE | A | 282 | 15.848 | 45.21 | −2.099 | 1.00 | 14.30 |
| 563 | O | PHE | A | 282 | 15.861 | 44.52 | −1.085 | 1.00 | 15.47 |
| 564 | N | HIS | A | 283 | 14.766 | 45.87 | −2.498 | 1.00 | 14.52 |
| 565 | CA | HIS | A | 283 | 13.517 | 45.76 | −1.761 | 1.00 | 15.32 |
| 566 | CB | HIS | A | 283 | 12.454 | 46.7 | −2.334 | 1.00 | 16.74 |
| 567 | CG | HIS | A | 283 | 11.139 | 46.6 | −1.628 | 1.00 | 18.93 |
| 568 | CD2 | HIS | A | 283 | 9.966 | 46.04 | −1.999 | 1.00 | 21.47 |
| 569 | ND1 | HIS | A | 283 | 10.95 | 47.07 | −0.345 | 1.00 | 19.40 |
| 570 | CE1 | HIS | A | 283 | 9.718 | 46.8 | 0.045 | 1.00 | 21.96 |
| 571 | NE2 | HIS | A | 283 | 9.1 | 46.17 | −0.94 | 1.00 | 23.33 |
| 572 | C | HIS | A | 283 | 13.03 | 44.31 | −1.877 | 1.00 | 14.53 |
| 573 | O | HIS | A | 283 | 12.606 | 43.71 | −0.9 | 1.00 | 15.83 |
| 574 | N | CYS | A | 284 | 13.089 | 43.77 | −3.086 | 1.00 | 14.60 |
| 575 | CA | CYS | A | 284 | 12.668 | 42.39 | −3.317 | 1.00 | 14.94 |
| 576 | CB | CYS | A | 284 | 12.713 | 42.06 | −4.81 | 1.00 | 14.09 |
| 577 | SG | CYS | A | 284 | 11.464 | 42.95 | −5.76 | 1.00 | 16.01 |
| 578 | C | CYS | A | 284 | 13.555 | 41.41 | −2.539 | 1.00 | 14.97 |
| 579 | O | CYS | A | 284 | 13.09 | 40.37 | −2.088 | 1.00 | 13.78 |
| 580 | N | CYS | A | 285 | 14.835 | 41.75 | −2.385 | 1.00 | 15.23 |
| 581 | CA | CYS | A | 285 | 15.741 | 40.88 | −1.626 | 1.00 | 13.47 |
| 582 | CB | CYS | A | 285 | 17.179 | 41.42 | −1.666 | 1.00 | 15.89 |
| 583 | SG | GYS | A | 285 | 17.998 | 41.25 | −3.256 | 1.00 | 15.55 |
| 584 | C | CYS | A | 285 | 15.266 | 40.84 | −0.179 | 1.00 | 15.09 |
| 585 | O | CYS | A | 285 | 15.27 | 39.79 | 0.466 | 1.00 | 15.43 |
| 586 | N | GLN | A | 286 | 14.857 | 42 | 0.327 | 1.00 | 13.05 |
| 587 | CA | GLN | A | 286 | 14.375 | 42.1 | 1.7 | 1.00 | 14.74 |
| 588 | CB | GLN | A | 286 | 14.151 | 43.56 | 2.098 | 1.00 | 16.57 |
| 589 | CG | GLN | A | 286 | 15.42 | 44.38 | 2.198 | 1.00 | 18.11 |
| 590 | CD | GLN | A | 286 | 15.234 | 45.59 | 3.08 | 1.00 | 20.26 |
| 591 | QEl | GLN | A | 286 | 14.974 | 45.47 | 4.278 | 1.00 | 22.83 |
| 592 | NE2 | GLN | A | 286 | 15.362 | 46.78 | 2.494 | 1.00 | 22.10 |
| 593 | C | GLN | A | 286 | 13.079 | 41.34 | 1.904 | 1.00 | 14.38 |
| 594 | O | GLN | A | 286 | 12.896 | 40.67 | 2.925 | 1.00 | 14.08 |
| 595 | N | CYS | A | 287 | 12.169 | 41.45 | 0.943 | 1.00 | 14.67 |
| 596 | CA | CYS | A | 287 | 10.905 | 40.74 | 1.063 | 1.00 | 15.04 |
| 597 | CB | CYS | A | 287 | 9.982 | 41.09 | −0.104 | 1.00 | 16.30 |
| 598 | SG | CYS | A | 287 | 9.397 | 42.8 | −0.06 | 1.00 | 22.25 |
| 599 | C | CYS | A | 287 | 11.187 | 39.24 | 1.091 | 1.00 | 14.70 |
| 600 | O | CYS | A | 287 | 10.587 | 38.5 | 1.864 | 1.00 | 14.34 |
| 601 | N | THR | A | 288 | 12.117 | 38.81 | 0.245 | 1.00 | 14.73 |
| 602 | CA | THR | A | 288 | 12.511 | 37.41 | 0.164 | 1.00 | 14.32 |
| 603 | CB | THR | A | 288 | 13.5 | 37.2 | −1.003 | 1.00 | 15.37 |
| 604 | OG1 | THR | A | 288 | 12.863 | 37.59 | −2.233 | 1.00 | 13.87 |
| 605 | CG2 | THR | A | 288 | 13.93 | 35.74 | −1.1 | 1.00 | 14.11 |
| 606 | C | THR | A | 288 | 13.132 | 36.94 | 1.485 | 1.00 | 14.08 |
| 607 | O | THR | A | 288 | 12.771 | 35.89 | 2.023 | 1.00 | 13.41 |
| 608 | N | SER | A | 289 | 14.05 | 37.73 | 2.03 | 1.00 | 12.61 |
| 609 | CA | SER | A | 289 | 14.664 | 37.37 | 3.305 | 1.00 | 12.61 |
| 610 | CB | SER | A | 289 | 15.769 | 38.36 | 3.659 | 1.00 | 12.19 |
| 611 | 00 | SER | A | 289 | 16.916 | 38.11 | 2.875 | 1.00 | 11.40 |
| 612 | C | SER | A | 289 | 13.652 | 37.31 | 4.453 | 1.00 | 12.67 |
| 613 | O | SER | A | 289 | 13.72 | 36.42 | 5.289 | 1.00 | 12.90 |
| 614 | N | VAL | A | 290 | 12.71 | 38.25 | 4.493 | 1.00 | 13.57 |
| 615 | CA | VAL | A | 290 | 11.712 | 38.25 | 5.564 | 1.00 | 13.84 |
| 616 | CB | VAL | A | 290 | 10.78 | 39.48 | 5.455 | 1.00 | 15.55 |
| 617 | CG1 | VAL | A | 290 | 9.541 | 39.31 | 6.333 | 1.00 | 17.55 |
| 618 | CG2 | VAL | A | 290 | 11.549 | 40.72 | 5.887 | 1.00 | 16.53 |
| 619 | C | VAL | A | 290 | 10.905 | 36.96 | 5.536 | 1.00 | 15.35 |
| 620 | O | VAL | A | 290 | 10.64 | 36.35 | 6.578 | 1.00 | 14.98 |
| 621 | N | GLU | A | 291 | 10.536 | 36.52 | 4.338 | 1.00 | 16.73 |
| 622 | CA | GLU | A | 291 | 9.771 | 35.28 | 4.185 | 1.00 | 16.47 |
| 623 | CB | OLU | A | 291 | 9.329 | 35.11 | 2.729 | 1.00 | 19.03 |
| 624 | CO | GLU | A | 291 | 8.339 | 36.16 | 2.244 | 1.00 | 22.45 |
| 625 | CD | GLU | A | 291 | 6.917 | 35.91 | 2.726 | 1.00 | 27.72 |
| 626 | OE1 | GLU | A | 291 | 6.712 | 35 | 3.567 | 1.00 | 28.03 |
| 627 | OE2 | GLU | A | 291 | 6.003 | 36.62 | 2.261 | 1.00 | 27.23 |
| 628 | C | GLU | A | 291 | 10.604 | 34.08 | 4.61 | 1.00 | 15.29 |
| 629 | O | GLU | A | 291 | 10.107 | 33.19 | 5.286 | 1.00 | 14.93 |
| 630 | N | THR | A | 292 | 11.877 | 34.07 | 4.228 | 1.00 | 13.72 |
| 631 | CA | THR | A | 292 | 12.731 | 32.94 | 4.584 | 1.00 | 13.42 |
| 632 | CB | THR | A | 292 | 14.074 | 33.01 | 3.839 | 1.00 | 13.57 |
| 633 | OG1 | THR | A | 292 | 13.825 | 33.11 | 2.428 | 1.00 | 13.65 |
| 634 | CG2 | THR | A | 292 | 14.885 | 31.74 | 4.092 | 1.00 | 13.97 |
| 635 | C | THR | A | 292 | 12.966 | 32.88 | 6.091 | 1.00 | 13.92 |
| 636 | O | THR | A | 292 | 12.963 | 31.8 | 6.68 | 1.00 | 15.10 |
| 637 | N | VAL | A | 293 | 13.16 | 34.04 | 6.718 | 1.00 | 14.12 |
| 638 | CA | VAL | A | 293 | 13.357 | 34.07 | 8.172 | 1.00 | 14.58 |
| 639 | CB | VAL | A | 293 | 13.612 | 35.5 | 8.676 | 1.00 | 14.45 |
| 640 | CG1 | VAL | A | 293 | 13.488 | 35.55 | 10.201 | 1.00 | 15.36 |
| 641 | CG2 | VAL | A | 293 | 14.986 | 35.97 | 8.243 | 1.00 | 15.78 |
| 642 | C | VAL | A | 293 | 12.095 | 33.54 | 8.855 | 1.00 | 14.26 |
| 643 | O | VAL | A | 293 | 12.164 | 32.82 | 9.866 | 1.00 | 14.04 |
| 644 | N | THR | A | 294 | 10.941 | 33.9 | 8.304 | 1.00 | 15.03 |
| 645 | CA | THR | A | 294 | 9.667 | 33.45 | 8.859 | 1.00 | 16.55 |
| 646 | CB | THR | A | 294 | 8.491 | 34.13 | 8.119 | 1.00 | 17.70 |
| 647 | OG1 | THR | A | 294 | 8.624 | 35.56 | 8.228 | 1.00 | 18.26 |
| 648 | CG2 | THR | A | 294 | 7.154 | 33.71 | 8.721 | 1.00 | 18.59 |
| 649 | C | THR | A | 294 | 9.553 | 31.93 | 8.779 | 1.00 | 16.67 |
| 650 | O | THR | A | 294 | 9.122 | 31.28 | 9.731 | 1.00 | 15.85 |
| 651 | N | GLU | A | 295 | 9.953 | 31.35 | 7.65 | 1.00 | 16.45 |
| 652 | CA | GLU | A | 295 | 9.896 | 29.89 | 7.495 | 1.00 | 14.59 |
| 653 | CB | GLU | A | 295 | 10.204 | 29.48 | 6.047 | 1.00 | 17.05 |
| 654 | CG | GLU | A | 295 | 9.207 | 29.97 | 5.031 | 1.00 | 19.53 |
| 655 | CD | GLU | A | 295 | 9.556 | 29.51 | 3.623 | 1.00 | 21.27 |
| 656 | OE1 | GLU | A | 295 | 10.759 | 29.44 | 3.297 | 1.00 | 24.29 |
| 657 | OE2 | GLU | A | 295 | 8.628 | 29.24 | 2.845 | 1.00 | 26.60 |
| 658 | C | GLU | A | 295 | 10.892 | 29.18 | 8.419 | 1.00 | 15.53 |
| 659 | O | GLU | A | 295 | 10.591 | 28.13 | 8.986 | 1.00 | 14.95 |
| 660 | N | LEU | A | 296 | 12.084 | 29.76 | 8.554 | 1.00 | 14.15 |
| 661 | CA | LEU | A | 296 | 13.121 | 29.18 | 9.402 | 1.00 | 14.54 |
| 662 | CB | LEU | A | 296 | 14.421 | 29.98 | 9.243 | 1.00 | 13.35 |
| 663 | CG | LEU | A | 296 | 15.288 | 29.53 | 8.06 | 1.00 | 13.79 |
| 664 | CD1 | LEU | A | 296 | 16.282 | 30.62 | 7.666 | 1.00 | 14.77 |
| 665 | CD2 | LEU | A | 296 | 16.026 | 28.25 | 8.448 | 1.00 | 15.41 |
| 666 | C | LEU | A | 296 | 12.687 | 29.18 | 10.863 | 1.00 | 15.65 |
| 667 | O | LEU | A | 296 | 13.026 | 28.28 | 11.629 | 1.00 | 15.76 |
| 668 | N | THR | A | 297 | 11.942 | 30.21 | 11.241 | 1.00 | 16.74 |
| 669 | CA | THR | A | 297 | 11.467 | 30.33 | 12.613 | 1.00 | 17.31 |
| 670 | CB | THR | A | 297 | 10.823 | 31.72 | 12.827 | 1.00 | 17.24 |
| 671 | OG1 | THR | A | 297 | 11.84 | 32.72 | 12.696 | 1.00 | 18.00 |
| 672 | CG2 | THR | A | 297 | 10.184 | 31.83 | 14.216 | 1.00 | 18.26 |
| 673 | C | THR | A | 297 | 10.48 | 29.2 | 12.91 | 1.00 | 18.94 |
| 674 | O | THR | A | 297 | 10.518 | 28.61 | 13.99 | 1.00 | 18.36 |
| 675 | N | GLU | A | 298 | 9.61 | 28.9 | 11.947 | 1.00 | 18.64 |
| 676 | CA | GLU | A | 298 | 8.647 | 27.82 | 12.123 | 1.00 | 19.64 |
| 677 | CB | GLU | A | 298 | 7.585 | 27.87 | 11.023 | 1.00 | 20.78 |
| 678 | CG | GLU | A | 298 | 6.701 | 29.09 | 11.109 | 1.00 | 24.51 |
| 679 | CD | GLU | A | 298 | 5.986 | 29.17 | 12.439 | 1.00 | 27.16 |
| 680 | OE1 | GLU | A | 298 | 5.16 | 28.28 | 12.715 | 1.00 | 28.65 |
| 681 | OE2 | GLU | A | 298 | 6.256 | 30.12 | 13.208 | 1.00 | 29.29 |
| 682 | C | GLU | A | 298 | 9.375 | 26.48 | 12.099 | 1.00 | 19.12 |
| 683 | O | GLU | A | 298 | 9.006 | 25.55 | 12.817 | 1.00 | 19.43 |
| 684 | N | PIlE | A | 299 | 10.403 | 26.38 | 11.258 | 1.00 | 18.63 |
| 685 | CA | PHE | A | 299 | 11.205 | 25.15 | 11.174 | 1.00 | 18.56 |
| 686 | CB | PHE | A | 299 | 12.284 | 25.3 | 10.094 | 1.00 | 18.22 |
| 687 | CG | PHE | A | 299 | 13.287 | 24.17 | 10.078 | 1.00 | 16.52 |
| 688 | OD1 | PHE | A | 299 | 12.909 | 22.89 | 9.711 | 1.00 | 15.73 |
| 689 | CD2 | PHE | A | 299 | 14.622 | 24.41 | 10.407 | 1.00 | 16.62 |
| 690 | CE1 | PHE | A | 299 | 13.842 | 21.85 | 9.667 | 1.00 | 16.62 |
| 691 | CE2 | PHE | A | 299 | 15.56 | 23.39 | 10.367 | 1.00 | 16.63 |
| 692 | CZ | PHE | A | 299 | 15.168 | 22.1 | 9.992 | 1.00 | 16.10 |
| 693 | C | PHE | A | 299 | 11.887 | 24.9 | 12.52 | 1.00 | 19.02 |
| 694 | O | PHE | A | 299 | 11.855 | 23.78 | 13.041 | 1.00 | 19.20 |
| 695 | N | ALA | A | 300 | 12.519 | 25.93 | 13.068 | 1.00 | 19.85 |
| 696 | CA | ALA | A | 300 | 13.218 | 25.8 | 14.348 | 1.00 | 19.89 |
| 697 | CB | ALA | A | 300 | 13.855 | 27.13 | 14.739 | 1.00 | 20.05 |
| 698 | C | ALA | A | 300 | 12.267 | 25.34 | 15.448 | 1.00 | 21.19 |
| 699 | O | ALA | A | 300 | 12.627 | 24.5 | 16.284 | 1.00 | 19.62 |
| 700 | N | LYS | A | 301 | 11.059 | 25.89 | 15.447 | 1.00 | 21.75 |
| 701 | CA | LYS | A | 301 | 10.052 | 25.54 | 16.444 | 1.00 | 23.62 |
| 702 | CB | LYS | A | 301 | 8.8 | 26.4 | 16.253 | 1.00 | 22.82 |
| 703 | CG | LYS | A | 301 | 8.959 | 27.85 | 16.717 | 1.00 | 27.26 |
| 704 | CD | LYS | A | 301 | 7.814 | 28.74 | 16.225 | 1.00 | 30.13 |
| 705 | CE | LYS | A | 301 | 6.447 | 28.16 | 16.557 | 1.00 | 32.35 |
| 706 | NZ | LYS | A | 301 | 6.238 | 27.99 | 18.021 | 1.00 | 36.87 |
| 707 | C | LYS | A | 301 | 9.685 | 24.07 | 16.352 | 1.00 | 24.64 |
| 708 | O | LYS | A | 301 | 9.158 | 23.49 | 17.304 | 1.00 | 24.87 |
| 709 | N | ALA | A | 302 | 9.973 | 23.46 | 15.204 | 1.00 | 24.73 |
| 710 | CA | ALA | A | 302 | 9.674 | 22.05 | 14.987 | 1.00 | 24.93 |
| 711 | CB | ALA | A | 302 | 9.181 | 21.84 | 13.563 | 1.00 | 24.81 |
| 712 | C | ALA | A | 302 | 10.864 | 21.13 | 15.277 | 1.00 | 24.71 |
| 713 | O | ALA | A | 302 | 10.746 | 19.91 | 15.187 | 1.00 | 23.86 |
| 714 | N | ILE | A | 303 | 12.014 | 21.71 | 15.613 | 1.00 | 23.98 |
| 715 | CA | ILE | A | 303 | 13.179 | 20.89 | 15.932 | 1.00 | 22.06 |
| 716 | CB | ILE | A | 303 | 14.503 | 21.67 | 15.776 | 1.00 | 21.52 |
| 717 | CG2 | ILE | A | 303 | 15.675 | 20.77 | 16.168 | 1.00 | 20.29 |
| 718 | CG1 | ILE | A | 303 | 14.681 | 22.13 | 14.325 | 1.00 | 18.78 |
| 719 | OD1 | ILE | A | 303 | 15.946 | 22.93 | 14.104 | 1.00 | 20.43 |
| 720 | C | ILE | A | 303 | 13.059 | 20.45 | 17.386 | 1.00 | 23.55 |
| 721 | O | ILE | A | 303 | 13.018 | 21.28 | 18.292 | 1.00 | 22.86 |
| 722 | N | PRO | A | 304 | 13 | 19.13 | 17.626 | 1.00 | 24.07 |
| 723 | CD | PRO | A | 304 | 13.119 | 18.03 | 16.644 | 1.00 | 23.94 |
| 724 | CA | PRO | A | 304 | 12.883 | 18.59 | 18.985 | 1.00 | 24.71 |
| 725 | CB | PRO | A | 304 | 13.275 | 17.13 | 18.804 | 1.00 | 24.70 |
| 726 | CG | PRO | A | 304 | 12.711 | 16.82 | 17.456 | 1.00 | 25.44 |
| 727 | C | PRO | A | 304 | 13.761 | 19.3 | 20.013 | 1.00 | 24.62 |
| 728 | O | PRO | A | 304 | 14.987 | 19.33 | 19.878 | 1.00 | 25.02 |
| 729 | N | ALA | A | 305 | 13.115 | 19.88 | 21.026 | 1.00 | 23.49 |
| 730 | CA | ALA | A | 305 | 13.788 | 20.58 | 22.124 | 1.00 | 23.80 |
| 731 | CB | ALA | A | 305 | 15.122 | 19.91 | 22.432 | 1.00 | 26.87 |
| 732 | C | ALA | A | 305 | 13.995 | 22.08 | 21.959 | 1.00 | 22.79 |
| 733 | O | ALA | A | 305 | 14.242 | 22.78 | 22.945 | 1.00 | 21.27 |
| 734 | N | PHE | A | 306 | 13.903 | 22.58 | 20.732 | 1.00 | 22.01 |
| 735 | CA | PHE | A | 306 | 14.101 | 24.01 | 20.515 | 1.00 | 22.24 |
| 736 | CB | PHE | A | 306 | 14.015 | 24.35 | 19.023 | 1.00 | 21.13 |
| 737 | CG | PHE | A | 306 | 14.301 | 25.8 | 18.714 | 1.00 | 20.73 |
| 738 | CD1 | PHE | A | 306 | 13.293 | 26.76 | 18.775 | 1.00 | 21.28 |
| 739 | CD2 | PHE | A | 306 | 15.591 | 26.21 | 18.393 | 1.00 | 20.25 |
| 740 | CE1 | PHE | A | 306 | 13.568 | 28.1 | 18.519 | 1.00 | 20.90 |
| 741 | CE2 | PHE | A | 306 | 15.875 | 27.55 | 18.136 | 1.00 | 19.14 |
| 742 | CZ | PHE | A | 306 | 14.864 | 28.5 | 18.199 | 1.00 | 19.75 |
| 743 | C | PHE | A | 306 | 13.088 | 24.85 | 21.285 | 1.00 | 22.26 |
| 744 | O | PHE | A | 306 | 13.448 | 25.84 | 21.936 | 1.00 | 22.51 |
| 745 | N | ALA | A | 307 | 11.821 | 24.46 | 21.215 | 1.00 | 23.86 |
| 746 | CA | ALA | A | 307 | 10.763 | 25.21 | 21.89 | 1.00 | 24.65 |
| 747 | CB | ALA | A | 307 | 9.399 | 24.7 | 21.441 | 1.00 | 25.58 |
| 748 | C | ALA | A | 307 | 10.868 | 25.16 | 23.413 | 1.00 | 25.96 |
| 749 | O | ALA | A | 307 | 10.238 | 25.96 | 24.102 | 1.00 | 26.50 |
| 750 | N | ASN | A | 308 | 11.667 | 24.23 | 23.932 | 1.00 | 26.32 |
| 751 | CA | ASN | A | 308 | 11.852 | 24.09 | 25.378 | 1.00 | 26.91 |
| 752 | CB | ASN | A | 308 | 12.291 | 22.67 | 25.717 | 1.00 | 27.32 |
| 753 | CG | ASN | A | 308 | 11.194 | 21.65 | 25.5 | 1.00 | 27.93 |
| 754 | OD1 | ASN | A | 308 | 11.455 | 20.45 | 25.428 | 1.00 | 31.25 |
| 755 | ND2 | ASN | A | 308 | 9.958 | 22.12 | 25.402 | 1.00 | 27.97 |
| 756 | C | ASN | A | 308 | 12.889 | 25.07 | 25.921 | 1.00 | 27.12 |
| 757 | O | ASN | A | 308 | 12.985 | 25.29 | 27.132 | 1.00 | 26.42 |
| 758 | N | LEU | A | 309 | 13.68 | 25.64 | 25.025 | 1.00 | 24.59 |
| 759 | CA | LEU | A | 309 | 14.702 | 26.6 | 25.428 | 1.00 | 23.00 |
| 760 | CB | LEU | A | 309 | 15.615 | 26.92 | 24.245 | 1.00 | 20.53 |
| 761 | CG | LEU | A | 309 | 16.484 | 25.8 | 23.676 | 1.00 | 20.80 |
| 762 | CD1 | LEU | A | 309 | 17.175 | 26.3 | 22.414 | 1.00 | 20.69 |
| 763 | CD2 | LEU | A | 309 | 17.512 | 25.37 | 24.714 | 1.00 | 21.60 |
| 764 | C | LEU | A | 309 | 14.041 | 27.87 | 25.91 | 1.00 | 21.59 |
| 765 | O | LEU | A | 309 | 12.89 | 28.13 | 25.582 | 1.00 | 20.49 |
| 766 | N | ASP | A | 310 | 14.767 | 28.65 | 26.701 | 1.00 | 23.45 |
| 767 | CA | ASP | A | 310 | 14.243 | 29.93 | 27.175 | 1.00 | 23.40 |
| 768 | CB | ASP | A | 310 | 15.263 | 30.64 | 28.062 | 1.00 | 22.34 |
| 769 | CG | ASP | A | 310 | 14.85 | 32.06 | 28.402 | 1.00 | 23.70 |
| 770 | OD1 | ASP | A | 310 | 13.85 | 32.23 | 29.127 | 1.00 | 24.73 |
| 771 | OD2 | ASP | A | 310 | 15.52 | 33 | 27.937 | 1.00 | 24.97 |
| 772 | C | ASP | A | 310 | 14.044 | 30.73 | 25.898 | 1.00 | 23.49 |
| 773 | O | ASP | A | 310 | 14.814 | 30.58 | 24.952 | 1.00 | 22.64 |
| 774 | N | LEU | A | 311 | 13.029 | 31.59 | 25.866 | 1.00 | 24.03 |
| 775 | CA | LEU | A | 311 | 12.762 | 32.38 | 24.669 | 1.00 | 24.61 |
| 776 | CB | LEU | A | 311 | 11.489 | 33.21 | 24.848 | 1.00 | 25.91 |
| 777 | CG | LEU | A | 311 | 11.312 | 34.15 | 26.038 | 1.00 | 29.90 |
| 778 | CD1 | LEU | A | 311 | 12.37 | 35.25 | 26.043 | 1.00 | 31.19 |
| 779 | CD2 | LEU | A | 311 | 9.925 | 34.77 | 25.94 | 1.00 | 32.22 |
| 780 | C | LEU | A | 311 | 13.916 | 33.28 | 24.225 | 1.00 | 23.78 |
| 781 | O | LEU | A | 311 | 14.075 | 33.54 | 23.035 | 1.00 | 22.46 |
| 782 | N | ASN | A | 312 | 14.713 | 33.77 | 25.17 | 1.00 | 22.87 |
| 783 | CA | ASN | A | 312 | 15.843 | 34.62 | 24.815 | 1.00 | 23.57 |
| 784 | CB | ASN | A | 312 | 16.501 | 35.19 | 26.072 | 1.00 | 24.41 |
| 785 | CG | ASN | A | 312 | 15.594 | 36.15 | 26.819 | 1.00 | 25.82 |
| 786 | OD1 | ASN | A | 312 | 15.333 | 37.27 | 26.359 | 1.00 | 24.76 |
| 787 | ND2 | ASN | A | 312 | 15.1 | 35.72 | 27.975 | 1.00 | 25.85 |
| 788 | C | ASN | A | 312 | 16.859 | 33.79 | 24.029 | 1.00 | 23.03 |
| 789 | O | ASN | A | 312 | 17.48 | 34.28 | 23.077 | 1.00 | 22.95 |
| 790 | N | ASP | A | 313 | 17.026 | 32.54 | 24.433 | 1.00 | 21.92 |
| 791 | CA | ASP | A | 313 | 17.959 | 31.66 | 23.751 | 1.00 | 21.37 |
| 792 | CB | ASP | A | 313 | 18.224 | 30.41 | 24.582 | 1.00 | 20.90 |
| 793 | CG | ASP | A | 313 | 19.281 | 30.63 | 25.649 | 1.00 | 20.90 |
| 794 | OD1 | ASP | A | 313 | 19.785 | 31.77 | 25.768 | 1.00 | 22.16 |
| 795 | OD2 | ASP | A | 313 | 19.609 | 29.67 | 26.364 | 1.00 | 22.62 |
| 796 | C | ASP | A | 313 | 17.411 | 31.28 | 22.383 | 1.00 | 20.42 |
| 797 | O | ASP | A | 313 | 18.176 | 31.07 | 21.442 | 1.00 | 21.27 |
| 798 | N | GLN | A | 314 | 16.088 | 31.2 | 22.268 | 1.00 | 19.98 |
| 799 | CA | GLN | A | 314 | 15.478 | 30.87 | 20.981 | 1.00 | 20.73 |
| 800 | CB | GLN | A | 314 | 13.971 | 30.64 | 21.124 | 1.00 | 20.41 |
| 801 | CG | GLN | A | 314 | 13.598 | 29.36 | 21.862 | 1.00 | 22.80 |
| 802 | CD | GLN | A | 314 | 12.1 | 29.12 | 21.906 | 1.00 | 24.47 |
| 803 | OE1 | GLN | A | 314 | 11.425 | 29.14 | 20.876 | 1.00 | 26.99 |
| 804 | NE2 | GLN | A | 314 | 11.571 | 28.89 | 23.104 | 1.00 | 23.97 |
| 805 | C | GLN | A | 314 | 15.74 | 32.04 | 20.03 | 1.00 | 19.47 |
| 806 | O | GLN | A | 314 | 16.099 | 31.84 | 18.869 | 1.00 | 19.20 |
| 807 | N | VAL | A | 315 | 15.561 | 33.25 | 20.541 | 1.00 | 19.92 |
| 808 | CA | VAL | A | 315 | 15.788 | 34.46 | 19.76 | 1.00 | 18.92 |
| 809 | CB | VAL | A | 315 | 15.414 | 35.72 | 20.583 | 1.00 | 19.64 |
| 810 | CG1 | VAL | A | 315 | 15.861 | 36.98 | 19.867 | 1.00 | 20.62 |
| 811 | CG2 | VAL | A | 315 | 13.91 | 35.75 | 20.794 | 1.00 | 19.50 |
| 812 | C | VAL | A | 315 | 17.246 | 34.55 | 19.321 | 1.00 | 18.13 |
| 813 | O | VAL | A | 315 | 17.538 | 34.84 | 18.157 | 1.00 | 17.71 |
| 814 | N | THR | A | 316 | 18.161 | 34.3 | 20.252 | 1.00 | 17.09 |
| 815 | CA | THR | A | 316 | 19.588 | 34.36 | 19.956 | 1.00 | 17.05 |
| 816 | CB | THR | A | 316 | 20.429 | 34.13 | 21.242 | 1.00 | 17.63 |
| 817 | OG1 | THR | A | 316 | 20.162 | 35.19 | 22.17 | 1.00 | 16.06 |
| 818 | CG2 | THR | A | 316 | 21.922 | 34.12 | 20.92 | 1.00 | 16.45 |
| 819 | C | THR | A | 316 | 20.007 | 33.34 | 18.889 | 1.00 | 16.61 |
| 820 | O | THR | A | 316 | 20.768 | 33.67 | 17.984 | 1.00 | 17.34 |
| 821 | N | LEU | A | 317 | 19.503 | 32.12 | 18.989 | 1.00 | 15.88 |
| 822 | CA | LEU | A | 317 | 19.864 | 31.09 | 18.018 | 1.00 | 16.20 |
| 823 | CB | LEU | A | 317 | 19.264 | 29.74 | 18.41 | 1.00 | 17.78 |
| 824 | CG | LEU | A | 317 | 19.886 | 29.11 | 19.656 | 1.00 | 16.63 |
| 825 | CD1 | LEU | A | 317 | 19.277 | 27.73 | 19.916 | 1.00 | 17.89 |
| 826 | CD2 | LEU | A | 317 | 21.393 | 29.01 | 19.46 | 1.00 | 17.96 |
| 827 | C | LEU | A | 317 | 19.417 | 31.48 | 16.616 | 1.00 | 16.44 |
| 828 | O | LEU | A | 317 | 20.147 | 31.27 | 15.643 | 1.00 | 17.08 |
| 829 | N | LEU | A | 318 | 18.221 | 32.05 | 16.507 | 1.00 | 16.59 |
| 830 | CA | LEU | A | 318 | 17.723 | 32.47 | 15.2 | 1.00 | 17.03 |
| 831 | CB | LEU | A | 318 | 16.209 | 32.72 | 15.255 | 1.00 | 16.97 |
| 832 | CG | LEU | A | 318 | 15.374 | 31.43 | 15.321 | 1.00 | 18.35 |
| 833 | CD1 | LEU | A | 318 | 13.916 | 31.77 | 15.589 | 1.00 | 23.00 |
| 834 | CD2 | LEU | A | 318 | 15.506 | 30.66 | 14.012 | 1.00 | 19.92 |
| 835 | C | LEU | A | 318 | 18.447 | 33.73 | 14.753 | 1.00 | 17.00 |
| 836 | O | LEU | A | 318 | 18.831 | 33.86 | 13.591 | 1.00 | 17.06 |
| 837 | N | LYS | A | 319 | 18.645 | 34.67 | 15.677 | 1.00 | 16.85 |
| 838 | CA | LYS | A | 319 | 19.332 | 35.91 | 15.335 | 1.00 | 17.88 |
| 839 | CB | LYS | A | 319 | 19.569 | 36.75 | 16.592 | 1.00 | 19.60 |
| 840 | CG | LYS | A | 319 | 20.244 | 38.09 | 16.315 | 1.00 | 21.39 |
| 841 | CD | LYS | A | 319 | 20.632 | 38.78 | 17.621 | 1.00 | 24.10 |
| 842 | CE | LYS | A | 319 | 21.26 | 40.14 | 17.374 | 1.00 | 24.44 |
| 843 | NZ | LYS | A | 319 | 20.287 | 41.09 | 16.751 | 1.00 | 26.22 |
| 844 | C | LYS | A | 319 | 20.671 | 35.66 | 14.634 | 1.00 | 18.39 |
| 845 | O | LYS | A | 319 | 20.961 | 36.28 | 13.609 | 1.00 | 19.24 |
| 846 | N | TYR | A | 320 | 21.475 | 34.75 | 15.177 | 1.00 | 16.44 |
| 847 | CA | TYR | A | 320 | 22.786 | 34.46 | 14.604 | 1.00 | 19.84 |
| 848 | CB | TYR | A | 320 | 23.781 | 34.13 | 15.721 | 1.00 | 23.67 |
| 849 | CG | TYR | A | 320 | 24.134 | 35.33 | 16.576 | 1.00 | 27.71 |
| 850 | CD1 | TYR | A | 320 | 24.989 | 36.32 | 16.1 | 1.00 | 30.38 |
| 851 | CE1 | TYR | A | 320 | 25.301 | 37.44 | 16.877 | 1.00 | 32.19 |
| 852 | CD2 | TYR | A | 320 | 23.598 | 35.48 | 17.852 | 1.00 | 30.97 |
| 853 | OE2 | TYR | A | 320 | 23.902 | 36.59 | 18.638 | 1.00 | 33.09 |
| 854 | CZ | TYR | A | 320 | 24.756 | 37.56 | 18.143 | 1.00 | 33.75 |
| 855 | OH | TYR | A | 320 | 25.075 | 38.65 | 18.916 | 1.00 | 36.85 |
| 856 | C | TYR | A | 320 | 22.8 | 33.33 | 13.577 | 1.00 | 17.92 |
| 857 | O | TYR | A | 320 | 23.708 | 33.26 | 12.747 | 1.00 | 20.86 |
| 858 | N | GLY | A | 321 | 21.795 | 32.47 | 13.612 | 1.00 | 16.75 |
| 859 | CA | GLY | A | 321 | 21.784 | 31.36 | 12.67 | 1.00 | 15.82 |
| 860 | C | GLY | A | 321 | 20.981 | 31.51 | 11.39 | 1.00 | 16.03 |
| 861 | O | GLY | A | 321 | 21.28 | 30.84 | 10.403 | 1.00 | 16.07 |
| 862 | N | VAL | A | 322 | 19.974 | 32.38 | 11.371 | 1.00 | 15.36 |
| 863 | CA | VAL | A | 322 | 19.162 | 32.5 | 10.167 | 1.00 | 16.30 |
| 864 | CB | VAL | A | 322 | 17.978 | 33.5 | 10.34 | 1.00 | 17.29 |
| 865 | CG1 | VAL | A | 322 | 18.478 | 34.9 | 10.603 | 1.00 | 16.70 |
| 866 | CG2 | VAL | A | 322 | 17.11 | 33.48 | 9.099 | 1.00 | 23.41 |
| 867 | C | VAL | A | 322 | 19.91 | 32.81 | 8.876 | 1.00 | 14.96 |
| 868 | O | VAL | A | 322 | 19.64 | 32.2 | 7.846 | 1.00 | 12.97 |
| 869 | N | TYR | A | 323 | 20.859 | 33.74 | 8.9 | 1.00 | 13.63 |
| 870 | CA | TYR | A | 323 | 21.544 | 34.05 | 7.651 | 1.00 | 14.50 |
| 871 | CB | TYR | A | 323 | 22.214 | 35.43 | 7.723 | 1.00 | 15.24 |
| 872 | CG | TYR | A | 323 | 21.209 | 36.52 | 7.416 | 1.00 | 15.95 |
| 873 | CD1 | TYR | A | 323 | 20.791 | 36.76 | 6.108 | 1.00 | 16.47 |
| 874 | CE1 | TYR | A | 323 | 19.768 | 37.66 | 5.83 | 1.00 | 17.10 |
| 875 | CD2 | TYR | A | 323 | 20.586 | 37.23 | 8.446 | 1.00 | 15.91 |
| 876 | CE2 | TYR | A | 323 | 19.569 | 38.14 | 8.182 | 1.00 | 15.61 |
| 877 | CZ | TYR | A | 323 | 19.161 | 38.35 | 6.875 | 1.00 | 16.58 |
| 878 | OH | TYR | A | 323 | 18.132 | 39.23 | 6.612 | 1.00 | 17.94 |
| 879 | C | TYR | A | 323 | 22.515 | 32.97 | 7.209 | 1.00 | 14.15 |
| 880 | O | TYR | A | 323 | 22.781 | 32.82 | 6.013 | 1.00 | 14.15 |
| 881 | N | GLU | A | 324 | 23.044 | 32.2 | 8.155 | 1.00 | 12.79 |
| 882 | CA | GLU | A | 324 | 23.94 | 31.11 | 7.77 | 1.00 | 13.63 |
| 883 | CB | GLU | A | 324 | 24.584 | 30.47 | 9.003 | 1.00 | 15.05 |
| 884 | OG | GLU | A | 324 | 25.608 | 31.38 | 9.689 | 1.00 | 15.42 |
| 885 | CD | GLU | A | 324 | 26.289 | 30.74 | 10.886 | 1.00 | 16.70 |
| 886 | OE1 | GLU | A | 324 | 26.102 | 29.53 | 11.119 | 1.00 | 17.95 |
| 887 | 0E2 | GLU | A | 324 | 27.021 | 31.46 | 11.592 | 1.00 | 16.57 |
| 888 | C | GLU | A | 324 | 23.055 | 30.1 | 7.03 | 1.00 | 14.04 |
| 889 | O | GLU | A | 324 | 23.448 | 29.55 | 6.004 | 1.00 | 12.76 |
| 890 | N | ALA | A | 325 | 21.842 | 29.9 | 7.545 | 1.00 | 13.17 |
| 891 | CA | ALA | A | 325 | 20.904 | 28.96 | 6.931 | 1.00 | 13.03 |
| 892 | CE | ALA | A | 325 | 19.699 | 28.73 | 7.849 | 1.00 | 13.75 |
| 893 | C | ALA | A | 325 | 20.434 | 29.49 | 5.59 | 1.00 | 13.24 |
| 894 | O | ALA | A | 325 | 20.326 | 28.75 | 4.614 | 1.00 | 12.79 |
| 895 | N | ILE | A | 326 | 20.147 | 30.79 | 5.542 | 1.00 | 12.44 |
| 896 | CA | ILE | A | 326 | 19.692 | 31.4 | 4.306 | 1.00 | 11.19 |
| 897 | CB | ILE | A | 326 | 19.357 | 32.9 | 4.539 | 1.00 | 10.99 |
| 898 | CG2 | ILE | A | 326 | 19.216 | 33.64 | 3.204 | 1.00 | 11.43 |
| 899 | CG1 | ILE | A | 326 | 18.052 | 33 | 5.334 | 1.00 | 12.81 |
| 900 | CD1 | ILE | A | 326 | 17.703 | 34.41 | 5.773 | 1.00 | 13.13 |
| 901 | C | ILE | A | 326 | 20.718 | 31.24 | 3.185 | 1.00 | 12.02 |
| 902 | O | ILE | A | 326 | 20.374 | 30.82 | 2.082 | 1.00 | 12.18 |
| 903 | N | PHE | A | 327 | 21.979 | 31.57 | 3.451 | 1.00 | 13.30 |
| 904 | CA | PHE | A | 327 | 22.982 | 31.43 | 2.395 | 1.00 | 13.60 |
| 905 | CB | PHE | A | 327 | 24.275 | 32.14 | 2.801 | 1.00 | 14.04 |
| 906 | CG | PHE | A | 327 | 24.093 | 33.61 | 3.059 | 1.00 | 13.96 |
| 907 | CD1 | PHE | A | 327 | 23.199 | 34.36 | 2.295 | 1.00 | 16.84 |
| 908 | CD2 | PHE | A | 327 | 24.819 | 34.25 | 4.054 | 1.00 | 15.29 |
| 909 | CE1 | PHE | A | 327 | 23.028 | 35.73 | 2.519 | 1.00 | 17.71 |
| 910 | CE2 | PHE | A | 327 | 24.657 | 35.62 | 4.284 | 1.00 | 18.04 |
| 911 | CZ | PHE | A | 327 | 23.755 | 36.35 | 3.511 | 1.00 | 15.92 |
| 912 | C | PHE | A | 327 | 23.232 | 29.96 | 2.027 | 1.00 | 13.74 |
| 913 | O | PHE | A | 327 | 23.537 | 29.64 | 0.878 | 1.00 | 13.80 |
| 914 | N | ALA | A | 328 | 23.104 | 29.05 | 2.994 | 1.00 | 13.03 |
| 915 | CA | ALA | A | 328 | 23.274 | 27.63 | 2.701 | 1.00 | 12.43 |
| 916 | CB | ALA | A | 328 | 23.255 | 26.8 | 4.003 | 1.00 | 11.53 |
| 917 | C | ALA | A | 328 | 22.127 | 27.18 | 1.783 | 1.00 | 13.70 |
| 918 | O | ALA | A | 328 | 22.342 | 26.48 | 0.788 | 1.00 | 13.68 |
| 919 | N | MET | A | 329 | 20.905 | 27.61 | 2.105 | 1.00 | 12.83 |
| 920 | CA | MET | A | 329 | 19.753 | 27.23 | 1.293 | 1.00 | 11.87 |
| 921 | CB | MET | A | 329 | 18.444 | 27.43 | 2.073 | 1.00 | 14.42 |
| 922 | CG | MET | A | 329 | 18.372 | 26.58 | 3.35 | 1.00 | 16.28 |
| 923 | SD | MET | A | 329 | 16.756 | 26.61 | 4.147 | 1.00 | 18.54 |
| 924 | CE | MET | A | 329 | 16.622 | 28.35 | 4.55 | 1.00 | 20.41 |
| 925 | C | MET | A | 329 | 19.691 | 27.94 | −0.056 | 1.00 | 13.87 |
| 926 | O | MET | A | 329 | 19.027 | 27.47 | −0.973 | 1.00 | 12.13 |
| 927 | N | LEU | A | 330 | 20.378 | 29.08 | −0.193 | 1.00 | 13.97 |
| 928 | CA | LEU | A | 330 | 20.373 | 29.77 | −1.484 | 1.00 | 15.41 |
| 929 | CB | LEU | A | 330 | 21.225 | 31.05 | −1.43 | 1.00 | 15.37 |
| 930 | CG | LEU | A | 330 | 20.549 | 32.29 | −0.84 | 1.00 | 18.20 |
| 931 | CD1 | LEU | A | 330 | 21.486 | 33.49 | −0.972 | 1.00 | 19.32 |
| 932 | CD2 | LEU | A | 330 | 19.227 | 32.56 | −1.56 | 1.00 | 18.93 |
| 933 | C | LEU | A | 330 | 20.934 | 28.85 | −2.557 | 1.00 | 13.60 |
| 934 | O | LEU | A | 330 | 20.493 | 28.86 | −3.707 | 1.00 | 15.79 |
| 935 | N | SER | A | 331 | 21.921 | 28.05 | −2.167 | 1.00 | 12.99 |
| 936 | CA | SER | A | 331 | 22.574 | 27.11 | −3.073 | 1.00 | 14.61 |
| 937 | CB | SER | A | 331 | 23.539 | 26.23 | −2.277 | 1.00 | 14.55 |
| 938 | OG | SER | A | 331 | 24.355 | 27.04 | −1.445 | 1.00 | 15.05 |
| 939 | C | SER | A | 331 | 21.553 | 26.24 | −3.8 | 1.00 | 15.16 |
| 940 | O | SER | A | 331 | 21.739 | 25.88 | −4.966 | 1.00 | 16.58 |
| 941 | N | SER | A | 332 | 20.474 | 25.89 | −3.105 | 1.00 | 14.05 |
| 942 | CA | SER | A | 332 | 19.441 | 25.04 | −3.685 | 1.00 | 14.37 |
| 943 | CB | SER | A | 332 | 18.391 | 24.7 | −2.63 | 1.00 | 16.17 |
| 944 | OG | SER | A | 332 | 18.992 | 24.05 | −1.527 | 1.00 | 14.97 |
| 945 | C | SER | A | 332 | 18.744 | 25.67 | −4.889 | 1.00 | 15.39 |
| 946 | O | SER | A | 332 | 18.262 | 24.95 | −5.768 | 1.00 | 15.48 |
| 947 | N | VAL | A | 333 | 18.677 | 26.99 | −4.925 | 1.00 | 16.08 |
| 948 | CA | VAL | A | 333 | 18.002 | 27.67 | −6.023 | 1.00 | 16.93 |
| 949 | CB | VAL | A | 333 | 16.985 | 28.72 | −5.502 | 1.00 | 18.06 |
| 950 | CG1 | VAL | A | 333 | 15.878 | 28.03 | −4.717 | 1.00 | 20.27 |
| 951 | CG2 | VAL | A | 333 | 17.692 | 29.76 | −4.636 | 1.00 | 19.05 |
| 952 | C | VAL | A | 333 | 18.968 | 28.36 | −6.971 | 1.00 | 16.26 |
| 953 | O | VAL | A | 333 | 18.55 | 29.14 | −7.819 | 1.00 | 17.98 |
| 954 | N | MET | A | 334 | 20.253 | 28.03 | −6.85 | 1.00 | 16.16 |
| 955 | CA | MET | A | 334 | 21.277 | 28.65 | −7.697 | 1.00 | 17.20 |
| 956 | CB | MET | A | 334 | 22.358 | 29.32 | −6.844 | 1.00 | 16.55 |
| 957 | CG | MET | A | 334 | 21.95 | 30.52 | −6.005 | 1.00 | 16.51 |
| 958 | SD | MET | A | 334 | 23.363 | 31.08 | −4.983 | 1.00 | 17.83 |
| 959 | CE | MET | A | 334 | 22.981 | 32.82 | −4.792 | 1.00 | 19.15 |
| 960 | C | MET | A | 334 | 22.02 | 27.67 | −8.596 | 1.00 | 19.13 |
| 961 | O | MET | A | 334 | 22.099 | 26.47 | −8.312 | 1.00 | 20.24 |
| 962 | N | ASN | A | 335 | 22.555 | 28.19 | −9.694 | 1.00 | 19.31 |
| 963 | CA | ASN | A | 335 | 23.426 | 27.42 | −10.57 | 1.00 | 20.10 |
| 964 | CB | ASN | A | 335 | 22.721 | 26.85 | −11.81 | 1.00 | 21.23 |
| 965 | CG | ASN | A | 335 | 22.146 | 27.9 | −12.73 | 1.00 | 20.10 |
| 966 | OD1 | ASN | A | 335 | 22.65 | 29.02 | −12.83 | 1.00 | 21.27 |
| 967 | ND2 | ASN | A | 335 | 21.089 | 27.53 | −13.45 | 1.00 | 23.09 |
| 968 | C | ASN | A | 335 | 24.501 | 28.46 | −10.9 | 1.00 | 21.45 |
| 969 | O | ASN | A | 335 | 24.433 | 29.58 | −10.42 | 1.00 | 19.65 |
| 970 | N | LYS | A | 336 | 25.49 | 28.1 | −11.71 | 1.00 | 21.83 |
| 971 | CA | LYS | A | 336 | 26.563 | 29.04 | −12.01 | 1.00 | 23.64 |
| 972 | CB | LYS | A | 336 | 27.652 | 28.36 | −12.84 | 1.00 | 26.31 |
| 973 | CG | LYS | A | 336 | 27.281 | 28.12 | −14.3 | 1.00 | 30.33 |
| 974 | CD | LYS | A | 336 | 28.501 | 27.65 | −15.09 | 1.00 | 34.09 |
| 975 | CE | LYS | A | 336 | 28.258 | 27.65 | −16.6 | 1.00 | 36.35 |
| 976 | NZ | LYS | A | 336 | 27.211 | 26.68 | −17.02 | 1.00 | 39.18 |
| 977 | G | LYS | A | 336 | 26.161 | 30.34 | −12.7 | 1.00 | 22.90 |
| 978 | O | LYS | A | 336 | 26.927 | 31.31 | −12.67 | 1.00 | 22.33 |
| 979 | N | ASP | A | 337 | 24.968 | 30.39 | −13.28 | 1.00 | 21.50 |
| 980 | CA | ASP | A | 337 | 24.547 | 31.58 | −14.01 | 1.00 | 21.46 |
| 981 | CB | ASP | A | 337 | 24.108 | 31.19 | −15.42 | 1.00 | 22.79 |
| 982 | CG | ASP | A | 337 | 25.223 | 30.54 | −16.21 | 1.00 | 24.81 |
| 983 | OD1 | ASP | A | 337 | 26.326 | 31.12 | −16.28 | 1.00 | 28.16 |
| 984 | OD2 | ASP | A | 337 | 24.997 | 29.45 | −16.76 | 1.00 | 26.38 |
| 985 | C | ASP | A | 337 | 23.466 | 32.47 | −13.4 | 1.00 | 19.81 |
| 986 | O | ASP | A | 337 | 23.137 | 33.51 | −13.96 | 1.00 | 18.99 |
| 987 | N | GLY | A | 338 | 22.91 | 32.06 | −12.26 | 1.00 | 18.40 |
| 988 | CA | GLY | A | 338 | 21.877 | 32.88 | −11.65 | 1.00 | 17.35 |
| 989 | C | GLY | A | 338 | 21.094 | 32.14 | −10.58 | 1.00 | 16.28 |
| 990 | O | GLY | A | 338 | 21.473 | 31.04 | −10.18 | 1.00 | 15.47 |
| 991 | N | MET | A | 339 | 20.001 | 32.74 | −10.13 | 1.00 | 17.05 |
| 992 | CA | MET | A | 339 | 19.183 | 32.11 | −9.093 | 1.00 | 17.55 |
| 993 | CB | MET | A | 339 | 19.562 | 32.65 | −7.705 | 1.00 | 19.90 |
| 994 | CG | MET | A | 339 | 19.227 | 34.1 | −7.44 | 1.00 | 22.04 |
| 995 | SD | MET | A | 339 | 19.422 | 34.52 | −5.667 | 1.00 | 24.36 |
| 996 | CE | MET | A | 339 | 17.862 | 34.13 | −5.025 | 1.00 | 23.38 |
| 997 | C | MET | A | 339 | 17.69 | 32.3 | −9.315 | 1.00 | 17.11 |
| 998 | O | MET | A | 339 | 17.25 | 33.26 | −9.943 | 1.00 | 17.45 |
| 999 | N | LEU | A | 340 | 16.91 | 31.35 | −8.803 | 1.00 | 16.64 |
| 1000 | CA | LEU | A | 340 | 15.462 | 31.41 | −8.914 | 1.00 | 16.30 |
| 1001 | CB | LEU | A | 340 | 14.848 | 30.05 | −8.591 | 1.00 | 17.42 |
| 1002 | CG | LEU | A | 340 | 15.023 | 28.95 | −9.62 | 1.00 | 19.04 |
| 1003 | CD1 | LEU | A | 340 | 14.407 | 27.65 | −9.08 | 1.00 | 18.84 |
| 1004 | CD2 | LEU | A | 340 | 14.353 | 29.37 | −10.92 | 1.00 | 17.82 |
| 1005 | C | LEU | A | 340 | 14.93 | 32.41 | −7.908 | 1.00 | 16.49 |
| 1006 | O | LEU | A | 340 | 15.41 | 32.46 | −6.776 | 1.00 | 17.61 |
| 1007 | N | VAL | A | 341 | 13.937 | 33.2 | −8.314 | 1.00 | 14.86 |
| 1008 | CA | VAL | A | 341 | 13.333 | 34.17 | −7.42 | 1.00 | 15.11 |
| 1009 | CB | VAL | A | 341 | 13.879 | 35.61 | −7.673 | 1.00 | 14.89 |
| 1010 | CG1 | VAL | A | 341 | 15.402 | 35.61 | −7.55 | 1.00 | 17.85 |
| 1011 | CG2 | VAL | A | 341 | 13.446 | 36.11 | −9.045 | 1.00 | 16.47 |
| 1012 | C | VAL | A | 341 | 11.821 | 34.19 | −7.593 | 1.00 | 14.36 |
| 1013 | O | VAL | A | 341 | 11.279 | 33.55 | −8.499 | 1.00 | 15.87 |
| 1014 | N | ALA | A | 342 | 11.15 | 34.93 | −6.717 | 1.00 | 13.97 |
| 1015 | CA | ALA | A | 342 | 9.701 | 35.07 | −6.753 | 1.00 | 14.91 |
| 1016 | CB | ALA | A | 342 | 9.29 | 35.9 | −7.966 | 1.00 | 15.58 |
| 1017 | C | ALA | A | 342 | 8.981 | 33.72 | −6.768 | 1.00 | 14.63 |
| 1018 | O | ALA | A | 342 | 8.176 | 33.44 | −7.664 | 1.00 | 14.60 |
| 1019 | N | TYR | A | 343 | 9.281 | 32.9 | −5.769 | 1.00 | 14.26 |
| 1020 | CA | TYR | A | 343 | 8.655 | 31.59 | −5.627 | 1.00 | 15.27 |
| 1021 | CB | TYR | A | 343 | 7.191 | 31.78 | −5.224 | 1.00 | 15.76 |
| 1022 | CG | TYR | A | 343 | 7.085 | 32.36 | −3.832 | 1.00 | 18.04 |
| 1023 | CD1 | TYR | A | 343 | 7.013 | 31.53 | −2.715 | 1.00 | 19.51 |
| 1024 | CE1 | TYR | A | 343 | 7.059 | 32.05 | −1.422 | 1.00 | 20.29 |
| 1025 | CD2 | TYR | A | 343 | 7.191 | 33.74 | −3.626 | 1.00 | 19.02 |
| 1026 | CE2 | TYR | A | 343 | 7.245 | 34.27 | −2.339 | 1.00 | 20.93 |
| 1027 | GZ | TYR | A | 343 | 7.183 | 33.43 | −1.244 | 1.00 | 20.76 |
| 1028 | OH | TYR | A | 343 | 7.283 | 33.95 | 0.027 | 1.00 | 21.61 |
| 1029 | C | TYR | A | 343 | 8.791 | 30.72 | −6.869 | 1.00 | 14.54 |
| 1030 | O | TYR | A | 343 | 7.847 | 30.04 | −7.289 | 1.00 | 15.22 |
| 1031 | N | GLY | A | 344 | 9.991 | 30.75 | −7.439 | 1.00 | 15.09 |
| 1032 | CA | GLY | A | 344 | 10.308 | 29.95 | −8.61 | 1.00 | 16.73 |
| 1033 | C | GLY | A | 344 | 9.804 | 30.45 | −9.945 | 1.00 | 18.37 |
| 1034 | O | GLY | A | 344 | 9.989 | 29.78 | −10.96 | 1.00 | 18.69 |
| 1035 | N | ASN | A | 345 | 9.187 | 31.63 | −9.969 | 1.00 | 18.57 |
| 1036 | CA | ASN | A | 345 | 8.659 | 32.13 | −11.23 | 1.00 | 18.81 |
| 1037 | CB | ASN | A | 345 | 7.317 | 32.82 | −11 | 1.00 | 21.87 |
| 1038 | CG | ASN | A | 345 | 6.156 | 31.83 | −11 | 1.00 | 24.72 |
| 1039 | OD1 | ASN | A | 345 | 5.003 | 32.23 | −10.97 | 1.00 | 32.52 |
| 1040 | ND2 | ASN | A | 345 | 6.465 | 30.55 | −11.03 | 1.00 | 29.15 |
| 1041 | C | ASN | A | 345 | 9.596 | 33.06 | −11.99 | 1.00 | 18.54 |
| 1042 | O | ASN | A | 345 | 9.275 | 33.49 | −13.09 | 1.00 | 16.73 |
| 1043 | N | GLY | A | 346 | 10.75 | 33.34 | −11.41 | 1.00 | 16.20 |
| 1044 | CA | GLY | A | 346 | 11.716 | 34.19 | −12.07 | 1.00 | 16.52 |
| 1045 | C | GLY | A | 346 | 13.11 | 33.64 | −11.89 | 1.00 | 16.47 |
| 1046 | O | GLY | A | 346 | 13.336 | 32.8 | −11.01 | 1.00 | 15.40 |
| 1047 | N | PHE | A | 347 | 14.042 | 34.09 | −12.73 | 1.00 | 15.82 |
| 1048 | CA | PHE | A | 347 | 15.435 | 33.66 | −12.65 | 1.00 | 15.40 |
| 1049 | CB | FIfE | A | 347 | 15.719 | 32.53 | −13.65 | 1.00 | 16.65 |
| 1050 | CG | PI1E | A | 347 | 17.154 | 32.06 | −13.64 | 1.00 | 20.70 |
| 1051 | CD1 | PHE | A | 347 | 18.106 | 32.65 | −14.48 | 1.00 | 19.82 |
| 1052 | CD2 | PI1E | A | 347 | 17.553 | 31.02 | −12.81 | 1.00 | 19.54 |
| 1053 | CE1 | PHE | A | 347 | 19.427 | 32.21 | −14.48 | 1.00 | 22.34 |
| 1054 | CE2 | PI1E | A | 347 | 18.879 | 30.57 | −12.8 | 1.00 | 22.09 |
| 1055 | CZ | PHE | A | 347 | 19.817 | 31.17 | −13.63 | 1.00 | 22.09 |
| 1056 | C | PHE | A | 347 | 16.261 | 34.9 | −13 | 1.00 | 16.41 |
| 1057 | O | PHE | A | 347 | 16.199 | 35.4 | −14.12 | 1.00 | 15.80 |
| 1058 | N | ILE | A | 348 | 17.012 | 35.39 | −12.02 | 1.00 | 14.27 |
| 1059 | CA | ILE | A | 348 | 17.825 | 36.58 | −12.24 | 1.00 | 15.49 |
| 1060 | CB | ILE | A | 348 | 17.683 | 37.55 | −11.03 | 1.00 | 15.76 |
| 1061 | CG2 | ILE | A | 348 | 18.184 | 36.9 | −9.764 | 1.00 | 17.30 |
| 1062 | CG1 | ILE | A | 348 | 18.433 | 38.85 | −11.31 | 1.00 | 15.24 |
| 1063 | CD1 | ILE | A | 348 | 17.966 | 40 | −10.42 | 1.00 | 16.30 |
| 1064 | C | ILE | A | 348 | 19.272 | 36.15 | −12.46 | 1.00 | 16.57 |
| 1065 | O | ILE | A | 348 | 19.833 | 35.38 | −11.68 | 1.00 | 16.99 |
| 1066 | N | THR | A | 349 | 19.884 | 36.65 | −13.53 | 1.00 | 15.69 |
| 1067 | CA | THR | A | 349 | 21.254 | 36.24 | −13.83 | 1.00 | 17.54 |
| 1068 | CB | THR | A | 349 | 21.628 | 36.5 | −15.32 | 1.00 | 18.08 |
| 1069 | OG1 | THR | A | 349 | 21.684 | 37.91 | −15.57 | 1.00 | 18.08 |
| 1070 | CG2 | THR | A | 349 | 20.615 | 35.86 | −16.23 | 1.00 | 19.40 |
| 1071 | C | THR | A | 349 | 22.34 | 36.85 | −12.97 | 1.00 | 17.79 |
| 1072 | O | THR | A | 349 | 22.279 | 38.01 | −12.56 | 1.00 | 17.03 |
| 1073 | N | ARG | A | 350 | 23.344 | 36.02 | −12.72 | 1.00 | 18.29 |
| 1074 | CA | ARG | A | 350 | 24.501 | 36.39 | −11.94 | 1.00 | 20.41 |
| 1075 | CB | ARG | A | 350 | 25.467 | 35.21 | −11.91 | 1.00 | 20.86 |
| 1076 | CG | ARG | A | 350 | 26.714 | 35.44 | −11.11 | 1.00 | 23.49 |
| 1077 | CD | ARG | A | 350 | 27.498 | 34.15 | −11.01 | 1.00 | 22.29 |
| 1078 | NE | ARG | A | 350 | 28.617 | 34.28 | −10.09 | 1.00 | 25.39 |
| 1079 | CZ | ARG | A | 350 | 29.426 | 33.28 | −9.755 | 1.00 | 25.74 |
| 1080 | NH1 | ARG | A | 350 | 29.237 | 32.07 | −10.26 | 1.00 | 25.82 |
| 1081 | NH2 | ARG | A | 350 | 30.417 | 33.5 | −8.906 | 1.00 | 25.63 |
| 1082 | C | ARG | A | 350 | 25.173 | 37.61 | −12.58 | 1.00 | 20.85 |
| 1083 | O | ARG | A | 350 | 25.624 | 38.52 | −11.89 | 1.00 | 20.39 |
| 1084 | N | GLU | A | 351 | 25.226 | 37.61 | −13.91 | 1.00 | 21.71 |
| 1085 | CA | GLU | A | 351 | 25.86 | 38.71 | −14.63 | 1.00 | 23.42 |
| 1086 | CB | GLU | A | 351 | 26.045 | 38.34 | −16.1 | 1.00 | 26.93 |
| 1087 | CG | GLU | A | 351 | 27.152 | 37.31 | −16.32 | 1.00 | 31.26 |
| 1088 | CD | GLU | A | 351 | 28.504 | 37.81 | −15.83 | 1.00 | 33.51 |
| 1089 | OE1 | GLU | A | 351 | 28.97 | 38.86 | −16.3 | 1.00 | 36.23 |
| 1090 | 0E2 | GLU | A | 351 | 29.103 | 37.14 | −14.96 | 1.00 | 36.72 |
| 1091 | C | GLU | A | 351 | 25.106 | 40.04 | −14.51 | 1.00 | 21.78 |
| 1092 | O | GLU | A | 351 | 25.723 | 41.1 | −14.42 | 1.00 | 23.51 |
| 1093 | N | PHE | A | 352 | 23.779 | 39.98 | −14.49 | 1.00 | 20.79 |
| 1094 | CA | PHE | A | 352 | 22.988 | 41.19 | −14.35 | 1.00 | 20.13 |
| 1095 | CB | PHE | A | 352 | 21.496 | 40.88 | −14.53 | 1.00 | 21.21 |
| 1096 | CG | PHE | A | 352 | 20.603 | 42.07 | −14.3 | 1.00 | 22.31 |
| 1097 | CD1 | PHE | A | 352 | 20.806 | 43.26 | −14.99 | 1.00 | 22.58 |
| 1098 | CD2 | PHE | A | 352 | 19.561 | 42 | −13.38 | 1.00 | 22.12 |
| 1099 | CE1 | PHE | A | 352 | 19.985 | 44.36 | −14.77 | 1.00 | 22.50 |
| 1100 | CE2 | PHE | A | 352 | 18.734 | 43.1 | −13.15 | 1.00 | 21.07 |
| 1101 | CZ | PHE | A | 352 | 18.947 | 44.28 | −13.85 | 1.00 | 23.38 |
| 1102 | C | PHE | A | 352 | 23.246 | 41.79 | −12.97 | 1.00 | 20.06 |
| 1103 | O | PHE | A | 352 | 23.411 | 43 | −12.82 | 1.00 | 18.10 |
| 1104 | N | LEU | A | 353 | 23.291 | 40.93 | −11.95 | 1.00 | 19.09 |
| 1105 | CA | LEU | A | 353 | 23.537 | 41.39 | −10.6 | 1.00 | 19.98 |
| 1106 | CB | LEU | A | 353 | 23.431 | 40.22 | −9.622 | 1.00 | 18.81 |
| 1107 | CG | LEU | A | 353 | 21.998 | 39.71 | −9.458 | 1.00 | 17.36 |
| 1108 | CD1 | LEU | A | 353 | 22.019 | 38.3 | −8.885 | 1.00 | 17.66 |
| 1109 | CD2 | LEU | A | 353 | 21.208 | 40.67 | −8.55 | 1.00 | 18.64 |
| 1110 | C | LEU | A | 353 | 24.895 | 42.08 | −10.45 | 1.00 | 20.25 |
| 1111 | O | LEU | A | 353 | 25.003 | 43.12 | −9.792 | 1.00 | 20.17 |
| 1112 | N | LYS | A | 354 | 25.931 | 41.53 | −11.07 | 1.00 | 22.58 |
| 1113 | CA | LYS | A | 354 | 27.248 | 42.15 | −10.97 | 1.00 | 25.12 |
| 1114 | CB | LYS | A | 354 | 28.334 | 41.15 | −11.39 | 1.00 | 28.30 |
| 1115 | CG | LYS | A | 354 | 28.091 | 40.46 | −12.71 | 1.00 | 31.49 |
| 1116 | CD | LYS | A | 354 | 28.929 | 39.19 | −12.83 | 1.00 | 34.20 |
| 1117 | CE | LYS | A | 354 | 30.416 | 39.46 | −12.65 | 1.00 | 32.93 |
| 1118 | NZ | LYS | A | 354 | 31.234 | 38.26 | −12.95 | 1.00 | 33.54 |
| 1119 | C | LYS | A | 354 | 27.349 | 43.45 | −11.77 | 1.00 | 26.38 |
| 1120 | O | LYS | A | 354 | 28.23 | 44.28 | −11.51 | 1.00 | 28.05 |
| 1121 | N | SER | A | 355 | 26.425 | 43.64 | −12.71 | 1.00 | 25.99 |
| 1122 | CA | SER | A | 355 | 26.404 | 44.84 | −13.55 | 1.00 | 25.78 |
| 1123 | CB | SER | A | 355 | 25.604 | 44.6 | −14.83 | 1.00 | 27.38 |
| 1124 | OG | SER | A | 355 | 24.213 | 44.75 | −14.59 | 1.00 | 27.61 |
| 1125 | C | SER | A | 355 | 25.786 | 46.03 | −12.82 | 1.00 | 25.54 |
| 1126 | O | SER | A | 355 | 25.874 | 47.17 | −13.28 | 1.00 | 24.80 |
| 1127 | N | LEU | A | 356 | 25.148 | 45.76 | −11.69 | 1.00 | 21.74 |
| 1128 | CA | LEU | A | 356 | 24.524 | 46.82 | −10.92 | 1.00 | 21.47 |
| 1129 | CB | LEU | A | 356 | 23.682 | 46.23 | −9.789 | 1.00 | 20.68 |
| 1130 | CG | LEU | A | 356 | 22.535 | 45.31 | −10.21 | 1.00 | 17.94 |
| 1131 | CD1 | LEU | A | 356 | 21.93 | 44.68 | −8.965 | 1.00 | 17.43 |
| 1132 | CD2 | LEU | A | 356 | 21.487 | 46.11 | −10.99 | 1.00 | 18.91 |
| 1133 | C | LEU | A | 356 | 25.576 | 47.75 | −10.34 | 1.00 | 22.34 |
| 1134 | O | LEU | A | 356 | 26.765 | 47.43 | −10.3 | 1.00 | 21.32 |
| 1135 | N | ARG | A | 357 | 25.115 | 48.9 | −9.876 | 1.00 | 23.03 |
| 1136 | CA | ARG | A | 357 | 25.973 | 49.9 | −9.272 | 1.00 | 24.90 |
| 1137 | CB | ARG | A | 357 | 25.177 | 51.21 | −9.192 | 1.00 | 26.16 |
| 1138 | CG | ARG | A | 357 | 25.46 | 52.09 | −7.996 | 1.00 | 28.63 |
| 1139 | CD | ARG | A | 357 | 24.766 | 53.44 | −8.141 | 1.00 | 27.93 |
| 1140 | NE | ARG | A | 357 | 23.501 | 53.56 | −7.416 | 1.00 | 25.84 |
| 1141 | CZ | ARG | A | 357 | 23.381 | 53.49 | −6.094 | 1.00 | 25.53 |
| 1142 | NH1 | ARG | A | 357 | 24.449 | 53.28 | −5.332 | 1.00 | 26.92 |
| 1143 | NH2 | ARG | A | 357 | 22.199 | 53.68 | −5.527 | 1.00 | 26.63 |
| 1144 | C | ARG | A | 357 | 26.422 | 49.46 | −7.879 | 1.00 | 23.21 |
| 1145 | O | ARG | A | 357 | 25.675 | 48.78 | −7.177 | 1.00 | 22.61 |
| 1146 | N | LYS | A | 358 | 27.646 | 49.81 | −7.483 | 1.00 | 23.12 |
| 1147 | CA | LYS | A | 358 | 28.109 | 49.46 | −6.141 | 1.00 | 22.49 |
| 1148 | CB | LYS | A | 358 | 29.576 | 49.87 | −5.923 | 1.00 | 23.58 |
| 1149 | CG | LYS | A | 358 | 30.609 | 49.05 | −6.695 | 1.00 | 22.91 |
| 1150 | CD | LYS | A | 358 | 32.035 | 49.59 | −6.461 | 1.00 | 25.76 |
| 1151 | CE | LYS | A | 358 | 32.702 | 48.99 | −5.225 | 1.00 | 26.68 |
| 1152 | NZ | LYS | A | 358 | 33.206 | 47.61 | −5.476 | 1.00 | 28.29 |
| 1153 | C | LYS | A | 358 | 27.219 | 50.27 | −5.198 | 1.00 | 22.76 |
| 1154 | O | LYS | A | 358 | 26.764 | 51.36 | −5.548 | 1.00 | 24.51 |
| 1155 | N | PRO | A | 359 | 26.982 | 49.76 | −3.98 | 1.00 | 21.69 |
| 1156 | CD | PRO | A | 359 | 26.263 | 50.5 | −2.93 | 1.00 | 23.55 |
| 1157 | CA | PRO | A | 359 | 27.492 | 48.49 | −3.451 | 1.00 | 21.20 |
| 1158 | CB | PRO | A | 359 | 27.626 | 48.8 | −1.973 | 1.00 | 23.13 |
| 1159 | CG | PRO | A | 359 | 26.366 | 49.56 | −1.719 | 1.00 | 22.88 |
| 1160 | C | PRO | A | 359 | 26.548 | 47.32 | −3.7 | 1.00 | 20.65 |
| 1161 | O | PRO | A | 359 | 26.81 | 46.19 | −3.257 | 1.00 | 20.59 |
| 1162 | N | PHE | A | 360 | 25.454 | 47.57 | −4.404 | 1.00 | 19.16 |
| 1163 | CA | PI1E | A | 360 | 24.473 | 46.52 | −4.671 | 1.00 | 18.85 |
| 1164 | CB | PHE | A | 360 | 23.25 | 47.14 | −5.355 | 1.00 | 20.28 |
| 1165 | CG | PHE | A | 360 | 22.536 | 48.14 | −4.492 | 1.00 | 19.94 |
| 1166 | CD1 | PHE | A | 360 | 21.728 | 47.72 | −3.435 | 1.00 | 18.19 |
| 1167 | CD2 | PI1E | A | 360 | 22.72 | 49.51 | −4.692 | 1.00 | 18.89 |
| 1168 | CE1 | PHE | A | 360 | 21.115 | 48.65 | −2.586 | 1.00 | 19.37 |
| 1169 | CE2 | PHE | A | 360 | 22.113 | 50.43 | −3.851 | 1.00 | 19.54 |
| 1170 | CZ | PHE | A | 360 | 21.309 | 50 | −2.794 | 1.00 | 19.87 |
| 1171 | C | PHE | A | 360 | 25.038 | 45.36 | −5.473 | 1.00 | 19.99 |
| 1172 | O | PHE | A | 360 | 24.618 | 44.21 | −5.292 | 1.00 | 19.11 |
| 1173 | N | GYS | A | 361 | 26.009 | 45.64 | −6.338 | 1.00 | 17.57 |
| 1174 | CA | CYS | A | 361 | 26.619 | 44.58 | −7.14 | 1.00 | 18.30 |
| 1175 | CB | CYS | A | 361 | 27.353 | 45.19 | −8.34 | 1.00 | 18.99 |
| 1176 | SG | CYS | A | 361 | 28.657 | 46.36 | −7.884 | 1.00 | 20.67 |
| 1177 | C | CYS | A | 361 | 27.598 | 43.76 | −6.305 | 1.00 | 18.14 |
| 1178 | O | CYS | A | 361 | 28.112 | 42.75 | −6.769 | 1.00 | 17.82 |
| 1179 | N | ASP | A | 362 | 27.837 | 44.19 | −5.068 | 1.00 | 18.21 |
| 1180 | CA | ASP | A | 362 | 28.777 | 43.48 | −4.205 | 1.00 | 18.60 |
| 1181 | CB | ASP | A | 362 | 29.631 | 44.49 | −3.435 | 1.00 | 20.06 |
| 1182 | CG | ASP | A | 362 | 30.428 | 45.4 | −4.359 | 1.00 | 20.29 |
| 1183 | OD1 | ASP | A | 362 | 31.017 | 44.88 | −5.319 | 1.00 | 21.00 |
| 1184 | OD2 | ASP | A | 362 | 30.464 | 46.62 | −4.121 | 1.00 | 22.31 |
| 1185 | C | ASP | A | 362 | 28.105 | 42.54 | −3.22 | 1.00 | 19.14 |
| 1186 | O | ASP | A | 362 | 28.767 | 41.93 | −2.374 | 1.00 | 20.18 |
| 1187 | N | ILE | A | 363 | 26.791 | 42.41 | −3.344 | 1.00 | 17.15 |
| 1188 | CA | ILE | A | 363 | 26.021 | 41.56 | −2.446 | 1.00 | 18.64 |
| 1189 | CB | ILE | A | 363 | 24.536 | 42 | −2.418 | 1.00 | 19.28 |
| 1190 | CG2 | ILE | A | 363 | 23.729 | 41.06 | −1.525 | 1.00 | 17.16 |
| 1191 | CG1 | ILE | A | 363 | 24.424 | 43.44 | −1.922 | 1.00 | 19.43 |
| 1192 | CD1 | ILE | A | 363 | 23.004 | 43.98 | −1.928 | 1.00 | 19.79 |
| 1193 | C | ILE | A | 363 | 26.039 | 40.08 | −2.781 | 1.00 | 18.41 |
| 1194 | O | ILE | A | 363 | 26.419 | 39.25 | −1.954 | 1.00 | 17.58 |
| 1195 | N | MET | A | 364 | 25.635 | 39.75 | −4.002 | 1.00 | 20.04 |
| 1196 | CA | MET | A | 364 | 25.511 | 38.35 | −4.409 | 1.00 | 18.56 |
| 1197 | CB | MET | A | 364 | 24.434 | 38.24 | −5.491 | 1.00 | 19.49 |
| 1198 | CG | MET | A | 364 | 23.037 | 38.63 | −5.018 | 1.00 | 20.24 |
| 1199 | SD | MET | A | 364 | 22.485 | 37.68 | −3.579 | 1.00 | 20.20 |
| 1200 | CE | MET | A | 364 | 22.609 | 36.01 | −4.236 | 1.00 | 16.96 |
| 1201 | C | MET | A | 364 | 26.706 | 37.5 | −4.823 | 1.00 | 18.99 |
| 1202 | O | MET | A | 364 | 26.654 | 36.29 | −4.644 | 1.00 | 17.53 |
| 1203 | N | GLU | A | 365 | 27.771 | 38.09 | −5.369 | 1.00 | 19.55 |
| 1204 | CA | GLU | A | 365 | 28.91 | 37.27 | −5.794 | 1.00 | 19.92 |
| 1205 | CB | GLU | A | 365 | 30.078 | 38.13 | −6.287 | 1.00 | 21.97 |
| 1206 | CG | GLU | A | 365 | 30.022 | 38.5 | −7.756 | 1.00 | 26.26 |
| 1207 | CD | GLU | A | 365 | 29.832 | 37.29 | −8.656 | 1.00 | 27.30 |
| 1208 | OE1 | GLU | A | 365 | 28.693 | 37.06 | −9.104 | 1.00 | 26.68 |
| 1209 | OE2 | GLU | A | 365 | 30.817 | 36.57 | −8.909 | 1.00 | 29.91 |
| 1210 | C | GLU | A | 365 | 29.436 | 36.29 | −4.748 | 1.00 | 18.52 |
| 1211 | O | GLU | A | 365 | 29.628 | 35.11 | −5.042 | 1.00 | 18.23 |
| 1212 | N | PRO | A | 366 | 29.689 | 36.76 | −3.516 | 1.00 | 18.98 |
| 1213 | CD | PRO | A | 366 | 29.537 | 38.13 | −2.997 | 1.00 | 20.10 |
| 1214 | CA | PRO | A | 366 | 30.198 | 35.86 | −2.47 | 1.00 | 19.09 |
| 1215 | CB | PRO | A | 366 | 30.29 | 36.78 | −1.251 | 1.00 | 20.02 |
| 1216 | CG | PRO | A | 366 | 30.52 | 38.14 | −1.862 | 1.00 | 20.53 |
| 1217 | C | PRO | A | 366 | 29.278 | 34.66 | −2.211 | 1.00 | 17.84 |
| 1218 | O | PRO | A | 366 | 29.736 | 33.57 | −1.851 | 1.00 | 15.96 |
| 1219 | N | LYS | A | 367 | 27.98 | 34.88 | −2.388 | 1.00 | 17.85 |
| 1220 | CA | LYS | A | 367 | 26.992 | 33.82 | −2.17 | 1.00 | 16.69 |
| 1221 | CB | LYS | A | 367 | 25.603 | 34.44 | −1.964 | 1.00 | 17.55 |
| 1222 | CG | LYS | A | 367 | 25.415 | 35.17 | −0.624 | 1.00 | 16.13 |
| 1223 | CD | LYS | A | 367 | 26.254 | 36.45 | −0.534 | 1.00 | 18.57 |
| 1224 | CE | LYS | A | 367 | 25.819 | 37.34 | 0.626 | 1.00 | 16.06 |
| 1225 | NZ | LYS | A | 367 | 26.671 | 38.58 | 0.743 | 1.00 | 15.29 |
| 1226 | C | LYS | A | 367 | 26.963 | 32.84 | −3.337 | 1.00 | 17.68 |
| 1227 | O | LYS | A | 367 | 26.783 | 31.63 | −3.142 | 1.00 | 17.04 |
| 1228 | N | PHE | A | 368 | 27.127 | 33.35 | −4.555 | 1.00 | 17.39 |
| 1229 | CA | PHE | A | 368 | 27.156 | 32.47 | −5.719 | 1.00 | 16.62 |
| 1230 | CB | PHE | A | 368 | 27.169 | 33.28 | −7.023 | 1.00 | 16.38 |
| 1231 | CG | PHE | A | 368 | 25.796 | 33.65 | −7.527 | 1.00 | 18.08 |
| 1232 | CD1 | PHE | A | 368 | 24.936 | 32.68 | −8.039 | 1.00 | 17.39 |
| 1233 | CD2 | PHE | A | 368 | 25.364 | 34.97 | −7.495 | 1.00 | 16.89 |
| 1234 | CE1 | PI1E | A | 368 | 23.67 | 33.02 | −8.507 | 1.00 | 17.41 |
| 1235 | CE2 | PHE | A | 368 | 24.102 | 35.32 | −7.96 | 1.00 | 16.42 |
| 1236 | CZ | PHE | A | 368 | 23.253 | 34.35 | −8.467 | 1.00 | 16.76 |
| 1237 | C | PHE | A | 368 | 28.426 | 31.63 | −5.6 | 1.00 | 16.15 |
| 1238 | O | PHE | A | 368 | 28.407 | 30.42 | −5.842 | 1.00 | 16.24 |
| 1239 | N | ASP | A | 369 | 29.53 | 32.26 | −5.206 | 1.00 | 18.27 |
| 1240 | CA | ASP | A | 369 | 30.783 | 31.52 | −5.049 | 1.00 | 18.69 |
| 1241 | CB | ASP | A | 369 | 31.952 | 32.46 | −4.709 | 1.00 | 20.51 |
| 1242 | CG | ASP | A | 369 | 32.444 | 33.24 | −5.922 | 1.00 | 22.65 |
| 1243 | OD1 | ASP | A | 369 | 32.327 | 32.72 | −7.05 | 1.00 | 23.09 |
| 1244 | OD2 | ASP | A | 369 | 32.959 | 34.36 | −5.748 | 1.00 | 27.87 |
| 1245 | C | ASP | A | 369 | 30.638 | 30.44 | −3.979 | 1.00 | 18.16 |
| 1246 | O | ASP | A | 369 | 31.117 | 29.32 | −4.159 | 1.00 | 17.43 |
| 1247 | N | PHE | A | 370 | 29.969 | 30.76 | −2.872 | 1.00 | 16.51 |
| 1248 | CA | PHE | A | 370 | 29.785 | 29.75 | −1.833 | 1.00 | 17.11 |
| 1249 | CB | PHE | A | 370 | 29.146 | 30.33 | −0.571 | 1.00 | 17.43 |
| 1250 | CG | PHE | A | 370 | 28.76 | 29.28 | 0.443 | 1.00 | 16.12 |
| 1251 | CD1 | PHE | A | 370 | 27.548 | 28.61 | 0.339 | 1.00 | 15.11 |
| 1252 | CD2 | PHE | A | 370 | 29.643 | 28.93 | 1.461 | 1.00 | 16.71 |
| 1253 | CE1 | PHE | A | 370 | 27.219 | 27.58 | 1.231 | 1.00 | 14.11 |
| 1254 | CE2 | PHE | A | 370 | 29.323 | 27.9 | 2.356 | 1.00 | 15.23 |
| 1255 | GZ | PHE | A | 370 | 28.109 | 27.23 | 2.238 | 1.00 | 11.27 |
| 1256 | C | PHE | A | 370 | 28.902 | 28.61 | −2.342 | 1.00 | 16.98 |
| 1257 | O | PHE | A | 370 | 29.21 | 27.44 | −2.136 | 1.00 | 16.87 |
| 1258 | N | ALA | A | 371 | 27.802 | 28.97 | −2.995 | 1.00 | 16.32 |
| 1259 | CA | ALA | A | 371 | 26.854 | 27.99 | −3.518 | 1.00 | 19.25 |
| 1260 | CB | ALA | A | 371 | 25.659 | 28.71 | −4.147 | 1.00 | 17.22 |
| 1261 | C | ALA | A | 371 | 27.467 | 27.02 | −4.529 | 1.00 | 19.60 |
| 1262 | O | ALA | A | 371 | 27.09 | 25.85 | −4.58 | 1.00 | 20.69 |
| 1263 | N | MET | A | 372 | 28.401 | 27.51 | −5.337 | 1.00 | 19.90 |
| 1264 | CA | MET | A | 372 | 29.029 | 26.66 | −6.342 | 1.00 | 23.22 |
| 1265 | CB | MET | A | 372 | 29.948 | 27.48 | −7.242 | 1.00 | 25.16 |
| 1266 | CG | MET | A | 372 | 29.599 | 27.38 | −8.714 | 1.00 | 31.86 |
| 1267 | SD | MET | A | 372 | 27.844 | 27.66 | −9.043 | 1.00 | 35.24 |
| 1268 | CE | MET | A | 372 | 27.242 | 26 | −9.114 | 1.00 | 37.40 |
| 1269 | C | MET | A | 372 | 29.817 | 25.54 | −5.671 | 1.00 | 22.48 |
| 1270 | O | MET | A | 372 | 29.732 | 24.38 | −6.074 | 1.00 | 22.07 |
| 1271 | N | LYS | A | 373 | 30.577 | 25.89 | −4.641 | 1.00 | 21.68 |
| 1272 | CA | LYS | A | 373 | 31.347 | 24.88 | −3.929 | 1.00 | 22.55 |
| 1273 | CB | LYS | A | 373 | 32.445 | 25.56 | −3.098 | 1.00 | 24.59 |
| 1274 | CG | LYS | A | 373 | 33.599 | 26.04 | −3.977 | 1.00 | 28.52 |
| 1275 | CD | LYS | A | 373 | 34.722 | 26.71 | −3.205 | 1.00 | 31.12 |
| 1276 | CE | LYS | A | 373 | 34.309 | 28.07 | −2.681 | 1.00 | 33.08 |
| 1277 | NZ | LYS | A | 373 | 35.503 | 28.85 | −2.253 | 1.00 | 34.91 |
| 1278 | C | LYS | A | 373 | 30.432 | 24.02 | −3.059 | 1.00 | 22.72 |
| 1279 | O | LYS | A | 373 | 30.685 | 22.83 | −2.874 | 1.00 | 22.63 |
| 1280 | N | PHE | A | 374 | 29.355 | 24.61 | −2.545 | 1.00 | 21.24 |
| 1281 | CA | PHE | A | 374 | 28.425 | 23.86 | −1.708 | 1.00 | 20.87 |
| 1282 | CB | PHE | A | 374 | 27.417 | 24.79 | −1.031 | 1.00 | 19.09 |
| 1283 | CG | PHE | A | 374 | 26.647 | 24.15 | 0.097 | 1.00 | 19.22 |
| 1284 | CD1 | PHE | A | 374 | 27.258 | 23.91 | 1.321 | 1.00 | 19.37 |
| 1285 | CD2 | PHE | A | 374 | 25.306 | 23.81 | −0.064 | 1.00 | 19.20 |
| 1286 | CE1 | PHE | A | 374 | 26.543 | 23.33 | 2.376 | 1.00 | 18.07 |
| 1287 | CE2 | PHE | A | 374 | 24.579 | 23.23 | 0.98 | 1.00 | 18.27 |
| 1288 | GZ | PHE | A | 374 | 25.2 | 22.99 | 2.203 | 1.00 | 18.45 |
| 1289 | C | PHE | A | 374 | 27.667 | 22.82 | −2.536 | 1.00 | 20.34 |
| 1290 | O | PHE | A | 374 | 27.503 | 21.67 | −2.115 | 1.00 | 19.46 |
| 1291 | N | ASN | A | 375 | 27.204 | 23.23 | −3.714 | 1.00 | 20.08 |
| 1292 | CA | ASN | A | 375 | 26.458 | 22.35 | −4.607 | 1.00 | 21.22 |
| 1293 | CB | ASN | A | 375 | 25.903 | 23.15 | −5.791 | 1.00 | 21.85 |
| 1294 | CG | ASN | A | 375 | 24.613 | 23.89 | −5.449 | 1.00 | 22.68 |
| 1295 | ODI | ASN | A | 375 | 24.238 | 24.85 | −6.119 | 1.00 | 22.40 |
| 1296 | ND2 | ASN | A | 375 | 23.922 | 23.42 | −4.415 | 1.00 | 20.32 |
| 1297 | C | ASN | A | 375 | 27.322 | 21.2 | −5.119 | 1.00 | 21.97 |
| 1298 | O | ASN | A | 375 | 26.807 | 20.14 | −5.483 | 1.00 | 22.05 |
| 1299 | N | ALA | A | 376 | 28.632 | 21.41 | −5.142 | 1.00 | 20.80 |
| 1300 | CA | ALA | A | 376 | 29.561 | 20.38 | −5.597 | 1.00 | 22.52 |
| 1301 | CB | ALA | A | 376 | 30.974 | 20.95 | −5.677 | 1.00 | 22.39 |
| 1302 | C | ALA | A | 376 | 29.52 | 19.2 | −4.643 | 1.00 | 23.80 |
| 1303 | O | ALA | A | 376 | 30.008 | 18.11 | −4.971 | 1.00 | 24.70 |
| 1304 | N | LEU | A | 377 | 28.943 | 19.4 | −3.459 | 1.00 | 22.37 |
| 1305 | CA | LEU | A | 377 | 28.824 | 18.34 | −2.466 | 1.00 | 23.08 |
| 1306 | CB | LEU | A | 377 | 28.577 | 18.93 | −1.072 | 1.00 | 23.25 |
| 1307 | CG | LEU | A | 377 | 29.719 | 19.77 | −0.485 | 1.00 | 23.11 |
| 1308 | CD1 | LEU | A | 377 | 29.376 | 20.19 | 0.94 | 1.00 | 22.57 |
| 1309 | CD2 | LEU | A | 377 | 31.001 | 18.95 | −0.488 | 1.00 | 24.48 |
| 1310 | C | LEU | A | 377 | 27.686 | 17.39 | −2.833 | 1.00 | 22.79 |
| 1311 | O | LEU | A | 377 | 27.549 | 16.31 | −2.249 | 1.00 | 22.44 |
| 1312 | N | GLU | A | 378 | 26.865 | 17.8 | −3.793 | 1.00 | 22.82 |
| 1313 | CA | GLU | A | 378 | 25.75 | 16.99 | −4.256 | 1.00 | 24.48 |
| 1314 | CB | GLU | A | 378 | 26.291 | 15.74 | −4.949 | 1.00 | 26.99 |
| 1315 | CG | GLU | A | 378 | 26.137 | 15.73 | −6.449 | 1.00 | 33.00 |
| 1316 | CD | GLU | A | 378 | 26.938 | 14.62 | −7.093 | 1.00 | 35.36 |
| 1317 | OE1 | GLU | A | 378 | 27.012 | 13.52 | −6.502 | 1.00 | 37.48 |
| 1318 | OE2 | GLU | A | 378 | 27.487 | 14.84 | −8.188 | 1.00 | 36.92 |
| 1319 | C | GLU | A | 378 | 24.786 | 16.57 | −3.153 | 1.00 | 22.38 |
| 1320 | O | GLU | A | 378 | 24.3 | 15.44 | −3.142 | 1.00 | 23.39 |
| 1321 | N | LEU | A | 379 | 24.511 | 17.47 | −2.219 | 1.00 | 21.57 |
| 1322 | CA | LEU | A | 379 | 23.589 | 17.16 | −1.134 | 1.00 | 18.06 |
| 1323 | CB | LEU | A | 379 | 23.714 | 18.18 | −0.004 | 1.00 | 18.81 |
| 1324 | CG | LEU | A | 379 | 25.061 | 18.39 | 0.688 | 1.00 | 16.97 |
| 1325 | CD1 | LEU | A | 379 | 24.863 | 19.34 | 1.858 | 1.00 | 16.61 |
| 1326 | GD2 | LEU | A | 379 | 25.599 | 17.06 | 1.19 | 1.00 | 18.25 |
| 1327 | C | LEU | A | 379 | 22.153 | 17.19 | −1.64 | 1.00 | 18.75 |
| 1328 | O | LEU | A | 379 | 21.855 | 17.83 | −2.651 | 1.00 | 19.00 |
| 1329 | N | ASP | A | 380 | 21.273 | 16.48 | −0.947 | 1.00 | 19.17 |
| 1330 | CA | ASP | A | 380 | 19.862 | 16.48 | −1.296 | 1.00 | 18.60 |
| 1331 | CB | ASP | A | 380 | 19.339 | 15.06 | −1.59 | 1.00 | 19.63 |
| 1332 | CG | ASP | A | 380 | 19.486 | 14.12 | −0.42 | 1.00 | 21.42 |
| 1333 | ODI | ASP | A | 380 | 19.266 | 14.55 | 0.728 | 1.00 | 18.99 |
| 1334 | OD2 | ASP | A | 380 | 19.801 | 12.93 | −0.657 | 1.00 | 24.09 |
| 1335 | C | ASP | A | 380 | 19.153 | 17.09 | −0.092 | 1.00 | 18.66 |
| 1336 | O | ASP | A | 380 | 19.791 | 17.39 | 0.917 | 1.00 | 19.31 |
| 1337 | N | ASP | A | 381 | 17.846 | 17.28 | −0.191 | 1.00 | 18.35 |
| 1338 | CA | ASP | A | 381 | 17.097 | 17.9 | 0.894 | 1.00 | 18.08 |
| 1339 | CB | ASP | A | 381 | 15.64 | 18.08 | 0.473 | 1.00 | 20.09 |
| 1340 | CG | ASP | A | 381 | 15.489 | 19.09 | −0.658 | 1.00 | 21.21 |
| 1341 | OD1 | ASP | A | 381 | 15.934 | 20.25 | −0.482 | 1.00 | 20.02 |
| 1342 | OD2 | ASP | A | 381 | 14.932 | 18.73 | −1.719 | 1.00 | 22.56 |
| 1343 | C | ASP | A | 381 | 17.185 | 17.17 | 2.234 | 1.00 | 17.57 |
| 1344 | O | ASP | A | 381 | 17.159 | 17.81 | 3.284 | 1.00 | 17.01 |
| 1345 | N | SER | A | 382 | 17.297 | 15.84 | 2.219 | 1.00 | 16.66 |
| 1346 | CA | SER | A | 382 | 17.403 | 15.13 | 3.49 | 1.00 | 17.74 |
| 1347 | CB | SER | A | 382 | 17.364 | 13.6 | 3.284 | 1.00 | 16.26 |
| 1348 | OG | SER | A | 382 | 18.502 | 13.1 | 2.606 | 1.00 | 18.96 |
| 1349 | G | SER | A | 382 | 18.695 | 15.55 | 4.197 | 1.00 | 17.27 |
| 1350 | O | SER | A | 382 | 18.722 | 15.69 | 5.419 | 1.00 | 19.27 |
| 1351 | N | ASP | A | 383 | 19.759 | 15.77 | 3.427 | 1.00 | 16.48 |
| 1352 | CA | ASP | A | 383 | 21.042 | 16.2 | 4.003 | 1.00 | 15.88 |
| 1353 | CB | ASP | A | 383 | 22.172 | 16.15 | 2.967 | 1.00 | 15.19 |
| 1354 | CG | ASP | A | 383 | 22.32 | 14.8 | 2.306 | 1.00 | 18.19 |
| 1355 | OD1 | ASP | A | 383 | 22.365 | 13.78 | 3.025 | 1.00 | 19.73 |
| 1356 | OD2 | ASP | A | 383 | 22.414 | 14.78 | 1.059 | 1.00 | 17.35 |
| 1357 | C | ASP | A | 383 | 20.944 | 17.64 | 4.494 | 1.00 | 15.34 |
| 1358 | O | ASP | A | 383 | 21.351 | 17.96 | 5.61 | 1.00 | 13.16 |
| 1359 | N | ILE | A | 384 | 20.416 | 18.5 | 3.631 | 1.00 | 14.16 |
| 1360 | CA | ILE | A | 384 | 20.279 | 19.92 | 3.925 | 1.00 | 14.77 |
| 1361 | CB | ILE | A | 384 | 19.661 | 20.66 | 2.712 | 1.00 | 14.32 |
| 1362 | CG2 | ILE | A | 384 | 19.454 | 22.13 | 3.047 | 1.00 | 13.42 |
| 1363 | CG1 | ILE | A | 384 | 20.594 | 20.52 | 1.508 | 1.00 | 12.81 |
| 1364 | CD1 | ILE | A | 384 | 19.943 | 20.83 | 0.161 | 1.00 | 10.35 |
| 1365 | C | ILE | A | 384 | 19.452 | 20.19 | 5.18 | 1.00 | 15.58 |
| 1366 | O | ILE | A | 384 | 19.813 | 21.03 | 6.001 | 1.00 | 15.62 |
| 1367 | N | SER | A | 385 | 18.355 | 19.45 | 5.344 | 1.00 | 15.94 |
| 1368 | CA | SER | A | 385 | 17.513 | 19.66 | 6.516 | 1.00 | 17.39 |
| 1369 | CB | SER | A | 385 | 16.323 | 18.7 | 6.508 | 1.00 | 18.46 |
| 1370 | OG | SER | A | 385 | 16.747 | 17.36 | 6.665 | 1.00 | 19.88 |
| 1371 | C | SER | A | 385 | 18.313 | 19.48 | 7.799 | 1.00 | 16.76 |
| 1372 | O | SER | A | 385 | 18.146 | 20.24 | 8.735 | 1.00 | 15.46 |
| 1373 | N | LEU | A | 386 | 19.179 | 18.47 | 7.841 | 1.00 | 17.17 |
| 1374 | CA | LEU | A | 386 | 19.987 | 18.22 | 9.04 | 1.00 | 18.15 |
| 1375 | CB | LEU | A | 386 | 20.649 | 16.83 | 8.973 | 1.00 | 18.31 |
| 1376 | CG | LEU | A | 386 | 19.688 | 15.64 | 8.899 | 1.00 | 20.80 |
| 1377 | OD1 | LEU | A | 386 | 20.461 | 14.33 | 8.89 | 1.00 | 22.15 |
| 1378 | CD2 | LEU | A | 386 | 18.741 | 15.68 | 10.089 | 1.00 | 22.01 |
| 1379 | C | LEU | A | 386 | 21.062 | 19.29 | 9.21 | 1.00 | 17.32 |
| 1380 | O | LEU | A | 386 | 21.347 | 19.72 | 10.326 | 1.00 | 17.55 |
| 1381 | N | PHE | A | 387 | 21.655 | 19.7 | 8.094 | 1.00 | 15.68 |
| 1382 | CA | PHE | A | 387 | 22.701 | 20.72 | 8.093 | 1.00 | 15.42 |
| 1383 | CB | PHE | A | 387 | 23.191 | 20.94 | 6.658 | 1.00 | 14.83 |
| 1384 | CG | PHE | A | 387 | 24.356 | 21.88 | 6.546 | 1.00 | 17.91 |
| 1385 | CD1 | PHE | A | 387 | 25.631 | 21.48 | 6.933 | 1.00 | 18.12 |
| 1386 | CD2 | PHE | A | 387 | 24.174 | 23.17 | 6.06 | 1.00 | 17.17 |
| 1387 | CE1 | PHE | A | 387 | 26.711 | 22.36 | 6.835 | 1.00 | 19.84 |
| 1388 | CE2 | PHE | A | 387 | 25.248 | 24.06 | 5.96 | 1.00 | 17.27 |
| 1389 | CZ | PHE | A | 387 | 26.519 | 23.64 | 6.349 | 1.00 | 18.98 |
| 1390 | C | PHE | A | 387 | 22.14 | 22.03 | 8.667 | 1.00 | 14.65 |
| 1391 | O | PHE | A | 387 | 22.78 | 22.7 | 9.488 | 1.00 | 14.80 |
| 1392 | N | VAL | A | 388 | 20.937 | 22.4 | 8.235 | 1.00 | 14.52 |
| 1393 | CA | VAL | A | 388 | 20.302 | 23.62 | 8.715 | 1.00 | 13.14 |
| 1394 | CB | VAL | A | 388 | 19.061 | 23.96 | 7.856 | 1.00 | 14.31 |
| 1395 | CG1 | VAL | A | 388 | 18.253 | 25.08 | 8.481 | 1.00 | 13.46 |
| 1396 | CG2 | VAL | A | 388 | 19.531 | 24.38 | 6.456 | 1.00 | 13.68 |
| 1397 | C | VAL | A | 388 | 19.941 | 23.53 | 10.205 | 1.00 | 14.97 |
| 1398 | O | VAL | A | 388 | 20.1 | 24.5 | 10.946 | 1.00 | 14.16 |
| 1399 | N | ALA | A | 389 | 19.468 | 22.37 | 10.651 | 1.00 | 14.60 |
| 1400 | CA | ALA | A | 389 | 19.132 | 22.21 | 12.068 | 1.00 | 15.75 |
| 1401 | CB | ALA | A | 389 | 18.534 | 20.83 | 12.333 | 1.00 | 14.49 |
| 1402 | C | ALA | A | 389 | 20.409 | 22.39 | 12.884 | 1.00 | 17.48 |
| 1403 | O | ALA | A | 389 | 20.39 | 22.96 | 13.974 | 1.00 | 18.76 |
| 1404 | N | ALA | A | 390 | 21.516 | 21.89 | 12.345 | 1.00 | 17.47 |
| 1405 | CA | ALA | A | 390 | 22.806 | 21.99 | 13.012 | 1.00 | 18.52 |
| 1406 | CB | ALA | A | 390 | 23.838 | 21.13 | 12.278 | 1.00 | 18.08 |
| 1407 | C | ALA | A | 390 | 23.295 | 23.44 | 13.117 | 1.00 | 18.95 |
| 1408 | O | ALA | A | 390 | 23.835 | 23.84 | 14.146 | 1.00 | 18.98 |
| 1409 | N | ILE | A | 391 | 23.113 | 24.24 | 12.069 | 1.00 | 18.97 |
| 1410 | CA | ILE | A | 391 | 23.579 | 25.62 | 12.165 | 1.00 | 19.61 |
| 1411 | CB | ILE | A | 391 | 23.595 | 26.35 | 10.777 | 1.00 | 22.32 |
| 1412 | CG2 | ILE | A | 391 | 24.049 | 25.41 | 9.693 | 1.00 | 22.00 |
| 1413 | CG1 | ILE | A | 391 | 22.233 | 26.94 | 10.455 | 1.00 | 25.81 |
| 1414 | CD1 | ILE | A | 391 | 22.036 | 28.31 | 11.06 | 1.00 | 26.92 |
| 1415 | C | ILE | A | 391 | 22.718 | 26.41 | 13.158 | 1.00 | 19.24 |
| 1416 | O | ILE | A | 391 | 23.218 | 27.27 | 13.876 | 1.00 | 18.21 |
| 1417 | N | ILE | A | 392 | 21.429 | 26.09 | 13.212 | 1.00 | 17.90 |
| 1418 | CA | ILE | A | 392 | 20.514 | 26.77 | 14.118 | 1.00 | 18.91 |
| 1419 | CB | ILE | A | 392 | 19.047 | 26.43 | 13.79 | 1.00 | 20.25 |
| 1420 | CG2 | ILE | A | 392 | 18.119 | 26.92 | 14.901 | 1.00 | 20.50 |
| 1421 | CG1 | ILE | A | 392 | 18.658 | 27.05 | 12.449 | 1.00 | 19.74 |
| 1422 | CD1 | ILE | A | 392 | 17.232 | 26.77 | 12.028 | 1.00 | 20.27 |
| 1423 | C | ILE | A | 392 | 20.785 | 26.43 | 15.583 | 1.00 | 19.83 |
| 1424 | O | 11E | A | 392 | 20.872 | 27.32 | 16.435 | 1.00 | 18.98 |
| 1425 | N | CYS | A | 393 | 20.935 | 25.15 | 15.874 | 1.00 | 21.71 |
| 1426 | CA | CYS | A | 393 | 21.175 | 24.73 | 17.249 | 1.00 | 24.63 |
| 1427 | CB | CYS | A | 393 | 20.59 | 23.34 | 17.457 | 1.00 | 26.05 |
| 1428 | SG | CYS | A | 393 | 18.833 | 23.3 | 17.09 | 1.00 | 27.14 |
| 1429 | C | CYS | A | 393 | 22.655 | 24.76 | 17.576 | 1.00 | 25.29 |
| 1430 | O | CYS | A | 393 | 23.279 | 23.73 | 17.837 | 1.00 | 26.58 |
| 1431 | N | CYS | A | 394 | 23.196 | 25.97 | 17.561 | 1.00 | 27.00 |
| 1432 | CA | CYS | A | 394 | 24.602 | 26.23 | 17.818 | 1.00 | 28.30 |
| 1433 | CB | CYS | A | 394 | 25.095 | 27.25 | 16.792 | 1.00 | 30.14 |
| 1434 | SG | CYS | A | 394 | 26.804 | 27.71 | 16.962 | 1.00 | 32.84 |
| 1435 | C | CYS | A | 394 | 24.818 | 26.77 | 19.237 | 1.00 | 27.82 |
| 1436 | O | CYS | A | 394 | 24.279 | 27.82 | 19.602 | 1.00 | 27.12 |
| 1437 | N | GLY | A | 395 | 25.619 | 26.06 | 20.025 | 1.00 | 28.23 |
| 1438 | CA | GLY | A | 395 | 25.869 | 26.48 | 21.394 | 1.00 | 28.09 |
| 1439 | C | GLY | A | 395 | 26.924 | 27.55 | 21.575 | 1.00 | 28.17 |
| 1440 | O | GLY | A | 395 | 27.155 | 28.01 | 22.693 | 1.00 | 30.05 |
| 1441 | N | ASP | A | 396 | 27.556 | 27.97 | 20.483 | 1.00 | 29.00 |
| 1442 | CA | ASP | A | 396 | 28.603 | 28.98 | 20.54 | 1.00 | 29.78 |
| 1443 | CB | ASP | A | 396 | 29.678 | 28.7 | 19.49 | 1.00 | 33.93 |
| 1444 | CG | ASP | A | 396 | 30.174 | 27.28 | 19.544 | 1.00 | 37.57 |
| 1445 | OD1 | ASP | A | 396 | 30.417 | 26.78 | 20.664 | 1.00 | 40.66 |
| 1446 | OD2 | ASP | A | 396 | 30.329 | 26.66 | 18.466 | 1.00 | 39.44 |
| 1447 | C | ASP | A | 396 | 28.108 | 30.41 | 20.326 | 1.00 | 27.38 |
| 1448 | O | ASP | A | 396 | 28.888 | 31.35 | 20.404 | 1.00 | 27.02 |
| 1449 | N | ARG | A | 397 | 26.821 | 30.57 | 20.046 | 1.00 | 25.37 |
| 1450 | CA | ARG | A | 397 | 26.27 | 31.89 | 19.804 | 1.00 | 24.60 |
| 1451 | CB | ARG | A | 397 | 24.784 | 31.79 | 19.463 | 1.00 | 23.34 |
| 1452 | CG | ARG | A | 397 | 24.477 | 30.85 | 18.313 | 1.00 | 19.60 |
| 1453 | CD | ARG | A | 397 | 25.267 | 31.21 | 17.066 | 1.00 | 20.49 |
| 1454 | NE | ARG | A | 397 | 24.765 | 30.48 | 15.905 | 1.00 | 14.79 |
| 1455 | CZ | ARG | A | 397 | 25.326 | 30.5 | 14.703 | 1.00 | 16.61 |
| 1456 | NH1 | ARG | A | 397 | 26.427 | 31.22 | 14.486 | 1.00 | 14.39 |
| 1457 | NH2 | ARG | A | 397 | 24.786 | 29.8 | 13.717 | 1.00 | 14.57 |
| 1458 | C | ARG | A | 397 | 26.456 | 32.84 | 20.985 | 1.00 | 26.30 |
| 1459 | O | ARG | A | 397 | 26.283 | 32.45 | 22.14 | 1.00 | 26.77 |
| 1460 | N | PRO | A | 398 | 26.822 | 34.1 | 20.704 | 1.00 | 27.02 |
| 1461 | CD | PRO | A | 398 | 27.21 | 34.63 | 19.386 | 1.00 | 26.91 |
| 1462 | CA | PRO | A | 398 | 27.026 | 35.1 | 21.752 | 1.00 | 27.46 |
| 1463 | CB | PRO | A | 398 | 27.449 | 36.34 | 20.964 | 1.00 | 28.18 |
| 1464 | CG | PRO | A | 398 | 28.124 | 35.77 | 19.765 | 1.00 | 28.30 |
| 1465 | C | PRO | A | 398 | 25.735 | 35.35 | 22.534 | 1.00 | 28.08 |
| 1466 | O | PRO | A | 398 | 24.643 | 35.27 | 21.975 | 1.00 | 28.60 |
| 1467 | N | GLY | A | 399 | 25.872 | 35.63 | 23.826 | 1.00 | 27.99 |
| 1468 | CA | GLY | A | 399 | 24.716 | 35.92 | 24.66 | 1.00 | 27.31 |
| 1469 | C | GLY | A | 399 | 23.795 | 34.79 | 25.072 | 1.00 | 26.48 |
| 1470 | O | GLY | A | 399 | 22.714 | 35.04 | 25.603 | 1.00 | 25.44 |
| 1471 | N | LEU | A | 400 | 24.197 | 33.54 | 24.833 | 1.00 | 25.96 |
| 1472 | CA | LEU | A | 400 | 23.365 | 32.4 | 25.209 | 1.00 | 26.11 |
| 1473 | CB | LEU | A | 400 | 23.807 | 31.14 | 24.474 | 1.00 | 26.09 |
| 1474 | CG | LEU | A | 400 | 23.406 | 30.99 | 23.006 | 1.00 | 25.05 |
| 1475 | CD1 | LEU | A | 400 | 24.037 | 29.73 | 22.44 | 1.00 | 23.61 |
| 1476 | CD2 | LEU | A | 400 | 21.892 | 30.93 | 22.889 | 1.00 | 23.97 |
| 1477 | C | LEU | A | 400 | 23.431 | 32.14 | 26.706 | 1.00 | 27.86 |
| 1478 | O | LEU | A | 400 | 24.473 | 32.34 | 27.334 | 1.00 | 26.88 |
| 1479 | N | LEU | A | 401 | 22.318 | 31.67 | 27.261 | 1.00 | 28.14 |
| 1480 | CA | LEU | A | 401 | 22.233 | 31.38 | 28.684 | 1.00 | 31.00 |
| 1481 | CB | LEU | A | 401 | 20.865 | 31.8 | 29.228 | 1.00 | 30.68 |
| 1482 | CG | LEU | A | 401 | 20.578 | 31.45 | 30.691 | 1.00 | 32.59 |
| 1483 | CD1 | LEU | A | 401 | 21.62 | 32.1 | 31.597 | 1.00 | 32.90 |
| 1484 | CD2 | LEU | A | 401 | 19.179 | 31.92 | 31.06 | 1.00 | 31.69 |
| 1485 | C | LEU | A | 401 | 22.429 | 29.89 | 28.943 | 1.00 | 31.60 |
| 1486 | O | LEU | A | 401 | 23.369 | 29.48 | 29.622 | 1.00 | 33.05 |
| 1487 | N | ASN | A | 402 | 21.532 | 29.09 | 28.383 | 1.00 | 31.62 |
| 1488 | CA | ASN | A | 402 | 21.56 | 27.65 | 28.559 | 1.00 | 32.32 |
| 1489 | CB | ASN | A | 402 | 20.142 | 27.12 | 28.394 | 1.00 | 34.00 |
| 1490 | CG | ASN | A | 402 | 19.94 | 25.79 | 29.062 | 1.00 | 36.18 |
| 1491 | OD1 | ASN | A | 402 | 18.817 | 25.3 | 29.135 | 1.00 | 38.80 |
| 1492 | ND2 | ASN | A | 402 | 21.022 | 25.2 | 29.558 | 1.00 | 37.30 |
| 1493 | C | ASN | A | 402 | 22.508 | 26.95 | 27.584 | 1.00 | 31.82 |
| 1494 | O | ASN | A | 402 | 22.123 | 25.98 | 26.925 | 1.00 | 30.71 |
| 1495 | N | VAL | A | 403 | 23.746 | 27.42 | 27.514 | 1.00 | 31.16 |
| 1496 | CA | VAL | A | 403 | 24.752 | 26.85 | 26.624 | 1.00 | 31.48 |
| 1497 | CB | VAL | A | 403 | 26.143 | 27.48 | 26.903 | 1.00 | 31.26 |
| 1498 | CG1 | VAL | A | 403 | 26.484 | 27.35 | 28.377 | 1.00 | 32.59 |
| 1499 | CG2 | VAL | A | 403 | 27.211 | 26.82 | 26.045 | 1.00 | 31.72 |
| 1500 | C | VAL | A | 403 | 24.847 | 25.32 | 26.73 | 1.00 | 31.80 |
| 1501 | O | VAL | A | 403 | 24.987 | 24.63 | 25.72 | 1.00 | 32.39 |
| 1502 | N | GLY | A | 404 | 24.756 | 24.8 | 27.949 | 1.00 | 30.80 |
| 1503 | CA | GLY | A | 404 | 24.836 | 23.36 | 28.141 | 1.00 | 30.87 |
| 1504 | C | GLY | A | 404 | 23.746 | 22.57 | 27.442 | 1.00 | 31.11 |
| 1505 | O | GLY | A | 404 | 24.032 | 21.62 | 26.71 | 1.00 | 31.28 |
| 1506 | N | HIS | A | 405 | 22.493 | 22.95 | 27.661 | 1.00 | 30.39 |
| 1507 | CA | HIS | A | 405 | 21.37 | 22.26 | 27.051 | 1.00 | 30.64 |
| 1508 | CB | HIS | A | 405 | 20.045 | 22.78 | 27.623 | 1.00 | 33.97 |
| 1509 | CG | HIS | A | 405 | 19.876 | 22.51 | 29.09 | 1.00 | 38.64 |
| 1510 | CD2 | HIS | A | 405 | 18.885 | 22.85 | 29.952 | 1.00 | 40.99 |
| 1511 | ND1 | HIS | A | 405 | 20.82 | 21.84 | 29.836 | 1.00 | 39.58 |
| 1512 | CE1 | HIS | A | 405 | 20.42 | 21.77 | 31.094 | 1.00 | 41.27 |
| 1513 | NE2 | HIS | A | 405 | 19.25 | 22.38 | 31.191 | 1.00 | 41.61 |
| 1514 | C | HIS | A | 405 | 21.385 | 22.38 | 25.53 | 1.00 | 29.36 |
| 1515 | O | HIS | A | 405 | 20.963 | 21.47 | 24.827 | 1.00 | 28.36 |
| 1516 | N | ILE | A | 406 | 21.881 | 23.5 | 25.024 | 1.00 | 27.89 |
| 1517 | CA | ILE | A | 406 | 21.943 | 23.71 | 23.58 | 1.00 | 27.60 |
| 1518 | CB | ILE | A | 406 | 22.21 | 25.19 | 23.238 | 1.00 | 25.64 |
| 1519 | CG2 | ILE | A | 406 | 22.473 | 25.36 | 21.741 | 1.00 | 25.46 |
| 1520 | CG1 | ILE | A | 406 | 20.993 | 26.03 | 23.636 | 1.00 | 23.43 |
| 1521 | CD1 | ILE | A | 406 | 21.23 | 27.53 | 23.575 | 1.00 | 23.81 |
| 1522 | C | ILE | A | 406 | 23.023 | 22.82 | 22.961 | 1.00 | 28.42 |
| 1523 | O | ILE | A | 406 | 22.848 | 22.3 | 21.86 | 1.00 | 28.52 |
| 1524 | N | GLU | A | 407 | 24.132 | 22.63 | 23.673 | 1.00 | 28.38 |
| 1525 | CA | GLU | A | 407 | 25.21 | 21.79 | 23.165 | 1.00 | 30.86 |
| 1526 | CB | GLU | A | 407 | 26.435 | 21.85 | 24.087 | 1.00 | 31.87 |
| 1527 | CG | GLU | A | 407 | 27.059 | 23.22 | 24.208 | 1.00 | 35.51 |
| 1528 | CD | GLU | A | 407 | 28.343 | 23.21 | 25.016 | 1.00 | 37.24 |
| 1529 | OE1 | GLU | A | 407 | 28.346 | 22.62 | 26.121 | 1.00 | 38.47 |
| 1530 | OE2 | GLU | A | 407 | 29.345 | 23.79 | 24.548 | 1.00 | 39.11 |
| 1531 | C | GLU | A | 407 | 24.754 | 20.34 | 23.033 | 1.00 | 30.96 |
| 1532 | O | GLU | A | 407 | 25.116 | 19.65 | 22.081 | 1.00 | 32.65 |
| 1533 | N | LYS | A | 408 | 23.959 | 19.87 | 23.991 | 1.00 | 31.74 |
| 1534 | CA | LYS | A | 408 | 23.47 | 18.49 | 23.962 | 1.00 | 32.12 |
| 1535 | CB | LYS | A | 408 | 22.748 | 18.16 | 25.275 | 1.00 | 34.57 |
| 1536 | CG | LYS | A | 408 | 21.294 | 18.6 | 25.333 | 1.00 | 37.40 |
| 1537 | CD | LYS | A | 408 | 20.363 | 17.48 | 24.898 | 1.00 | 39.89 |
| 1538 | CE | LYS | A | 408 | 19.082 | 18.02 | 24.279 | 1.00 | 40.38 |
| 1539 | NZ | LYS | A | 408 | 18.438 | 19.07 | 25.118 | 1.00 | 42.06 |
| 1540 | C | LYS | A | 408 | 22.527 | 18.33 | 22.771 | 1.00 | 30.88 |
| 1541 | O | LYS | A | 408 | 22.44 | 17.26 | 22.168 | 1.00 | 29.64 |
| 1542 | N | MET | A | 409 | 21.827 | 19.41 | 22.436 | 1.00 | 30.20 |
| 1543 | CA | MET | A | 409 | 20.907 | 19.4 | 21.304 | 1.00 | 30.04 |
| 1544 | CB | MET | A | 409 | 20.125 | 20.71 | 21.259 | 1.00 | 31.30 |
| 1545 | CG | MET | A | 409 | 18.627 | 20.56 | 21.382 | 1.00 | 34.42 |
| 1546 | SD | MET | A | 409 | 17.79 | 22.14 | 21.137 | 1.00 | 37.50 |
| 1547 | CE | MET | A | 409 | 17.603 | 22.66 | 22.784 | 1.00 | 35.32 |
| 1548 | C | MET | A | 409 | 21.703 | 19.25 | 20.01 | 1.00 | 28.14 |
| 1549 | O | MET | A | 409 | 21.397 | 18.4 | 19.172 | 1.00 | 27.06 |
| 1550 | N | GLN | A | 410 | 22.726 | 20.09 | 19.851 | 1.00 | 27.24 |
| 1551 | CA | GLN | A | 410 | 23.555 | 20.04 | 18.652 | 1.00 | 27.85 |
| 1552 | CB | GLN | A | 410 | 24.58 | 21.19 | 18.647 | 1.00 | 29.46 |
| 1553 | CG | GLN | A | 410 | 25.441 | 21.21 | 17.38 | 1.00 | 32.43 |
| 1554 | CD | GLN | A | 410 | 26.288 | 22.47 | 17.245 | 1.00 | 34.32 |
| 1555 | OE1 | GLN | A | 410 | 27.097 | 22.79 | 18.115 | 1.00 | 34.44 |
| 1556 | NE2 | GLN | A | 410 | 26.105 | 23.19 | 16.14 | 1.00 | 32.43 |
| 1557 | C | GLN | A | 410 | 24.275 | 18.71 | 18.545 | 1.00 | 27.48 |
| 1558 | O | GLN | A | 410 | 24.483 | 18.19 | 17.449 | 1.00 | 26.07 |
| 1559 | N | GLU | A | 411 | 24.645 | 18.14 | 19.691 | 1.00 | 27.34 |
| 1560 | CA | GLU | A | 411 | 25.339 | 16.86 | 19.731 | 1.00 | 28.25 |
| 1561 | CB | GLU | A | 411 | 25.621 | 16.46 | 21.186 | 1.00 | 30.37 |
| 1562 | CG | GLU | A | 411 | 26.281 | 15.09 | 21.341 | 1.00 | 35.80 |
| 1563 | CD | GLU | A | 411 | 26.58 | 14.74 | 22.791 | 1.00 | 38.57 |
| 1564 | OE1 | GLU | A | 411 | 27.532 | 15.32 | 23.359 | 1.00 | 39.33 |
| 1565 | OE2 | GLU | A | 411 | 25.858 | 13.89 | 23.364 | 1.00 | 39.79 |
| 1566 | C | GLU | A | 411 | 24.508 | 15.78 | 19.043 | 1.00 | 26.37 |
| 1567 | O | GLU | A | 411 | 25.022 | 15.03 | 18.213 | 1.00 | 26.12 |
| 1568 | N | GLY | A | 412 | 23.226 | 15.71 | 19.396 | 1.00 | 22.95 |
| 1569 | CA | GLY | A | 412 | 22.335 | 14.73 | 18.811 | 1.00 | 22.23 |
| 1570 | C | GLY | A | 412 | 22.12 | 14.94 | 17.323 | 1.00 | 22.14 |
| 1571 | O | GLY | A | 412 | 22.074 | 13.99 | 16.55 | 1.00 | 22.69 |
| 1572 | N | ILE | A | 413 | 21.984 | 16.2 | 16.918 | 1.00 | 20.27 |
| 1573 | CA | ILE | A | 413 | 21.785 | 16.53 | 15.507 | 1.00 | 20.28 |
| 1574 | CB | ILE | A | 413 | 21.5 | 18.06 | 15.334 | 1.00 | 18.92 |
| 1575 | CG2 | ILE | A | 413 | 21.591 | 18.46 | 13.857 | 1.00 | 18.08 |
| 1576 | CG1 | ILE | A | 413 | 20.124 | 18.38 | 15.919 | 1.00 | 20.64 |
| 1577 | CD1 | ILE | A | 413 | 19.761 | 19.87 | 15.944 | 1.00 | 21.12 |
| 1578 | C | ILE | A | 413 | 23.01 | 16.13 | 14.686 | 1.00 | 20.45 |
| 1579 | O | ILE | A | 413 | 22.886 | 15.5 | 13.637 | 1.00 | 19.39 |
| 1580 | N | VAL | A | 414 | 24.191 | 16.49 | 15.177 | 1.00 | 22.04 |
| 1581 | CA | VAL | A | 414 | 25.438 | 16.16 | 14.493 | 1.00 | 24.01 |
| 1582 | CB | VAL | A | 414 | 26.648 | 16.76 | 15.237 | 1.00 | 24.63 |
| 1583 | CG1 | VAL | A | 414 | 27.944 | 16.24 | 14.633 | 1.00 | 26.70 |
| 1584 | CG2 | VAL | A | 414 | 26.604 | 18.28 | 15.152 | 1.00 | 25.08 |
| 1585 | C | VAL | A | 414 | 25.621 | 14.66 | 14.382 | 1.00 | 24.35 |
| 1586 | O | VAL | A | 414 | 26.059 | 14.15 | 13.352 | 1.00 | 24.50 |
| 1587 | N | HIS | A | 415 | 25.289 | 13.95 | 15.457 | 1.00 | 24.73 |
| 1588 | CA | HIS | A | 415 | 25.398 | 12.5 | 15.496 | 1.00 | 25.40 |
| 1589 | CB | HIS | A | 415 | 24.877 | 11.98 | 16.836 | 1.00 | 27.91 |
| 1590 | CG | HIS | A | 415 | 24.706 | 10.49 | 16.882 | 1.00 | 29.97 |
| 1591 | CD2 | HIS | A | 415 | 23.603 | 9.718 | 16.752 | 1.00 | 31.72 |
| 1592 | ND1 | HIS | A | 415 | 25.765 | 9.623 | 17.036 | 1.00 | 32.64 |
| 1593 | CE1 | HIS | A | 415 | 25.321 | 8.379 | 16.999 | 1.00 | 31.24 |
| 1594 | NE2 | HIS | A | 415 | 24.013 | 8.408 | 16.826 | 1.00 | 32.29 |
| 1595 | C | HIS | A | 415 | 24.576 | 11.89 | 14.363 | 1.00 | 24.78 |
| 1596 | O | HIS | A | 415 | 25.076 | 11.09 | 13.57 | 1.00 | 23.70 |
| 1597 | N | VAL | A | 416 | 23.308 | 12.29 | 14.302 | 1.00 | 24.16 |
| 1598 | CA | VAL | A | 416 | 22.393 | 11.8 | 13.28 | 1.00 | 24.60 |
| 1599 | CB | VAL | A | 416 | 20.96 | 12.32 | 13.548 | 1.00 | 26.47 |
| 1600 | CG1 | VAL | A | 416 | 20.056 | 12.01 | 12.371 | 1.00 | 29.88 |
| 1601 | CG2 | VAL | A | 416 | 20.411 | 11.66 | 14.809 | 1.00 | 27.86 |
| 1602 | C | VAL | A | 416 | 22.868 | 12.22 | 11.889 | 1.00 | 23.32 |
| 1603 | O | VAL | A | 416 | 22.743 | 11.46 | 10.927 | 1.00 | 21.28 |
| 1604 | N | LEU | A | 417 | 23.43 | 13.42 | 11.788 | 1.00 | 21.40 |
| 1605 | CA | LEU | A | 417 | 23.936 | 13.92 | 10.512 | 1.00 | 20.88 |
| 1606 | CB | LEU | A | 417 | 24.389 | 15.38 | 10.638 | 1.00 | 20.25 |
| 1607 | CG | LEU | A | 417 | 25.166 | 15.98 | 9.461 | 1.00 | 19.60 |
| 1608 | CD1 | LEU | A | 417 | 24.323 | 15.92 | 8.189 | 1.00 | 18.77 |
| 1609 | CD2 | LEU | A | 417 | 25.549 | 17.41 | 9.782 | 1.00 | 20.66 |
| 1610 | C | LEU | A | 417 | 25.103 | 13.06 | 10.031 | 1.00 | 21.76 |
| 1611 | O | LEU | A | 417 | 25.134 | 12.64 | 8.877 | 1.00 | 21.23 |
| 1612 | N | ARG | A | 418 | 26.056 | 12.78 | 10.921 | 1.00 | 22.31 |
| 1613 | CA | ARG | A | 418 | 27.214 | 11.97 | 10.551 | 1.00 | 23.13 |
| 1614 | CB | ARG | A | 418 | 28.166 | 11.78 | 11.738 | 1.00 | 25.75 |
| 1615 | CG | ARG | A | 418 | 29.481 | 11.11 | 11.325 | 1.00 | 30.84 |
| 1616 | CD | ARG | A | 418 | 30.375 | 10.73 | 12.502 | 1.00 | 34.64 |
| 1617 | NE | ARG | A | 418 | 30.554 | 11.83 | 13.446 | 1.00 | 36.72 |
| 1618 | CZ | ARG | A | 418 | 29.916 | 11.92 | 14.608 | 1.00 | 38.12 |
| 1619 | NH1 | ARG | A | 418 | 29.061 | 10.98 | 14.969 | 1.00 | 40.03 |
| 1620 | NH2 | ARG | A | 418 | 30.131 | 12.96 | 15.406 | 1.00 | 40.43 |
| 1621 | C | ARG | A | 418 | 26.795 | 10.6 | 10.036 | 1.00 | 23.39 |
| 1622 | O | ARG | A | 418 | 27.332 | 10.11 | 9.043 | 1.00 | 22.61 |
| 1623 | N | LEU | A | 419 | 25.837 | 9.988 | 10.721 | 1.00 | 22.60 |
| 1624 | CA | LEU | A | 419 | 25.35 | 8.67 | 10.333 | 1.00 | 24.37 |
| 1625 | CB | LEU | A | 419 | 24.453 | 8.101 | 11.439 | 1.00 | 24.59 |
| 1626 | CG | LEU | A | 419 | 25.209 | 7.675 | 12.708 | 1.00 | 27.68 |
| 1627 | CDL | LEU | A | 419 | 24.232 | 7.27 | 13.802 | 1.00 | 27.24 |
| 1628 | CD2 | LEU | A | 419 | 26.141 | 6.522 | 12.37 | 1.00 | 26.90 |
| 1629 | C | LEU | A | 419 | 24.594 | 8.736 | 9.007 | 1.00 | 23.58 |
| 1630 | O | LEU | A | 419 | 24.753 | 7.87 | 8.141 | 1.00 | 22.71 |
| 1631 | N | HIS | A | 420 | 23.782 | 9.772 | 8.845 | 1.00 | 22.55 |
| 1632 | CA | HIS | A | 420 | 23.011 | 9.943 | 7.619 | 1.00 | 22.81 |
| 1633 | CB | HIS | A | 420 | 22.098 | 11.17 | 7.748 | 1.00 | 22.24 |
| 1634 | CG | HIS | A | 420 | 21.152 | 11.34 | 6.6 | 1.00 | 22.11 |
| 1635 | CD2 | HIS | A | 420 | 19.934 | 10.8 | 6.361 | 1.00 | 23.05 |
| 1636 | NDL | HIS | A | 420 | 21.435 | 12.14 | 5.51 | 1.00 | 23.65 |
| 1637 | CE1 | HIS | A | 420 | 20.435 | 12.08 | 4.649 | 1.00 | 20.84 |
| 1638 | NE2 | HIS | A | 420 | 19.512 | 11.27 | 5.141 | 1.00 | 24.45 |
| 1639 | C | HIS | A | 420 | 23.929 | 10.09 | 6.407 | 1.00 | 22.74 |
| 1640 | O | HIS | A | 420 | 23.687 | 9.488 | 5.355 | 1.00 | 21.42 |
| 1641 | N | LEU | A | 421 | 24.985 | 10.89 | 6.553 | 1.00 | 20.60 |
| 1642 | CA | LEU | A | 421 | 25.926 | 11.09 | 5.458 | 1.00 | 22.09 |
| 1643 | CB | LEU | A | 421 | 26.967 | 12.15 | 5.837 | 1.00 | 21.12 |
| 1644 | CG | LEU | A | 421 | 26.44 | 13.58 | 6.022 | 1.00 | 20.72 |
| 1645 | CD1 | LEU | A | 421 | 27.573 | 14.49 | 6.475 | 1.00 | 20.64 |
| 1646 | CD2 | LEU | A | 421 | 25.832 | 14.07 | 4.708 | 1.00 | 21.54 |
| 1647 | C | LEU | A | 421 | 26.636 | 9.806 | 5.062 | 1.00 | 22.50 |
| 1648 | O | LEU | A | 421 | 26.916 | 9.584 | 3.885 | 1.00 | 22.76 |
| 1649 | N | GLN | A | 422 | 26.941 | 8.962 | 6.042 | 1.00 | 24.45 |
| 1650 | CA | GLN | A | 422 | 27.621 | 7.706 | 5.75 | 1.00 | 26.41 |
| 1651 | CB | GLN | A | 422 | 28.016 | 6.992 | 7.047 | 1.00 | 28.87 |
| 1652 | CG | GLN | A | 422 | 29.241 | 7.583 | 7.717 | 1.00 | 32.50 |
| 1653 | CD | GLN | A | 422 | 30.157 | 6.52 | 8.293 | 1.00 | 31.70 |
| 1654 | OE1 | GLN | A | 422 | 29.819 | 5.852 | 9.27 | 1.00 | 36.19 |
| 1655 | NE2 | GLN | A | 422 | 31.325 | 6.353 | 7.68 | 1.00 | 38.34 |
| 1656 | C | GLN | A | 422 | 26.765 | 6.781 | 4.888 | 1.00 | 26.96 |
| 1657 | O | GLN | A | 422 | 27.271 | 6.136 | 3.968 | 1.00 | 26.69 |
| 1658 | N | SER | A | 423 | 25.468 | 6.729 | 5.176 | 1.00 | 26.98 |
| 1659 | CA | SER | A | 423 | 24.555 | 5.88 | 4.417 | 1.00 | 27.71 |
| 1660 | CB | SER | A | 423 | 23.377 | 5.458 | 5.298 | 1.00 | 29.19 |
| 1661 | OG | SER | A | 423 | 22.558 | 6.566 | 5.623 | 1.00 | 33.45 |
| 1662 | C | SER | A | 423 | 24.02 | 6.524 | 3.137 | 1.00 | 26.94 |
| 1663 | O | SER | A | 423 | 23.724 | 5.825 | 2.17 | 1.00 | 27.54 |
| 1664 | N | ASN | A | 424 | 23.892 | 7.851 | 3.124 | 1.00 | 25.54 |
| 1665 | CA | ASN | A | 424 | 23.371 | 8.557 | 1.947 | 1.00 | 24.76 |
| 1666 | CB | ASN | A | 424 | 22.674 | 9.86 | 2.377 | 1.00 | 24.73 |
| 1667 | CG | ASN | A | 424 | 21.705 | 10.4 | 1.318 | 1.00 | 26.54 |
| 1668 | OD1 | ASN | A | 424 | 21.319 | 11.57 | 1.348 | 1.00 | 25.37 |
| 1669 | ND2 | ASN | A | 424 | 21.295 | 9.532 | 0.392 | 1.00 | 24.83 |
| 1670 | C | ASN | A | 424 | 24.468 | 8.873 | 0.928 | 1.00 | 24.23 |
| 1671 | O | ASN | A | 424 | 24.201 | 8.95 | −0.271 | 1.00 | 23.51 |
| 1672 | N | HIS | A | 425 | 25.698 | 9.06 | 1.405 | 1.00 | 23.19 |
| 1673 | CA | HIS | A | 425 | 26.833 | 9.36 | 0.53 | 1.00 | 24.23 |
| 1674 | CB | HIS | A | 425 | 27.272 | 10.83 | 0.682 | 1.00 | 22.51 |
| 1675 | CG | HIS | A | 425 | 26.239 | 11.82 | 0.242 | 1.00 | 21.91 |
| 1676 | CD2 | HIS | A | 425 | 26.002 | 12.38 | −0.967 | 1.00 | 19.28 |
| 1677 | ND1 | HIS | A | 425 | 25.284 | 12.33 | 1.096 | 1.00 | 22.80 |
| 1678 | CE1 | HIS | A | 425 | 24.503 | 13.16 | 0.431 | 1.00 | 17.33 |
| 1679 | NE2 | HIS | A | 425 | 24.917 | 13.21 | −0.823 | 1.00 | 20.76 |
| 1680 | C | HIS | A | 425 | 28.016 | 8.451 | 0.862 | 1.00 | 26.11 |
| 1681 | O | HIS | A | 425 | 29.073 | 8.918 | 1.285 | 1.00 | 24.94 |
| 1682 | N | PRO | A | 426 | 27.856 | 7.136 | 0.65 | 1.00 | 29.11 |
| 1683 | CD | PRO | A | 426 | 26.708 | 6.467 | 0.008 | 1.00 | 29.49 |
| 1684 | CA | PRO | A | 426 | 28.922 | 6.174 | 0.94 | 1.00 | 30.85 |
| 1685 | CB | PRO | A | 426 | 28.248 | 4.831 | 0.67 | 1.00 | 31.26 |
| 1686 | CG | PRO | A | 426 | 27.321 | 5.157 | −0.46 | 1.00 | 30.77 |
| 1687 | C | PRO | A | 426 | 30.193 | 6.361 | 0.119 | 1.00 | 33.62 |
| 1688 | O | PRO | A | 426 | 31.268 | 5.919 | 0.529 | 1.00 | 35.12 |
| 1689 | N | ASP | A | 427 | 30.08 | 7.013 | −1.034 | 1.00 | 34.60 |
| 1690 | CA | ASP | A | 427 | 31.243 | 7.22 | −1.883 | 1.00 | 36.67 |
| 1691 | CB | ASP | A | 427 | 30.827 | 7.268 | −3.357 | 1.00 | 39.63 |
| 1692 | CG | ASP | A | 427 | 30.146 | 5.986 | −3.814 | 1.00 | 41.06 |
| 1693 | OD1 | ASP | A | 427 | 30.565 | 4.897 | −3.366 | 1.00 | 42.57 |
| 1694 | OD2 | ASP | A | 427 | 29.201 | 6.068 | −4.629 | 1.00 | 43.11 |
| 1695 | C | ASP | A | 427 | 32.053 | 8.465 | −1.542 | 1.00 | 37.42 |
| 1696 | O | ASP | A | 427 | 33.263 | 8.498 | −1.774 | 1.00 | 37.62 |
| 1697 | N | ASP | A | 428 | 31.402 | 9.486 | −0.99 | 1.00 | 37.02 |
| 1698 | CA | ASP | A | 428 | 32.126 | 10.71 | −0.649 | 1.00 | 37.69 |
| 1699 | CB | ASP | A | 428 | 31.198 | 11.9 | −0.46 | 1.00 | 36.87 |
| 1700 | CG | ASP | A | 428 | 31.972 | 13.21 | −0.347 | 1.00 | 37.47 |
| 1701 | OD1 | ASP | A | 428 | 33.072 | 13.21 | 0.248 | 1.00 | 36.42 |
| 1702 | OD2 | ASP | A | 428 | 31.484 | 14.25 | −0.847 | 1.00 | 37.98 |
| 1703 | C | ASP | A | 428 | 32.96 | 10.55 | 0.602 | 1.00 | 38.20 |
| 1704 | O | ASP | A | 428 | 32.451 | 10.39 | 1.714 | 1.00 | 37.99 |
| 1705 | N | ILE | A | 429 | 34.261 | 10.63 | 0.385 | 1.00 | 39.15 |
| 1706 | CA | ILE | A | 429 | 35.269 | 10.51 | 1.414 | 1.00 | 37.95 |
| 1707 | CB | ILE | A | 429 | 36.628 | 10.91 | 0.814 | 1.00 | 39.91 |
| 1708 | CG2 | ILE | A | 429 | 37.047 | 9.889 | −0.236 | 1.00 | 40.60 |
| 1709 | OG1 | ILE | A | 429 | 36.508 | 12.29 | 0.139 | 1.00 | 41.10 |
| 1710 | CD1 | ILE | A | 429 | 37.785 | 12.79 | −0.515 | 1.00 | 42.96 |
| 1711 | C | ILE | A | 429 | 35.027 | 11.31 | 2.702 | 1.00 | 35.07 |
| 1712 | O | ILE | A | 429 | 34.534 | 10.78 | 3.698 | 1.00 | 35.43 |
| 1713 | N | PHE | A | 430 | 35.379 | 12.58 | 2.67 | 1.00 | 31.63 |
| 1714 | CA | PHE | A | 430 | 35.263 | 13.44 | 3.831 | 1.00 | 26.48 |
| 1715 | CB | PHE | A | 430 | 36.493 | 14.35 | 3.926 | 1.00 | 29.04 |
| 1716 | CG | PHE | A | 430 | 37.806 | 13.62 | 3.916 | 1.00 | 30.93 |
| 1717 | CD1 | PHE | A | 430 | 38.349 | 13.15 | 2.729 | 1.00 | 33.42 |
| 1718 | GD2 | PHE | A | 430 | 38.518 | 13.44 | 5.093 | 1.00 | 31.75 |
| 1719 | CE1 | PHE | A | 430 | 39.592 | 12.51 | 2.714 | 1.00 | 34.29 |
| 1720 | CE2 | PHE | A | 430 | 39.758 | 12.8 | 5.091 | 1.00 | 31.88 |
| 1721 | CZ | PHE | A | 430 | 40.296 | 12.34 | 3.9 | 1.00 | 33.09 |
| 1722 | C | PHE | A | 430 | 34.035 | 14.34 | 3.817 | 1.00 | 23.47 |
| 1723 | O | PHE | A | 430 | 34.15 | 15.51 | 4.167 | 1.00 | 21.09 |
| 1724 | N | LEU | A | 431 | 32.864 | 13.83 | 3.444 | 1.00 | 20.27 |
| 1725 | CA | LEU | A | 431 | 31.7 | 14.72 | 3.403 | 1.00 | 17.44 |
| 1726 | CB | LEU | A | 431 | 30.468 | 13.98 | 2.853 | 1.00 | 15.99 |
| 1727 | CG | LEU | A | 431 | 29.23 | 14.85 | 2.603 | 1.00 | 14.82 |
| 1728 | CD1 | LEU | A | 431 | 29.603 | 16.1 | 1.815 | 1.00 | 15.78 |
| 1729 | CD2 | LEU | A | 431 | 28.183 | 14.05 | 1.862 | 1.00 | 16.91 |
| 1730 | C | LEU | A | 431 | 31.378 | 15.38 | 4.747 | 1.00 | 17.17 |
| 1731 | O | LEU | A | 431 | 31 | 16.56 | 4.789 | 1.00 | 15.16 |
| 1732 | N | PHE | A | 432 | 31.533 | 14.65 | 5.852 | 1.00 | 15.85 |
| 1733 | CA | PHE | A | 432 | 31.243 | 15.25 | 7.156 | 1.00 | 15.61 |
| 1734 | CB | PUB | A | 432 | 31.332 | 14.19 | 8.267 | 1.00 | 17.14 |
| 1735 | CO | PHE | A | 432 | 30.971 | 14.71 | 9.629 | 1.00 | 18.64 |
| 1736 | CD1 | PUB | A | 432 | 29.695 | 15.2 | 9.889 | 1.00 | 20.33 |
| 1737 | CD2 | PHE | A | 432 | 31.906 | 14.71 | 10.657 | 1.00 | 20.31 |
| 1738 | CE1 | PHE | A | 432 | 29.355 | 15.68 | 11.157 | 1.00 | 20.29 |
| 1739 | CE2 | PUB | A | 432 | 31.576 | 15.18 | 11.93 | 1.00 | 21.44 |
| 1740 | CZ | PHE | A | 432 | 30.299 | 15.67 | 12.179 | 1.00 | 22.31 |
| 1741 | C | PHE | A | 432 | 32.196 | 16.41 | 7.451 | 1.00 | 16.28 |
| 1742 | O | PHE | A | 432 | 31.754 | 17.54 | 7.719 | 1.00 | 14.24 |
| 1743 | N | PRO | A | 433 | 33.52 | 16.17 | 7.422 | 1.00 | 17.45 |
| 1744 | CD | PRO | A | 433 | 34.282 | 14.91 | 7.339 | 1.00 | 17.91 |
| 1745 | CA | PRO | A | 433 | 34.396 | 17.31 | 7.704 | 1.00 | 16.84 |
| 1746 | CB | PRO | A | 433 | 35.796 | 16.68 | 7.766 | 1.00 | 19.13 |
| 1747 | CO | PRO | A | 433 | 35.663 | 15.4 | 6.995 | 1.00 | 18.96 |
| 1748 | C | PRO | A | 433 | 34.255 | 18.44 | 6.663 | 1.00 | 15.92 |
| 1749 | O | PRO | A | 433 | 34.466 | 19.61 | 6.974 | 1.00 | 15.44 |
| 1750 | N | LYS | A | 434 | 33.882 | 18.08 | 5.435 | 1.00 | 16.05 |
| 1751 | CA | LYS | A | 434 | 33.678 | 19.1 | 4.401 | 1.00 | 15.22 |
| 1752 | CB | LYS | A | 434 | 33.261 | 18.45 | 3.076 | 1.00 | 15.46 |
| 1753 | CG | LYS | A | 434 | 34.363 | 17.72 | 2.337 | 1.00 | 16.19 |
| 1754 | CD | LYS | A | 434 | 33.806 | 17.12 | 1.056 | 1.00 | 17.44 |
| 1755 | CE | LYS | A | 434 | 34.848 | 16.33 | 0.281 | 1.00 | 19.89 |
| 1756 | NZ | LYS | A | 434 | 34.259 | 15.78 | −0.979 | 1.00 | 18.37 |
| 1757 | C | LYS | A | 434 | 32.561 | 20.03 | 4.857 | 1.00 | 14.66 |
| 1758 | O | LYS | A | 434 | 32.659 | 21.26 | 4.722 | 1.00 | 13.26 |
| 1759 | N | LEU | A | 435 | 31.495 | 19.45 | 5.397 | 1.00 | 13.97 |
| 1760 | CA | LEU | A | 435 | 30.351 | 20.22 | 5.869 | 1.00 | 14.01 |
| 1761 | CB | LEU | A | 435 | 29.154 | 19.31 | 6.134 | 1.00 | 14.60 |
| 1762 | CG | LEU | A | 435 | 28.489 | 18.73 | 4.885 | 1.00 | 18.11 |
| 1763 | CD1 | LEU | A | 435 | 27.366 | 17.78 | 5.291 | 1.00 | 19.14 |
| 1764 | CD2 | LEU | A | 435 | 27.947 | 19.86 | 4.019 | 1.00 | 19.11 |
| 1765 | C | LEU | A | 435 | 30.695 | 21.02 | 7.118 | 1.00 | 15.51 |
| 1766 | O | LEU | A | 435 | 30.2 | 22.14 | 7.303 | 1.00 | 16.69 |
| 1767 | N | LEU | A | 436 | 31.546 | 20.47 | 7.977 | 1.00 | 16.13 |
| 1768 | CA | LEU | A | 436 | 31.947 | 21.22 | 9.163 | 1.00 | 17.20 |
| 1769 | CB | LEU | A | 436 | 32.875 | 20.39 | 10.053 | 1.00 | 19.20 |
| 1770 | CG | LEU | A | 436 | 32.224 | 19.18 | 10.73 | 1.00 | 21.00 |
| 1771 | CD1 | LEU | A | 436 | 33.252 | 18.44 | 11.581 | 1.00 | 22.86 |
| 1772 | CD2 | LEU | A | 436 | 31.057 | 19.64 | 11.589 | 1.00 | 22.35 |
| 1773 | C | LEU | A | 436 | 32.667 | 22.47 | 8.693 | 1.00 | 17.44 |
| 1774 | O | LEU | A | 436 | 32.488 | 23.55 | 9.265 | 1.00 | 15.95 |
| 1775 | N | GLN | A | 437 | 33.481 | 22.35 | 7.647 | 1.00 | 16.16 |
| 1776 | CA | GLN | A | 437 | 34.183 | 23.53 | 7.137 | 1.00 | 16.84 |
| 1777 | CB | GLN | A | 437 | 35.235 | 23.15 | 6.09 | 1.00 | 17.05 |
| 1778 | CG | GLN | A | 437 | 36.012 | 24.37 | 5.576 | 1.00 | 20.71 |
| 1779 | CD | GLN | A | 437 | 36.985 | 24.04 | 4.46 | 1.00 | 22.77 |
| 1780 | OE1 | GLN | A | 437 | 36.622 | 23.4 | 3.475 | 1.00 | 25.06 |
| 1781 | NE2 | GLN | A | 437 | 38.227 | 24.5 | 4.604 | 1.00 | 26.03 |
| 1782 | C | GLN | A | 437 | 33.179 | 24.51 | 6.529 | 1.00 | 17.18 |
| 1783 | O | GLN | A | 437 | 33.329 | 25.72 | 6.668 | 1.00 | 17.33 |
| 1784 | N | LYS | A | 438 | 32.15 | 23.99 | 5.858 | 1.00 | 17.54 |
| 1785 | CA | LYS | A | 438 | 31.136 | 24.87 | 5.262 | 1.00 | 17.07 |
| 1786 | CB | LYS | A | 438 | 30.083 | 24.06 | 4.502 | 1.00 | 17.70 |
| 1787 | CG | LYS | A | 438 | 30.623 | 23.31 | 3.309 | 1.00 | 19.51 |
| 1788 | CD | LYS | A | 438 | 31.345 | 24.23 | 2.341 | 1.00 | 22.06 |
| 1789 | CE | LYS | A | 438 | 31.902 | 23.44 | 1.155 | 1.00 | 22.64 |
| 1790 | NZ | LYS | A | 438 | 32.819 | 24.28 | 0.343 | 1.00 | 21.64 |
| 1791 | C | LYS | A | 438 | 30.443 | 25.7 | 6.329 | 1.00 | 18.54 |
| 1792 | O | LYS | A | 438 | 30.07 | 26.85 | 6.084 | 1.00 | 16.97 |
| 1793 | N | MET | A | 439 | 30.257 | 25.12 | 7.512 | 1.00 | 18.74 |
| 1794 | CA | MET | A | 439 | 29.619 | 25.85 | 8.599 | 1.00 | 19.35 |
| 1795 | CB | MET | A | 439 | 29.426 | 24.95 | 9.814 | 1.00 | 22.86 |
| 1796 | CG | MET | A | 439 | 28.564 | 23.73 | 9.519 | 1.00 | 25.60 |
| 1797 | SD | MET | A | 439 | 28.191 | 22.76 | 10.972 | 1.00 | 29.86 |
| 1798 | CE | MET | A | 439 | 26.648 | 23.51 | 11.476 | 1.00 | 30.44 |
| 1799 | C | MET | A | 439 | 30.503 | 27.04 | 8.97 | 1.00 | 19.48 |
| 1800 | O | MET | A | 439 | 30.009 | 28.13 | 9.217 | 1.00 | 17.93 |
| 1801 | N | ALA | A | 440 | 31.815 | 26.82 | 9.007 | 1.00 | 19.56 |
| 1802 | CA | ALA | A | 440 | 32.762 | 27.88 | 9.34 | 1.00 | 18.57 |
| 1803 | CB | ALA | A | 440 | 34.153 | 27.29 | 9.549 | 1.00 | 20.87 |
| 1804 | C | ALA | A | 440 | 32.796 | 28.93 | 8.232 | 1.00 | 19.19 |
| 1805 | O | ALA | A | 440 | 32.903 | 30.13 | 8.496 | 1.00 | 18.72 |
| 1806 | N | ASP | A | 441 | 32.713 | 28.47 | 6.987 | 1.00 | 17.33 |
| 1807 | CA | ASP | A | 441 | 32.722 | 29.38 | 5.848 | 1.00 | 17.47 |
| 1808 | CB | ASP | A | 441 | 32.749 | 28.6 | 4.533 | 1.00 | 18.81 |
| 1809 | CG | ASP | A | 441 | 34.084 | 27.92 | 4.279 | 1.00 | 22.11 |
| 1810 | OD1 | ASP | A | 441 | 35.081 | 28.28 | 4.93 | 1.00 | 23.74 |
| 1811 | OD2 | ASP | A | 441 | 34.135 | 27.02 | 3.409 | 1.00 | 23.76 |
| 1812 | C | ASP | A | 441 | 31.479 | 30.26 | 5.888 | 1.00 | 17.16 |
| 1813 | O | ASP | A | 441 | 31.543 | 31.45 | 5.56 | 1.00 | 16.05 |
| 1814 | N | LEU | A | 442 | 30.351 | 29.69 | 6.299 | 1.00 | 15.92 |
| 1815 | CA | LEU | A | 442 | 29.1 | 30.44 | 6.373 | 1.00 | 15.51 |
| 1816 | CB | LEU | A | 442 | 27.921 | 29.5 | 6.648 | 1.00 | 14.97 |
| 1817 | CG | LEU | A | 442 | 27.461 | 28.65 | 5.457 | 1.00 | 16.25 |
| 1818 | CD1 | LEU | A | 442 | 26.459 | 27.62 | 5.923 | 1.00 | 15.43 |
| 1819 | CD2 | LEU | A | 442 | 26.835 | 29.54 | 4.389 | 1.00 | 16.30 |
| 1820 | C | LEU | A | 442 | 29.149 | 31.53 | 7.441 | 1.00 | 16.51 |
| 1821 | O | LEU | A | 442 | 28.64 | 32.62 | 7.243 | 1.00 | 15.91 |
| 1822 | N | ARG | A | 443 | 29.758 | 31.21 | 8.578 | 1.00 | 17.51 |
| 1823 | CA | ARG | A | 443 | 29.859 | 32.19 | 9.656 | 1.00 | 19.49 |
| 1824 | CB | ARG | A | 443 | 30.545 | 31.56 | 10.868 | 1.00 | 20.19 |
| 1825 | CG | ARG | A | 443 | 30.518 | 32.44 | 12.104 | 1.00 | 24.32 |
| 1826 | CD | ARG | A | 443 | 30.919 | 31.66 | 13.334 | 1.00 | 27.77 |
| 1827 | NE | ARG | A | 443 | 29.938 | 30.63 | 13.68 | 1.00 | 30.42 |
| 1828 | CZ | ARG | A | 443 | 29.983 | 29.92 | 14.799 | 1.00 | 32.49 |
| 1829 | NH1 | ARG | A | 443 | 30.963 | 30.12 | 15.669 | 1.00 | 33.93 |
| 1830 | NH2 | ARG | A | 443 | 29.052 | 29.01 | 15.058 | 1.00 | 32.62 |
| 1831 | C | ARG | A | 443 | 30.649 | 33.4 | 9.171 | 1.00 | 19.24 |
| 1832 | O | ARG | A | 443 | 30.288 | 34.54 | 9.451 | 1.00 | 20.11 |
| 1833 | N | GLN | A | 444 | 31.729 | 33.15 | 8.441 | 1.00 | 20.35 |
| 1834 | CA | GLN | A | 444 | 32.555 | 34.23 | 7.906 | 1.00 | 21.41 |
| 1835 | CB | GLN | A | 444 | 33.831 | 33.66 | 7.278 | 1.00 | 23.62 |
| 1836 | CG | GLN | A | 444 | 34.618 | 34.65 | 6.433 | 1.00 | 28.57 |
| 1837 | CD | GLN | A | 444 | 35.508 | 35.57 | 7.25 | 1.00 | 33.29 |
| 1838 | OE1 | GLN | A | 444 | 35.105 | 36.09 | 8.293 | 1.00 | 36.07 |
| 1839 | NE2 | GLN | A | 444 | 36.728 | 35.79 | 6.768 | 1.00 | 36.08 |
| 1840 | C | GLN | A | 444 | 31.761 | 34.99 | 6.852 | 1.00 | 20.82 |
| 1841 | O | GLN | A | 444 | 31.776 | 36.22 | 6.815 | 1.00 | 19.43 |
| 1842 | N | LEU | A | 445 | 31.062 | 34.25 | 5.997 | 1.00 | 19.72 |
| 1843 | CA | LEU | A | 445 | 30.255 | 34.85 | 4.943 | 1.00 | 18.86 |
| 1844 | CB | LEU | A | 445 | 29.577 | 33.75 | 4.112 | 1.00 | 18.95 |
| 1845 | CG | LEU | A | 445 | 28.856 | 34.16 | 2.826 | 1.00 | 20.08 |
| 1846 | CD1 | LEU | A | 445 | 29.888 | 34.62 | 1.793 | 1.00 | 20.86 |
| 1847 | CD2 | LEU | A | 445 | 28.06 | 32.97 | 2.275 | 1.00 | 21.90 |
| 1848 | C | LEU | A | 445 | 29.19 | 35.78 | 5.53 | 1.00 | 18.74 |
| 1849 | O | LEU | A | 445 | 28.909 | 36.84 | 4.963 | 1.00 | 17.29 |
| 1850 | N | VAL | A | 446 | 28.596 | 35.39 | 6.658 | 1.00 | 17.24 |
| 1851 | CA | VAL | A | 446 | 27.565 | 36.23 | 7.287 | 1.00 | 17.47 |
| 1852 | CB | VAL | A | 446 | 26.781 | 35.45 | 8.367 | 1.00 | 17.76 |
| 1853 | CG1 | VAL | A | 446 | 25.852 | 36.4 | 9.126 | 1.00 | 18.67 |
| 1854 | CG2 | VAL | A | 446 | 25.955 | 34.34 | 7.711 | 1.00 | 1452 |
| 1855 | C | VAL | A | 446 | 28.171 | 37.48 | 7.927 | 1.00 | 18.46 |
| 1856 | O | VAL | A | 446 | 27.612 | 38.57 | 7.832 | 1.00 | 18.68 |
| 1857 | N | THR | A | 447 | 29.311 | 37.3 | 8.584 | 1.00 | 18.45 |
| 1858 | CA | THR | A | 447 | 29.984 | 38.42 | 9.232 | 1.00 | 19.82 |
| 1859 | CB | THR | A | 447 | 31.313 | 37.97 | 9.872 | 1.00 | 20.76 |
| 1860 | OG1 | THR | A | 447 | 31.048 | 36.96 | 10.85 | 1.00 | 22.73 |
| 1861 | CG2 | THR | A | 447 | 32.008 | 39.14 | 10.542 | 1.00 | 23.28 |
| 1862 | C | THR | A | 447 | 30.272 | 39.5 | 8.197 | 1.00 | 20.08 |
| 1863 | O | THR | A | 447 | 30.04 | 40.69 | 8.434 | 1.00 | 20.49 |
| 1864 | N | GLU | A | 448 | 30.777 | 39.07 | 7.044 | 1.00 | 18.53 |
| 1865 | CA | GLU | A | 448 | 31.104 | 39.98 | 5.963 | 1.00 | 19.18 |
| 1866 | CB | GLU | A | 448 | 31.901 | 39.23 | 4.889 | 1.00 | 19.51 |
| 1867 | CG | GLU | A | 448 | 33.064 | 38.45 | 5.485 | 1.00 | 23.78 |
| 1868 | CD | GLU | A | 448 | 33.905 | 37.72 | 4.454 | 1.00 | 25.56 |
| 1869 | OE1 | GLU | A | 448 | 33.331 | 37.13 | 3.518 | 1.00 | 27.66 |
| 1870 | OE2 | GLU | A | 448 | 35.147 | 37.73 | 4.592 | 1.00 | 27.05 |
| 1871 | C | GLU | A | 448 | 29.85 | 40.61 | 5.358 | 1.00 | 17.55 |
| 1872 | O | GLU | A | 448 | 29.86 | 41.77 | 4.961 | 1.00 | 18.18 |
| 1873 | N | HIS | A | 449 | 28.771 | 39.84 | 5.283 | 1.00 | 16.47 |
| 1874 | CA | HIS | A | 449 | 27.526 | 40.35 | 4.729 | 1.00 | 15.35 |
| 1875 | CB | HIS | A | 449 | 26.509 | 39.22 | 4.559 | 1.00 | 13.45 |
| 1876 | CG | HIS | A | 449 | 25.167 | 39.67 | 4.061 | 1.00 | 13.44 |
| 1877 | CD2 | HIS | A | 449 | 24 | 39.87 | 4.715 | 1.00 | 14.46 |
| 1878 | ND1 | HIS | A | 449 | 24.914 | 39.94 | 2.734 | 1.00 | 14.54 |
| 1879 | CE1 | HIS | A | 449 | 23.65 | 40.3 | 2.593 | 1.00 | 14.39 |
| 1880 | NE2 | HIS | A | 449 | 23.072 | 40.26 | 3.78 | 1.00 | 14.22 |
| 1881 | C | HIS | A | 449 | 26.949 | 41.44 | 5.633 | 1.00 | 15.34 |
| 1882 | O | HIS | A | 449 | 26.508 | 42.48 | 5.145 | 1.00 | 16.88 |
| 1883 | N | ALA | A | 450 | 26.953 | 41.22 | 6.942 | 1.00 | 16.12 |
| 1884 | CA | ALA | A | 450 | 26.422 | 42.2 | 7.883 | 1.00 | 17.96 |
| 1885 | CB | ALA | A | 450 | 26.507 | 41.67 | 9.31 | 1.00 | 17.75 |
| 1886 | C | ALA | A | 450 | 27.191 | 43.52 | 7.776 | 1.00 | 19.46 |
| 1887 | O | ALA | A | 450 | 26.623 | 44.6 | 7.958 | 1.00 | 18.94 |
| 1888 | N | GLN | A | 451 | 28.482 | 43.43 | 7.481 | 1.00 | 21.32 |
| 1889 | CA | GLN | A | 451 | 29.31 | 44.63 | 7.348 | 1.00 | 22.42 |
| 1890 | CB | GLN | A | 451 | 30.779 | 44.25 | 7.151 | 1.00 | 26.37 |
| 1891 | CG | GLN | A | 451 | 31.721 | 45.44 | 7.069 | 1.00 | 31.72 |
| 1892 | CD | GLN | A | 451 | 33.179 | 45.03 | 6.974 | 1.00 | 34.48 |
| 1893 | OE1 | GLN | A | 451 | 33.653 | 44.21 | 7.757 | 1.00 | 37.93 |
| 1894 | NE2 | GLN | A | 451 | 33.897 | 45.6 | 6.016 | 1.00 | 35.81 |
| 1895 | C | GLN | A | 451 | 28.831 | 45.45 | 6.161 | 1.00 | 21.76 |
| 1896 | O | GLN | A | 451 | 28.707 | 46.67 | 6.247 | 1.00 | 20.34 |
| 1897 | N | LEU | A | 452 | 28.556 | 44.77 | 5.05 | 1.00 | 19.43 |
| 1898 | CA | LEU | A | 452 | 28.086 | 45.46 | 3.855 | 1.00 | 20.58 |
| 1899 | CB | LEU | A | 452 | 28.063 | 44.5 | 2.662 | 1.00 | 21.26 |
| 1900 | CG | LEU | A | 452 | 27.566 | 45.09 | 1.338 | 1.00 | 23.72 |
| 1901 | GD1 | LEU | A | 452 | 28.481 | 46.24 | 0.906 | 1.00 | 23.27 |
| 1902 | GD2 | LEU | A | 452 | 27.531 | 44 | 0.277 | 1.00 | 23.43 |
| 1903 | C | LEU | A | 452 | 26.688 | 46.01 | 4.107 | 1.00 | 19.34 |
| 1904 | O | LEU | A | 452 | 26.369 | 47.12 | 3.676 | 1.00 | 20.88 |
| 1905 | N | VAL | A | 453 | 25.857 | 45.25 | 4.811 | 1.00 | 19.86 |
| 1906 | CA | VAL | A | 453 | 24.501 | 45.69 | 5.125 | 1.00 | 19.89 |
| 1907 | CB | VAL | A | 453 | 23.719 | 44.61 | 5.923 | 1.00 | 21.23 |
| 1908 | CG1 | VAL | A | 453 | 22.35 | 45.14 | 6.342 | 1.00 | 22.52 |
| 1909 | CG2 | VAL | A | 453 | 23.538 | 43.36 | 5.063 | 1.00 | 20.41 |
| 1910 | C | VAL | A | 453 | 24.557 | 46.99 | 5.938 | 1.00 | 21.15 |
| 1911 | O | VAL | A | 453 | 23.745 | 47.89 | 5.733 | 1.00 | 20.95 |
| 1912 | N | GLN | A | 454 | 25.522 | 47.08 | 6.847 | 1.00 | 21.46 |
| 1913 | CA | GLN | A | 454 | 25.669 | 48.28 | 7.667 | 1.00 | 23.89 |
| 1914 | CB | GLN | A | 454 | 26.729 | 48.05 | 8.749 | 1.00 | 27.62 |
| 1915 | CG | GLN | A | 454 | 26.848 | 49.2 | 9.754 | 1.00 | 32.28 |
| 1916 | CD | GLN | A | 454 | 25.615 | 49.35 | 10.635 | 1.00 | 35.43 |
| 1917 | OE1 | GLN | A | 454 | 25.592 | 50.18 | 11.546 | 1.00 | 38.17 |
| 1918 | NE2 | GLN | A | 454 | 24.586 | 48.55 | 10.371 | 1.00 | 37.92 |
| 1919 | C | GLN | A | 454 | 26.066 | 49.47 | 6.789 | 1.00 | 24.56 |
| 1920 | O | GLN | A | 454 | 25.59 | 50.58 | 6.993 | 1.00 | 22.97 |
| 1921 | N | ILE | A | 455 | 26.938 | 49.22 | 5.817 | 1.00 | 23.13 |
| 1922 | CA | ILE | A | 455 | 27.383 | 50.27 | 4.915 | 1.00 | 24.75 |
| 1923 | CB | ILE | A | 455 | 28.474 | 49.75 | 3.948 | 1.00 | 24.82 |
| 1924 | CG2 | ILE | A | 455 | 28.746 | 50.76 | 2.846 | 1.00 | 24.01 |
| 1925 | CG1 | ILE | A | 455 | 29.755 | 49.45 | 4.738 | 1.00 | 25.40 |
| 1926 | CD1 | ILE | A | 455 | 30.846 | 48.78 | 3.935 | 1.00 | 25.53 |
| 1927 | C | ILE | A | 455 | 26.203 | 50.79 | 4.109 | 1.00 | 25.90 |
| 1928 | O | ILE | A | 455 | 26 | 52 | 3.969 | 1.00 | 26.44 |
| 1929 | N | ILE | A | 456 | 25.41 | 49.86 | 3.578 | 1.00 | 26.77 |
| 1930 | CA | ILE | A | 456 | 24.232 | 50.2 | 2.798 | 1.00 | 29.88 |
| 1931 | CB | ILE | A | 456 | 23.559 | 48.93 | 2.21 | 1.00 | 30.75 |
| 1932 | CG2 | ILE | A | 456 | 22.188 | 49.27 | 1.69 | 1.00 | 33.39 |
| 1933 | CG1 | ILE | A | 456 | 24.366 | 48.35 | 1.059 | 1.00 | 31.69 |
| 1934 | OD1 | ILE | A | 456 | 23.79 | 47.03 | 0.547 | 1.00 | 34.09 |
| 1935 | C | ILE | A | 456 | 23.244 | 50.94 | 3.689 | 1.00 | 30.49 |
| 1936 | O | ILE | A | 456 | 22.611 | 51.92 | 3.278 | 1.00 | 30.43 |
| 1937 | N | LYS | A | 457 | 23.101 | 50.5 | 4.922 | 1.00 | 32.72 |
| 1938 | CA | LYS | A | 457 | 22.172 | 51.15 | 5.818 | 1.00 | 36.04 |
| 1939 | CB | LYS | A | 457 | 22.139 | 50.37 | 7.131 | 1.00 | 37.66 |
| 1940 | CG | LYS | A | 457 | 21.215 | 50.95 | 8.145 | 1.00 | 40.69 |
| 1941 | CD | LYS | A | 457 | 20.92 | 49.91 | 9.19 | 1.00 | 41.72 |
| 1942 | CE | LYS | A | 457 | 19.917 | 50.43 | 10.191 | 1.00 | 44.25 |
| 1943 | NZ | LYS | A | 457 | 20.495 | 51.43 | 11.136 | 1.00 | 45.65 |
| 1944 | C | LYS | A | 457 | 22.479 | 52.63 | 6.094 | 1.00 | 36.68 |
| 1945 | O | LYS | A | 457 | 21.564 | 53.45 | 6.176 | 1.00 | 36.26 |
| 1946 | N | LYS | A | 458 | 23.756 | 52.97 | 6.213 | 1.00 | 37.99 |
| 1947 | CA | LYS | A | 458 | 24.132 | 54.35 | 6.501 | 1.00 | 39.24 |
| 1948 | CB | LYS | A | 458 | 25.327 | 54.38 | 7.465 | 1.00 | 41.14 |
| 1949 | CG | LYS | A | 458 | 26.689 | 54.41 | 6.785 | 1.00 | 43.28 |
| 1950 | GD | LYS | A | 458 | 27.822 | 54.42 | 7.806 | 1.00 | 44.36 |
| 1951 | CE | LYS | A | 458 | 27.936 | 53.09 | 8.516 | 1.00 | 45.26 |
| 1952 | NZ | LYS | A | 458 | 28.212 | 51.99 | 7.55 | 1.00 | 44.67 |
| 1953 | C | LYS | A | 458 | 24.459 | 55.18 | 5.258 | 1.00 | 39.55 |
| 1954 | O | LYS | A | 458 | 24.528 | 56.41 | 5.321 | 1.00 | 39.79 |
| 1955 | N | THR | A | 459 | 24.65 | 54.5 | 4.132 | 1.00 | 39.02 |
| 1956 | CA | THR | A | 459 | 24.988 | 55.16 | 2.88 | 1.00 | 39.78 |
| 1957 | CB | THR | A | 459 | 26.164 | 54.43 | 2.182 | 1.00 | 40.83 |
| 1958 | OG1 | THR | A | 459 | 27.404 | 54.92 | 2.715 | 1.00 | 41.96 |
| 1959 | CG2 | THR | A | 459 | 26.136 | 54.65 | 0.68 | 1.00 | 42.44 |
| 1960 | C | THR | A | 459 | 23.823 | 55.29 | 1.901 | 1.00 | 39.33 |
| 1961 | O | THR | A | 459 | 23.734 | 56.27 | 1.16 | 1.00 | 39.15 |
| 1962 | N | GLU | A | 460 | 22.934 | 54.31 | 1.892 | 1.00 | 38.93 |
| 1963 | CA | GLU | A | 460 | 21.792 | 54.33 | 0.989 | 1.00 | 39.36 |
| 1964 | CB | GLU | A | 460 | 21.621 | 52.97 | 0.312 | 1.00 | 37.13 |
| 1965 | CG | GLU | A | 460 | 22.799 | 52.51 | −0.549 | 1.00 | 33.20 |
| 1966 | CD | GLU | A | 460 | 22.988 | 53.33 | −1.812 | 1.00 | 31.38 |
| 1967 | OE1 | GLU | A | 460 | 21.988 | 53.86 | −2.345 | 1.00 | 31.23 |
| 1968 | OE2 | GLU | A | 460 | 24.137 | 53.44 | −2.288 | 1.00 | 27.05 |
| 1969 | C | GLU | A | 460 | 20.514 | 54.69 | 1.742 | 1.00 | 41.61 |
| 1970 | O | GLU | A | 460 | 19.856 | 53.82 | 2.308 | 1.00 | 41.95 |
| 1971 | N | SER | A | 461 | 20.167 | 55.97 | 1.755 | 1.00 | 44.12 |
| 1972 | CA | SER | A | 461 | 18.958 | 56.41 | 2.439 | 1.00 | 46.91 |
| 1973 | CB | SER | A | 461 | 19.07 | 57.89 | 2.821 | 1.00 | 47.79 |
| 1974 | OG | SER | A | 461 | 20.075 | 58.09 | 3.805 | 1.00 | 49.02 |
| 1975 | C | SER | A | 461 | 17.769 | 56.2 | 1.516 | 1.00 | 48.12 |
| 1976 | O | SER | A | 461 | 16.613 | 56.29 | 1.928 | 1.00 | 48.49 |
| 1977 | N | ASP | A | 462 | 18.077 | 55.91 | 0.257 | 1.00 | 49.25 |
| 1978 | CA | ASP | A | 462 | 17.068 | 55.65 | −0.758 | 1.00 | 50.01 |
| 1979 | CB | ASP | A | 462 | 17.751 | 55.26 | −2.067 | 1.00 | 51.19 |
| 1980 | CG | ASP | A | 462 | 19.027 | 54.46 | −1.839 | 1.00 | 51.21 |
| 1981 | OD1 | ASP | A | 462 | 19.959 | 55.01 | −1.215 | 1.00 | 52.21 |
| 1982 | OD2 | ASP | A | 462 | 19.103 | 53.3 | −2.279 | 1.00 | 52.20 |
| 1983 | C | ASP | A | 462 | 16.161 | 54.53 | −0.285 | 1.00 | 49.54 |
| 1984 | O | ASP | A | 462 | 14.948 | 54.57 | −0.482 | 1.00 | 50.36 |
| 1985 | N | ALA | A | 463 | 16.769 | 53.54 | 0.346 | 1.00 | 49.32 |
| 1986 | CA | ALA | A | 463 | 16.044 | 52.39 | 0.864 | 1.00 | 47.69 |
| 1987 | CB | ALA | A | 463 | 16.147 | 51.24 | −0.105 | 1.00 | 48.66 |
| 1988 | C | ALA | A | 463 | 16.65 | 52.01 | 2.209 | 1.00 | 46.49 |
| 1989 | O | ALA | A | 463 | 17.867 | 52.01 | 2.365 | 1.00 | 47.87 |
| 1990 | N | ALA | A | 464 | 15.797 | 51.69 | 3.177 | 1.00 | 43.89 |
| 1991 | CA | ALA | A | 464 | 16.251 | 51.34 | 4.514 | 1.00 | 41.22 |
| 1992 | CB | ALA | A | 464 | 15.312 | 51.94 | 5.553 | 1.00 | 41.01 |
| 1993 | C | ALA | A | 464 | 16.349 | 49.83 | 4.714 | 1.00 | 39.82 |
| 1994 | O | ALA | A | 464 | 17.077 | 49.14 | 3.987 | 1.00 | 41.52 |
| 1995 | N | LEU | A | 465 | 15.628 | 49.33 | 5.715 | 1.00 | 36.01 |
| 1996 | CA | LEU | A | 465 | 15.588 | 47.9 | 6.058 | 1.00 | 32.30 |
| 1997 | CB | LEU | A | 465 | 16.754 | 47.51 | 6.977 | 1.00 | 33.09 |
| 1998 | CG | LEU | A | 465 | 18.085 | 47.02 | 6.399 | 1.00 | 33.05 |
| 1999 | CD1 | LEU | A | 465 | 18.95 | 46.51 | 7.545 | 1.00 | 32.55 |
| 2000 | CD2 | LEU | A | 465 | 17.854 | 45.9 | 5.398 | 1.00 | 31.55 |
| 2001 | C | LEU | A | 465 | 14.284 | 47.56 | 6.771 | 1.00 | 29.76 |
| 2002 | O | LEU | A | 465 | 13.78 | 48.34 | 7.59 | 1.00 | 26.72 |
| 2003 | N | HIS | A | 466 | 13.749 | 46.38 | 6.458 | 1.00 | 26.87 |
| 2004 | CA | HIS | A | 466 | 12.511 | 45.9 | 7.057 | 1.00 | 25.80 |
| 2005 | CB | HIS | A | 466 | 12.115 | 44.56 | 6.418 | 1.00 | 24.73 |
| 2006 | CG | HIS | A | 466 | 10.849 | 43.98 | 6.964 | 1.00 | 25.28 |
| 2007 | CD2 | HIS | A | 466 | 10.576 | 43.4 | 8.157 | 1.00 | 24.58 |
| 2008 | ND1 | HIS | A | 466 | 9.678 | 43.92 | 6.237 | 1.00 | 26.52 |
| 2009 | CE1 | HIS | A | 466 | 8.739 | 43.34 | 6.959 | 1.00 | 25.66 |
| 2010 | NE2 | HIS | A | 466 | 9.259 | 43.01 | 8.13 | 1.00 | 26.42 |
| 2011 | C | HIS | A | 466 | 12.716 | 45.72 | 8.563 | 1.00 | 25.90 |
| 2012 | O | HIS | A | 466 | 13.796 | 45.33 | 9.009 | 1.00 | 25.20 |
| 2013 | N | PRO | A | 467 | 11.674 | 46 | 9.364 | 1.00 | 25.60 |
| 2014 | CD | PRO | A | 467 | 10.372 | 46.55 | 8.935 | 1.00 | 26.08 |
| 2015 | CA | PRO | A | 467 | 11.724 | 45.87 | 10.823 | 1.00 | 25.42 |
| 2016 | CE | PRO | A | 467 | 10.262 | 46.04 | 11.223 | 1.00 | 25.91 |
| 2017 | CG | PRO | A | 467 | 9.78 | 47.04 | 10.238 | 1.00 | 27.38 |
| 2018 | C | PRO | A | 467 | 12.317 | 44.56 | 11.335 | 1.00 | 25.07 |
| 2019 | O | PRO | A | 467 | 13.16 | 44.56 | 12.233 | 1.00 | 25.29 |
| 2020 | N | LEU | A | 468 | 11.873 | 43.44 | 10.774 | 1.00 | 23.71 |
| 2021 | CA | LEU | A | 468 | 12.378 | 42.15 | 11.215 | 1.00 | 22.76 |
| 2022 | CB | LEU | A | 468 | 11.611 | 41 | 10.538 | 1.00 | 22.31 |
| 2023 | CG | LEU | A | 468 | 12.092 | 39.58 | 10.878 | 1.00 | 22.34 |
| 2024 | CD1 | LEU | A | 468 | 11.953 | 39.32 | 12.379 | 1.00 | 23.76 |
| 2025 | CD2 | LEU | A | 468 | 11.277 | 38.57 | 10.09 | 1.00 | 22.76 |
| 2026 | C | LEU | A | 468 | 13.866 | 42 | 10.927 | 1.00 | 22.18 |
| 2027 | O | LEU | A | 468 | 14.617 | 41.49 | 11.756 | 1.00 | 21.97 |
| 2028 | N | LEU | A | 469 | 14.296 | 42.44 | 9.752 | 1.00 | 21.66 |
| 2029 | CA | LEU | A | 469 | 15.704 | 42.34 | 9.394 | 1.00 | 21.60 |
| 2030 | CE | LEU | A | 469 | 15.885 | 42.62 | 7.899 | 1.00 | 20.70 |
| 2031 | CG | LEU | A | 469 | 14.968 | 41.76 | 7.024 | 1.00 | 19.18 |
| 2032 | CD1 | LEU | A | 469 | 15.329 | 41.95 | 5.56 | 1.00 | 20.00 |
| 2033 | CD2 | LEU | A | 469 | 15.104 | 40.29 | 7.424 | 1.00 | 16.85 |
| 2034 | C | LEU | A | 469 | 16.534 | 43.3 | 10.231 | 1.00 | 23.25 |
| 2035 | O | LEU | A | 469 | 17.671 | 43.01 | 10.613 | 1.00 | 21.71 |
| 2036 | N | GLN | A | 470 | 15.953 | 44.46 | 10.531 | 1.00 | 23.59 |
| 2037 | CA | GLN | A | 470 | 16.649 | 45.44 | 11.332 | 1.00 | 26.43 |
| 2038 | CB | GLN | A | 470 | 15.819 | 46.72 | 11.436 | 1.00 | 28.61 |
| 2039 | CG | GLN | A | 470 | 16.52 | 47.84 | 12.192 | 1.00 | 34.25 |
| 2040 | CD | GLN | A | 470 | 17.805 | 48.3 | 11.52 | 1.00 | 36.90 |
| 2041 | OE1 | GLN | A | 470 | 18.567 | 49.08 | 12.092 | 1.00 | 40.50 |
| 2042 | NE2 | GLN | A | 470 | 18.048 | 47.82 | 10.303 | 1.00 | 38.17 |
| 2043 | C | GLN | A | 470 | 16.95 | 44.9 | 12.727 | 1.00 | 25.75 |
| 2044 | O | GLN | A | 470 | 18.057 | 45.07 | 13.229 | 1.00 | 27.57 |
| 2045 | N | GLU | A | 471 | 15.986 | 44.22 | 13.354 | 1.00 | 25.39 |
| 2046 | CA | GLU | A | 471 | 16.239 | 43.7 | 14.693 | 1.00 | 26.81 |
| 2047 | CB | GLU | A | 471 | 14.929 | 43.29 | 15.397 | 1.00 | 28.93 |
| 2048 | CG | GLU | A | 471 | 14.136 | 42.15 | 14.784 | 1.00 | 30.21 |
| 2049 | CD | GLU | A | 471 | 12.837 | 41.88 | 15.551 | 1.00 | 32.16 |
| 2050 | QE1 | GLU | A | 471 | 11.987 | 42.79 | 15.629 | 1.00 | 32.22 |
| 2051 | 0E2 | GLU | A | 471 | 12.665 | 40.76 | 16.077 | 1.00 | 30.73 |
| 2052 | C | GLU | A | 471 | 17.24 | 42.55 | 14.681 | 1.00 | 25.84 |
| 2053 | O | GLU | A | 471 | 17.978 | 42.34 | 15.648 | 1.00 | 25.91 |
| 2054 | N | ILE | A | 472 | 17.287 | 41.79 | 13.586 | 1.00 | 23.23 |
| 2055 | CA | ILE | A | 472 | 18.238 | 40.7 | 13.498 | 1.00 | 22.62 |
| 2056 | CB | ILE | A | 472 | 17.941 | 39.78 | 12.282 | 1.00 | 23.65 |
| 2057 | CG2 | ILE | A | 472 | 19.106 | 38.81 | 12.049 | 1.00 | 21.96 |
| 2058 | CG1 | ILE | A | 472 | 16.642 | 39.01 | 12.531 | 1.00 | 22.25 |
| 2059 | CD1 | ILE | A | 472 | 16.172 | 38.18 | 11.345 | 1.00 | 24.20 |
| 2060 | C | ILE | A | 472 | 19.656 | 41.27 | 13.392 | 1.00 | 23.04 |
| 2061 | O | ILE | A | 472 | 20.567 | 40.79 | 14.061 | 1.00 | 20.73 |
| 2062 | N | TYR | A | 473 | 19.836 | 42.3 | 12.57 | 1.00 | 23.82 |
| 2063 | CA | TYR | A | 473 | 21.159 | 42.9 | 12.394 | 1.00 | 25.85 |
| 2064 | CB | TYR | A | 473 | 21.233 | 43.63 | 11.057 | 1.00 | 24.50 |
| 2065 | CG | TYR | A | 473 | 21.41 | 42.69 | 9.887 | 1.00 | 22.75 |
| 2066 | CD1 | TYR | A | 473 | 22.574 | 41.94 | 9.749 | 1.00 | 23.07 |
| 2067 | CE1 | TYR | A | 473 | 22.723 | 41.03 | 8.695 | 1.00 | 22.58 |
| 2068 | CD2 | TYR | A | 473 | 20.401 | 42.53 | 8.945 | 1.00 | 23.44 |
| 2069 | CE2 | TYR | A | 473 | 20.539 | 41.63 | 7.896 | 1.00 | 20.95 |
| 2070 | CZ | TYR | A | 473 | 21.698 | 40.89 | 7.777 | 1.00 | 22.05 |
| 2071 | OH | TYR | A | 473 | 21.822 | 39.99 | 6.74 | 1.00 | 21.31 |
| 2072 | C | TYR | A | 473 | 21.608 | 43.82 | 13.519 | 1.00 | 27.80 |
| 2073 | O | TYR | A | 473 | 22.805 | 44.05 | 13.69 | 1.00 | 28.04 |
| 2074 | N | AkO | A | 474 | 20.661 | 44.36 | 14.282 | 1.00 | 29.91 |
| 2075 | CA | ARG | A | 474 | 21.006 | 45.25 | 15.391 | 1.00 | 33.29 |
| 2076 | CB | ARG | A | 474 | 19.737 | 45.78 | 16.068 | 1.00 | 35.76 |
| 2077 | CG | ARG | A | 474 | 19.987 | 46.55 | 17.366 | 1.00 | 38.99 |
| 2078 | CD | ARG | A | 474 | 18.675 | 47.02 | 17.984 | 1.00 | 42.46 |
| 2079 | NE | ARG | A | 474 | 18.862 | 47.7 | 19.265 | 1.00 | 44.44 |
| 2080 | CZ | ARG | A | 474 | 17.879 | 48.28 | 19.953 | 1.00 | 46.85 |
| 2081 | NH1 | ARG | A | 474 | 16.637 | 48.26 | 19.485 | 1.00 | 47.83 |
| 2082 | NH2 | ARG | A | 474 | 18.134 | 48.87 | 21.111 | 1.00 | 47.38 |
| 2083 | C | ARG | A | 474 | 21.859 | 44.51 | 16.42 | 1.00 | 34.75 |
| 2084 | O | ARG | A | 474 | 21.409 | 43.53 | 17.025 | 1.00 | 34.47 |
| 2085 | N | ASP | A | 475 | 23.094 | 44.97 | 16.604 | 1.00 | 36.62 |
| 2086 | CA | ASP | A | 475 | 24.019 | 44.37 | 17.563 | 1.00 | 38.83 |
| 2087 | CB | ASP | A | 475 | 23.447 | 44.45 | 18.982 | 1.00 | 39.46 |
| 2088 | CG | ASP | A | 475 | 23.228 | 45.88 | 19.434 | 1.00 | 40.61 |
| 2089 | OD1 | ASP | A | 475 | 24.135 | 46.72 | 19.217 | 1.00 | 40.51 |
| 2090 | OD2 | ASP | A | 475 | 22.156 | 46.17 | 20.014 | 1.00 | 40.94 |
| 2091 | C | ASP | A | 475 | 24.396 | 42.92 | 17.274 | 1.00 | 39.96 |
| 2092 | O | ASP | A | 475 | 24.768 | 42.18 | 18.187 | 1.00 | 39.85 |
| 2093 | N | MET | A | 476 | 24.303 | 42.5 | 16.015 | 1.00 | 41.66 |
| 2094 | CA | MET | A | 476 | 24.664 | 41.13 | 15.66 | 1.00 | 43.69 |
| 2095 | CB | MET | A | 476 | 24.33 | 40.85 | 14.196 | 1.00 | 43.01 |
| 2096 | CG | MET | A | 476 | 24.632 | 39.42 | 13.777 | 1.00 | 43.53 |
| 2097 | SD | MET | A | 476 | 24.318 | 39.13 | 12.035 | 1.00 | 45.18 |
| 2098 | CE | MET | A | 476 | 22.532 | 38.88 | 12.057 | 1.00 | 44.23 |
| 2099 | C | MET | A | 476 | 26.163 | 40.97 | 15.882 | 1.00 | 45.56 |
| 2100 | O | MET | A | 476 | 26.603 | 40.18 | 16.718 | 1.00 | 46.26 |
| 2101 | N | TYR | A | 477 | 26.942 | 41.73 | 15.12 | 1.00 | 46.99 |
| 2102 | CA | TYR | A | 477 | 28.394 | 41.7 | 15.225 | 1.00 | 48.84 |
| 2103 | CB | TYR | A | 477 | 29.023 | 41.13 | 13.949 | 1.00 | 48.71 |
| 2104 | CG | TYR | A | 477 | 28.735 | 39.65 | 13.725 | 1.00 | 48.95 |
| 2105 | CD1 | TYR | A | 477 | 27.925 | 39.23 | 12.67 | 1.00 | 49.23 |
| 2106 | CE1 | TYR | A | 477 | 27.653 | 37.88 | 12.466 | 1.00 | 49.07 |
| 2107 | CD2 | TYR | A | 477 | 29.268 | 38.68 | 14.574 | 1.00 | 49.57 |
| 2108 | CE2 | TYR | A | 477 | 29.003 | 37.33 | 14.381 | 1.00 | 49.37 |
| 2109 | GZ | TYR | A | 477 | 28.196 | 36.93 | 13.327 | 1.00 | 49.98 |
| 2110 | OH | TYR | A | 477 | 27.937 | 35.59 | 13.14 | 1.00 | 49.98 |
| 2111 | C | TYR | A | 477 | 28.888 | 43.13 | 15.459 | 1.00 | 49.62 |
| 2112 | O | TYR | A | 477 | 29.046 | 43.86 | 14.465 | 1.00 | 50.21 |
| 2113 | OT | TYR | A | 477 | 29.08 | 43.5 | 16.638 | 1.00 | 50.66 |
| 2114 | CB | GLU | B | 685 | 18.563 | 43.31 | 21.966 | 1.00 | 63.65 |
| 2115 | CG | GLU | B | 685 | 18.355 | 43.18 | 23.466 | 1.00 | 63.87 |
| 2116 | CD | GLU | B | 685 | 18.5 | 41.75 | 23.944 | 1.00 | 64.23 |
| 2117 | OE1 | GLU | B | 685 | 17.602 | 40.93 | 23.645 | 1.00 | 64.28 |
| 2118 | OE2 | GLU | B | 685 | 19.509 | 41.44 | 24.611 | 1.00 | 64.53 |
| 2119 | C | GLU | B | 685 | 16.485 | 44.44 | 21.146 | 1.00 | 61.94 |
| 2120 | O | GLU | B | 685 | 15.976 | 44.7 | 20.055 | 1.00 | 62.27 |
| 2121 | N | GLU | B | 685 | 18.315 | 45.76 | 22.219 | 1.00 | 62.90 |
| 2122 | CA | GLU | B | 685 | 17.989 | 44.59 | 21.355 | 1.00 | 62.73 |
| 2123 | N | ARG | B | 686 | 15.783 | 44.01 | 22.191 | 1.00 | 60.58 |
| 2124 | CA | ARG | B | 686 | 14.335 | 43.82 | 22.134 | 1.00 | 59.13 |
| 2125 | CB | ARG | B | 686 | 13.674 | 45.08 | 21.56 | 1.00 | 60.07 |
| 2126 | CG | ARG | B | 686 | 12.151 | 45.06 | 21.524 | 1.00 | 61.27 |
| 2127 | CD | ARG | B | 686 | 11.543 | 45.61 | 22.805 | 1.00 | 62.16 |
| 2128 | NE | ARG | B | 686 | 10.168 | 46.05 | 22.594 | 1.00 | 63.43 |
| 2129 | CZ | ARG | B | 686 | 9.147 | 45.24 | 22.329 | 1.00 | 64.44 |
| 2130 | NH1 | ARG | B | 686 | 9.339 | 43.93 | 22.247 | 1.00 | 64.89 |
| 2131 | NH2 | ARG | B | 686 | 7.936 | 45.75 | 22.128 | 1.00 | 65.34 |
| 2132 | C | ARG | B | 686 | 13.932 | 42.6 | 21.299 | 1.00 | 57.39 |
| 2133 | O | ARG | B | 686 | 13.69 | 41.52 | 21.841 | 1.00 | 58.48 |
| 2134 | N | HIS | B | 687 | 13.867 | 42.78 | 19.982 | 1.00 | 54.71 |
| 2135 | CA | HIS | B | 687 | 13.479 | 41.72 | 19.052 | 1.00 | 51.16 |
| 2136 | CB | HIS | B | 687 | 14.316 | 40.46 | 19.28 | 1.00 | 51.72 |
| 2137 | CG | HIS | B | 687 | 15.776 | 40.64 | 19.004 | 1.00 | 51.47 |
| 2138 | CD2 | HIS | B | 687 | 16.561 | 40.16 | 18.01 | 1.00 | 51.61 |
| 2139 | ND1 | HIS | B | 687 | 16.596 | 41.41 | 19.802 | 1.00 | 52.04 |
| 2140 | CE1 | HIS | B | 687 | 17.822 | 41.39 | 19.311 | 1.00 | 51.83 |
| 2141 | NE2 | HIS | B | 687 | 17.828 | 40.64 | 18.225 | 1.00 | 51.34 |
| 2142 | C | HIS | B | 687 | 11.998 | 41.39 | 19.21 | 1.00 | 48.80 |
| 2143 | O | HIS | B | 687 | 11.628 | 40.24 | 19.412 | 1.00 | 48.44 |
| 2144 | N | ALA | B | 688 | 11.157 | 42.41 | 19.108 | 1.00 | 45.79 |
| 2145 | CA | ALA | B | 688 | 9.714 | 42.25 | 19.251 | 1.00 | 43.75 |
| 2146 | CB | ALA | B | 688 | 9.027 | 43.61 | 19.108 | 1.00 | 43.70 |
| 2147 | C | ALA | B | 688 | 9.106 | 41.26 | 18.265 | 1.00 | 41.94 |
| 2148 | O | ALA | B | 688 | 8.488 | 40.27 | 18.669 | 1.00 | 40.94 |
| 2149 | N | ILE | B | 689 | 9.282 | 41.52 | 16.973 | 1.00 | 40.75 |
| 2150 | CA | ILE | B | 689 | 8.728 | 40.65 | 15.939 | 1.00 | 39.42 |
| 2151 | CB | ILE | B | 689 | 9.093 | 41.17 | 14.532 | 1.00 | 39.12 |
| 2152 | G02 | ILE | B | 689 | 8.624 | 40.18 | 13.468 | 1.00 | 38.63 |
| 2153 | CG1 | ILE | B | 689 | 8.444 | 42.54 | 14.312 | 1.00 | 38.90 |
| 2154 | CD1 | ILE | B | 689 | 8.728 | 43.15 | 12.959 | 1.00 | 39.20 |
| 2155 | C | ILE | B | 689 | 9.18 | 39.2 | 16.077 | 1.00 | 38.83 |
| 2156 | O | ILE | B | 689 | 8.36 | 38.28 | 16.039 | 1.00 | 37.60 |
| 2157 | N | LEU | B | 690 | 10.482 | 39 | 16.24 | 1.00 | 38.40 |
| 2158 | CA | LEU | B | 690 | 11.027 | 37.66 | 16.389 | 1.00 | 38.63 |
| 2159 | CB | LEU | B | 690 | 12.54 | 37.74 | 16.596 | 1.00 | 39.36 |
| 2160 | CG | LEU | B | 690 | 13.378 | 36.57 | 16.066 | 1.00 | 40.30 |
| 2161 | CD1 | LEU | B | 690 | 14.853 | 36.95 | 16.107 | 1.00 | 39.81 |
| 2162 | CD2 | LEU | B | 690 | 13.111 | 35.32 | 16.884 | 1.00 | 41.50 |
| 2163 | C | LEU | B | 690 | 10.352 | 36.99 | 17.584 | 1.00 | 39.78 |
| 2164 | O | LEU | B | 690 | 9.892 | 35.85 | 17.491 | 1.00 | 38.83 |
| 2165 | N | HIS | B | 691 | 10.287 | 37.7 | 18.707 | 1.00 | 40.27 |
| 2166 | CA | HIS | B | 691 | 9.64 | 37.17 | 19.902 | 1.00 | 41.72 |
| 2167 | CB | HIS | B | 691 | 9.659 | 38.2 | 21.029 | 1.00 | 43.49 |
| 2168 | CG | HIS | B | 691 | 10.89 | 38.15 | 21.877 | 1.00 | 45.34 |
| 2169 | CD2 | HIS | B | 691 | 11.897 | 39.04 | 22.045 | 1.00 | 46.24 |
| 2170 | ND1 | HIS | B | 691 | 11.185 | 37.08 | 22.695 | 1.00 | 46.60 |
| 2171 | CE1 | HIS | B | 691 | 12.319 | 37.31 | 23.33 | 1.00 | 46.83 |
| 2172 | NE2 | HIS | B | 691 | 12.772 | 38.49 | 22.954 | 1.00 | 47.22 |
| 2173 | C | HIS | B | 691 | 8.198 | 36.79 | 19.584 | 1.00 | 41.36 |
| 2174 | O | HIS | B | 691 | 7.732 | 35.71 | 19.945 | 1.00 | 41.77 |
| 2175 | N | ARG | B | 692 | 7.497 | 37.69 | 18.904 | 1.00 | 40.51 |
| 2176 | CA | ARO | B | 692 | 6.108 | 37.46 | 18.531 | 1.00 | 40.58 |
| 2177 | CB | ARG | B | 692 | 5.585 | 38.62 | 17.68 | 1.00 | 41.97 |
| 2178 | CG | ARG | B | 692 | 4.103 | 38.54 | 17.334 | 1.00 | 44.31 |
| 2179 | CD | ARG | B | 692 | 3.748 | 39.58 | 16.287 | 1.00 | 46.39 |
| 2180 | NE | AkG | B | 692 | 4.433 | 39.31 | 15.024 | 1.00 | 48.87 |
| 2181 | CZ | ARG | B | 692 | 4.503 | 40.17 | 14.009 | 1.00 | 49.52 |
| 2182 | NH1 | ARG | B | 692 | 3.928 | 41.36 | 14.099 | 1.00 | 50.67 |
| 2183 | NH2 | ARG | B | 692 | 5.15 | 39.83 | 12.901 | 1.00 | 49.63 |
| 2184 | C | ARG | B | 692 | 5.99 | 36.15 | 17.748 | 1.00 | 39.77 |
| 2185 | O | ARG | B | 692 | 5.238 | 35.25 | 18.134 | 1.00 | 38.70 |
| 2186 | N | LEU | B | 693 | 6.739 | 36.05 | 16.652 | 1.00 | 38.99 |
| 2187 | CA | LEU | B | 693 | 6.718 | 34.85 | 15.816 | 1.00 | 39.44 |
| 2188 | CB | LEU | B | 693 | 7.804 | 34.92 | 14.735 | 1.00 | 38.36 |
| 2189 | CG | LEU | B | 693 | 7.65 | 35.94 | 13.602 | 1.00 | 38.71 |
| 2190 | CD1 | LEU | B | 693 | 8.863 | 35.86 | 12.69 | 1.00 | 37.17 |
| 2191 | CD2 | LEU | B | 693 | 6.38 | 35.65 | 12.815 | 1.00 | 37.74 |
| 2192 | C | LEU | B | 693 | 6.924 | 33.59 | 16.642 | 1.00 | 40.12 |
| 2193 | O | LEU | B | 693 | 6.275 | 32.57 | 16.412 | 1.00 | 40.20 |
| 2194 | N | LEU | B | 694 | 7.834 | 33.66 | 17.606 | 1.00 | 41.30 |
| 2195 | CA | LEU | B | 694 | 8.116 | 32.51 | 18.455 | 1.00 | 43.03 |
| 2196 | CB | LEU | B | 694 | 9.4 | 32.75 | 19.249 | 1.00 | 41.27 |
| 2197 | CG | LEU | B | 694 | 10.684 | 32.76 | 18.413 | 1.00 | 40.09 |
| 2198 | CD1 | LEU | B | 694 | 11.845 | 33.25 | 19.251 | 1.00 | 39.04 |
| 2199 | CD2 | LEU | B | 694 | 10.953 | 31.36 | 17.879 | 1.00 | 39.24 |
| 2200 | C | LEU | B | 694 | 6.967 | 32.19 | 19.407 | 1.00 | 45.64 |
| 2201 | O | LEU | B | 694 | 6.867 | 31.07 | 19.907 | 1.00 | 44.78 |
| 2202 | N | GLN | B | 695 | 6.098 | 33.17 | 19.645 | 1.00 | 49.29 |
| 2203 | CA | GLN | B | 695 | 4.964 | 32.98 | 20.55 | 1.00 | 53.51 |
| 2204 | CB | GLN | B | 695 | 4.348 | 34.33 | 20.927 | 1.00 | 53.94 |
| 2205 | CG | GLN | B | 695 | 5.333 | 35.37 | 21.451 | 1.00 | 54.96 |
| 2206 | CD | GLN | B | 695 | 6.079 | 34.93 | 22.698 | 1.00 | 55.61 |
| 2207 | OE1 | GLN | B | 695 | 6.874 | 35.68 | 23.258 | 1.00 | 55.52 |
| 2208 | NE2 | GLN | B | 695 | 5.828 | 33.7 | 23.138 | 1.00 | 55.62 |
| 2209 | C | GLN | B | 695 | 3.877 | 32.09 | 19.953 | 1.00 | 55.94 |
| 2210 | O | GLN | B | 695 | 3.046 | 31.55 | 20.681 | 1.00 | 55.76 |
| 2211 | N | GLU | B | 696 | 3.88 | 31.96 | 18.629 | 1.00 | 58.97 |
| 2212 | CA | GLU | B | 696 | 2.889 | 31.14 | 17.938 | 1.00 | 62.61 |
| 2213 | CB | GLU | B | 696 | 1.479 | 31.66 | 18.24 | 1.00 | 63.02 |
| 2214 | CG | GLU | B | 696 | 1.349 | 33.18 | 18.306 | 1.00 | 63.82 |
| 2215 | CD | GLU | B | 696 | 1.774 | 33.87 | 17.026 | 1.00 | 64.13 |
| 2216 | OE1 | GLU | B | 696 | 1.142 | 33.63 | 15.977 | 1.00 | 64.51 |
| 2217 | OE2 | GLU | B | 696 | 2.743 | 34.66 | 17.072 | 1.00 | 64.16 |
| 2218 | C | GLU | B | 696 | 3.104 | 31.1 | 16.429 | 1.00 | 64.98 |
| 2219 | O | GLU | B | 696 | 2.736 | 30.12 | 15.772 | 1.00 | 65.58 |
| 2220 | N | GLY | B | 697 | 3.699 | 32.15 | 15.883 | 1.00 | 67.35 |
| 2221 | CA | GLY | B | 697 | 3.946 | 32.2 | 14.454 | 1.00 | 70.03 |
| 2222 | C | GLY | B | 697 | 3.53 | 33.52 | 13.838 | 1.00 | 71.80 |
| 2223 | O | GLY | B | 697 | 3.13 | 34.45 | 14.543 | 1.00 | 71.76 |
| 2224 | O | HOH | S | 1 | 23.026 | 22.4 | −2.192 | 1.00 | 19.60 |
| 2225 | O | HOH | S | 2 | 16.83 | 36.19 | 0.675 | 1.00 | 18.07 |
| 2226 | O | HOH | S | 3 | 22.154 | 35.44 | 11.146 | 1.00 | 15.63 |
| 2227 | O | HOH | S | 4 | 24.973 | 20.25 | −2.261 | 1.00 | 20.14 |
| 2228 | O | HOH | S | 5 | 22.161 | 29.6 | 15.788 | 1.00 | 19.70 |
| 2229 | O | HOH | S | 6 | 24.965 | 31.5 | −0.66 | 1.00 | 16.40 |
| 2230 | O | HOH | S | 7 | 10.844 | 33.69 | −3.369 | 1.00 | 17.23 |
| 2231 | O | HOH | S | 8 | 22.447 | 38.7 | −17.98 | 1.00 | 31.92 |
| 2232 | O | HOH | S | 9 | 17.526 | 31.03 | 1.265 | 1.00 | 14.10 |
| 2233 | O | HOH | S | 10 | 29.027 | 37.84 | 2.226 | 1.00 | 16.59 |
| 2234 | O | HOH | S | 11 | 16.149 | 33.62 | 0.874 | 1.00 | 17.71 |
| 2235 | O | HOH | S | 12 | 21.554 | 23.97 | −0.428 | 1.00 | 13.97 |
| 2236 | O | HOH | S | 13 | 24.572 | 41.48 | −5.871 | 1.00 | 19.10 |
| 2237 | O | HOH | S | 14 | 34.349 | 22.18 | 2.662 | 1.00 | 23.28 |
| 2238 | O | HOH | S | 15 | 23.974 | 19.69 | −6.03 | 1.00 | 21.97 |
| 2239 | O | HOH | S | 16 | 12.346 | 31.08 | −5.777 | 1.00 | 18.26 |
| 2240 | O | HOH | S | 17 | 17.302 | 24.21 | 0.483 | 1.00 | 21.48 |
| 2241 | O | HOH | S | 18 | 12.779 | 35.67 | −4.261 | 1.00 | 15.87 |
| 2242 | O | HOH | S | 19 | 32.946 | 42.65 | 9.74 | 1.00 | 47.61 |
| 2243 | O | HOH | S | 20 | 6.647 | 45.31 | 0.256 | 1.00 | 32.40 |
| 2244 | O | HOH | S | 21 | 11.636 | 46.33 | 3.1 | 1.00 | 47.12 |
| 2245 | O | HOH | S | 22 | 15.387 | 32.46 | −1.723 | 1.00 | 24.92 |
| 2246 | O | HOH | S | 23 | 15.179 | 49.11 | −1.349 | 1.00 | 26.79 |
| 2247 | O | HOH | S | 24 | 17.323 | 27.72 | 27.462 | 1.00 | 23.87 |
| 2248 | O | HOH | S | 25 | 32.126 | 32.89 | −0.975 | 1.00 | 24.24 |
| 2249 | O | HOH | S | 26 | 17.22 | 13.39 | 7.072 | 1.00 | 30.98 |
| 2250 | O | HOH | S | 27 | 17.61 | 52.48 | −12.57 | 1.00 | 30.41 |
| 2251 | O | HOH | S | 28 | 11.598 | 28.45 | 0.826 | 1.00 | 27.74 |
| 2252 | O | HOH | S | 29 | 29.348 | 58.2 | 3.202 | 1.00 | 24.47 |
| 2253 | O | HOH | S | 30 | 20.33 | 34.13 | 24.733 | 1.00 | 27.25 |
| 2254 | O | HOH | S | 31 | 11.314 | 30.72 | −3.163 | 1.00 | 29.15 |
| 2255 | O | HOH | S | 32 | 14.534 | 17.66 | −8.919 | 1.00 | 23.07 |
| 2256 | O | HOH | S | 33 | 12.594 | 30.55 | 30.559 | 1.00 | 24.86 |
| 2257 | O | HOH | S | 34 | 4.997 | 18.18 | 3.778 | 1.00 | 28.69 |
| 2258 | O | HOH | S | 35 | 14.93 | 26.08 | 0.846 | 1.00 | 31.48 |
| 2259 | O | HOH | S | 36 | 26.37 | 38.41 | −9.188 | 1.00 | 23.21 |
| 2260 | O | HOH | S | 37 | 1.85 | 51.46 | −7.183 | 1.00 | 42.96 |
| 2261 | O | HOH | S | 38 | 30.499 | 11.23 | 5.931 | 1.00 | 32.46 |
| 2262 | O | HOH | S | 39 | 11.29 | 49.8 | 6.081 | 1.00 | 48.93 |
| 2263 | O | HOH | S | 40 | 20.535 | 16.89 | −4.929 | 1.00 | 32.28 |
| 2264 | O | HOH | S | 41 | 24.859 | 35.08 | −15.49 | 1.00 | 21.88 |
| 2265 | O | HOH | S | 42 | 7.686 | 40.68 | 3.298 | 1.00 | 37.33 |
| 2266 | O | HOH | S | 43 | 27.519 | 46.28 | −18.33 | 1.00 | 37.96 |
| 2267 | O | HOH | S | 44 | 22.661 | 13.79 | −4.577 | 1.00 | 49.60 |
| 2268 | O | HOH | S | 45 | 7.412 | 32.25 | 11.943 | 1.00 | 23.05 |
| 2269 | O | HOH | S | 46 | 31.273 | 40.98 | 1.303 | 1.00 | 31.68 |
| 2270 | O | HOH | S | 47 | 33.257 | 32.33 | 3.506 | 1.00 | 19.13 |
| 2271 | O | HOH | S | 48 | 6.534 | 16.95 | 5.915 | 1.00 | 23.85 |
| 2272 | O | HOH | S | 49 | 17.618 | 50.65 | 7.877 | 1.00 | 28.11 |
| 2273 | O | HOH | S | 50 | 20.823 | 9.387 | 10.717 | 1.00 | 28.29 |
| 2274 | O | HOH | S | 51 | 13.363 | 37.04 | 29.626 | 1.00 | 23.94 |
| 2275 | O | HOH | S | 52 | 9.174 | 18.86 | −1.964 | 1.00 | 33.23 |
| 2276 | O | HOH | S | 53 | 23.557 | 48.69 | −14.77 | 1.00 | 55.81 |
| 2277 | O | HOH | S | 54 | 33.983 | 34.75 | 2.856 | 1.00 | 26.86 |
| 2278 | O | HOH | S | 55 | 29.833 | 11 | 8.326 | 1.00 | 28.51 |
| 2279 | O | HOH | S | 57 | 11.766 | 42.24 | 23.635 | 1.00 | 42.57 |
| 2280 | O | HOH | S | 58 | 14.76 | 33.63 | −4.189 | 1.00 | 32.75 |
| 2281 | O | HOH | S | 59 | 12.5 | 34.34 | 29.874 | 1.00 | 22.65 |
| 2282 | O | HOH | S | 60 | 28.126 | 41.87 | −15.42 | 1.00 | 31.74 |
| 2283 | O | HOH | S | 61 | 26.365 | 40.53 | −7.535 | 1.00 | 22.73 |
| 2284 | O | HOH | S | 62 | 22.279 | 46.65 | −15.85 | 1.00 | 50.83 |
| 2285 | O | HOH | S | 63 | 7.178 | 15.42 | 0.05 | 1.00 | 44.46 |
| 2286 | O | HOH | S | 64 | 3.624 | 30.59 | −0.214 | 1.00 | 36.39 |
| 2287 | O | HOH | S | 65 | 6.139 | 27.86 | 3.768 | 1.00 | 52.56 |
| 2288 | O | HOH | S | 66 | 31.097 | 55.49 | 8.217 | 1.00 | 30.35 |
| 2289 | O | HOH | S | 67 | 18.605 | 58.14 | −1.051 | 1.00 | 47.14 |
| 2290 | O | HOH | S | 68 | 8.343 | 23.64 | 26.996 | 1.00 | 32.72 |
| 2291 | O | HOH | S | 69 | 19.685 | 57.59 | −3.396 | 1.00 | 43.61 |
| 2292 | O | HOH | S | 70 | 20.943 | 28.28 | 33.357 | 1.00 | 40.59 |
| 2293 | O | HOH | S | 71 | 21.649 | 36.71 | 27.743 | 1.00 | 35.00 |
| 2294 | O | HOH | S | 72 | 31.539 | 42.79 | 3.263 | 1.00 | 30.88 |
| 2295 | O | HOH | S | 73 | 13.289 | 50.24 | 2.509 | 1.00 | 38.75 |
| 2296 | O | HOH | S | 74 | 19.123 | 53.23 | 5.431 | 1.00 | 37.22 |
| 2297 | O | HOH | S | 75 | 10.856 | 22.35 | 19.47 | 1.00 | 19.26 |
| 2298 | O | HOH | S | 76 | 4.014 | 20.49 | 2.2 | 1.00 | 37.71 |
| 2299 | O | HOH | S | 78 | 32.803 | 10.68 | 9.175 | 1.00 | 22.60 |
| 2300 | O | HOH | S | 79 | 15.982 | 27.46 | −1.316 | 1.00 | 30.63 |
| 2301 | O | HOH | S | 80 | 6.778 | 41.32 | 9.974 | 1.00 | 45.09 |
| 2302 | O | HOH | S | 81 | 6.267 | 30.45 | 7.93 | 1.00 | 33.02 |
| 2303 | O | HOH | S | 82 | 19.319 | 14.91 | 21.654 | 1.00 | 42.45 |
| 2304 | O | HOH | S | 83 | 2.585 | 29.42 | 1.807 | 1.00 | 40.89 |
| 2305 | O | HOH | S | 84 | 2.194 | 49.14 | −16.66 | 1.00 | 53.37 |
| 2306 | O | HOH | S | 85 | 36.352 | 8.69 | 3.311 | 1.00 | 40.82 |
| 2307 | O | HOH | S | 86 | 22.685 | 19.53 | 29.701 | 1.00 | 47.69 |
| 2308 | O | HOH | S | 87 | 33.974 | 34.64 | 0.26 | 1.00 | 31.56 |
| 2309 | O | HOH | S | 88 | 31.744 | 38.24 | 1.513 | 1.00 | 22.56 |
| 2310 | O | HOH | S | 89 | 5.825 | 37.89 | 6.408 | 1.00 | 48.05 |
| 2311 | O | HOH | S | 90 | 19.49 | 17.4 | −8.965 | 1.00 | 38.26 |
| 2312 | O | HOH | S | 91 | 40.414 | 35.6 | 7.949 | 1.00 | 35.76 |
| 2313 | O | HOH | S | 92 | 15.402 | 46.87 | 15.762 | 1.00 | 39.90 |
| 2314 | O | HOH | S | 93 | 6.875 | 29.53 | −13.41 | 1.00 | 40.19 |
| 2315 | O | HOH | S | 94 | 8.648 | 28.6 | 20.645 | 1.00 | 29.73 |
| 2316 | O | HOH | S | 95 | 22.063 | 37.44 | 22.081 | 1.00 | 37.50 |
| 2317 | O | HOH | S | 96 | 27.61 | 30.94 | 23.964 | 1.00 | 32.74 |
| 2318 | O | HOH | S | 97 | 10.971 | 31.16 | 27.918 | 1.00 | 24.96 |
| 2319 | O | HOH | S | 98 | 26.229 | 25.48 | −11.89 | 1.00 | 41.41 |
| 2320 | O | HOH | S | 100 | 6.704 | 33.43 | −14.58 | 1.00 | 36.10 |
| 2321 | O | HOH | S | 101 | 15.544 | 8.896 | 10.723 | 1.00 | 33.88 |
| 2322 | O | HOH | S | 102 | 16.493 | 16.84 | −2.596 | 1.00 | 26.55 |
| 2323 | O | HOH | S | 103 | 28.351 | 28.27 | 12.338 | 1.00 | 43.77 |
| 2324 | O | HOH | S | 104 | 27.737 | 3.455 | 13.166 | 1.00 | 48.23 |
| 2325 | O | HOH | S | 105 | 24.873 | 39.51 | −19.21 | 1.00 | 53.25 |
| 2326 | O | HOH | S | 106 | 12.972 | 44.58 | −8.54 | 1.00 | 25.37 |
| 2327 | O | HOH | S | 107 | 20.643 | 61.57 | 2.015 | 1.00 | 57.43 |
| 2328 | O | HOH | S | 108 | 18.412 | 21.18 | −12.7 | 1.00 | 30.69 |
| 2329 | O | HOH | S | 109 | 21.691 | 40.71 | 28.501 | 1.00 | 31.23 |
| 2330 | O | HOH | S | 111 | 37.452 | 24.33 | 8.574 | 1.00 | 43.15 |
| 2331 | O | HOH | S | 112 | 28.082 | 12.03 | −2.83 | 1.00 | 35.13 |
| 2332 | O | HOH | S | 113 | 13.626 | 6.782 | 9.82 | 1.00 | 59.43 |
| 2333 | O | HOH | S | 114 | 29.219 | 47.39 | −11.5 | 1.00 | 33.93 |
| 2334 | O | HOH | S | 115 | 37.194 | 25.42 | 11.265 | 1.00 | 49.29 |
| 2335 | O | HOH | S | 116 | 32.453 | 24.1 | 12.149 | 1.00 | 37.91 |
| 2336 | O | HOH | S | 117 | 20.069 | 18.41 | −15.74 | 1.00 | 52.69 |
| 2337 | O | HOH | S | 118 | 37.303 | 36.57 | 3.605 | 1.00 | 50.95 |
| 2338 | O | HOH | S | 119 | 26.65 | 37.93 | 27.599 | 1.00 | 60.19 |
| 2339 | O | HOH | S | 120 | 40.042 | 37.14 | 6.054 | 1.00 | 62.83 |
| 2340 | O | HOH | S | 121 | 16.718 | 28.82 | 32.714 | 1.00 | 45.22 |
| 2341 | O | HOH | S | 122 | 15.637 | 14.39 | −3 | 1.00 | 32.52 |
| 2342 | O | HOH | S | 123 | 25.337 | 43.41 | 12.302 | 1.00 | 46.21 |
| 2343 | O | HOH | S | 124 | 35.978 | 27.43 | −0.068 | 1.00 | 39.51 |
| 2344 | O | HOH | S | 125 | 33.68 | 38.72 | −6.927 | 1.00 | 55.40 |
| 2345 | O | HOH | S | 126 | 2.654 | 22.83 | 6.016 | 1.00 | 32.62 |
| 2346 | O | HOH | S | 127 | 21.42 | 16.3 | 32.91 | 1.00 | 56.84 |
| 2347 | O | HOH | S | 128 | 2.525 | 25.69 | 7.475 | 1.00 | 49.14 |
| 2348 | O | HOH | S | 129 | 38.283 | 26.64 | 7.527 | 1.00 | 36.28 |
| 2349 | O | HOH | S | 130 | 24.339 | 56.08 | −3.205 | 1.00 | 42.51 |
| 2350 | O | HOH | S | 131 | 31.349 | 39.79 | −15.87 | 1.00 | 43.44 |
| 2351 | O | HOH | S | 132 | 14.264 | 49.95 | 9.76 | 1.00 | 46.86 |
| 2352 | O | HOH | S | 133 | 29.477 | 46.34 | −15.12 | 1.00 | 56.58 |
| 2353 | O | HOH | S | 134 | 13.24 | 46.59 | 14.288 | 1.00 | 30.39 |
| 2354 | O | HOH | S | 135 | 28.713 | 21.45 | 14.541 | 1.00 | 43.43 |
| 2355 | O | HOH | S | 136 | 29.863 | 19.83 | 16.145 | 1.00 | 53.77 |
| 2356 | O | HOH | S | 137 | 28.691 | 40.59 | 0.038 | 1.00 | 27.60 |
| 2357 | O | HOH | S | 138 | 31.992 | 29.57 | 17.933 | 1.00 | 52.12 |
| 2358 | O | HOH | S | 139 | 32.346 | 41.3 | −7.206 | 1.00 | 48.91 |
| 2359 | O | HOH | S | 140 | 18.153 | 37.55 | 22.932 | 1.00 | 31.27 |
| 2360 | O | HOH | S | 141 | 16.88 | 57.73 | −7.178 | 1.00 | 36.07 |
| 2361 | O | HOH | S | 142 | 3.616 | 38.62 | 3.9 | 1.00 | 63.76 |
| 2362 | O | HOH | S | 143 | 18.536 | 13.34 | −5.108 | 1.00 | 59.11 |
| 2363 | O | HOH | S | 144 | 6.14 | 50.93 | −15.59 | 1.00 | 38.96 |
| 2364 | O | HOH | S | 145 | 14.732 | 25.97 | 29.458 | 1.00 | 32.23 |
| 2365 | O | HOH | 5 | 146 | 21.729 | 19.32 | −8.314 | 1.00 | 36.70 |
| 2366 | O | HOH | S | 147 | 8.169 | 16.53 | 17.709 | 1.00 | 58.27 |
| 2367 | O | HOH | S | 148 | 11.709 | 32.98 | 0.499 | 1.00 | 36.22 |
| 2368 | O | HOH | S | 149 | 13.578 | 48.33 | 0.597 | 1.00 | 37.61 |
| 2369 | O | HOH | S | 150 | 12.376 | 12.47 | 17.052 | 1.00 | 62.89 |
| 2370 | O | HOH | S | 151 | 9.626 | 31.89 | 1.206 | 1.00 | 68.72 |
| 2371 | O | HOH | 5 | 152 | 28.153 | 19.15 | 18.731 | 1.00 | 42.30 |
| 2372 | O | HOH | S | 153 | 32.498 | 45.94 | −7.499 | 1.00 | 37.61 |
| 2373 | O | HOH | S | 154 | 24.755 | 0.891 | 18.958 | 1.00 | 66.75 |
| 2374 | O | HOH | S | 155 | 39.681 | 22.65 | 2.229 | 1.00 | 35.42 |
| 2375 | O | HOH | S | 156 | 13.536 | 52.85 | 2.389 | 1.00 | 37.57 |
| 2376 | O | HOH | S | 157 | 8.317 | 48.95 | 2.546 | 1.00 | 73.06 |
| 2377 | O | HOH | S | 158 | 8.644 | 28.04 | −12.59 | 1.00 | 33.91 |
| 2378 | O | HOH | S | 159 | 2.877 | 30.15 | 4.135 | 1.00 | 62.49 |
| 2379 | O | HOH | S | 160 | 31.522 | 7.355 | 4.7 | 1.00 | 35.95 |
| 2380 | O | HOH | S | 161 | 5.863 | 26.35 | 22.68 | 1.00 | 39.01 |
| 2381 | O | HOH | S | 162 | 7.589 | 46.35 | −19.67 | 1.00 | 50.76 |
| 2382 | O | HOH | S | 163 | 22.983 | 9.848 | −2.49 | 1.00 | 64.92 |
| 2383 | O | HOH | S | 164 | 6.674 | 37.22 | 8.925 | 1.00 | 35.74 |
| 2384 | O | HOH | S | 165 | 1.66 | 40.66 | 10.72 | 1.00 | 68.96 |
| 2385 | O | HOH | S | 166 | 37.85 | 28.01 | 11.075 | 1.00 | 29.28 |
| 2386 | O | HOH | S | 167 | 2.367 | 30.07 | 11.169 | 1.00 | 47.90 |
| 2387 | O | HOH | S | 168 | 22.807 | 6.884 | 24.267 | 1.00 | 50.27 |
| 2388 | O | HOH | S | 169 | 25.253 | 24.05 | −9.518 | 1.00 | 56.25 |
| 2389 | O | HOH | S | 170 | 31.929 | 40.72 | −4.78 | 1.00 | 43.99 |
| 2390 | O | HOH | S | 171 | 29.051 | 32.12 | −14.16 | 1.00 | 48.37 |
| 2391 | O | HOH | S | 172 | 6.854 | 24.41 | 13.632 | 1.00 | 30.86 |
| 2392 | O | HOH | S | 173 | 31.121 | 26.37 | −0.287 | 1.00 | 26.16 |
| 2393 | O | HOH | S | 174 | 26.425 | 4.757 | 16.289 | 1.00 | 46.62 |
| 2394 | O | HOH | 5 | 175 | 30.245 | 52.79 | −1.002 | 1.00 | 62.86 |
| 2395 | O | HOH | S | 176 | 11.266 | 25.3 | −15.97 | 1.00 | 30.67 |
| 2396 | O | HOH | S | 177 | 8.232 | 22.79 | −8.256 | 1.00 | 36.47 |
| 2397 | O | HOH | S | 178 | 32.73 | 30.85 | 1.133 | 1.00 | 29.48 |
| 2398 | O | HOH | S | 179 | 12.168 | 13.53 | −6.965 | 1.00 | 54.81 |
| 2399 | O | HOH | S | 181 | 9.981 | 18.58 | −15.66 | 1.00 | 52.49 |
| 2400 | O | HOH | S | 182 | 2.592 | 15.84 | 3.846 | 1.00 | 36.47 |
| 2401 | O | HOH | S | 184 | 16.07 | 21.67 | 25.099 | 1.00 | 36.38 |
| 2402 | O | HOH | S | 185 | 9.58 | 53.07 | 5.376 | 1.00 | 51.65 |
| 2403 | O | HOH | S | 186 | 9.863 | 33.55 | 29.136 | 1.00 | 41.08 |
| 2404 | O | HOH | S | 187 | 28.882 | 9.516 | −2.636 | 1.00 | 43.51 |
| 2405 | O | HOH | S | 188 | 28.982 | 14.1 | −1.733 | 1.00 | 52.94 |
| 2406 | O | HOH | S | 189 | 8.611 | 16.55 | 11.998 | 1.00 | 34.51 |
| 2407 | O | HOH | S | 190 | 12.85 | 16.63 | −1.121 | 1.00 | 38.29 |
| 2408 | O | HOH | S | 191 | 28.378 | 56.86 | −0.306 | 1.00 | 70.56 |
| 2409 | O | HOH | S | 192 | 21.342 | 14.62 | 28.328 | 1.00 | 75.81 |
| 2410 | O | HOH | S | 193 | 29.531 | 7.961 | 13.212 | 1.00 | 47.28 |
| 2411 | O | HOH | S | 194 | 32.953 | 32.46 | 15.308 | 1.00 | 53.06 |
| 2412 | O | HOH | S | 195 | 6.057 | 23.07 | 15.892 | 1.00 | 44.59 |
| 2413 | O | HOH | S | 196 | 32.03 | 19.855 | 17.068 | 1.00 | 61.54 |
| 2414 | O | HOH | S | 197 | 31.244 | 15.54 | 16.59 | 1.00 | 47.53 |
| 2415 | O | HOH | S | 198 | 25.439 | 9.787 | 20.81 | 1.00 | 55.35 |
| 2416 | O | HOH | S | 199 | 17.929 | 17.39 | −4.951 | 1.00 | 38.99 |
| 2417 | O | HOH | S | 200 | 20.119 | 56.57 | −12.07 | 1.00 | 34.77 |
| 2418 | O | HOH | S | 201 | 5.206 | 29.9 | 4.996 | 1.00 | 54.91 |
| 2419 | O | HOH | S | 202 | 14.253 | 59.12 | −6.767 | 1.00 | 57.78 |
| 2420 | O | HOH | S | 203 | 2.327 | 36.03 | 19.271 | 1.00 | 71.38 |
| 2421 | O | HOH | S | 205 | 25.908 | 29.01 | 30.674 | 1.00 | 45.68 |
| 2422 | O | HOH | S | 206 | 6.998 | 27.65 | 7.373 | 1.00 | 33.37 |
| 2423 | O | HOH | 5 | 207 | 29.909 | 10.26 | 3.633 | 1.00 | 28.69 |
| 2424 | O | HOH | 5 | 208 | 3.221 | 39.57 | 9.013 | 1.00 | 64.29 |
| 2425 | O | HOH | S | 209 | 11.662 | 26.03 | 31.453 | 1.00 | 30.92 |
| 2426 | O | HOH | S | 210 | 30.602 | 43.9 | −10.34 | 1.00 | 35.47 |
| 2427 | O | HOH | S | 211 | 5.674 | 19.99 | 12.478 | 1.00 | 36.93 |
| 2428 | O | HOH | S | 212 | 8.997 | 8.066 | 12.548 | 1.00 | 44.27 |
| 2429 | O | HOH | S | 213 | 18.565 | 1.299 | 23.321 | 1.00 | 53.87 |
| 2430 | O | HOH | S | 214 | 14.123 | 50.52 | −14.45 | 1.00 | 54.64 |
| 2431 | O | HOH | S | 215 | 18.959 | 15.48 | 28.395 | 1.00 | 71.59 |
| 2432 | O | HOH | S | 216 | 0.665 | 37.38 | 17.683 | 1.00 | 49.95 |
| 2433 | O | HOH | S | 217 | 26.863 | 55.57 | −2.207 | 1.00 | 46.16 |
| 2434 | O | HOH | S | 218 | 6.158 | 22.9 | 11.742 | 1.00 | 31.87 |
| 2435 | O | HOH | S | 219 | 9.888 | 16.38 | −1.719 | 1.00 | 36.68 |
| 2436 | O | HOH | S | 220 | 30.266 | 27.84 | 23.674 | 1.00 | 53.45 |
| 2437 | O | HOH | S | 221 | 30.769 | 47.55 | −1.614 | 1.00 | 42.34 |
| 2438 | O | HOH | S | 222 | 32.195 | 35.01 | 15.035 | 1.00 | 44.29 |
| 2439 | O | HOH | S | 223 | 10.286 | 34.65 | −0.758 | 1.00 | 25.15 |
| 2440 | O | HOH | S | 224 | 4.88 | 17.84 | 16.029 | 1.00 | 58.71 |
| 2441 | O | HOH | S | 225 | 5.737 | 41.57 | 7.23 | 1.00 | 41.48 |
| 2442 | O | HOH | S | 226 | 24.968 | 27.07 | −7.625 | 1.00 | 61.37 |
| 2443 | O | HOH | S | 227 | 26.43 | 10.73 | 26.093 | 1.00 | 47.70 |
| 2444 | O | HOH | S | 228 | 9.402 | 26.41 | 28.179 | 1.00 | 51.51 |
| 2445 | O | HOH | S | 229 | 22.501 | 23.96 | −9.399 | 1.00 | 43.79 |
| 2446 | O | HOH | S | 232 | 36.306 | 31.18 | 7.127 | 1.00 | 49.35 |
| 2447 | O | HOH | S | 233 | 16.063 | 56.54 | 4.731 | 1.00 | 47.51 |
| 2448 | O | HOH | S | 234 | 9.113 | 46 | 15.205 | 1.00 | 51.54 |
| 2449 | O | HOH | S | 236 | 22.85 | 16.02 | −6.845 | 1.00 | 48.61 |
| 2450 | O | HOH | S | 237 | 4.561 | 37.73 | 10.108 | 1.00 | 63.61 |
| 2451 | O | HOH | S | 238 | 8.317 | 20.22 | −8.658 | 1.00 | 40.62 |
| 2452 | O | HOH | S | 239 | 26.545 | 17.09 | 26.547 | 1.00 | 60.48 |
| 2453 | O | HOH | S | 240 | 18.438 | 52.12 | −15.97 | 1.00 | 68.74 |
| 2454 | O | HOH | S | 241 | 15.737 | 11.25 | 6.148 | 1.00 | 40.27 |
| 2455 | O | HOH | S | 242 | −2.46 | 33.66 | 15.319 | 1.00 | 56.63 |
| 2456 | O | HOH | S | 243 | 9.254 | 37.42 | 7.214 | 1.00 | 50.70 |
| 2457 | O | HOH | S | 244 | 22.874 | 5.448 | −0.368 | 1.00 | 56.93 |
| 2458 | O | HOH | S | 245 | 1.408 | 28.07 | 19.082 | 1.00 | 54.97 |
| 2459 | O | HOH | S | 246 | 29.407 | 5.692 | 12.371 | 1.00 | 46.23 |
| 2460 | O | HOH | S | 247 | 21.392 | 2.016 | 21.068 | 1.00 | 54.78 |
| 2461 | O | HOH | S | 248 | 3.142 | 52.17 | −19.3 | 1.00 | 61.95 |
| 2462 | O | HOH | S | 249 | 22.021 | 57.55 | −0.613 | 1.00 | 48.17 |
| 2463 | O | HOH | S | 250 | 13.662 | 3.611 | 13.009 | 1.00 | 43.82 |
| 2464 | O | HOH | S | 251 | 19.193 | 16.6 | 19.469 | 1.00 | 31.39 |
| 2465 | O | HOH | S | 252 | 3.331 | 33.52 | 6.6 | 1.00 | 47.67 |
| 2466 | O | HOH | S | 253 | 10.792 | 2.983 | 19.468 | 1.00 | 70.14 |
| 2467 | O | HOH | S | 254 | 16.787 | 17.52 | 20.507 | 1.00 | 63.73 |
| 2468 | O | HOH | S | 255 | 2.619 | 44.64 | 16.706 | 1.00 | 56.53 |
| 2469 | O | HOH | S | 256 | 27.869 | 14.32 | 18.171 | 1.00 | 41.98 |
| 2470 | O | HOH | S | 257 | 31.415 | 45.49 | −13.52 | 1.00 | 53.59 |
| 2471 | O | HOH | S | 258 | 31.255 | 35.57 | −11.97 | 1.00 | 33.65 |
| 2472 | O | HOH | S | 259 | 21.689 | 4.451 | 26.354 | 1.00 | 52.63 |
| 2473 | O | HOH | S | 260 | 10.441 | 20.46 | 21.585 | 1.00 | 35.08 |
| 2474 | O | HOH | S | 261 | 17.807 | 18.05 | 18.034 | 1.00 | 49.80 |
| 2475 | O | HOH | S | 262 | 12.939 | 41.2 | 25.86 | 1.00 | 49.78 |
| 2476 | O | HOH | S | 263 | 11.831 | 38.27 | 27.695 | 1.00 | 41.47 |
| 2477 | O | HOH | S | 264 | 21.694 | 14.49 | 25.644 | 1.00 | 49.41 |
| 2478 | O | HOH | S | 265 | 3.278 | 19.42 | −21.24 | 1.00 | 58.93 |
| 2479 | O | HOH | S | 266 | 32.139 | 44.55 | −1.767 | 1.00 | 63.10 |
| 2480 | O | HOH | S | 267 | 30.536 | 19.71 | 26.309 | 1.00 | 56.72 |
| 2481 | O | HOH | S | 268 | 1.033 | 38.18 | 8.349 | 1.00 | 69.65 |
| 2482 | O | HOH | S | 269 | 15.677 | 56.47 | −11.13 | 1.00 | 45.85 |
| 2483 | O | HOH | S | 270 | 9.631 | 49.19 | −12.73 | 1.00 | 31.76 |
| 2484 | O | HOH | S | 271 | 26.281 | 33.95 | 12.002 | 1.00 | 28.74 |
| 2485 | O | HOH | S | 272 | 16.307 | 55.36 | −6.008 | 1.00 | 48.50 |
| 2486 | O | HOH | S | 273 | 35.226 | 25.13 | 1.967 | 1.00 | 37.37 |
| 2487 | O | HOH | S | 274 | 34.15 | 31.09 | 10.684 | 1.00 | 35.88 |
| 2488 | O | HOH | S | 275 | 9.81 | 12.1 | −10.1 | 1.00 | 42.96 |
| 2489 | O | HOH | S | 277 | 31.321 | 17.79 | 15.555 | 1.00 | 54.02 |
| 2490 | O | HOH | S | 278 | 6.023 | 17.62 | 1.258 | 1.00 | 32.48 |
| 2491 | O | HOH | 5 | 279 | 10.646 | 28.67 | 26.826 | 1.00 | 33.27 |
| 2492 | O | HOH | S | 280 | 33.133 | 28.2 | 0.795 | 1.00 | 32.26 |
| 2493 | O | HOH | S | 281 | 4.679 | 49.52 | −17.39 | 1.00 | 56.01 |
| 2494 | O | HOH | S | 282 | 19.923 | 38.29 | 25.079 | 1.00 | 52.01 |
| 2495 | O | HOH | S | 283 | 17.06 | 16.72 | −7.694 | 1.00 | 44.99 |
| 2496 | O | HOH | S | 284 | 8.155 | 32.99 | 31.053 | 1.00 | 38.13 |
| 2497 | O | HOH | S | 285 | 15.353 | 23.01 | 31.936 | 1.00 | 47.35 |
| 2498 | O | HOH | S | 286 | 37.587 | 33.19 | 10.823 | 1.00 | 50.51 |
| 2499 | O | HOH | S | 287 | 26.366 | 26.07 | 14.494 | 1.00 | 43.81 |
| 2500 | O | HOH | S | 288 | 9.308 | 11.77 | 3.313 | 1.00 | 52.95 |
| 2501 | O | HOH | 5 | 289 | 29.889 | 5.039 | 4.211 | 1.00 | 39.94 |
| 2502 | O | HOH | S | 290 | 21.608 | 63.69 | 5.695 | 1.00 | 47.51 |
| 2503 | O | HOH | S | 291 | 5.878 | 51.06 | −5.441 | 1.00 | 57.81 |
| 2504 | O | HOH | S | 292 | 4.217 | 31.18 | 9.122 | 1.00 | 34.04 |
| 2505 | O | HOH | S | 293 | 20.204 | 52.12 | −12.15 | 1.00 | 32.14 |
| 2506 | O | HOH | S | 294 | 31.777 | 46.88 | −9.872 | 1.00 | 37.63 |
| 2507 | O | HOH | S | 295 | 29.367 | 16.37 | 18.828 | 1.00 | 47.88 |
| 2508 | O | HOH | S | 296 | 7.053 | 22.51 | 23.361 | 1.00 | 49.62 |
| 2509 | O | HOH | S | 297 | 10.448 | 17.83 | 24.905 | 1.00 | 45.38 |
| 2510 | O | HOH | S | 298 | 22.727 | 21.64 | −7.856 | 1.00 | 44.54 |
| 2511 | O | HOH | S | 299 | 41.752 | 38.42 | 4.331 | 1.00 | 51.42 |
| 2512 | O | HOH | S | 300 | 28.35 | 29.18 | 29.86 | 1.00 | 35.37 |
| 2513 | O | HOH | S | 301 | 19.875 | 8.686 | 8.347 | 1.00 | 36.88 |
| 2514 | O | HOH | S | 302 | 30.241 | 47.82 | 14.46 | 1.00 | 52.48 |
| 2515 | O | HOH | S | 303 | 38.107 | 31.69 | −0.089 | 1.00 | 48.80 |
| 2516 | O | HOH | S | 304 | 29.435 | 19.53 | 22.017 | 1.00 | 48.53 |
| 2517 | O | HOH | S | 305 | 28.231 | 17.78 | 23.534 | 1.00 | 49.19 |
| 2518 | O | HOH | S | 306 | 25.102 | 25.86 | 30.666 | 1.00 | 50.04 |
| 2519 | O | HOH | S | 307 | 20.946 | 24.31 | −13.66 | 1.00 | 35.02 |
| 2520 | O | HOH | S | 308 | 27.296 | 9.199 | 14.576 | 1.00 | 37.16 |
| 2521 | O | HOH | S | 309 | 5.593 | 44.21 | 15.509 | 1.00 | 46.00 |
| 2522 | O | HOH | S | 310 | 1.445 | 15.84 | 16.482 | 1.00 | 48.72 |
| 2523 | O | HOH | S | 311 | 31.898 | 52.12 | −9.291 | 1.00 | 36.65 |
| 2524 | O | HOH | S | 312 | 2.78 | 26.87 | 21.407 | 1.00 | 37.83 |
| 2525 | O | HOH | S | 313 | 10.829 | 21.85 | 1.47 | 1.00 | 38.42 |
| 2526 | O | HOH | S | 314 | 25.186 | 43.65 | 22.198 | 1.00 | 39.85 |
| 2527 | O | HOH | S | 315 | 10.029 | 44.29 | 3.158 | 1.00 | 61.53 |
| 2528 | O | HOH | S | 316 | 25.08 | 12.34 | −5.224 | 1.00 | 49.90 |
| 2529 | O | HOH | S | 317 | 24.701 | 26.06 | 34.199 | 1.00 | 51.37 |
| 2530 | O | HOH | S | 318 | 17.657 | 17.78 | −11.97 | 1.00 | 54.01 |
| 2531 | O | HOH | S | 319 | 21.669 | 9.232 | −4.594 | 1.00 | 50.75 |
| 2532 | O | HOH | S | 320 | 12.831 | 30.46 | −0.73 | 1.00 | 46.31 |
| 2533 | O | HOH | S | 321 | −0.839 | 31.74 | 16.05 | 1.00 | 49.15 |
| 2534 | O | HOH | S | 322 | 11.333 | 25.16 | 29.142 | 1.00 | 54.91 |
| 2535 | O | HOH | S | 323 | 38.448 | 33.36 | 4.415 | 1.00 | 47.87 |
| 2536 | O | HOH | S | 324 | 29.755 | 48.47 | −20.47 | 1.00 | 61.21 |
| 2537 | O | HOH | S | 325 | 19.876 | 22.91 | 34.487 | 1.00 | 53.69 |
| 2538 | O | HOH | 5 | 326 | 6.159 | 6.348 | 12.404 | 1.00 | 45.47 |
| 2539 | O | HOH | S | 327 | 24.803 | 24.04 | −13.67 | 1.00 | 41.36 |
| 2540 | O | HOH | S | 328 | 34.41 | 33.84 | 13.722 | 1.00 | 48.67 |
| 2541 | O | HOH | S | 330 | 28.488 | 24.85 | 15.172 | 1.00 | 42.44 |
| 2542 | O | HOH | S | 331 | 18.756 | 10.01 | −0.302 | 1.00 | 47.49 |
| 2543 | O | HOH | S | 332 | 27.919 | 33.32 | 15.964 | 1.00 | 46.60 |
| 2544 | O | HOH | S | 333 | 28.084 | 54.17 | −9.919 | 1.00 | 47.31 |
| 2545 | O | HOH | S | 334 | 6.66 | 20.06 | 17.321 | 1.00 | 66.41 |
| 2546 | O | HOH | S | 335 | 9.177 | 51.42 | −1.091 | 1.00 | 50.92 |
| 2547 | O | HOH | S | 336 | 26.133 | 49.98 | −13.04 | 1.00 | 44.73 |
| 2548 | O | HOH | S | 337 | 6.461 | 15 | 10.655 | 1.00 | 50.61 |
| 2549 | O | HOH | S | 338 | 27.448 | 21.32 | 29.977 | 1.00 | 46.70 |
| 2550 | O | HOH | S | 339 | −2.375 | 39.03 | 9.478 | 1.00 | 48.95 |
| 2551 | O | HOH | S | 340 | 39.666 | 34.71 | 11.042 | 1.00 | 54.77 |
| 2552 | O | HOH | S | 341 | 31.504 | 4.157 | 11.722 | 1.00 | 38.27 |
| 2553 | O | HOH | S | 342 | 30.223 | 43.44 | 12.537 | 1.00 | 56.22 |
| 2554 | O | HOH | S | 3433 | 1.793 | 6.842 | 14.029 | 1.00 | 49.62 |
| 2555 | O | HOH | S | 344 | 34.165 | 36.36 | −11.48 | 1.00 | 54.81 |
| 2556 | O | HOH | S | 345 | 16.345 | 29.91 | −0.844 | 1.00 | 50.86 |
| 2557 | O | HOH | S | 346 | 16.038 | 14.04 | −0.096 | 1.00 | 26.81 |
| 2558 | O | HOH | S | 347 | 35.56 | 39.29 | 6.896 | 1.00 | 58.70 |
| 2559 | O | HOH | S | 348 | 7.207 | 18.78 | −13.36 | 1.00 | 48.09 |
| 2560 | O | HOH | S | 349 | 4.347 | 25.73 | 17.608 | 1.00 | 48.32 |
| 2561 | O | HOH | S | 350 | 23.981 | 49.9 | 20.29 | 1.00 | 45.46 |
| 2562 | O | HOH | S | 351 | −1.311 | 39.18 | 20.561 | 1.00 | 47.06 |
| 2563 | O | HOH | S | 352 | 29.438 | 35.08 | −13.57 | 1.00 | 51.53 |
| 2564 | O | HOH | S | 353 | 22.157 | 11.96 | −8.267 | 1.00 | 58.38 |
| 2565 | O | HOH | S | 354 | 28.535 | 23.39 | −8.407 | 1.00 | 38.50 |
| 2566 | O | HOH | S | 355 | 16.02 | 7.615 | 25.043 | 1.00 | 63.84 |
| 2567 | O | HOH | S | 356 | 2.323 | 34.05 | 2.822 | 1.00 | 52.94 |
| 2568 | O | HOH | S | 357 | 14.88 | 19.56 | 25.915 | 1.00 | 44.98 |
| 2569 | O | HOH | S | 358 | 33.923 | 47.33 | −12.62 | 1.00 | 52.85 |
| 2570 | O | HOH | S | 359 | 27.496 | 24.78 | 30.413 | 1.00 | 48.33 |
| 2571 | O | HOH | S | 360 | 20.476 | 43.25 | 26.095 | 1.00 | 52.94 |
| 2572 | O | HOH | S | 361 | 28.282 | 37.79 | 30.539 | 1.00 | 62.99 |
| 2573 | O | HOH | S | 362 | 27.472 | 34.09 | −15.38 | 1.00 | 50.71 |
| 2574 | O | HOH | S | 363 | 25.666 | 34.54 | 30.416 | 1.00 | 54.08 |
| 2575 | O | HOH | S | 364 | 29.779 | 1.14 | 1.301 | 1.00 | 59.34 |
| 2576 | O | HOH | S | 365 | 5.434 | 39.02 | 22.161 | 1.00 | 47.10 |
| 2577 | O | HOH | S | 366 | 5.05 | 16.76 | 20.069 | 1.00 | 60.26 |
| 2578 | O | HOH | S | 367 | 18.636 | 55.72 | −4.804 | 1.00 | 55.62 |
| 2579 | O | HOH | S | 368 | 32.684 | 59.06 | 10.827 | 1.00 | 39.90 |
| 2580 | O | HOH | S | 369 | 28.244 | 51.07 | −11.91 | 1.00 | 52.57 |
| 2581 | O | HOH | S | 370 | 11.589 | 51.23 | −1.777 | 1.00 | 63.06 |
| 2582 | O | HOH | S | 371 | 2.98 | 19.43 | −24.51 | 1.00 | 45.57 |
| 2583 | O | HOH | S | 372 | 22.088 | 60 | −1.466 | 1.00 | 61.45 |
| 2584 | O | HOH | S | 373 | 22.665 | 43.26 | 31.048 | 1.00 | 47.28 |
| 2585 | O | HOH | S | 374 | 30.445 | 38.97 | 29.886 | 1.00 | 57.25 |
| 2586 | O | HOH | S | 375 | 35.859 | 38.01 | 1.977 | 1.00 | 61.81 |
| 2587 | O | HOH | S | 376 | 6.112 | 31.91 | −17.26 | 1.00 | 49.61 |
| 2588 | O | HOH | S | 377 | 6.721 | 15.6 | −22.4 | 1.00 | 59.42 |
| 2589 | O | HOH | S | 378 | 12.118 | 1.192 | 18.305 | 1.00 | 48.35 |
| 2590 | O | HOH | S | 379 | 4.24 | 32.09 | 1.805 | 1.00 | 44.18 |
| 2591 | O | HOH | S | 380 | 29.696 | 35.08 | 27.75 | 1.00 | 55.76 |
| 2592 | O | HOH | S | 381 | 26.359 | 7.481 | 25.027 | 1.00 | 51.90 |
| 2593 | O | HOH | S | 382 | 9.133 | 46.89 | 3.888 | 1.00 | 56.70 |
| 2594 | O | HOH | S | 383 | 10.643 | 46.44 | −20.34 | 1.00 | 37.21 |
| 2595 | O | HOH | S | 384 | 8.232 | 48.96 | 17.207 | 1.00 | 59.01 |
| 2596 | O | HOH | S | 385 | 20.326 | 15.01 | −8.821 | 1.00 | 55.36 |
| 2597 | O | HOH | S | 386 | 19.253 | −0.131 | 16.57 | 1.00 | 45.30 |
| 2598 | O | HOH | S | 387 | 7.34 | 49.8 | −0.68 | 1.00 | 55.02 |
| 2599 | O | HOH | S | 388 | 2.512 | 47.88 | 0.707 | 1.00 | 48.12 |
| 2600 | O | HOH | S | 389 | 13.153 | 54.6 | −2.131 | 1.00 | 53.44 |
| 2601 | O | HOH | S | 390 | 17.487 | 16.51 | 30.198 | 1.00 | 49.11 |
| 2602 | O | HOH | S | 391 | 20.116 | 10.78 | 23.265 | 1.00 | 55.34 |
| 2603 | O | HOH | S | 392 | 23.496 | 12.69 | 26.104 | 1.00 | 62.62 |
| 2604 | O | HOH | S | 393 | 20.396 | 47.11 | 12.135 | 1.00 | 51.92 |
| 2605 | O | HOH | S | 394 | 9.042 | 10.92 | 17.744 | 1.00 | 57.21 |
| 2606 | O | HOH | S | 395 | 15.086 | 15.2 | 30.474 | 1.00 | 56.74 |
| 2607 | O | HOH | S | 396 | 27.824 | 7.29 | −8.552 | 1.00 | 49.75 |
| 2608 | O | HOH | S | 397 | −1.167 | 31.36 | 18.711 | 1.00 | 53.14 |
| 2609 | O | HOH | S | 398 | 7.302 | 35.54 | 28.016 | 1.00 | 61.44 |
| 2610 | O | HOH | S | 399 | 14.214 | 16.7 | −13.22 | 1.00 | 42.98 |
| 2611 | O | HOH | S | 400 | 20.092 | 59.9 | −2.921 | 1.00 | 54.66 |
| 2612 | O | HOH | S | 401 | 28.927 | 1.994 | 14.98 | 1.00 | 47.04 |
| 2613 | O | HOH | S | 402 | 14.185 | 28.7 | 31.582 | 1.00 | 59.65 |
| 2614 | O | HOH | S | 403 | 20.834 | 52.48 | 14.019 | 1.00 | 61.68 |
| 2615 | O | HOH | S | 404 | 13.279 | 5.355 | 25.439 | 1.00 | 52.09 |
| 2616 | O | HOH | 5 | 405 | 26.886 | 59.65 | −2.846 | 1.00 | 46.54 |
| 2617 | O | HOH | S | 406 | 11.357 | 5.386 | 16.734 | 1.00 | 45.29 |
| 2618 | O | HOH | S | 407 | 30.634 | 57.68 | 11.14 | 1.00 | 63.95 |
| 2619 | O | HOH | S | 408 | 33.859 | 39.18 | −13.25 | 1.00 | 54.28 |
| 2620 | O | HOH | S | 409 | 16.114 | 14.41 | −7.014 | 1.00 | 46.14 |
| 2621 | O | HOH | S | 410 | 37.72 | 38.28 | 6.176 | 1.00 | 64.28 |
| 2622 | O | HOH | S | 411 | 6.555 | 42.04 | −19.44 | 1.00 | 48.56 |
| 2623 | C1A | 735 | C | 1 | 19.341 | 40.73 | 3.993 | 1.00 | 15.91 |
| 2624 | O1C | 735 | C | 1 | 18.239 | 40.2 | 4.31 | 1.00 | 17.26 |
| 2625 | O1B | 735 | C | 1 | 20.381 | 40.5 | 4.658 | 1.00 | 17.17 |
| 2626 | C1D | 735 | C | 1 | 19.457 | 41.69 | 2.755 | 1.00 | 14.73 |
| 2627 | C1X | 735 | C | 1 | 19.835 | 43.1 | 3.255 | 1.00 | 16.66 |
| 2628 | CIY | 735 | C | 1 | 18.085 | 41.87 | 1.978 | 1.00 | 16.99 |
| 2629 | O1E | 735 | C | 1 | 20.62 | 41.22 | 1.905 | 1.00 | 15.62 |
| 2630 | C1F | 735 | C | 1 | 20.412 | 40.1 | 1.046 | 1.00 | 12.70 |
| 2631 | G1G | 735 | C | 1 | 20.444 | 40.32 | −0.337 | 1.00 | 16.64 |
| 2632 | C11 | 735 | C | 1 | 20.235 | 39.25 | −1.206 | 1.00 | 15.86 |
| 2633 | C1K | 735 | C | 1 | 19.982 | 37.9 | −0.694 | 1.00 | 15.12 |
| 2634 | C1J | 735 | C | 1 | 19.956 | 37.68 | 0.701 | 1.00 | 15.25 |
| 2635 | C1H | 735 | C | 1 | 20.17 | 38.77 | 1.574 | 1.00 | 16.34 |
| 2636 | C1L | 735 | C | 1 | 19.737 | 36.71 | −1.661 | 1.00 | 16.46 |
| 2637 | N1M | 735 | C | 1 | 19.038 | 37.26 | −2.855 | 1.00 | 16.48 |
| 2638 | C2A | 735 | C | 1 | 17.688 | 37.38 | −3.024 | 1.00 | 19.45 |
| 2639 | O2A | 735 | C | 1 | 16.891 | 37 | −2.138 | 1.00 | 20.40 |
| 2640 | S2C | 735 | C | 1 | 15.434 | 38.08 | −4.377 | 1.00 | 18.50 |
| 2641 | C2B | 735 | C | 1 | 17.146 | 37.95 | −4.199 | 1.00 | 18.76 |
| 2642 | C2D | 735 | C | 1 | 17.789 | 38.48 | −5.386 | 1.00 | 19.70 |
| 2643 | C20 | 735 | C | 1 | 19.304 | 38.59 | −5.689 | 1.00 | 20.26 |
| 2644 | N2E | 735 | C | 1 | 16.929 | 38.94 | −6.305 | 1.00 | 17.68 |
| 2645 | C2F | 735 | C | 1 | 15.613 | 38.79 | −5.935 | 1.00 | 19.94 |
| 2646 | G2H | 735 | C | 1 | 14.527 | 39.19 | −6.734 | 1.00 | 21.71 |
| 2647 | C2J | 735 | C | 1 | 13.172 | 39 | −6.28 | 1.00 | 20.47 |
| 2648 | C2L | 735 | C | 1 | 12.08 | 39.39 | −7.09 | 1.00 | 22.73 |
| 2649 | C2M | 735 | C | 1 | 12.3 | 39.99 | −8.38 | 1.00 | 20.47 |
| 2650 | C2K | 735 | C | 1 | 13.648 | 40.19 | −8.827 | 1.00 | 23.90 |
| 2651 | C2I | 735 | C | 1 | 14.733 | 39.8 | −8.026 | 1.00 | 22.13 |
| 2652 | C2N | 735 | C | 1 | 11.109 | 40.43 | −9.297 | 1.00 | 25.97 |
| 2653 | F2P | 735 | C | 1 | 10.97 | 39.5 | −10.3 | 1.00 | 31.88 |
| 2654 | F2Q | 735 | C | 1 | 9.921 | 40.5 | −8.624 | 1.00 | 31.88 |
| 2655 | F2O | 735 | C | 1 | 11.341 | 41.65 | −9.859 | 1.00 | 31.88 |

TABLE 4

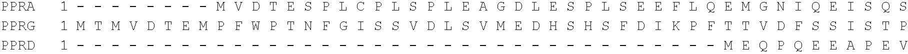

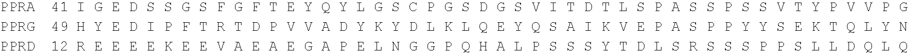

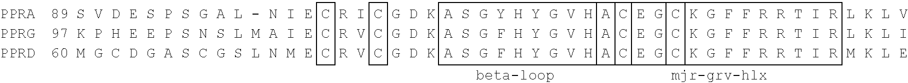

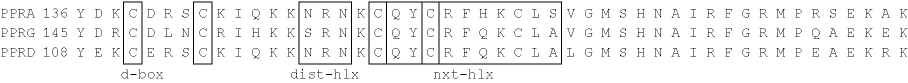

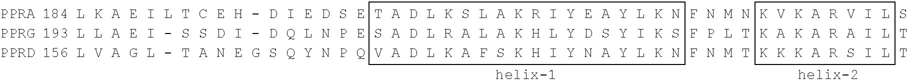

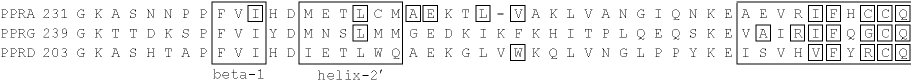

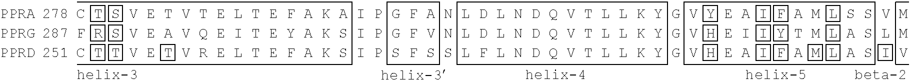

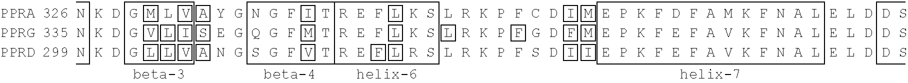

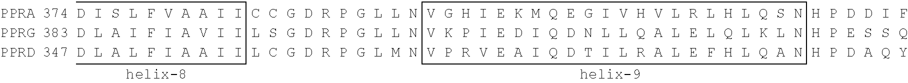

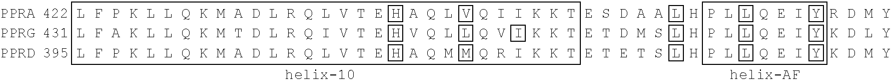

It will be understood that various details of the invention may be changed without departing from the scope of the invention. Furthermore, the foregoing description is for the purpose of illustration only, and not for the purpose of limitation—the invention being defined by the claims.

What is claimed is:

1. A substantially pure PPARα ligand binding domain polypeptide in crystalline form.

2. The polypeptide of claim 1, wherein the crystalline form has lattice constants of a=61.3 Å, b=103.5 Å, c=49.9 Å, α=90°, β=90°, γ=90°.

3. The polypeptide of claim 1 or 2, wherein the crystalline form is an orthorhombic crystalline form.

4. The polypeptide of claim 1 or 2, wherein the crystalline form has a space group of P2₁2₁2.

5. The polypeptide of claim 1 or 2, wherein the PPARα ligand binding domain polypeptide has the amino acid sequence shown in SEQ ID NO: 4.

6. The polypeptide of claim 1 or 2, wherein the PPARα ligand binding domain polypeptide is in complex with a ligand.

7. The polypeptide of claim 1 or 2, wherein the PPARα ligand binding domain has a crystalline structure further characterized by the coordinates corresponding to Table 2.

8. The polypeptide of claim 1 or 2, wherein the crystalline form contains one PPARα ligand binding domain polypeptide in the asymmetric unit.

9. The polypeptide of claim 1 or 2, wherein the crystalline form is such that the three-dimensional structure of the crystallized PPARα ligand binding domain polypeptide can be determined to a resolution of about 1.8 Åor better.

10. The polypeptide of claim 1 or 2, wherein the crystalline form contains one or more atoms having a molecular weight of 40 grams/mol or greater.

11. The polypeptide of claim 1, wherein the crystalline form has lattice constants of a=95.58 Å, b=122.06 Å, c=122.10 Å, α=90°, β=90°, γ=90°.

12. The polypeptide of claim 1 or 11, wherein the crystalline form is an orthorhombic crystalline form.

13. The polypeptide of claim 1 or 11, wherein the crystalline form has a space group of P2₁2₁2₁.

14. The polypeptide of claim 1 or 11, wherein the PPARα ligand binding domain polypeptide has the amino acid sequence shown in SEQ ID NO: 4.

15. The polypeptide of claim 1 or 11, wherein the crystalline form contains four PPARα ligand binding domain polypeptides in the asymmetric unit.

16. The polypeptide of claim 1 or 11, wherein the crystalline form is such that the three-dimensional structure of the crystallized PPARα ligand binding domain polypeptide can be determined to a resolution of about 2.5 Å or better.

17. The polypeptide of claim 1 or 11, wherein the crystalline form contains one or more atoms having a molecular weight of 40 grams/mol or greater.

18. A method for determining the three-dimensional structure of a crystallized PPARα ligand binding domain polypeptide to a resolution of about 1.8 Å or better, the method comprising:
(a) crystallizing a PPARα ligand binding domain polypeptide; and
(b) analyzing the PPARα ligand binding domain polypeptide to determine the three-dimensional structure of the crystallized PPARα ligand binding domain polypeptide, whereby the three-dimensional structure of a crystallized PPARα ligand binding domain polypeptide is determined to a resolution of about 1.8 Å or better.

19. The method of claim 18, wherein the analyzing is by X-ray diffraction.

20. The method of claim 18, wherein the crystallization is accomplished by the hanging drop vapor diffusion method, and wherein the PPARα ligand binding domain is mixed with an equal volume of reservoir.

21. The method of claim 20, wherein the reservoir comprises 4-8% PEG 3350, 100-200 mM NaF, and 12-16% 2,5 hexanediol.

22. The method of claim 20, wherein the reservoir comprises 50 mM bis-tris-propane, 4-6% PEG 3350, 150 mM NaNO₃, 16% 2,5 hexanediol and 1-3 mM YCl.

23. A method of generating a crystallized PPARα, ligand binding domain polypeptide, the method comprising:
(a) incubating a solution comprising a PPARα ligand binding domain with an equal volume of reservoir; and
(b) crystallizing the PPARα ligand binding domain polypeptide using the hanging drop method, whereby a crystallized PPARα ligand binding domain polypeptide is generated.

24. A crystallized PPARα ligand binding domain polypeptide produced by the method of claim 23.

25. A method of designing a modulator of a PPAR polypeptide, the method comprising:
(a) designing a potential modulator of a PPAR polypeptide that will make interactions with amino acids in the ligand binding site based upon a crystalline structure of a PPARα ligand binding domain polypeptide;
(b) synthesizing the modulator; and
(c) determining whether the potential modulator modulates the activity of the PPAR polypeptide, whereby a modulator of a PPAR polypeptide is designed.

26. A method of designing a modulator that selectively modulates the activity of a PPAR polypeptide the method comprising:
(a) obtaining a crystalline form of a PPARα ligand binding domain polypeptide;
(b) evaluating the three-dimensional structure of the crystallized PPARα ligand binding domain polypeptide; and
(c) synthesizing a potential modulator based on the three-dimensional crystal structure of the crystallized PPARα ligand binding domain polypeptide, whereby a modulator that selectively modulates the activity of a PPARα polypeptide is designed.

27. The method of claim 26, wherein the method further comprises contacting a PPARα ligand binding domain polypeptide with the potential modulator; and assaying the PPARα ligand binding domain polypeptide for binding of the potential modulator, for a change in activity of the PPARα ligand binding domain polypeptide, or both.

28. The method of claim 26, wherein the crystalline form is in orthorhombic form.

29. The method of claim 28, wherein the crystalline form is such that the three-dimensional structure of the crystallized PPARα ligand binding domain polypeptide can be determined to a resolution of about 1.8 Å or better.

30. A method of screening a plurality of compounds for a modulator of a PPAR ligand binding domain polypeptide, the method comprising:
(a) providing a library of test samples;
(b) contacting a crystalline PPARα ligand binding domain polypeptide with each test sample;
(c) detecting an interaction between a test sample and the crystalline PPARα ligand binding domain polypeptide;
(d) identifying a test sample that interacts with the crystalline PPARα ligand binding domain polypeptide; and
(e) isolating a test sample that interacts with the crystalline PPARαligand binding domain polypeptide, whereby a plurality of compounds is screened for a modulator of a PPAR ligand binding domain polypeptide.

31. The method of claim 30, wherein the test samples are bound to a substrate.

32. The method of claim 30, wherein the test samples are synthesized directly on a substrate.

33. A method for identifying a PPAR modulator, the method comprising:
(a) providing atomic coordinates of a PPARα ligand binding domain to a computerized modeling system; and
(b) modeling ligands that fit spatially into the binding pocket of the PPARα ligand binding domain to thereby identify a PPAR modulator, whereby a PPAR modulator is identified.

34. The method of claim 33, wherein the method further comprises identifying in an assay for PPAR-mediated activity a modeled ligand which increases or decreases the activity of the PPAR.

35. A method of identifying a PPARα modulator that selectively modulates the activity of a PPARα polypeptide compared to other polypeptides, the method comprising:
(a) providing atomic coordinates of a PPARα ligand binding domain to a computerized modeling system; and
(b) modeling a ligand that fits into the binding pocket of a PPARα ligand binding domain and that interacts with conformationally constrained residues of a PPARα conserved among PPAR subtypes, whereby a PPARα modulator that selectively modulates the activity of a PPARα polypeptide compared to other polypeptides.

36. The method of claim 35, wherein the method further comprises identifying in a biological assay for PPARα activity a modeled ligand that selectively binds to said PPARα and increases or decreases the activity of said PPARα.

37. A method of designing a modulator of a PPAR polypeptide, the method comprising:
(a) selecting a candidate PPAR ligand;
(b) determining which amino acid or amino acids of a PPAR polypeptide interact with the ligand using a three-dimensional model of a crystallized protein comprising a PPARα LBD;
(c) identifying in a biological assay for PPAR activity a degree to which the ligand modulates the activity of the PPAR polypeptide;
(d) selecting a chemical modification of the ligand wherein the interaction between the amino acids of the PPAR polypeptide and the ligand is predicted to be modulated by the chemical. modification;
(e) performing the chemical modification on the ligand to form a modified ligand;
(f) contacting the modified ligand with the PPAR polypeptide;
(g) identifying in a biological assay for PPAR activity a degree to which the modified ligand modulates the biological activity of the PPAR polypeptide; and
(h) comparing the biological activity of the PPAR polypeptide in the presence of modified ligand with the biological activity of the PPAR polypeptide in the presence of the unmodified ligand, whereby a modulator of a PPAR polypeptide is designed.

38. The method of claim 37, wherein the PPAR polypeptide is a PPARα polypeptide.

39. The method of claim 37, wherein the three-dimensional model of a crystallized protein is a PPARα LBD polypeptide with a bound ligand.

40. The method of claim 37, wherein the method further comprises repeating steps (a) through (f), if the biological activity of the PPAR polypeptide in the presence of the modified ligand varies from the biological activity of the PPAR polypeptide in the presence of the unmodified ligand.

41. An assay method for identifying a compound that inhibits binding of a ligand to a PPAR polypeptide, the assay method comprising:
(a) incubating a PPAR polypeptide with a ligand in the presence of a test inhibitor compound;
(b) determining an amount of ligand that is bound to the PPAR polypeptide, wherein decreased binding of ligand to the PPAR protein in the presence of the test inhibitor compound relative to binding of ligand in the absence of the test inhibitor compound is indicative of inhibition; and
(c) identifying the test compound as an inhibitor of ligand binding if decreased ligand binding is observed, whereby a compound that inhibits binding of a ligand to a PPAR polypeptide is identified.

42. A method of identifying a PPAR modulator that selectively modulates the biological activity of one PPAR subtype compared to PPARα, the method comprising:
(a) providing an atomic structure coordinate set describing a PPARα ligand binding domain structure and at least one other atomic structure coordinate set describing a PPAR ligand binding domain, each ligand binding domain comprising a ligand binding site;
(b) comparing the PPAR atomic structure coordinate sets to identify at least one difference between the sets;
(c) designing a candidate ligand predicted to interact with the difference of step (b);
(d) synthesizing the candidate ligand; and
(e) testing the synthesized candidate ligand for an ability to selectively modulate a PPAR subtype as compared to PPARα, whereby a PPAR modulator that selectively modulates the biological activity of one PPAR subtype compared to PPARα is identified.
